SEQUENCE LISTING (167 sequences)

SEQ ID NO: 1 - protein, 7 aa, synthetic construct
TASNIKS

SEQ ID NO: 2 - protein, 7 aa, synthetic construct
YLCDNKF

SEQ ID NO: 3 - protein, 7 aa, synthetic construct
KSSNIKS

SEQ ID NO: 4 - protein, 7 aa, synthetic construct
YICDNKF

SEQ ID NO: 5 - protein, 7 aa, synthetic construct
TAVNIKS

SEQ ID NO: 6 - protein, 7 aa, synthetic construct
KAANIKS

SEQ ID NO: 7 - protein, 7 aa, synthetic construct
KASNIKS

SEQ ID NO: 8 - protein, 7 aa, synthetic construct
YYCGERF

SEQ ID NO: 9 - protein, 7 aa, synthetic construct
TAESIKS

SEQ ID NO: 10 - protein, 7 aa, synthetic construct
YFCDPQF

SEQ ID NO: 11 - protein, 7 aa, synthetic construct
EAASIKD

SEQ ID NO: 12 - protein, 7 aa, synthetic construct
KATNIKD

SEQ ID NO: 13 - protein, 7 aa, synthetic construct
YFCDSKF

SEQ ID NO: 14 - protein, 7 aa, synthetic construct
FDFDAES

SEQ ID NO: 15 - protein, 7 aa, synthetic construct
TLIKPQF

SEQ ID NO: 16 - protein, 7 aa, synthetic construct
TGDKIKS

SEQ ID NO: 17 - protein, 7 aa, synthetic construct
TIIKNDF

SEQ ID NO: 18 - protein, 7 aa, synthetic construct
EAASIQD

SEQ ID NO: 19 - protein, 7 aa, synthetic construct
FFCDNYF

SEQ ID NO: 20 - protein, 7 aa, synthetic construct
FVIVNKF

SEQ ID NO: 21 - protein, 7 aa, synthetic construct
AAQNIKS

SEQ ID NO: 22 - protein, 7 aa, synthetic construct
YFCGGKF

SEQ ID NO: 23 - protein, 7 aa, synthetic construct
QANSIKD

SEQ ID NO: 24 - protein, 7 aa, synthetic construct
YRCDSKF

SEQ ID NO: 25 - protein, 7 aa, synthetic construct
GAASIKN

SEQ ID NO: 26 - protein, 7 aa, synthetic construct
YSCNTIF

SEQ ID NO: 27 - protein, 7 aa, synthetic construct
SAQNIKS

SEQ ID NO: 28 - protein, 7 aa, synthetic construct
YYCDNRF

SEQ ID NO: 29 - protein, 7 aa, synthetic construct
SAGNIKS

SEQ ID NO: 30 - protein, 7 aa, synthetic construct
YFCDNSF

SEQ ID NO: 31 - protein, 7 aa, synthetic construct
TAQNIKS

SEQ ID NO: 32 - protein, 7 aa, synthetic construct
SAQSIKS

SEQ ID NO: 33 - protein, 7 aa, synthetic construct
AANNIKS

SEQ ID NO: 34 - protein, 7 aa, synthetic construct
YNCSGKF

SEQ ID NO: 35 - protein, 7 aa, synthetic construct
QAQNIKS

SEQ ID NO: 36 - protein, 7 aa, synthetic construct
QADCIKS

SEQ ID NO: 37 - protein, 7 aa, synthetic construct
YSCDGVF

SEQ ID NO: 38 - protein, 7 aa, synthetic construct
RAQNIKS

SEQ ID NO: 39 - protein, 7 aa, synthetic construct
FLCENTF

SEQ ID NO: 40 - protein, 437 aa, synthetic construct
MDNEVEKNIEIWKVKKLVQSLEKARGNGTSMISLVIPPKGQIPLYQKMLTDEYGTASNIKSRVNRLSVLSAITSTQQKLKLYNTLPKNGLVLYCGDIITEDGKEKKVTFDIEPYKPINTSLYLCDNKFHTEVLSELLQADDKFGFIVMDGQGTLFGSVSGNTRTVLHKFTVDLPKKHGRGGQSALRFARLREEKRHNYVRKVAEVAVQNFITNDKVNVKGLILAGSADFKTDLAKSELFDPRLACKVISIVDVSYGGENGFNQAIELSAEALANVKYVQEKKLLEAYFDEISQDTGKFCYGIDDTLKALDLGAVEKLIVFENLETIRYTFKDAEDNEVIKFAEPEAKDKSFAIDKATGQEMDVVSEEPLIEWLAANYKNFGATLEFITDKSSEGAQFVTGFGGIGAMLRYKVNFEQLVDESEDEYYDEDEGSDYDFI

SEQ ID NO: 41 - protein, 437 aa, synthetic construct
MDNEVEKNIEIWKVKKLVQSLEKARGNGTSMISLVIPPKGQIPLYQKMLTDEYGKSSNIKSRVNRLSVLSAITSTQQKLKLYNTLPKNGLVLYCGDIITEDGKEKKVTFDIEPYKPINTSLYICDNKFHTEVLSELLQADDKFGFIVMDGQGTLFGSVSGNTRTVLHKFTVDLPKKHGRGGQSALRFARLREEKRHNYVRKVAEVAVQNFITNDKVNVKGLILAGSADFKTDLAKSELFDPRLACKVISIVDVSYGGENGFNQAIELSAEALANVKYVQEKKLLEAYFDEISQDTGKFCYGIDDTLKALDLGAVEKLIVFENLETIRYTFKDAEDNEVIKFAEPEAKDKSFAIDKATGQEMDVVSEEPLIEWLAANYKNFGATLEFITDKSSEGAQFVTGFGGIGAMLRYKVNFEQLVDESEDEYYDEDEGSDYDFI

SEQ ID NO: 42 - protein, 437 aa, synthetic construct
MDNEVEKNIEIWKVKKLVQSLEKARGNGTSMISLVIPPKGQIPLYQKMLTDEYGTAVNIKSRVNRLSVLSAITSTQQKLKLYNTLPKNGLVLYCGDIITEDGKEKKVTFDIEPYKPINTSLYICDNKFHTEVLSELLQADDKFGFIVMDGQGTLFGSVSGNTRTVLHKFTVDLPKKHGRGGQSALRFARLREEKRHNYVRKVAEVAVQNFITNDKVNVKGLILAGSADFKTDLAKSELFDPRLACKVISIVDVSYGGENGFNQAIELSAEALANVKYVQEKKLLEAYFDEISQDTGKFCYGIDDTLKALDLGAVEKLIVFENLETIRYTFKDAEDNEVIKFAEPEAKDKSFAIDKATGQEMDVVSEEPLIEWLAANYKNFGATLEFITDKSSEGAQFVTGFGGIGAMLRYKVNFEQLVDESEDEYYDEDEGSDYDFI

SEQ ID NO: 43 - protein, 437 aa, synthetic construct
MDNEVEKNIEIWKVKKLVQSLEKARGNGTSMISLVIPPKGQIPLYQKMLTDEYGKAANIKSRVNRLSVLSAITSTQQKLKLYNTLPKNGLVLYCGDIITEDGKEKKVTFDIEPYKPINTSLYLCDNKFHTEVLSELLQADDKFGFIVMDGQGTLFGSVSGNTRTVLHKFTVDLPKKHGRGGQSALRFARLREEKRHNYVRKVAEVAVQNFITNDKVNVKGLILAGSADFKTDLAKSELFDPRLACKVISIVDVSYGGENGFNQAIELSAEALANVKYVQEKKLLEAYFDEISQDTGKFCYGIDDTLKALDLGAVEKLIVFENLETIRYTFKDAEDNEVIKFAEPEAKDKSFAIDKATGQEMDVVSEEPLIEWLAANYKNFGATLEFITDKSSEGAQFVTGFGGIGAMLRYKVNFEQLVDESEDEYYDEDEGSDYDFI

SEQ ID NO: 44 - protein, 437 aa, synthetic construct
MDNEVEKNIEIWKVKKLVQSLEKARGNGTSMISLVIPPKGQIPLYQKMLTDEYGKASNIKSRVNRLSVLSAITSTQQKLKLYNTLPKNGLVLYCGDIITEDGKEKKVTFDIEPYKPINTSLYYCGERFHTEVLSELLQADDKFGFIVMDGQGTLFGSVSGNTRTVLHKFTVDLPKKHGRGGQSALRFARLREEKRHNYVRKVAEVAVQNFITNDKVNVKGLILAGSADFKTDLAKSELFDPRLACKVISIVDVSYGGENGFNQAIELSAEALANVKYVQEKKLLEAYFDEISQDTGKFCYGIDDTLKALDLGAVEKLIVFENLETIRYTFKDAEDNEVIKFAEPEAKDKSFAIDKATGQEMDVVSEEPLIEWLAANYKNFGATLEFITDKSSEGAQFVTGFGGIGAMLRYKVNFEQLVDESEDEYYDEDEGSDYDFI

SEQ ID NO: 45 - protein, 437 aa, synthetic construct
MDNEVEKNIEIWKVKKLVQSLEKARGNGTSMISLVIPPKGQIPLYQKMLTDEYGTAESIKSRVNRLSVLSAITSTQQKLKLYNTLPKNGLVLYCGDIITEDGKEKKVTFDIEPYKPINTSLYICDNKFHTEVLSELLQADDKFGFIVMDGQGTLFGSVSGNTRTVLHKFTVDLPKKHGRGGQSALRFARLREEKRHNYVRKVAEVAVQNFITNDKVNVKGLILAGSADFKTDLAKSELFDPRLACKVISIVDVSYGGENGFNQAIELSAEALANVKYVQEKKLLEAYFDEISQDTGKFCYGIDDTLKALDLGAVEKLIVFENLETIRYTFKDAEDNEVIKFAEPEAKDKSFAIDKATGQEMDVVSEEPLIEWLAANYKNFGATLEFITDKSSEGAQFVTGFGGIGAMLRYKVNFEQLVDESEDEYYDEDEGSDYDFI

SEQ ID NO: 46 - protein, 437 aa, synthetic construct
MDNEVEKNIEIWKVKKLVQSLEKARGNGTSMISLVIPPKGQIPLYQKMLTDEYGTASNIKSRVNRLSVLSAITSTQQKLKLYNTLPKNGLVLYCGDIITEDGKEKKVTFDIEPYKPINTSLYFCDPQFHTEVLSELLQADDKFGFIVMDGQGTLFGSVSGNTRTVLHKFTVDLPKKHGRGGQSALRFARLREEKRHNYVRKVAEVAVQNFITNDKVNVKGLILAGSADFKTDLAKSELFDPRLACKVISIVDVSYGGENGFNQAIELSAEALANVKYVQEKKLLEAYFDEISQDTGKFCYGIDDTLKALDLGAVEKLIVFENLETIRYTFKDAEDNEVIKFAEPEAKDKSFAIDKATGQEMDVVSEEPLIEWLAANYKNFGATLEFITDKSSEGAQFVTGFGGIGAMLRYKVNFEQLVDESEDEYYDEDEGSDYDFI

SEQ ID NO: 47 - protein, 437 aa, synthetic construct
MDNEVEKNIEIWKVKKLVQSLEKARGNGTSMISLVIPPKGQIPLYQKMLTDEYGEAASIKDRVNRLSVLSAITSTQQKLKLYNTLPKNGLVLYCGDIITEDGKEKKVTFDIEPYKPINTSLYFCDPQFHTEVLSELLQADDKFGFIVMDGQGTLFGSVSGNTRTVLHKFTVDLPKKHGRGGQSALRFARLREEKRHNYVRKVAEVAVQNFITNDKVNVKGLILAGSADFKTDLAKSELFDPRLACKVISIVDVSYGGENGFNQAIELSAEALANVKYVQEKKLLEAYFDEISQDTGKFCYGIDDTLKALDLGAVEKLIVFENLETIRYTFKDAEDNEVIKFAEPEAKDKSFAIDKATGQEMDVVSEEPLIEWLAANYKNFGATLEFITDKSSEGAQFVTGFGGIGAMLRYKVNFEQLVDESEDEYYDEDEGSDYDFI

SEQ ID NO: 48 - protein, 437 aa, synthetic construct
MDNEVEKNIEIWKVKKLVQSLEKARGNGTSMISLVIPPKGQIPLYQKMLTDEYGKATNIKDRVNRLSVLSAITSTQQKLKLYNTLPKNGLVLYCGDIITEDGKEKKVTFDIEPYKPINTSLYFCDSKFHTEVLSELLQADDKFGFIVMDGQGTLFGSVSGNTRTVLHKFTVDLPKKHGRGGQSALRFARLREEKRHNYVRKVAEVAVQNFITNDKVNVKGLILAGSADFKTDLAKSELFDPRLACKVISIVDVSYGGENGFNQAIELSAEALANVKYVQEKKLLEAYFDEISQDTGKFCYGIDDTLKALDLGAVEKLIVFENLETIRYTFKDAEDNEVIKFAEPEAKDKSFAIDKATGQEMDVVSEEPLIEWLAANYKNFGATLEFITDKSSEGAQFVTGFGGIGAMLRYKVNFEQLVDESEDEYYDEDEGSDYDFI

SEQ ID NO: 49 - protein, 437 aa, synthetic construct
MDNEVEKNIEIWKVKKLVQSLEKARGNGTSMISLVIPPKGQIPLYQKMLTDEYGFDFDAESRVNRLSVLSAITSTQQKLKLYNTLPKNGLVLYCGDIITEDGKEKKVTFDIEPYKPINTSLTLIKPQFHTEVLSELLQADDKFGFIVMDGQGTLFGSVSGNTRTVLHKFTVDLPKKHGRGGQSALRFARLREEKRHNYVRKVAEVAVQNFITNDKVNVKGLILAGSADFKTDLAKSELFDPRLACKVISIVDVSYGGENGFNQAIELSAEALANVKYVQEKKLLEAYFDEISQDTGKFCYGIDDTLKALDLGAVEKLIVFENLETIRYTFKDAEDNEVIKFAEPEAKDKSFAIDKATGQEMDVVSEEPLIEWLAANYKNFGATLEFITDKSSEGAQFVTGFGGIGAMLRYKVNFEQLVDESEDEYYDEDEGSDYDFI

SEQ ID NO: 50 - protein, 437 aa, synthetic construct
MDNEVEKNIEIWKVKKLVQSLEKARGNGTSMISLVIPPKGQIPLYQKMLTDEYGTGDKIKSRVNRLSVLSAITSTQQKLKLYNTLPKNGLVLYCGDIITEDGKEKKVTFDIEPYKPINTSLTIIKNDFHTEVLSELLQADDKFGFIVMDGQGTLFGSVSGNTRTVLHKFTVDLPKKHGRGGQSALRFARLREEKRHNYVRKVAEVAVQNFITNDKVNVKGLILAGSADFKTDLAKSELFDPRLACKVISIVDVSYGGENGFNQAIELSAEALANVKYVQEKKLLEAYFDEISQDTGKFCYGIDDTLKALDLGAVEKLIVFENLETIRYTFKDAEDNEVIKFAEPEAKDKSFAIDKATGQEMDVVSEEPLIEWLAANYKNFGATLEFITDKSSEGAQFVTGFGGIGAMLRYKVNFEQLVDESEDEYYDEDEGSDYDFI

SEQ ID NO: 51 - protein, 437 aa, synthetic construct
MDNEVEKNIEIWKVKKLVQSLEKARGNGTSMISLVIPPKGQIPLYQKMLTDEYGEAASIQDRVNRLSVLSAITSTQQKLKLYNTLPKNGLVLYCGDIITEDGKEKKVTFDIEPYKPINTSLFFCDNYFHTEVLSELLQADDKFGFIVMDGQGTLFGSVSGNTRTVLHKFTVDLPKKHGRGGQSALRFARLREEKRHNYVRKVAEVAVQNFITNDKVNVKGLILAGSADFKTDLAKSELFDPRLACKVISIVDVSYGGENGFNQAIELSAEALANVKYVQEKKLLEAYFDEISQDTGKFCYGIDDTLKALDLGAVEKLIVFENLETIRYTFKDAEDNEVIKFAEPEAKDKSFAIDKATGQEMDVVSEEPLIEWLAANYKNFGATLEFITDKSSEGAQFVTGFGGIGAMLRYKVNFEQLVDESEDEYYDEDEGSDYDFI

SEQ ID NO: 52 - protein, 437 aa, synthetic construct
MDNEVEKNIEIWKVKKLVQSLEKARGNGTSMISLVIPPKGQIPLYQKMLTDEYGKATNIKDRVNRLSVLSAITSTQQKLKLYNTLPKNGLVLYCGDIITEDGKEKKVTFDIEPYKPINTSLFVIVNKFHTEVLSELLQADDKFGFIVMDGQGTLFGSVSGNTRTVLHKFTVDLPKKHGRGGQSALRFARLREEKRHNYVRKVAEVAVQNFITNDKVNVKGLILAGSADFKTDLAKSELFDPRLACKVISIVDVSYGGENGFNQAIELSAEALANVKYVQEKKLLEAYFDEISQDTGKFCYGIDDTLKALDLGAVEKLIVFENLETIRYTFKDAEDNEVIKFAEPEAKDKSFAIDKATGQEMDVVSEEPLIEWLAANYKNFGATLEFITDKSSEGAQFVTGFGGIGAMLRYKVNFEQLVDESEDEYYDEDEGSDYDFI

SEQ ID NO: 53 - protein, 437 aa, synthetic construct
MDNEVEKNIEIWKVKKLVQSLEKARGNGTSMISLVIPPKGQIPLYQKMLTDEYGAAQNIKSRVNRLSVLSAITSTQQKLKLYNTLPKNGLVLYCGDIITEDGKEKKVTFDIEPYKPINTSLYFCGGKFHTEVLSELLQADDKFGFIVMDGQGTLFGSVSGNTRTVLHKFTVDLPKKHGRGGQSALRFARLREEKRHNYVRKVAEVAVQNFITNDKVNVKGLILAGSADFKTDLAKSELFDPRLACKVISIVDVSYGGENGFNQAIELSAEALANVKYVQEKKLLEAYFDEISQDTGKFCYGIDDTLKALDLGAVEKLIVFENLETIRYTFKDAEDNEVIKFAEPEAKDKSFAIDKATGQEMDVVSEEPLIEWLAANYKNFGATLEFITDKSSEGAQFVTGFGGIGAMLRYKVNFEQLVDESEDEYYDEDEGSDYDFI

SEQ ID NO: 54 - protein, 437 aa, synthetic construct
MDNEVEKNIEIWKVKKLVQSLEKARGNGTSMISLVIPPKGQIPLYQKMLTDEYGQANSIKDRVNRLSVLSAITSTQQKLKLYNTLPKNGLVLYCGDIITEDGKEKKVTFDIEPYKPINTSLYRCDSKFHTEVLSELLQADDKFGFIVMDGQGTLFGSVSGNTRTVLHKFTVDLPKKHGRGGQSALRFARLREEKRHNYVRKVAEVAVQNFITNDKVNVKGLILAGSADFKTDLAKSELFDPRLACKVISIVDVSYGGENGFNQAIELSAEALANVKYVQEKKLLEAYFDEISQDTGKFCYGIDDTLKALDLGAVEKLIVFENLETIRYTFKDAEDNEVIKFAEPEAKDKSFAIDKATGQEMDVVSEEPLIEWLAANYKNFGATLEFITDKSSEGAQFVTGFGGIGAMLRYKVNFEQLVDESEDEYYDEDEGSDYDFI

SEQ ID NO: 55 - protein, 437 aa, synthetic construct
MDNEVEKNIEIWKVKKLVQSLEKARGNGTSMISLVIPPKGQIPLYQKMLTDEYGGAASIKNRVNRLSVLSAITSTQQKLKLYNTLPKNGLVLYCGDIITEDGKEKKVTFDIEPYKPINTSLYSCNTIFHTEVLSELLQADDKFGFIVMDGQGTLFGSVSGNTRTVLHKFTVDLPKKHGRGGQSALRFARLREEKRHNYVRKVAEVAVQNFITNDKVNVKGLILAGSADFKTDLAKSELFDPRLACKVISIVDVSYGGENGFNQAIELSAEALANVKYVQEKKLLEAYFDEISQDTGKFCYGIDDTLKALDLGAVEKLIVFENLETIRYTFKDAEDNEVIKFAEPEAKDKSFAIDKATGQEMDVVSEEPLIEWLAANYKNFGATLEFITDKSSEGAQFVTGFGGIGAMLRYKVNFEQLVDESEDEYYDEDEGSDYDFI

SEQ ID NO: 56 - protein, 437 aa, synthetic construct
MDNEVEKNIEIWKVKKLVQSLEKARGNGTSMISLVIPPKGQIPLYQKMLTDEYGSAQNIKSRVNRLSVLSAITSTQQKLKLYNTLPKNGLVLYCGDIITEDGKEKKVTFDIEPYKPINTSLYYCDNRFHTEVLSELLQADDKFGFIVMDGQGTLFGSVSGNTRTVLHKFTVDLPKKHGRGGQSALRFARLREEKRHNYVRKVAEVAVQNFITNDKVNVKGLILAGSADFKTDLAKSELFDPRLACKVISIVDVSYGGENGFNQAIELSAEALANVKYVQEKKLLEAYFDEISQDTGKFCYGIDDTLKALDLGAVEKLIVFENLETIRYTFKDAEDNEVIKFAEPEAKDKSFAIDKATGQEMDVVSEEPLIEWLAANYKNFGATLEFITDKSSEGAQFVTGFGGIGAMLRYKVNFEQLVDESEDEYYDEDEGSDYDFI

SEQ ID NO: 57 - protein, 437 aa, synthetic construct
MDNEVEKNIEIWKVKKLVQSLEKARGNGTSMISLVIPPKGQIPLYQKMLTDEYGSAGNIKSRVNRLSVLSAITSTQQKLKLYNTLPKNGLVLYCGDIITEDGKEKKVTFDIEPYKPINTSLYFCDNSFHTEVLSELLQADDKFGFIVMDGQGTLFGSVSGNTRTVLHKFTVDLPKKHGRGGQSALRFARLREEKRHNYVRKVAEVAVQNFITNDKVNVKGLILAGSADFKTDLAKSELFDPRLACKVISIVDVSYGGENGFNQAIELSAEALANVKYVQEKKLLEAYFDEISQDTGKFCYGIDDTLKALDLGAVEKLIVFENLETIRYTFKDAEDNEVIKFAEPEAKDKSFAIDKATGQEMDVVSEEPLIEWLAANYKNFGATLEFITDKSSEGAQFVTGFGGIGAMLRYKVNFEQLVDESEDEYYDEDEGSDYDFI

SEQ ID NO: 58 - protein, 437 aa, synthetic construct
MDNEVEKNIEIWKVKKLVQSLEKARGNGTSMISLVIPPKGQIPLYQKMLTDEYGTAQNIKSRVNRLSVLSAITSTQQKLKLYNTLPKNGLVLYCGDIITEDGKEKKVTFDIEPYKPINTSLYFCGGKFHTEVLSELLQADDKFGFIVMDGQGTLFGSVSGNTRTVLHKFTVDLPKKHGRGGQSALRFARLREEKRHNYVRKVAEVAVQNFITNDKVNVKGLILAGSADFKTDLAKSELFDPRLACKVISIVDVSYGGENGFNQAIELSAEALANVKYVQEKKLLEAYFDEISQDTGKFCYGIDDTLKALDLGAVEKLIVFENLETIRYTFKDAEDNEVIKFAEPEAKDKSFAIDKATGQEMDVVSEEPLIEWLAANYKNFGATLEFITDKSSEGAQFVTGFGGIGAMLRYKVNFEQLVDESEDEYYDEDEGSDYDFI

SEQ ID NO: 59 - protein, 437 aa, synthetic construct
MDNEVEKNIEIWKVKKLVQSLEKARGNGTSMISLVIPPKGQIPLYQKMLTDEYGSAQSIKSRVNRLSVLSAITSTQQKLKLYNTLPKNGLVLYCGDIITEDGKEKKVTFDIEPYKPINTSLYFCDNSFHTEVLSELLQADDKFGFIVMDGQGTLFGSVSGNTRTVLHKFTVDLPKKHGRGGQSALRFARLREEKRHNYVRKVAEVAVQNFITNDKVNVKGLILAGSADFKTDLAKSELFDPRLACKVISIVDVSYGGENGFNQAIELSAEALANVKYVQEKKLLEAYFDEISQDTGKFCYGIDDTLKALDLGAVEKLIVFENLETIRYTFKDAEDNEVIKFAEPEAKDKSFAIDKATGQEMDVVSEEPLIEWLAANYKNFGATLEFITDKSSEGAQFVTGFGGIGAMLRYKVNFEQLVDESEDEYYDEDEGSDYDFI

SEQ ID NO: 60 - protein, 437 aa, synthetic construct
MDNEVEKNIEIWKVKKLVQSLEKARGNGTSMISLVIPPKGQIPLYQKMLTDEYGAANNIKSRVNRLSVLSAITSTQQKLKLYNTLPKNGLVLYCGDIITEDGKEKKVTFDIEPYKPINTSLYFCGGKFHTEVLSELLQADDKFGFIVMDGQGTLFGSVSGNTRTVLHKFTVDLPKKHGRGGQSALRFARLREEKRHNYVRKVAEVAVQNFITNDKVNVKGLILAGSADFKTDLAKSELFDPRLACKVISIVDVSYGGENGFNQAIELSAEALANVKYVQEKKLLEAYFDEISQDTGKFCYGIDDTLKALDLGAVEKLIVFENLETIRYTFKDAEDNEVIKFAEPEAKDKSFAIDKATGQEMDVVSEEPLIEWLAANYKNFGATLEFITDKSSEGAQFVTGFGGIGAMLRYKVNFEQLVDESEDEYYDEDEGSDYDFI

SEQ ID NO: 61 - protein, 437 aa, synthetic construct
MDNEVEKNIEIWKVKKLVQSLEKARGNGTSMISLVIPPKGQIPLYQKMLTDEYGTASNIKSRVNRLSVLSAITSTQQKLKLYNTLPKNGLVLYCGDIITEDGKEKKVTFDIEPYKPINTSLYNCSGKFHTEVLSELLQADDKFGFIVMDGQGTLFGSVSGNTRTVLHKFTVDLPKKHGRGGQSALRFARLREEKRHNYVRKVAEVAVQNFITNDKVNVKGLILAGSADFKTDLAKSELFDPRLACKVISIVDVSYGGENGFNQAIELSAEALANVKYVQEKKLLEAYFDEISQDTGKFCYGIDDTLKALDLGAVEKLIVFENLETIRYTFKDAEDNEVIKFAEPEAKDKSFAIDKATGQEMDVVSEEPLIEWLAANYKNFGATLEFITDKSSEGAQFVTGFGGIGAMLRYKVNFEQLVDESEDEYYDEDEGSDYDFI

SEQ ID NO: 62 - protein, 437 aa, synthetic construct
MDNEVEKNIEIWKVKKLVQSLEKARGNGTSMISLVIPPKGQIPLYQKMLTDEYGQAQNIKSRVNRLSVLSAITSTQQKLKLYNTLPKNGLVLYCGDIITEDGKEKKVTFDIEPYKPINTSLYFCGGKFHTEVLSELLQADDKFGFIVMDGQGTLFGSVSGNTRTVLHKFTVDLPKKHGRGGQSALRFARLREEKRHNYVRKVAEVAVQNFITNDKVNVKGLILAGSADFKTDLAKSELFDPRLACKVISIVDVSYGGENGFNQAIELSAEALANVKYVQEKKLLEAYFDEISQDTGKFCYGIDDTLKALDLGAVEKLIVFENLETIRYTFKDAEDNEVIKFAEPEAKDKSFAIDKATGQEMDVVSEEPLIEWLAANYKNFGATLEFITDKSSEGAQFVTGFGGIGAMLRYKVNFEQLVDESEDEYYDEDEGSDYDFI

SEQ ID NO: 63 - protein, 437 aa, synthetic construct
MDNEVEKNIEIWKVKKLVQSLEKARGNGTSMISLVIPPKGQIPLYQKMLTDEYGQADCIKSRVNRLSVLSAITSTQQKLKLYNTLPKNGLVLYCGDIITEDGKEKKVTFDIEPYKPINTSLYSCDGVFHTEVLSELLQADDKFGFIVMDGQGTLFGSVSGNTRTVLHKFTVDLPKKHGRGGQSALRFARLREEKRHNYVRKVAEVAVQNFITNDKVNVKGLILAGSADFKTDLAKSELFDPRLACKVISIVDVSYGGENGFNQAIELSAEALANVKYVQEKKLLEAYFDEISQDTGKFCYGIDDTLKALDLGAVEKLIVFENLETIRYTFKDAEDNEVIKFAEPEAKDKSFAIDKATGQEMDVVSEEPLIEWLAANYKNFGATLEFITDKSSEGAQFVTGFGGIGAMLRYKVNFEQLVDESEDEYYDEDEGSDYDFI

SEQ ID NO: 64 - protein, 437 aa, synthetic construct
MDNEVEKNIEIWKVKKLVQSLEKARGNGTSMISLVIPPKGQIPLYQKMLTDEYGRAQNIKSRVNRLSVLSAITSTQQKLKLYNTLPKNGLVLYCGDIITEDGKEKKVTFDIEPYKPINTSLFLCENTFHTEVLSELLQADDKFGFIVMDGQGTLFGSVSGNTRTVLHKFTVDLPKKHGRGGQSALRFARLREEKRHNYVRKVAEVAVQNFITNDKVNVKGLILAGSADFKTDLAKSELFDPRLACKVISIVDVSYGGENGFNQAIELSAEALANVKYVQEKKLLEAYFDEISQDTGKFCYGIDDTLKALDLGAVEKLIVFENLETIRYTFKDAEDNEVIKFAEPEAKDKSFAIDKATGQEMDVVSEEPLIEWLAANYKNFGATLEFITDKSSEGAQFVTGFGGIGAMLRYKVNFEQLVDESEDEYYDEDEGSDYDFI

SEQ ID NO: 65 - protein, 446 aa, synthetic construct
MSIIDSNVETWKIKRIIKNLERLRGNGTSMISLLLSPRDAIPKVQGMLAGEYGTAESIKSKINRLAVQGAITSAKERLKLYNRTPPNGLVIYCGIVIGEDKSEKKYCIDFEPFRPLNTFKYICDNKFYTKPLFELLENDDVFGFVIVDGSGCLFGTLQGNTKKIIQNITVSLPKKHGRGGQSAPRFGRIREEKRHNYVRKVAEFATQHFITEDKPNVKGIILAGSANFKNDLSESDLFDKRLSEIVLKIVDVSYGGENGFSQAITLAEDTLSNVKFVEEKNLISKYFEEIAQDTGMVVFGIEDTLNSLELGAVGTIICFENLEINRYEIRNPSTEEIKVIHLCKDQQNDTRYKMIDNNYSYFIDQNTGLDLEILSCVPLTEWLCENYSKYGVRLEFITDKSQEGFQFVNGFGGIGGFLRFKLEIENIDYEGEDVGGEEFDADEDFI

SEQ ID NO: 66 - protein, 437 aa, synthetic construct
MAKLDDNVETWRIKRLIKNLEKLRGDGTSMISLLLSPRDQISKVQAMLAGEAGTAVNIKSRVNRQAVLSAITSAKERLKLYSKTPTNGLVVYCGTVIGEDDSEKKYTIDFEPFRPLNTFKYICDNKFCTEPLFELLENDDVFGFVIVDGNGCLFGTLQGNTKKILQQITVSLPKKHGRGGQSAPRFGRIREEKRHNYVRKVAELATQHFITDDRPNVKGLVLAGSANFKNDLSESDLFDKRLSEVVIKIVDVSYGGENGFSQAISLAEDALSNVKFVEEKNLISKYFEEIALDSGMIVFGVEDTLHSLEVGALDLLMCFENLEINRYEIRDPANDEIKIYNLNKEQEKDSKYFKNEKTGTDLEIVKCVALSEWLCENYSKYGVKLEFITDKSQEGFQFVNGFGGIGGFLRYKLEMENHDYDKEDVGGEEFNPDEDFI

SEQ ID NO: 67 - protein, 436 aa, synthetic construct
MEGDELTQNIEQWKIKRLIDNLDKARGNGTSLISLIIPPREQLPIINKMITEEYGKSSNIKSRIVRQAVQSALTSTKERLKLYNNRLPANGLILYCGEVINEEGVCEKKYTIDFQPYRAINTTLYICDNKFHTQPLKDLLVMDDKFGFIIIDGNGALFGTLQGNTREVLHKFSVDLPKKHRRGGQSALRFARLRMESRNNYLRKVAEQAVVQFISNDKVNVAGLIVAGSAEFKNVLVQSDLFDQRLAAKVLKIVDVAYGGENGFTQAIELSADTLSNIKFIREKKVMSKFFEEVAQDTKKYCYGVEDTMKTLIMGAVEVILLFENLNFTRYVLKNPTTGVEKTLYLTPEQEENHDNFMENGEELEALEKGPLPEWIVDNYMKFGAGLEFITDRSQEGAQFVRGFGGLGAFLRYQVDMAHLNAGEEELDEEWDDDFM

SEQ ID NO: 68 - protein, 435 aa, synthetic construct
MEEKDQRQRNIEHFKIKKLMTRLRNTRGSGTSMVSLIIPPKKQINDSTKLISDEFSKATNIKDRVNRQSVQDAMVSALQRLKLYQRTPNNGLILYCGKVLNEEGKEIKLLIDFEPYKPINTSLYFCDSKFHVDELGSLLETDPPFGFIVMDGQGALYANLQGNTKTVLNKFSVELPKKHGRGGQSSVRFARLRVEKRHNYLRKVCEVATQTFISQDKINVQGLVLAGSGDFKNELSTTQMFDPRLACKIIKIVDVSYGGENGLNQAIELAQESLTNVKFVQEKNVISKFFDCIAIDSGTVVYGVQDTMQLLLDGVIENILCFEELTTLRVTRKNKVTEQITHIFIPPNELNNPKHFKDGEHELEKIEVENLTEWLAEHYSEFGAELYFITDKSAEGCQFVKGFSGIGGFLRYKVDLEHIVNPNDEYNYEEEEGFI

SEQ ID NO: 69 - protein, 485 aa, synthetic construct
MEQKPPFQNPLQKLQDRGTKMDQSSGSCMSKQAEEQKRLQISQYQLRKQLQMLRNMRGEQTSCVSLYIPERKKLYEVVNYLQQEESGAASIKNTQNRKSVQSALSMLRERLKNFNLHKKYPKGMIFFCADSLDSKRLLIEILDPPKAVQSFRYSCNTIFYLDDLEYMLKDQPTYGFVVADGHGYLIATVCGFDIQILQSKQEDLPNKHNKGGQSSLRFSRLCDAARERLVKNIADAMRRCYANENGTQTNLSGIVLCGMSDIKDKVQKELQQLCPCIENKIVASYDVSYSGQAGLKQALQMSTEMLKLDQLFQEMNLLSDFFANFSLETSKVVYGGELTVRALEEGNVKKLILCQDSELQRVTVYNSKTQEETIQYLMPSQVKALQDSISKTSDQEANNKKNQLQVYSQQNINEWIVENISSFSQDLEIVFVSDKTQQGVQFSKSFQGVGAYLKYSLDYSSLHAQEKENDQLEQEYCYDDEEGFI

SEQ ID NO: 70 - protein, 452 aa, synthetic construct
MIKNIFKLLPISLRAIPLKQQQNSFSQICSLYNTKLFKVINLIQTNNKCFFSFRAKETFKKKTSSLEIETHFQVSDLTRCIYRRMKQFHNEYTDIQKILSQEQQQADINLEQLRKKINVLQPLNDVFEKLEQNIKTLQELQKQKEESASDPEMLALIEEEMENSKQLIDELQDECLEQLLPKGKHDDCSEITLEVRGGAGGSESSLFAEEVFKMYQAFFAQQGYQFSIDSFQVDMAINKGCKLGVLKVSGTNIYKKMMNESGVHKVIRVPETESKGRLHSSTISVVVMPVVPMDFKVDEKDLKFEFMRSQGAGGQHVNKVESACRVTHLPTGISVLCQDDRQQERNKQRALKLLTEKLFQVEVEKSNQQQSDQRKSQIGGGDRSDKIRTYNFPQGRITDHRTNLTLFGIEKMMKGEFLEEFIDEYEEKVNNELIESVLKQLEEDENQSQPKN

SEQ ID NO: 71 - protein, 437 aa, synthetic construct
MDQKLNDAEIALEQFRLKKLIKTLSQERTAGTSVVSVYIPPKRIISDITNRLNTQYAEAASIKDKGNRISVQEAIQAAILRLRPYNKAPNNGLVVFCGIVQQADGKGEKKISVVIEPYRPLDLSLYFCDPQFHVEELRALLNIDPPFGFIIMDGNGSLFATIQGNSKQIIKSFDVDLPKKHNKGGQSSVRFARLRMEKRHNYLRKVCETATTCFIAEDRPNVKGLVLAGSADFKNDLAGSQFFDKRLQPLIISVVDINYGGEQGLNQAVQLSQESLLEVKYIREKNLVGQFFENIDKDTGLVVYGVQDTMRAVESQTIKTLVCVDTLQYLRLECQSKQTEQKAIKYIKGNEGYEAGSLIEEKNGEQFVILVKEDLVEHLSEKFKDYGLDFQLITDHSVEGNQFMKGFSGLGGFLRFKMDMDYLVQQEDWKDEDEDFI

SEQ ID NO: 72 - protein, 437 aa, synthetic construct
MNQNQIQEQELEIEQFRLSKIIKTLSKTKVIGTSAVSLYIPPKKIISDITNRLNTQFSEAASIQDKVNRTSVQDSIQGAVLKLKKYTKAPASGLVLFSGLVEFEKGQKKISYVIEPFRPLQLSLFFCDNYFHIEQLEPLLKLEPSYGFIIMDGNGALFGKVQGISKETLKSFNVDLPKKHNKGGQSSLRFSRIRYWARHNYLIKVSEQAKNCFISDDKPTIKGLVLAGIADFKNKLAESPALDKRLQPLILSIVDVNYGGENGFNQAIQYSQEVLQNQKLQREKDLVAKFFLSLDLDNGKSVYGVVDTMKAIEQELVKQVICIQTLEYSRVECISKQTGVKSIKYLKGLDLYEQGSLFEDNKGEQFQVTSCQDLVEYLAENYREKGIDFQLISDNSAEGHQFYKGFGGMAGFFRFSMKMQYNMDSEEEWKSEDDEFI

SEQ ID NO: 73 - protein, 444 aa, synthetic construct
MVESIAAGQVGDNKHIEMWKIKRLINKLENCKGNGTSMVSLIIPPKEDINKSGKLLVGELSAAQNIKSRITRQSVITAITSTKEKLKLYRQTPTNGLCIYCGVILMEDGKTEKKINFDFEPFRPINQFMYFCGGKFQTEPLTTLLADDDKFGFIIVDGNGALYATLQGNSREILQKITVELPKKHRKGGQSSVRFARLREEKRHNYLRKVAELAGSNFITNDKPNVTGLVLAGNAGFKNELSETDMLDKRLLPIIVSIVDVSYGGENGLNEAITLSADALTNVKFVAEKKLVSKFFEEISLDTGMIVFGVQDTMKALELGAVETILLFEELEITRYVIKNPVKGDTRTLFLNPTQQKDSKYFKDQASGLDMDVISEDQLAEWLCHNYQNYAQVEFITDKSQEGYQFVKGFGGIGGFLRYKVDMEEALGDVGDGGDDFDPDTDFI

SEQ ID NO: 74 - protein, 437 aa, synthetic construct
MDEAKLLQLEMWRLRKQLQKLDNTNTNSTSVVSLVMPPGEDINKMVQMLNQEATQADCIKSRQNRQAVQTAIILAANRCKLYPKMPKNGLAVFAGEVYVDGKIKKIAVHFSPCKPIGNFMYSCDGVFHTQEVKDLLTVEEVYGFIIMDGHGTLIATLCGSHREIKHRMLVDLPKKHGRGGQSSVRFARLRMEARGNYVKIITELCTKYFITGDRLNVSGVILAGSADFKDVLAGSDFMDPRIKEGIIKQVDIGYGMEQGLSQAIEQAGDILKDVRLFKEVKIINEFLDHISRDTRKYCFGIIDTLRCLEMGSVEHLVVWEDFPWDRVQCRNSEGTEYYKLVNTVTGAINLVDNDESIRQGEEEIEAELFVEWLAQNIEKFGAEIHLVTENSNEGTQFCSGFGGLGGILRWQMDLNELGKYMDIGKENNDLDDLEDFM

SEQ ID NO: 75 - protein, 437 aa, synthetic construct; identical to SEQ ID NO: 40

SEQ ID NO: 76 - protein, 685 aa, synthetic construct
MSDSNQGNNQQNYQQYSQNGNQQQGNNRYQGYQAYNAQAQPAGGYYQNYQGYSGYQQGGYQQYNPDAGYQQQYNPQGGYQQYNPQGGYQQQFNPQGGRGNYKNFNYNNNLQGYQAGFQPQSQGMSLNDFQKQQKQAAPKPKKTLKLVSSSGIKLANATKKVGTKPAESDKKEEEKSAETKEPTKEPTKVEEPVKKEEKPVQTEEKTEEKSELPKVEDLKISESTHNTNNANVTSADALIKEQEEEVDDEVVNDMFGGKDHVSLIFMGHVDAGKSTMGGNLLYLTGSVDKRTIEKYEREAKDAGRQGWYLSWVMDTNKEERNDGKTIEVGKAYFETEKRRYTILDAPGHKMYVSEMIGGASQADVGVLVISARKGEYETGFERGGQTREHALLAKTQGVNKMVVVVNKMDDPTVNWSKERYDQCVSNVSNFLRAIGYNIKTDVVFMPVSGYSGANLKDHVDPKECPWYTGPTLLEYLDTMNHVDRHINAPFMLPIAAKMKDLGTIVEGKIESGHIKKGQSTLLMPNKTAVEIQNIYNETENEVDMAMCGEQVKLRIKGVEEEDISPGFVLTSPKNPIKSVTKFVAQIAIVELKSIIAAGFSCVMHVHTAIEEVHIVKLLHKLEKGTNRKSKKPPAFAKKGMKVIAVLETEAPVCVETYQDYPQLGRFTLRDQGTTIAIGKIVKIAE

SEQ ID NO: 77 - protein, 446 aa, synthetic construct; identical to SEQ ID NO: 65

SEQ ID NO: 78 - protein, 799 aa, synthetic construct
MDKNSDFPALGSLKVQNQSSKDKNKKKEKEVKKEDEKKDEVKDEPQNNEEAKKTKLEATGRVFAAKSFTPKEPVKINYYIPHEEPTLSEMDFFPTLGGPASTAPVVSEAQKRHAEFLERHELFKPYICLIPKELWYVPDPYFDMFGYPVMALLPGLYAYLWNTYNIFFTPEGAWNTNKETVMSTLGELWTHAEWRDRIIAKEQKETEEWERQMREWEEENAEDDEGLSIDDMENYGKGKKKNKNKKKKDKAKRPPPPPPKSLSSYKRFEKKKEDVVVPKKNIGFKEVSEITFEEEVVEVDETRQPSSLVFIGPVDAVKSTICGNLMFMTGMVDERTIEKFKQEAKEKNRDSWWLAYVMDINDDEKSKGKTVEVGRATMETPTKRYTIFDAPGHKNYVPDMIMGAAMADVAALVISARKGEFEAGFERDGQTREHAQLARSLGVNKLVVVVNEMDEETVQWSEERYNDILSGVTPFLIDQCGYKREDLIFVPISGLNGHNIDKLASCCPWYTGPTLLEILDCIEPPKRNIDGPLRVPVLDKMKDRGVVAFGKVESGVIRIGPKLAVMPNNTKCQVVGIYNCKLELVRYANPGENIQIKVRMIEDENQINKGDVLCPYDNLAPITDLFEAELTILELLPHRPIITPGYKSMMHLHTISDEIVIQTLTGIYELDGSGKEYVKKNPKYCKSGSKVIVKISTRVPVCLEKYEFIVHMGRFTLRDEGKTIALGKVLRYKPAVIKKVEEIPPGVGDEGQAKLEESEEFSGSRGDSPSKDDNKYEVITYDPEEDTIIASSTGSENAE

SEQ ID NO: 79 - protein, 437 aa, synthetic construct; identical to SEQ ID NO: 66

SEQ ID NO: 80 - protein, 799 aa, synthetic construct; identical to SEQ ID NO: 78

SEQ ID NO: 81 - protein, 436 aa, synthetic construct; identical to SEQ ID NO: 67

SEQ ID NO: 82 - protein, 409 aa, synthetic construct
MVDSGKSTSCGHLIYKCGGIDKRTIEKYEKEAKEMGKSSFKYAWVLDKLKAERERGITIDISLFKFQTDKFYFTIIDAPGHRDFIKNMITGTSQADVAILIIAAGKGEFEAGYSKNGQTREHALLAFTLGVKQMVVGVNKMDDKSAEWKQDRYLEIKQEVSEYLKKVGYNPAKVPFIPISGWLGDNMVEKSTNMPWYDGPTLLGALDNVQPPKRHVDKPLRLPVQDVYKISGIGTVPVGRVETGVLRPGTVVVFAPSGISTEVKSVEMHHESLEEALPGDNVGFNIKNIAVNQIKRGYVASDSRSDPARESIDFTAQVIILHHPGQISVGYTPVLDCHTAHIACRFNDLKQKVDRRSGAVLEQEPKFVKSGDAAIVTLIPTKSMCVEPFTEYPPLGRFAVRDMRQTVAV

SEQ ID NO: 83 - protein, 435 aa, synthetic construct; identical to SEQ ID NO: 68

SEQ ID NO: 84 - protein, 646 aa, synthetic construct
MDYQEAKRLAKEEKLRKLKAKIQENKVEDFQVTEQQGPLPPYDDQGNQQGKWLTTKYKFGEWYYDMNGNPVFISPDDEYDQNGSLYDAESNPYLDYAYDKFGNPCPVILMTQDNEYFLFTPIFEIQIPQSIIKDINQKQEKPATQKVETVAKEKKAPIQKAPAPPKIPKRLLKEQEAANSSAVVADYKHEKLVTVQEAIDFESKNFKEGQEMVKVDRERDSVNIVFIGHVDAGKSTLSGRILKNCGEVDETEIRKFELEAKEKNRESWVLAYIMDINEEERSKGITVECGKAHFQLANKRFVLLDAPGHKNYVPNMIAGACQADVAALIISARQGEFEAGFEGGQTQEHAHLAKALGVQHMICVVSKMDEVNWDKKRYDHIHDSVEPFLRNQVGIQSIEWVPINGFLNENIDTPIPTERCEWYKGDTLFDKFNKVPVPLRDPNGPVRIPVLDKLKDQGQFLFGKIESGTIRDDLWVTLMPYRKQFQILSIYNTKDQRVLYASAGENVKIKLKGLEDKDIERGYMVCSTEDLCPITQLFIAEITILQLPEHKPIMSQGYSCVLHMHTSVAEIEIEEVEAVQNPENKKLTKNTFLKSNQTGVVKIGIKGGLMCLEKFETISQLGRFTLRDEEKTIGFGRVMKIKPYKV

SEQ ID NO: 85 - protein, 485 aa, synthetic construct; identical to SEQ ID NO: 69

SEQ ID NO: 86 - protein, 646 aa, synthetic construct; identical to SEQ ID NO: 84

SEQ ID NO: 87 - protein, 452 aa, synthetic construct; identical to SEQ ID NO: 70

SEQ ID NO: 88 - protein, 646 aa, synthetic construct; identical to SEQ ID NO: 84

SEQ ID NO: 89 - protein, 437 aa, synthetic construct; identical to SEQ ID NO: 71

SEQ ID NO: 90 - protein, 756 aa, synthetic construct
MSYQYGQQMGQYPYDPNMNMMGFDPQMYQEYAYYYLGGPPTPPPPKGPYPGITHEDYESFDINKQILFQRFLGETAAYYAKHLPKYQKEMEEFLNTNTAYQMNESEKQLMQSYLDFKKKEKEYESFLKQLEQQALNPELEQQKKLEQQKLEQQKIEQQKLEEQKKQQQLEQQKQQQQQQQQQPQQEQPKEGATTAVRPKKKLNLNAKPLEIALNPPKMPNFPKHPDFLDFDKFWNNYSTYISLYNPSCEDEYKNYPKPEQLKKKEADEEAKRKKKQEEIERAIKKRQDAQERAKDKPAQSVNLVEQVVKLEGEVDLQKYVDPDETRQPVNLVFIGHVDAGKSTLCGRLLLELGEVSEADIKKYEQEAVQNNRDSWWLAYVMDQNEEEKQKGKTVECGKAQFVTKQKRFILADAPGHKNYVPNMIMGACQADLAGLIVSAKTGEFESGFEKGGQTQEHALLAKSLGVDHIIIIVTKMDTIDWNQDRFNLISQNIQEFVLKQCKFDNIYVIPIDALSGSNIKSRVDESKCNWYKGPSLIDLIDTVSIPKRNEEGPIRMPILDKFKDMGSLYIYGKLESGKIIEGLDVSIYPKKQPFQITELYNMKDQKMKYAKAGENIKIKVKNIEEEEIKRGYMMCNLTSNPCLVSQEFQAKIRLLDLPESRRIFSEGYQCIMHLHSAVEEIEISCVEAVIDAETKKSIKQNFLKSFNEGIAKISIKNPVCMEKYETLAQLGRFALRDDGKTIGFGEILKVKPVKQG

SEQ ID NO: 91 - protein, 437 aa, synthetic construct; identical to SEQ ID NO: 72

SEQ ID NO: 92 - protein, 756 aa, synthetic construct; identical to SEQ ID NO: 90

SEQ ID NO: 93 - protein, 446 aa, synthetic construct; identical to SEQ ID NO: 65

SEQ ID NO: 94 - protein, 755 aa, synthetic construct
MDKNSDQGNNQQNYQQYSQNGNQQQGNNRYQGYQAYNAQAQPAGGYYQNYQGYSGYQQGGYQQYNPDAGYQQQYNPQGGYQQYNPQGGYQQQFNPQGGRGNYKNFNYNNNLQGYQAGFQPQSQGMSLNDFQKQQKQAAPKPKKTLKLVSSSGIKLANATKKVDTKPAESDKKEEEKSAETKEPTKEPTKVEEPVKKEEKPVQTEEKTEEKSELPKVEDLKISESTHNTNNANVTSADALIKEQEEEVDDEVVNDVDETRQPSSLVFIGPVDAVKSTICGNLMFMTGMVDERTIEKFKQEAKEKNRDSWWLAYVMDINDDEKSKGKTVEVGRATMETPTKRYTIFDAPGHKNYVPDMIMGAAMADVAALVISARKGEFEAGFERDGQTREHAQLARSLGVNKLVVVVNEMDEETVQWSEERYNDILSGVTPFLIDQCGYKREDLIFVPISGLNGHNIDKLASCCPWYTGPTLLEILDCIEPPKRNIDGPLRVPVLDKMKDRGVVAFGKVESGVIRIGPKLAVMPNNTKCQVVGIYNCKLELVRYANPGENIQIKVRMIEDENQINKGDVLCPYDNLAPITDLFEAELTILELLPHRPIITPGYKSMMHLHTISDEIVIQTLTGIYELDGSGKEYVKKNPKYCKSGSKVIVKISTRVPVCLEKYEFIVHMGRFTLRDEGKTIALGKVLRYKPAVIKKVEEIPPGVGDEGQAKLEESEEFSGSRGDSPSKDDNKYEVITYDPEEDTIIASSTGSENAE

SEQ ID NO: 95 - protein, 437 aa, synthetic construct; identical to SEQ ID NO: 66

SEQ ID NO: 96 - protein, 754 aa, synthetic construct
MSDSNQGNNQQNYQQYSQNGNQQQGNNRYQGYQAYNAQAQPAGGYYQNYQGYSGYQQGGYQQYNPDAGYQQQYNPQGGYQQYNPQGGYQQQFNPQGGRGNYKNFNYNNNLQGYQAGFQPQSQGMSLNDFQKQQKQAAPKPKKTLKLVSSSGIKLANATKKVDTKPAESDKKEEEKSAETKEPTKEPTKVEEPVKKEEKPVQTEEKTEEKSELPKVEDLKISESTHNTNNANVTSADALIKEQEEEVDDEVVNDVDETRQPSSLVFIGPVDAVKSTICGNLMFMTGMVDERTIEKFKQEAKEKNRDSWWLAYVMDINDDEKSKGKTVEVGRATMETPTKRYTIFDAPGHKNYVPDMIMGAAMADVAALVISARKGEFEAGFERDGQTREHAQLARSLGVNKLVVVVNEMDEETVQWSEERYNDILSGVTPFLIDQCGYKREDLIFVPISGLNGHNIDKLASCCPWYTGPTLLEILDCIEPPKRNIDGPLRVPVLDKMKDRGVVAFGKVESGVIRIGPKLAVMPNNTKCQVVGIYNCKLELVRYANPGENIQIKVRMIEDENQINKGDVLCPYDNLAPITDLFEAELTILELLPHRPIITPGYKSMMHLHTISDEIVIQTLTGIYELDGSGKEYVKKNPKYCKSGSKVIVKISTRVPVCLEKYEFIVHMGRFTLRDEGKTIALGKVLRYKPAVIKKVEEIPPGVGDEGQAKLEESEEFSGSRGDSPSKDDNKYEVITYDPEEDTIIASSTGSENAE

SEQ ID NO: 97 - protein, 437 aa, synthetic construct; identical to SEQ ID NO: 71

SEQ ID NO: 98 - protein, 688 aa, synthetic construct
MSDSNQGNNQQNYQQYSQNGNQQQGNNRYQGYQAYNAQAQPAGGYYQNYQGYSGYQQGGYQQYNPDAGYQQQYNPQGGYQQYNPQGGYQQQFNPQGGRGNYKNFNYNNNLQGYQAGFQPQSQGMSLNDFQKQQKQAAPKPKKTLKLVSSSGIKLANATKKVDTKPAESDKKEEEKSAETKEPTKEPTKVEEPVKKEEKPVQTEEKTEEKSELPKVEDLKISESTHNTNNANVTSADALIKEQEEEVDDEVVNDPDETRQPVNLVFIGHVDAGKSTLCGRLLLELGEVSEADIKKYEQEAVQNNRDSWWLAYVMDQNEEEKQKGKTVECGKAQFVTKQKRFILADAPGHKNYVPNMIMGACQADLAGLIVSAKTGEFESGFEKGGQTQEHALLAKSLGVDHIIIIVTKMDTIDWNQDRFNLISQNIQEFVLKQCKFDNIYVIPIDALSGSNIKSRVDESKCNWYKGPSLIDLIDTVSIPKRNEEGPIRMPILDKFKDMGSLYIYGKLESGKIIEGLDVSIYPKKQPFQITELYNMKDQKMKYAKAGENIKIKVKNIEEEEIKRGYMMCNLTSNPCLVSQEFQAKIRLLDLPESRRIFSEGYQCIMHLHSAVEEIEISCVEAVIDAETKKSIKQNFLKSFNEGIAKISIKNPVCMEKYETLAQLGRFALRDDGKTIGFGEILKVKPVKQG

SEQ ID NO: 99 - protein, 437 aa, synthetic construct; identical to SEQ ID NO: 72

SEQ ID NO: 100 - protein, 688 aa, synthetic construct; identical to SEQ ID NO: 98

SEQ ID NO: 101 - DNA, 1314 bp, synthetic construct
  source 1..1314: mol_type = other DNA
atggataacgaggttgaaaaaaatattgagatctggaaggtcaagaagttggtccaatctttagaaaaagctagaggtaatggtacttctatgatttccttagttattcctcctaagggtcaaattccactgtaccaaaaaatgttaacagatgaatatggtactgcctcgaatattaaatctagggttaatcgtctttccgttttatctgctatcacttccacccaacaaaagttgaagctatataatactttgcccaagaacggtttagttttatattgtggtgatatcatcactgaagatggtaaagaaaaaaaggtcacttttgacatcgaaccttacaaacctatcaacacatccttatatttgtgtgataacaaatttcatacagaagttctttcggaattgcttcaagctgacgacaagttcggttttatagtcatggacggtcaaggtactttgtttggttctgtgtccggtaatacgagaactgttttacataaatttactgtcgatctgccaaaaaagcatggtagaggtggtcaatctgcgcttcgttttgctcgtttaagagaagaaaaaagacataattatgtgagaaaggtcgccgaagttgctgttcaaaattttattactaatgacaaagtcaatgttaagggtttaattttagctggttctgctgactttaagaccgatttggctaaatctgaattattcgatccaagactagcatgtaaggttatttccatcgtggatgtttcttatggtggtgaaaacggtttcaaccaggctatcgaactttctgccgaagcgttggccaatgtcaagtatgttcaagaaaagaaattattggaggcatattttgacgaaatttcccaggacactggtaaattctgttatggtatagatgatactttaaaggcattggatttaggtgcagtcgaaaaattaattgttttcgaaaatttggaaactatcagatatacatttaaagatgccgaggataatgaggttataaaattcgctgaaccagaagccaaggacaagtcgtttgctattgacaaagctaccggccaagaaatggacgttgtctccgaagaacctttaattgaatggctagcagctaactacaaaaacttcggtgctaccttggaattcatcacagacaaatcttcagaaggtgcccaatttgtcacaggttttggtggtattggtgccatgctgcgttacaaagttaattttgaacaactagttgatgaatctgaggatgaatattatgacgaagatgaaggatccgactatgatttcatttaa

SEQ ID NO: 102 - DNA, 1314 bp, synthetic construct
  source 1..1314: mol_type = other DNA
atggataacgaggttgaaaaaaatattgagatctggaaggtcaagaagttggtccaatctttagaaaaagctagaggtaatggtacttctatgatttccttagttattcctcctaagggtcaaattccactgtaccaaaaaatgttaacagatgaatatggtaagtcttctaacatcaagtctagagttaacagattgtctgttttgtctgctatcacctctacccaacaaaagttgaagttgtacaacaccttgccaaagaacggtttggttttgtactgtggtgacatcatcaccgaagacggtaaggaaaagaaggttaccttcgacatcgaaccatacaagccaatcaacacctctttgtacatctgtgacaacaagttccatacagaagttctttcggaattgcttcaagctgacgacaagttcggttttatagtcatggacggtcaaggtactttgtttggttctgtgtccggtaatacgagaactgttttacataaatttactgtcgatctgccaaaaaagcatggtagaggtggtcaatctgcgcttcgttttgctcgtttaagagaagaaaaaagacataattatgtgagaaaggtcgccgaagttgctgttcaaaattttattactaatgacaaagtcaatgttaagggtttaattttagctggttctgctgactttaagaccgatttggctaaatctgaattattcgatccaagactagcatgtaaggttatttccatcgtggatgtttcttatggtggtgaaaacggtttcaaccaggctatcgaactttctgccgaagcgttggccaatgtcaagtatgttcaagaaaagaaattattggaggcatattttgacgaaatttcccaggacactggtaaattctgttatggtatagatgatactttaaaggcattggatttaggtgcagtcgaaaaattaattgttttcgaaaatttggaaactatcagatatacatttaaagatgccgaggataatgaggttataaaattcgctgaaccagaagccaaggacaagtcgtttgctattgacaaagctaccggccaagaaatggacgttgtctccgaagaacctttaattgaatggctagcagctaactacaaaaacttcggtgctaccttggaattcatcacagacaaatcttcagaaggtgcccaatttgtcacaggttttggtggtattggtgccatgctgcgttacaaagttaattttgaacaactagttgatgaatctgaggatgaatattatgacgaagatgaaggatccgactatgatttcatttaa

SEQ ID NO: 103 - DNA, 1314 bp, synthetic construct
  source 1..1314: mol_type = other DNA
atggataacgaggttgaaaaaaatattgagatctggaaggtcaagaagttggtccaatctttagaaaaagctagaggtaatggtacttctatgatttccttagttattcctcctaagggtcaaattccactgtaccaaaaaatgttaacagatgaatatggtaccgctgttaacatcaagtctagagttaacagattgtctgttttgtctgctatcacctctacccaacaaaagttgaagttgtacaacaccttgccaaagaacggtttggttttgtactgtggtgacatcatcaccgaagacggtaaggaaaagaaggttaccttcgacatcgaaccatacaagccaatcaacacctctttgtacatctgtgacaacaagttccatacagaagttctttcggaattgcttcaagctgacgacaagttcggttttatagtcatggacggtcaaggtactttgtttggttctgtgtccggtaatacgagaactgttttacataaatttactgtcgatctgccaaaaaagcatggtagaggtggtcaatctgcgcttcgttttgctcgtttaagagaagaaaaaagacataattatgtgagaaaggtcgccgaagttgctgttcaaaattttattactaatgacaaagtcaatgttaagggtttaattttagctggttctgctgactttaagaccgatttggctaaatctgaattattcgatccaagactagcatgtaaggttatttccatcgtggatgtttcttatggtggtgaaaacggtttcaaccaggctatcgaactttctgccgaagcgttggccaatgtcaagtatgttcaagaaaagaaattattggaggcatattttgacgaaatttcccaggacactggtaaattctgttatggtatagatgatactttaaaggcattggatttaggtgcagtcgaaaaattaattgttttcgaaaatttggaaactatcagatatacatttaaagatgccgaggataatgaggttataaaattcgctgaaccagaagccaaggacaagtcgtttgctattgacaaagctaccggccaagaaatggacgttgtctccgaagaacctttaattgaatggctagcagctaactacaaaaacttcggtgctaccttggaattcatcacagacaaatcttcagaaggtgcccaatttgtcacaggttttggtggtattggtgccatgctgcgttacaaagttaattttgaacaactagttgatgaatctgaggatgaatattatgacgaagatgaaggatccgactatgatttcatttaa

SEQ ID NO: 104 - DNA, 1314 bp, synthetic construct
  source 1..1314: mol_type = other DNA
atggataacgaggttgaaaaaaatattgagatctggaaggtcaagaagttggtccaatctttagaaaaagctagaggtaatggtacttctatgatttccttagttattcctcctaagggtcaaattccactgtaccaaaaaatgttaacagatgaatatggtaaggctgctaacatcaagtctagagttaacagattgtctgttttgtctgctatcacctctacccaacaaaagttgaagttgtacaacaccttgccaaagaacggtttggttttgtactgtggtgacatcatcaccgaagacggtaaggaaaagaaggttaccttcgacatcgaaccatacaagccaatcaacacctctttgtacttgtgtgacaacaagttccatacagaagttctttcggaattgcttcaagctgacgacaagttcggttttatagtcatggacggtcaaggtactttgtttggttctgtgtccggtaatacgagaactgttttacataaatttactgtcgatctgccaaaaaagcatggtagaggtggtcaatctgcgcttcgttttgctcgtttaagagaagaaaaaagacataattatgtgagaaaggtcgccgaagttgctgttcaaaattttattactaatgacaaagtcaatgttaagggtttaattttagctggttctgctgactttaagaccgatttggctaaatctgaattattcgatccaagactagcatgtaaggttatttccatcgtggatgtttcttatggtggtgaaaacggtttcaaccaggctatcgaactttctgccgaagcgttggccaatgtcaagtatgttcaagaaaagaaattattggaggcatattttgacgaaatttcccaggacactggtaaattctgttatggtatagatgatactttaaaggcattggatttaggtgcagtcgaaaaattaattgttttcgaaaatttggaaactatcagatatacatttaaagatgccgaggataatgaggttataaaattcgctgaaccagaagccaaggacaagtcgtttgctattgacaaagctaccggccaagaaatggacgttgtctccgaagaacctttaattgaatggctagcagctaactacaaaaacttcggtgctaccttggaattcatcacagacaaatcttcagaaggtgcccaatttgtcacaggttttggtggtattggtgccatgctgcgttacaaagttaattttgaacaactagttgatgaatctgaggatgaatattatgacgaagatgaaggatccgactatgatttcatttaa

SEQ ID NO: 105 - DNA, 1314 bp, synthetic construct
  source 1..1314: mol_type = other DNA
atggataacgaggttgaaaaaaatattgagatctggaaggtcaagaagttggtccaatctttagaaaaagctagaggtaatggtacttctatgatttccttagttattcctcctaagggtcaaattccactgtaccaaaaaatgttaacagatgaatatggtaaggcttctaacatcaagtctagagttaacagattgtctgttttgtctgctatcacctctacccaacaaaagttgaagttgtacaacaccttgccaaagaacggtttggttttgtactgtggtgacatcatcaccgaagacggtaaggaaaagaaggttaccttcgacatcgaaccatacaagccaatcaacacctctttgtactactgtggtgaaagattccatacagaagttctttcggaattgcttcaagctgacgacaagttcggttttatagtcatggacggtcaaggtactttgtttggttctgtgtccggtaatacgagaactgttttacataaatttactgtcgatctgccaaaaaagcatggtagaggtggtcaatctgcgcttcgttttgctcgtttaagagaagaaaaaagacataattatgtgagaaaggtcgccgaagttgctgttcaaaattttattactaatgacaaagtcaatgttaagggtttaattttagctggttctgctgactttaagaccgatttggctaaatctgaattattcgatccaagactagcatgtaaggttatttccatcgtggatgtttcttatggtggtgaaaacggtttcaaccaggctatcgaactttctgccgaagcgttggccaatgtcaagtatgttcaagaaaagaaattattggaggcatattttgacgaaatttcccaggacactggtaaattctgttatggtatagatgatactttaaaggcattggatttaggtgcagtcgaaaaattaattgttttcgaaaatttggaaactatcagatatacatttaaagatgccgaggataatgaggttataaaattcgctgaaccagaagccaaggacaagtcgtttgctattgacaaagctaccggccaagaaatggacgttgtctccgaagaacctttaattgaatggctagcagctaactacaaaaacttcggtgctaccttggaattcatcacagacaaatcttcagaaggtgcccaatttgtcacaggttttggtggtattggtgccatgctgcgttacaaagttaattttgaacaactagttgatgaatctgaggatgaatattatgacgaagatgaaggatccgactatgatttcatttaa

SEQ ID NO: 106 - DNA, 1314 bp, synthetic construct
  source 1..1314: mol_type = other DNA
atggataacgaggttgaaaaaaatattgagatctggaaggtcaagaagttggtccaatctttagaaaaagctagaggtaatggtacttctatgatttccttagttattcctcctaagggtcaaattccactgtaccaaaaaatgttaacagatgaatatggtaccgctgaatctatcaagtctagagttaacagattgtctgttttgtctgctatcacctctacccaacaaaagttgaagttgtacaacaccttgccaaagaacggtttggttttgtactgtggtgacatcatcaccgaagacggtaaggaaaagaaggttaccttcgacatcgaaccatacaagccaatcaacacctctttgtacatctgtgacaacaagttccatacagaagttctttcggaattgcttcaagctgacgacaagttcggttttatagtcatggacggtcaaggtactttgtttggttctgtgtccggtaatacgagaactgttttacataaatttactgtcgatctgccaaaaaagcatggtagaggtggtcaatctgcgcttcgttttgctcgtttaagagaagaaaaaagacataattatgtgagaaaggtcgccgaagttgctgttcaaaattttattactaatgacaaagtcaatgttaagggtttaattttagctggttctgctgactttaagaccgatttggctaaatctgaattattcgatccaagactagcatgtaaggttatttccatcgtggatgtttcttatggtggtgaaaacggtttcaaccaggctatcgaactttctgccgaagcgttggccaatgtcaagtatgttcaagaaaagaaattattggaggcatattttgacgaaatttcccaggacactggtaaattctgttatggtatagatgatactttaaaggcattggatttaggtgcagtcgaaaaattaattgttttcgaaaatttggaaactatcagatatacatttaaagatgccgaggataatgaggttataaaattcgctgaaccagaagccaaggacaagtcgtttgctattgacaaagctaccggccaagaaatggacgttgtctccgaagaacctttaattgaatggctagcagctaactacaaaaacttcggtgctaccttggaattcatcacagacaaatcttcagaaggtgcccaatttgtcacaggttttggtggtattggtgccatgctgcgttacaaagttaattttgaacaactagttgatgaatctgaggatgaatattatgacgaagatgaaggatccgactatgatttcatttaa

SEQ ID NO: 107 - DNA, 1314 bp, synthetic construct
  source 1..1314: mol_type = other DNA
atggataacgaggttgaaaaaaatattgagatctggaaggtcaagaagttggtccaatctttagaaaaagctagaggtaatggtacttctatgatttccttagttattcctcctaagggtcaaattccactgtaccaaaaaatgttaacagatgaatatggtaccgcttctaacatcaagtctagagttaacagattgtctgttttgtctgctatcacctctacccaacaaaagttgaagttgtacaacaccttgccaaagaacggtttggttttgtactgtggtgacatcatcaccgaagacggtaaggaaaagaaggttaccttcgacatcgaaccatacaagccaatcaacacctctttgtacttctgtgacccacaattccatacagaagttctttcggaattgcttcaagctgacgacaagttcggttttatagtcatggacggtcaaggtactttgtttggttctgtgtccggtaatacgagaactgttttacataaatttactgtcgatctgccaaaaaagcatggtagaggtggtcaatctgcgcttcgttttgctcgtttaagagaagaaaaaagacataattatgtgagaaaggtcgccgaagttgctgttcaaaattttattactaatgacaaagtcaatgttaagggtttaattttagctggttctgctgactttaagaccgatttggctaaatctgaattattcgatccaagactagcatgtaaggttatttccatcgtggatgtttcttatggtggtgaaaacggtttcaaccaggctatcgaactttctgccgaagcgttggccaatgtcaagtatgttcaagaaaagaaattattggaggcatattttgacgaaatttcccaggacactggtaaattctgttatggtatagatgatactttaaaggcattggatttaggtgcagtcgaaaaattaattgttttcgaaaatttggaaactatcagatatacatttaaagatgccgaggataatgaggttataaaattcgctgaaccagaagccaaggacaagtcgtttgctattgacaaagctaccggccaagaaatggacgttgtctccgaagaacctttaattgaatggctagcagctaactacaaaaacttcggtgctaccttggaattcatcacagacaaatcttcagaaggtgcccaatttgtcacaggttttggtggtattggtgccatgctgcgttacaaagttaattttgaacaactagttgatgaatctgaggatgaatattatgacgaagatgaaggatccgactatgatttcatttaa

SEQ ID NO: 108 - DNA, 1314 bp, synthetic construct
  source 1..1314: mol_type = other DNA
atggataacgaggttgaaaaaaatattgagatctggaaggtcaagaagttggtccaatctttagaaaaagctagaggtaatggtacttctatgatttccttagttattcctcctaagggtcaaattccactgtaccaaaaaatgttaacagatgaatatggtgaagctgcttctatcaaggacagagttaacagattgtctgttttgtctgctatcacctctacccaacaaaagttgaagttgtacaacaccttgccaaagaacggtttggttttgtactgtggtgacatcatcaccgaagacggtaaggaaaagaaggttaccttcgacatcgaaccatacaagccaatcaacacctctttgtacttctgtgacccacaattccatacagaagttctttcggaattgcttcaagctgacgacaagttcggttttatagtcatggacggtcaaggtactttgtttggttctgtgtccggtaatacgagaactgttttacataaatttactgtcgatctgccaaaaaagcatggtagaggtggtcaatctgcgcttcgttttgctcgtttaagagaagaaaaaagacataattatgtgagaaaggtcgccgaagttgctgttcaaaattttattactaatgacaaagtcaatgttaagggtttaattttagctggttctgctgactttaagaccgatttggctaaatctgaattattcgatccaagactagcatgtaaggttatttccatcgtggatgtttcttatggtggtgaaaacggtttcaaccaggctatcgaactttctgccgaagcgttggccaatgtcaagtatgttcaagaaaagaaattattggaggcatattttgacgaaatttcccaggacactggtaaattctgttatggtatagatgatactttaaaggcattggatttaggtgcagtcgaaaaattaattgttttcgaaaatttggaaactatcagatatacatttaaagatgccgaggataatgaggttataaaattcgctgaaccagaagccaaggacaagtcgtttgctattgacaaagctaccggccaagaaatggacgttgtctccgaagaacctttaattgaatggctagcagctaactacaaaaacttcggtgctaccttggaattcatcacagacaaatcttcagaaggtgcccaatttgtcacaggttttggtggtattggtgccatgctgcgttacaaagttaattttgaacaactagttgatgaatctgaggatgaatattatgacgaagatgaaggatccgactatgatttcatttaa

SEQ ID NO: 109 - DNA, 1314 bp, synthetic construct
  source 1..1314: mol_type = other DNA
atggataacgaggttgaaaaaaatattgagatctggaaggtcaagaagttggtccaatctttagaaaaagctagaggtaatggtacttctatgatttccttagttattcctcctaagggtcaaattccactgtaccaaaaaatgttaacagatgaatatggtaaggctaccaacatcaaggacagagttaacagattgtctgttttgtctgctatcacctctacccaacaaaagttgaagttgtacaacaccttgccaaagaacggtttggttttgtactgtggtgacatcatcaccgaagacggtaaggaaaagaaggttaccttcgacatcgaaccatacaagccaatcaacacctctttgtacttctgtgactctaagttccatacagaagttctttcggaattgcttcaagctgacgacaagttcggttttatagtcatggacggtcaaggtactttgtttggttctgtgtccggtaatacgagaactgttttacataaatttactgtcgatctgccaaaaaagcatggtagaggtggtcaatctgcgcttcgttttgctcgtttaagagaagaaaaaagacataattatgtgagaaaggtcgccgaagttgctgttcaaaattttattactaatgacaaagtcaatgttaagggtttaattttagctggttctgctgactttaagaccgatttggctaaatctgaattattcgatccaagactagcatgtaaggttatttccatcgtggatgtttcttatggtggtgaaaacggtttcaaccaggctatcgaactttctgccgaagcgttggccaatgtcaagtatgttcaagaaaagaaattattggaggcatattttgacgaaatttcccaggacactggtaaattctgttatggtatagatgatactttaaaggcattggatttaggtgcagtcgaaaaattaattgttttcgaaaatttggaaactatcagatatacatttaaagatgccgaggataatgaggttataaaattcgctgaaccagaagccaaggacaagtcgtttgctattgacaaagctaccggccaagaaatggacgttgtctccgaagaacctttaattgaatggctagcagctaactacaaaaacttcggtgctaccttggaattcatcacagacaaatcttcagaaggtgcccaatttgtcacaggttttggtggtattggtgccatgctgcgttacaaagttaattttgaacaactagttgatgaatctgaggatgaatattatgacgaagatgaaggatccgactatgatttcatttaa

SEQ ID NO: 110 - DNA, 1314 bp, synthetic construct
  source 1..1314: mol_type = other DNA
atggataacgaggttgaaaaaaatattgagatctggaaggtcaagaagttggtccaatctttagaaaaagctagaggtaatggtacttctatgatttccttagttattcctcctaagggtcaaattccactgtaccaaaaaatgttaacagatgaatatggtttcgacttcgacgctgaatctagagttaacagattgtctgttttgtctgctatcacctctacccaacaaaagttgaagttgtacaacaccttgccaaagaacggtttggttttgtactgtggtgacatcatcaccgaagacggtaaggaaaagaaggttaccttcgacatcgaaccatacaagccaatcaacacctctttgaccttgatcaagccacaattccatacagaagttctttcggaattgcttcaagctgacgacaagttcggttttatagtcatggacggtcaaggtactttgtttggttctgtgtccggtaatacgagaactgttttacataaatttactgtcgatctgccaaaaaagcatggtagaggtggtcaatctgcgcttcgttttgctcgtttaagagaagaaaaaagacataattatgtgagaaaggtcgccgaagttgctgttcaaaattttattactaatgacaaagtcaatgttaagggtttaattttagctggttctgctgactttaagaccgatttggctaaatctgaattattcgatccaagactagcatgtaaggttatttccatcgtggatgtttcttatggtggtgaaaacggtttcaaccaggctatcgaactttctgccgaagcgttggccaatgtcaagtatgttcaagaaaagaaattattggaggcatattttgacgaaatttcccaggacactggtaaattctgttatggtatagatgatactttaaaggcattggatttaggtgcagtcgaaaaattaattgttttcgaaaatttggaaactatcagatatacatttaaagatgccgaggataatgaggttataaaattcgctgaaccagaagccaaggacaagtcgtttgctattgacaaagctaccggccaagaaatggacgttgtctccgaagaacctttaattgaatggctagcagctaactacaaaaacttcggtgctaccttggaattcatcacagacaaatcttcagaaggtgcccaatttgtcacaggttttggtggtattggtgccatgctgcgttacaaagttaattttgaacaactagttgatgaatctgaggatgaatattatgacgaagatgaaggatccgactatgatttcatttaa

SEQ ID NO: 111 - DNA, 1314 bp, synthetic construct
  source 1..1314: mol_type = other DNA
atggataacgaggttgaaaaaaatattgagatctggaaggtcaagaagttggtccaatctttagaaaaagctagaggtaatggtacttctatgatttccttagttattcctcctaagggtcaaattccactgtaccaaaaaatgttaacagatgaatatggtaccggtgacaagatcaagtctagagttaacagattgtctgttttgtctgctatcacctctacccaacaaaagttgaagttgtacaacaccttgccaaagaacggtttggttttgtactgtggtgacatcatcaccgaagacggtaaggaaaagaaggttaccttcgacatcgaaccatacaagccaatcaacacctctttgaccatcatcaagaacgacttccatacagaagttctttcggaattgcttcaagctgacgacaagttcggttttatagtcatggacggtcaaggtactttgtttggttctgtgtccggtaatacgagaactgttttacataaatttactgtcgatctgccaaaaaagcatggtagaggtggtcaatctgcgcttcgttttgctcgtttaagagaagaaaaaagacataattatgtgagaaaggtcgccgaagttgctgttcaaaattttattactaatgacaaagtcaatgttaagggtttaattttagctggttctgctgactttaagaccgatttggctaaatctgaattattcgatccaagactagcatgtaaggttatttccatcgtggatgtttcttatggtggtgaaaacggtttcaaccaggctatcgaactttctgccgaagcgttggccaatgtcaagtatgttcaagaaaagaaattattggaggcatattttgacgaaatttcccaggacactggtaaattctgttatggtatagatgatactttaaaggcattggatttaggtgcagtcgaaaaattaattgttttcgaaaatttggaaactatcagatatacatttaaagatgccgaggataatgaggttataaaattcgctgaaccagaagccaaggacaagtcgtttgctattgacaaagctaccggccaagaaatggacgttgtctccgaagaacctttaattgaatggctagcagctaactacaaaaacttcggtgctaccttggaattcatcacagacaaatcttcagaaggtgcccaatttgtcacaggttttggtggtattggtgccatgctgcgttacaaagttaattttgaacaactagttgatgaatctgaggatgaatattatgacgaagatgaaggatccgactatgatttcatttaa

SEQ ID NO: 112 - DNA, 1314 bp, synthetic construct
  source 1..1314: mol_type = other DNA
atggataacgaggttgaaaaaaatattgagatctggaaggtcaagaagttggtccaatctttagaaaaagctagaggtaatggtacttctatgatttccttagttattcctcctaagggtcaaattccactgtaccaaaaaatgttaacagatgaatatggtgaagctgcttctatccaagacagagttaacagattgtctgttttgtctgctatcacctctacccaacaaaagttgaagttgtacaacaccttgccaaagaacggtttggttttgtactgtggtgacatcatcaccgaagacggtaaggaaaagaaggttaccttcgacatcgaaccatacaagccaatcaacacctctttgttcttctgtgacaactacttccatacagaagttctttcggaattgcttcaagctgacgacaagttcggttttatagtcatggacggtcaaggtactttgtttggttctgtgtccggtaatacgagaactgttttacataaatttactgtcgatctgccaaaaaagcatggtagaggtggtcaatctgcgcttcgttttgctcgtttaagagaagaaaaaagacataattatgtgagaaaggtcgccgaagttgctgttcaaaattttattactaatgacaaagtcaatgttaagggtttaattttagctggttctgctgactttaagaccgatttggctaaatctgaattattcgatccaagactagcatgtaaggttatttccatcgtggatgtttcttatggtggtgaaaacggtttcaaccaggctatcgaactttctgccgaagcgttggccaatgtcaagtatgttcaagaaaagaaattattggaggcatattttgacgaaatttcccaggacactggtaaattctgttatggtatagatgatactttaaaggcattggatttaggtgcagtcgaaaaattaattgttttcgaaaatttggaaactatcagatatacatttaaagatgccgaggataatgaggttataaaattcgctgaaccagaagccaaggacaagtcgtttgctattgacaaagctaccggccaagaaatggacgttgtctccgaagaacctttaattgaatggctagcagctaactacaaaaacttcggtgctaccttggaattcatcacagacaaatcttcagaaggtgcccaatttgtcacaggttttggtggtattggtgccatgctgcgttacaaagttaattttgaacaactagttgatgaatctgaggatgaatattatgacgaagatgaaggatccgactatgatttcatttaa

SEQ ID NO: 113 - DNA, 1314 bp, synthetic construct
  source 1..1314: mol_type = other DNA
atggataacgaggttgaaaaaaatattgagatctggaaggtcaagaagttggtccaatctttagaaaaagctagaggtaatggtacttctatgatttccttagttattcctcctaagggtcaaattccactgtaccaaaaaatgttaacagatgaatatggtaaggctaccaacatcaaggacagagttaacagattgtctgttttgtctgctatcacctctacccaacaaaagttgaagttgtacaacaccttgccaaagaacggtttggttttgtactgtggtgacatcatcaccgaagacggtaaggaaaagaaggttaccttcgacatcgaaccatacaagccaatcaacacctctttgttcgttatcgttaacaagttccatacagaagttctttcggaattgcttcaagctgacgacaagttcggttttatagtcatggacggtcaaggtactttgtttggttctgtgtccggtaatacgagaactgttttacataaatttactgtcgatctgccaaaaaagcatggtagaggtggtcaatctgcgcttcgttttgctcgtttaagagaagaaaaaagacataattatgtgagaaaggtcgccgaagttgctgttcaaaattttattactaatgacaaagtcaatgttaagggtttaattttagctggttctgctgactttaagaccgatttggctaaatctgaattattcgatccaagactagcatgtaaggttatttccatcgtggatgtttcttatggtggtgaaaacggtttcaaccaggctatcgaactttctgccgaagcgttggccaatgtcaagtatgttcaagaaaagaaattattggaggcatattttgacgaaatttcccaggacactggtaaattctgttatggtatagatgatactttaaaggcattggatttaggtgcagtcgaaaaattaattgttttcgaaaatttggaaactatcagatatacatttaaagatgccgaggataatgaggttataaaattcgctgaaccagaagccaaggacaagtcgtttgctattgacaaagctaccggccaagaaatggacgttgtctccgaagaacctttaattgaatggctagcagctaactacaaaaacttcggtgctaccttggaattcatcacagacaaatcttcagaaggtgcccaatttgtcacaggttttggtggtattggtgccatgctgcgttacaaagttaattttgaacaactagttgatgaatctgaggatgaatattatgacgaagatgaaggatccgactatgatttcatttaa

SEQ ID NO: 114 - DNA, 1314 bp, synthetic construct
  source 1..1314: mol_type = other DNA
atggataacgaggttgaaaaaaatattgagatctggaaggtcaagaagttggtccaatctttagaaaaagctagaggtaatggtacttctatgatttccttagttattcctcctaagggtcaaattccactgtaccaaaaaatgttaacagatgaatatggtgctgctcaaaacatcaagtctagagttaacagattgtctgttttgtctgctatcacctctacccaacaaaagttgaagttgtacaacaccttgccaaagaacggtttggttttgtactgtggtgacatcatcaccgaagacggtaaggaaaagaaggttaccttcgacatcgaaccatacaagccaatcaacacctctttgtacttctgtggtggtaagttccatacagaagttctttcggaattgcttcaagctgacgacaagttcggttttatagtcatggacggtcaaggtactttgtttggttctgtgtccggtaatacgagaactgttttacataaatttactgtcgatctgccaaaaaagcatggtagaggtggtcaatctgcgcttcgttttgctcgtttaagagaagaaaaaagacataattatgtgagaaaggtcgccgaagttgctgttcaaaattttattactaatgacaaagtcaatgttaagggtttaattttagctggttctgctgactttaagaccgatttggctaaatctgaattattcgatccaagactagcatgtaaggttatttccatcgtggatgtttcttatggtggtgaaaacggtttcaaccaggctatcgaactttctgccgaagcgttggccaatgtcaagtatgttcaagaaaagaaattattggaggcatattttgacgaaatttcccaggacactggtaaattctgttatggtatagatgatactttaaaggcattggatttaggtgcagtcgaaaaattaattgttttcgaaaatttggaaactatcagatatacatttaaagatgccgaggataatgaggttataaaattcgctgaaccagaagccaaggacaagtcgtttgctattgacaaagctaccggccaagaaatggacgttgtctccgaagaacctttaattgaatggctagcagctaactacaaaaacttcggtgctaccttggaattcatcacagacaaatcttcagaaggtgcccaatttgtcacaggttttggtggtattggtgccatgctgcgttacaaagttaattttgaacaactagttgatgaatctgaggatgaatattatgacgaagatgaaggatccgactatgatttcatttaa

SEQ ID NO: 115 - DNA, 1314 bp, synthetic construct
  source 1..1314: mol_type = other DNA
atggataacgaggttgaaaaaaatattgagatctggaaggtcaagaagttggtccaatctttagaaaaagctagaggtaatggtacttctatgatttccttagttattcctcctaagggtcaaattccactgtaccaaaaaatgttaacagatgaatatggtcaagctaactctatcaaggacagagttaacagattgtctgttttgtctgctatcacctctacccaacaaaagttgaagttgtacaacaccttgccaaagaacggtttggttttgtactgtggtgacatcatcaccgaagacggtaaggaaaagaaggttaccttcgacatcgaaccatacaagccaatcaacacctctttgtacagatgtgactctaagttccatacagaagttctttcggaattgcttcaagctgacgacaagttcggttttatagtcatggacggtcaaggtactttgtttggttctgtgtccggtaatacgagaactgttttacataaatttactgtcgatctgccaaaaaagcatggtagaggtggtcaatctgcgcttcgttttgctcgtttaagagaagaaaaaagacataattatgtgagaaaggtcgccgaagttgctgttcaaaattttattactaatgacaaagtcaatgttaagggtttaattttagctggttctgctgactttaagaccgatttggctaaatctgaattattcgatccaagactagcatgtaaggttatttccatcgtggatgtttcttatggtggtgaaaacggtttcaaccaggctatcgaactttctgccgaagcgttggccaatgtcaagtatgttcaagaaaagaaattattggaggcatattttgacgaaatttcccaggacactggtaaattctgttatggtatagatgatactttaaaggcattggatttaggtgcagtcgaaaaattaattgttttcgaaaatttggaaactatcagatatacatttaaagatgccgaggataatgaggttataaaattcgctgaaccagaagccaaggacaagtcgtttgctattgacaaagctaccggccaagaaatggacgttgtctccgaagaacctttaattgaatggctagcagctaactacaaaaacttcggtgctaccttggaattcatcacagacaaatcttcagaaggtgcccaatttgtcacaggttttggtggtattggtgccatgctgcgttacaaagttaattttgaacaactagttgatgaatctgaggatgaatattatgacgaagatgaaggatccgactatgatttcatttaa

SEQ ID NO: 116 - DNA, 1314 bp, synthetic construct
  source 1..1314: mol_type = other DNA
atggataacgaggttgaaaaaaatattgagatctggaaggtcaagaagttggtccaatctttagaaaaagctagaggtaatggtacttctatgatttccttagttattcctcctaagggtcaaattccactgtaccaaaaaatgttaacagatgaatatggtggtgctgcttctatcaagaacagagttaacagattgtctgttttgtctgctatcacctctacccaacaaaagttgaagttgtacaacaccttgccaaagaacggtttggttttgtactgtggtgacatcatcaccgaagacggtaaggaaaagaaggttaccttcgacatcgaaccatacaagccaatcaacacctctttgtactcttgtaacaccatcttccatacagaagttctttcggaattgcttcaagctgacgacaagttcggttttatagtcatggacggtcaaggtactttgtttggttctgtgtccggtaatacgagaactgttttacataaatttactgtcgatctgccaaaaaagcatggtagaggtggtcaatctgcgcttcgttttgctcgtttaagagaagaaaaaagacataattatgtgagaaaggtcgccgaagttgctgttcaaaattttattactaatgacaaagtcaatgttaagggtttaattttagctggttctgctgactttaagaccgatttggctaaatctgaattattcgatccaagactagcatgtaaggttatttccatcgtggatgtttcttatggtggtgaaaacggtttcaaccaggctatcgaactttctgccgaagcgttggccaatgtcaagtatgttcaagaaaagaaattattggaggcatattttgacgaaatttcccaggacactggtaaattctgttatggtatagatgatactttaaaggcattggatttaggtgcagtcgaaaaattaattgttttcgaaaatttggaaactatcagatatacatttaaagatgccgaggataatgaggttataaaattcgctgaaccagaagccaaggacaagtcgtttgctattgacaaagctaccggccaagaaatggacgttgtctccgaagaacctttaattgaatggctagcagctaactacaaaaacttcggtgctaccttggaattcatcacagacaaatcttcagaaggtgcccaatttgtcacaggttttggtggtattggtgccatgctgcgttacaaagttaattttgaacaactagttgatgaatctgaggatgaatattatgacgaagatgaaggatccgactatgatttcatttaa

SEQ ID NO: 117 - DNA, 1314 bp, synthetic construct
  source 1..1314: mol_type = other DNA
atggataacgaggttgaaaaaaatattgagatctggaaggtcaagaagttggtccaatctttagaaaaagctagaggtaatggtacttctatgatttccttagttattcctcctaagggtcaaattccactgtaccaaaaaatgttaacagatgaatatggttctgctcaaaacatcaagtctagagttaacagattgtctgttttgtctgctatcacctctacccaacaaaagttgaagttgtacaacaccttgccaaagaacggtttggttttgtactgtggtgacatcatcaccgaagacggtaaggaaaagaaggttaccttcgacatcgaaccatacaagccaatcaacacctctttgtactactgtgacaacagattccatacagaagttctttcggaattgcttcaagctgacgacaagttcggttttatagtcatggacggtcaaggtactttgtttggttctgtgtccggtaatacgagaactgttttacataaatttactgtcgatctgccaaaaaagcatggtagaggtggtcaatctgcgcttcgttttgctcgtttaagagaagaaaaaagacataattatgtgagaaaggtcgccgaagttgctgttcaaaattttattactaatgacaaagtcaatgttaagggtttaattttagctggttctgctgactttaagaccgatttggctaaatctgaattattcgatccaagactagcatgtaaggttatttccatcgtggatgtttcttatggtggtgaaaacggtttcaaccaggctatcgaactttctgccgaagcgttggccaatgtcaagtatgttcaagaaaagaaattattggaggcatattttgacgaaatttcccaggacactggtaaattctgttatggtatagatgatactttaaaggcattggatttaggtgcagtcgaaaaattaattgttttcgaaaatttggaaactatcagatatacatttaaagatgccgaggataatgaggttataaaattcgctgaaccagaagccaaggacaagtcgtttgctattgacaaagctaccggccaagaaatggacgttgtctccgaagaacctttaattgaatggctagcagctaactacaaaaacttcggtgctaccttggaattcatcacagacaaatcttcagaaggtgcccaatttgtcacaggttttggtggtattggtgccatgctgcgttacaaagttaattttgaacaactagttgatgaatctgaggatgaatattatgacgaagatgaaggatccgactatgatttcatttaa

SEQ ID NO: 118 - DNA, 1314 bp, synthetic construct
  source 1..1314: mol_type = other DNA
atggataacgaggttgaaaaaaatattgagatctggaaggtcaagaagttggtccaatctttagaaaaagctagaggtaatggtacttctatgatttccttagttattcctcctaagggtcaaattccactgtaccaaaaaatgttaacagatgaatatggttctgctggtaacatcaagtctagagttaacagattgtctgttttgtctgctatcacctctacccaacaaaagttgaagttgtacaacaccttgccaaagaacggtttggttttgtactgtggtgacatcatcaccgaagacggtaaggaaaagaaggttaccttcgacatcgaaccatacaagccaatcaacacctctttgtacttctgtgacaactctttccatacagaagttctttcggaattgcttcaagctgacgacaagttcggttttatagtcatggacggtcaaggtactttgtttggttctgtgtccggtaatacgagaactgttttacataaatttactgtcgatctgccaaaaaagcatggtagaggtggtcaatctgcgcttcgttttgctcgtttaagagaagaaaaaagacataattatgtgagaaaggtcgccgaagttgctgttcaaaattttattactaatgacaaagtcaatgttaagggtttaattttagctggttctgctgactttaagaccgatttggctaaatctgaattattcgatccaagactagcatgtaaggttatttccatcgtggatgtttcttatggtggtgaaaacggtttcaaccaggctatcgaactttctgccgaagcgttggccaatgtcaagtatgttcaagaaaagaaattattggaggcatattttgacgaaatttcccaggacactggtaaattctgttatggtatagatgatactttaaaggcattggatttaggtgcagtcgaaaaattaattgttttcgaaaatttggaaactatcagatatacatttaaagatgccgaggataatgaggttataaaattcgctgaaccagaagccaaggacaagtcgtttgctattgacaaagctaccggccaagaaatggacgttgtctccgaagaacctttaattgaatggctagcagctaactacaaaaacttcggtgctaccttggaattcatcacagacaaatcttcagaaggtgcccaatttgtcacaggttttggtggtattggtgccatgctgcgttacaaagttaattttgaacaactagttgatgaatctgaggatgaatattatgacgaagatgaaggatccgactatgatttcatttaa

SEQ ID NO: 119 - DNA, 1314 bp, synthetic construct
  source 1..1314: mol_type = other DNA
atggataacgaggttgaaaaaaatattgagatctggaaggtcaagaagttggtccaatctttagaaaaagctagaggtaatggtacttctatgatttccttagttattcctcctaagggtcaaattccactgtaccaaaaaatgttaacagatgaatatggtaccgctcaaaacatcaagtctagagttaacagattgtctgttttgtctgctatcacctctacccaacaaaagttgaagttgtacaacaccttgccaaagaacggtttggttttgtactgtggtgacatcatcaccgaagacggtaaggaaaagaaggttaccttcgacatcgaaccatacaagccaatcaacacctctttgtacttctgtggtggtaagttccatacagaagttctttcggaattgcttcaagctgacgacaagttcggttttatagtcatggacggtcaaggtactttgtttggttctgtgtccggtaatacgagaactgttttacataaatttactgtcgatctgccaaaaaagcatggtagaggtggtcaatctgcgcttcgttttgctcgtttaagagaagaaaaaagacataattatgtgagaaaggtcgccgaagttgctgttcaaaattttattactaatgacaaagtcaatgttaagggtttaattttagctggttctgctgactttaagaccgatttggctaaatctgaattattcgatccaagactagcatgtaaggttatttccatcgtggatgtttcttatggtggtgaaaacggtttcaaccaggctatcgaactttctgccgaagcgttggccaatgtcaagtatgttcaagaaaagaaattattggaggcatattttgacgaaatttcccaggacactggtaaattctgttatggtatagatgatactttaaaggcattggatttaggtgcagtcgaaaaattaattgttttcgaaaatttggaaactatcagatatacatttaaagatgccgaggataatgaggttataaaattcgctgaaccagaagccaaggacaagtcgtttgctattgacaaagctaccggccaagaaatggacgttgtctccgaagaacctttaattgaatggctagcagctaactacaaaaacttcggtgctaccttggaattcatcacagacaaatcttcagaaggtgcccaatttgtcacaggttttggtggtattggtgccatgctgcgttacaaagttaattttgaacaactagttgatgaatctgaggatgaatattatgacgaagatgaaggatccgactatgatttcatttaa

SEQ ID NO: 120 - DNA, 1314 bp, synthetic construct
  source 1..1314: mol_type = other DNA
atggataacgaggttgaaaaaaatattgagatctggaaggtcaagaagttggtccaatctttagaaaaagctagaggtaatggtacttctatgatttccttagttattcctcctaagggtcaaattccactgtaccaaaaaatgttaacagatgaatatggttctgctcaatctatcaagtctagagttaacagattgtctgttttgtctgctatcacctctacccaacaaaagttgaagttgtacaacaccttgccaaagaacggtttggttttgtactgtggtgacatcatcaccgaagacggtaaggaaaagaaggttaccttcgacatcgaaccatacaagccaatcaacacctctttgtacttctgtgacaactctttccatacagaagttctttcggaattgcttcaagctgacgacaagttcggttttatagtcatggacggtcaaggtactttgtttggttctgtgtccggtaatacgagaactgttttacataaatttactgtcgatctgccaaaaaagcatggtagaggtggtcaatctgcgcttcgttttgctcgtttaagagaagaaaaaagacataattatgtgagaaaggtcgccgaagttgctgttcaaaattttattactaatgacaaagtcaatgttaagggtttaattttagctggttctgctgactttaagaccgatttggctaaatctgaattattcgatccaagactagcatgtaaggttatttccatcgtggatgtttcttatggtggtgaaaacggtttcaaccaggctatcgaactttctgccgaagcgttggccaatgtcaagtatgttcaagaaaagaaattattggaggcatattttgacgaaatttcccaggacactggtaaattctgttatggtatagatgatactttaaaggcattggatttaggtgcagtcgaaaaattaattgttttcgaaaatttggaaactatcagatatacatttaaagatgccgaggataatgaggttataaaattcgctgaaccagaagccaaggacaagtcgtttgctattgacaaagctaccggccaagaaatggacgttgtctccgaagaacctttaattgaatggctagcagctaactacaaaaacttcggtgctaccttggaattcatcacagacaaatcttcagaaggtgcccaatttgtcacaggttttggtggtattggtgccatgctgcgttacaaagttaattttgaacaactagttgatgaatctgaggatgaatattatgacgaagatgaaggatccgactatgatttcatttaa

SEQ ID NO: 121 - DNA, 1314 bp, synthetic construct
  source 1..1314: mol_type = other DNA
atggataacgaggttgaaaaaaatattgagatctggaaggtcaagaagttggtccaatctttagaaaaagctagaggtaatggtacttctatgatttccttagttattcctcctaagggtcaaattccactgtaccaaaaaatgttaacagatgaatatggtgctgctaacaacatcaagtctagagttaacagattgtctgttttgtctgctatcacctctacccaacaaaagttgaagttgtacaacaccttgccaaagaacggtttggttttgtactgtggtgacatcatcaccgaagacggtaaggaaaagaaggttaccttcgacatcgaaccatacaagccaatcaacacctctttgtacttctgtggtggtaagttccatacagaagttctttcggaattgcttcaagctgacgacaagttcggttttatagtcatggacggtcaaggtactttgtttggttctgtgtccggtaatacgagaactgttttacataaatttactgtcgatctgccaaaaaagcatggtagaggtggtcaatctgcgcttcgttttgctcgtttaagagaagaaaaaagacataattatgtgagaaaggtcgccgaagttgctgttcaaaattttattactaatgacaaagtcaatgttaagggtttaattttagctggttctgctgactttaagaccgatttggctaaatctgaattattcgatccaagactagcatgtaaggttatttccatcgtggatgtttcttatggtggtgaaaacggtttcaaccaggctatcgaactttctgccgaagcgttggccaatgtcaagtatgttcaagaaaagaaattattggaggcatattttgacgaaatttcccaggacactggtaaattctgttatggtatagatgatactttaaaggcattggatttaggtgcagtcgaaaaattaattgttttcgaaaatttggaaactatcagatatacatttaaagatgccgaggataatgaggttataaaattcgctgaaccagaagccaaggacaagtcgtttgctattgacaaagctaccggccaagaaatggacgttgtctccgaagaacctttaattgaatggctagcagctaactacaaaaacttcggtgctaccttggaattcatcacagacaaatcttcagaaggtgcccaatttgtcacaggttttggtggtattggtgccatgctgcgttacaaagttaattttgaacaactagttgatgaatctgaggatgaatattatgacgaagatgaaggatccgactatgatttcatttaa

SEQ ID NO: 122 - DNA, 1314 bp, synthetic construct
  source 1..1314: mol_type = other DNA
atggataacgaggttgaaaaaaatattgagatctggaaggtcaagaagttggtccaatctttagaaaaagctagaggtaatggtacttctatgatttccttagttattcctcctaagggtcaaattccactgtaccaaaaaatgttaacagatgaatatggtaccgcttctaacatcaagtctagagttaacagattgtctgttttgtctgctatcacctctacccaacaaaagttgaagttgtacaacaccttgccaaagaacggtttggttttgtactgtggtgacatcatcaccgaagacggtaaggaaaagaaggttaccttcgacatcgaaccatacaagccaatcaacacctctttgtacaactgttctggtaagttccatacagaagttctttcggaattgcttcaagctgacgacaagttcggttttatagtcatggacggtcaaggtactttgtttggttctgtgtccggtaatacgagaactgttttacataaatttactgtcgatctgccaaaaaagcatggtagaggtggtcaatctgcgcttcgttttgctcgtttaagagaagaaaaaagacataattatgtgagaaaggtcgccgaagttgctgttcaaaattttattactaatgacaaagtcaatgttaagggtttaattttagctggttctgctgactttaagaccgatttggctaaatctgaattattcgatccaagactagcatgtaaggttatttccatcgtggatgtttcttatggtggtgaaaacggtttcaaccaggctatcgaactttctgccgaagcgttggccaatgtcaagtatgttcaagaaaagaaattattggaggcatattttgacgaaatttcccaggacactggtaaattctgttatggtatagatgatactttaaaggcattggatttaggtgcagtcgaaaaattaattgttttcgaaaatttggaaactatcagatatacatttaaagatgccgaggataatgaggttataaaattcgctgaaccagaagccaaggacaagtcgtttgctattgacaaagctaccggccaagaaatggacgttgtctccgaagaacctttaattgaatggctagcagctaactacaaaaacttcggtgctaccttggaattcatcacagacaaatcttcagaaggtgcccaatttgtcacaggttttggtggtattggtgccatgctgcgttacaaagttaattttgaacaactagttgatgaatctgaggatgaatattatgacgaagatgaaggatccgactatgatttcatttaa

SEQ ID NO: 123 - DNA, 1314 bp, synthetic construct
  source 1..1314: mol_type = other DNA
atggataacgaggttgaaaaaaatattgagatctggaaggtcaagaagttggtccaatctttagaaaaagctagaggtaatggtacttctatgatttccttagttattcctcctaagggtcaaattccactgtaccaaaaaatgttaacagatgaatatggtcaagctcaaaacatcaagtctagagttaacagattgtctgttttgtctgctatcacctctacccaacaaaagttgaagttgtacaacaccttgccaaagaacggtttggttttgtactgtggtgacatcatcaccgaagacggtaaggaaaagaaggttaccttcgacatcgaaccatacaagccaatcaacacctctttgtacttctgtggtggtaagttccatacagaagttctttcggaattgcttcaagctgacgacaagttcggttttatagtcatggacggtcaaggtactttgtttggttctgtgtccggtaatacgagaactgttttacataaatttactgtcgatctgccaaaaaagcatggtagaggtggtcaatctgcgcttcgttttgctcgtttaagagaagaaaaaagacataattatgtgagaaaggtcgccgaagttgctgttcaaaattttattactaatgacaaagtcaatgttaagggtttaattttagctggttctgctgactttaagaccgatttggctaaatctgaattattcgatccaagactagcatgtaaggttatttccatcgtggatgtttcttatggtggtgaaaacggtttcaaccaggctatcgaactttctgccgaagcgttggccaatgtcaagtatgttcaagaaaagaaattattggaggcatattttgacgaaatttcccaggacactggtaaattctgttatggtatagatgatactttaaaggcattggatttaggtgcagtcgaaaaattaattgttttcgaaaatttggaaactatcagatatacatttaaagatgccgaggataatgaggttataaaattcgctgaaccagaagccaaggacaagtcgtttgctattgacaaagctaccggccaagaaatggacgttgtctccgaagaacctttaattgaatggctagcagctaactacaaaaacttcggtgctaccttggaattcatcacagacaaatcttcagaaggtgcccaatttgtcacaggttttggtggtattggtgccatgctgcgttacaaagttaattttgaacaactagttgatgaatctgaggatgaatattatgacgaagatgaaggatccgactatgatttcatttaa

SEQ ID NO: 124 - DNA, 1314 bp, synthetic construct
  source 1..1314: mol_type = other DNA
atggataacgaggttgaaaaaaatattgagatctggaaggtcaagaagttggtccaatctttagaaaaagctagaggtaatggtacttctatgatttccttagttattcctcctaagggtcaaattccactgtaccaaaaaatgttaacagatgaatatggtcaagctgactgtatcaagtctagagttaacagattgtctgttttgtctgctatcacctctacccaacaaaagttgaagttgtacaacaccttgccaaagaacggtttggttttgtactgtggtgacatcatcaccgaagacggtaaggaaaagaaggttaccttcgacatcgaaccatacaagccaatcaacacctctttgtactcttgtgacggtgttttccatacagaagttctttcggaattgcttcaagctgacgacaagttcggttttatagtcatggacggtcaaggtactttgtttggttctgtgtccggtaatacgagaactgttttacataaatttactgtcgatctgccaaaaaagcatggtagaggtggtcaatctgcgcttcgttttgctcgtttaagagaagaaaaaagacataattatgtgagaaaggtcgccgaagttgctgttcaaaattttattactaatgacaaagtcaatgttaagggtttaattttagctggttctgctgactttaagaccgatttggctaaatctgaattattcgatccaagactagcatgtaaggttatttccatcgtggatgtttcttatggtggtgaaaacggtttcaaccaggctatcgaactttctgccgaagcgttggccaatgtcaagtatgttcaagaaaagaaattattggaggcatattttgacgaaatttcccaggacactggtaaattctgttatggtatagatgatactttaaaggcattggatttaggtgcagtcgaaaaattaattgttttcgaaaatttggaaactatcagatatacatttaaagatgccgaggataatgaggttataaaattcgctgaaccagaagccaaggacaagtcgtttgctattgacaaagctaccggccaagaaatggacgttgtctccgaagaacctttaattgaatggctagcagctaactacaaaaacttcggtgctaccttggaattcatcacagacaaatcttcagaaggtgcccaatttgtcacaggttttggtggtattggtgccatgctgcgttacaaagttaattttgaacaactagttgatgaatctgaggatgaatattatgacgaagatgaaggatccgactatgatttcatttaa

SEQ ID NO: 125 - DNA, 1314 bp, synthetic construct
  source 1..1314: mol_type = other DNA
atggataacgaggttgaaaaaaatattgagatctggaaggtcaagaagttggtccaatctttagaaaaagctagaggtaatggtacttctatgatttccttagttattcctcctaagggtcaaattccactgtaccaaaaaatgttaacagatgaatatggtagagctcaaaacatcaagtctagagttaacagattgtctgttttgtctgctatcacctctacccaacaaaagttgaagttgtacaacaccttgccaaagaacggtttggttttgtactgtggtgacatcatcaccgaagacggtaaggaaaagaaggttaccttcgacatcgaaccatacaagccaatcaacacctctttgttcttgtgtgaaaacaccttccatacagaagttctttcggaattgcttcaagctgacgacaagttcggttttatagtcatggacggtcaaggtactttgtttggttctgtgtccggtaatacgagaactgttttacataaatttactgtcgatctgccaaaaaagcatggtagaggtggtcaatctgcgcttcgttttgctcgtttaagagaagaaaaaagacataattatgtgagaaaggtcgccgaagttgctgttcaaaattttattactaatgacaaagtcaatgttaagggtttaattttagctggttctgctgactttaagaccgatttggctaaatctgaattattcgatccaagactagcatgtaaggttatttccatcgtggatgtttcttatggtggtgaaaacggtttcaaccaggctatcgaactttctgccgaagcgttggccaatgtcaagtatgttcaagaaaagaaattattggaggcatattttgacgaaatttcccaggacactggtaaattctgttatggtatagatgatactttaaaggcattggatttaggtgcagtcgaaaaattaattgttttcgaaaatttggaaactatcagatatacatttaaagatgccgaggataatgaggttataaaattcgctgaaccagaagccaaggacaagtcgtttgctattgacaaagctaccggccaagaaatggacgttgtctccgaagaacctttaattgaatggctagcagctaactacaaaaacttcggtgctaccttggaattcatcacagacaaatcttcagaaggtgcccaatttgtcacaggttttggtggtattggtgccatgctgcgttacaaagttaattttgaacaactagttgatgaatctgaggatgaatattatgacgaagatgaaggatccgactatgatttcatttaa

SEQ ID NO: 126 - DNA, 1344 bp, synthetic construct
  source 1..1344: mol_type = other DNA
atgtctatcatcgactctaacgttgaaacctggaagatcaagagaatcatcaagaacttggaaagattgagaggtaacggtacctctatgatctctttgttgttgtctccacgcgacgctatcccaaaggttcaaggtatgttggctggtgaatacggtaccgctgaatctatcaagtctaagatcaacagattggctgttcaaggtgctatcacctctgctaaggaaagattgaagttgtacaacagaaccccaccaaacggtttggttatctactgtggtatcgttatcggtgaagacaagtctgaaaagaagtactgtatcgacttcgaaccattcagaccattgaacaccttcaagtacatctgtgacaacaagttctacaccaagccattgttcgaattgttggaaaacgacgacgttttcggtttcgttatcgttgacggttctggttgtttgttcggtaccttgcaaggtaacaccaagaagatcatccaaaacatcaccgtttctttgccaaagaagcacggtagaggtggtcaatctgctccaagattcggtagaatcagagaagaaaagagacacaactacgttagaaaggttgctgaattcgctacccaacacttcatcaccgaagacaagccaaacgttaagggtatcatcttggctggttctgctaacttcaagaacgacttgtctgaatctgacttgttcgacaagagattgtctgaaatcgttttgaagatcgttgacgtttcttacggtggtgaaaacggtttctctcaagctatcaccttggctgaagacaccttgtctaacgttaagttcgttgaagaaaagaacttgatctctaagtacttcgaagaaatcgctcaagacaccggtatggttgttttcggtatcgaagacaccttgaactctttggaattgggtgctgttggtaccatcatctgtttcgaaaacttggaaatcaacagatacgaaatcagaaacccatctaccgaagaaatcaaggttatccacttgtgtaaggaccaacaaaacgacaccagatacaagatgatcgacaacaactactcttacttcatcgaccaaaacaccggtttggacttggaaatcttgtcttgtgttccattgaccgaatggttgtgtgaaaactactctaagtacggtgttagattggaattcatcaccgacaagtctcaagaaggtttccaattcgttaacggtttcggtggtatcggtggtttcttgagattcaagttggaaatcgaaaacatcgactacgaaggtgaagacgttggtggtgaagaattcgacgctgacgaagacttcatctaatag

SEQ ID NO: 127 - DNA, 1317 bp, synthetic construct
  source 1..1317: mol_type = other DNA
atggctaagttggacgacaacgttgaaacctggagaatcaagagattgatcaagaacttggaaaagttgagaggtgacggtacctctatgatctctttgttgttgtctccacgcgaccaaatctctaaggttcaagctatgttggctggtgaagctggtaccgctgttaacatcaagtctagagttaacagacaagctgttttgtctgctatcacctctgctaaggaaagattgaagttgtactctaagaccccaaccaacggtttggttgtttactgtggtaccgttatcggtgaagacgactctgaaaagaagtacaccatcgacttcgaaccattcagaccattgaacaccttcaagtacatctgtgacaacaagttctgtaccgaaccattgttcgaattgttggaaaacgacgacgttttcggtttcgttatcgttgacggtaacggttgtttgttcggtaccttgcaaggtaacaccaagaagatcttgcaacaaatcaccgtttctttgccaaagaagcacggtagaggtggtcaatctgctccaagattcggtagaatcagagaagaaaagagacacaactacgttagaaaggttgctgaattggctacccaacacttcatcaccgacgacagaccaaacgttaagggtttggttttggctggttctgctaacttcaagaacgacttgtctgaatctgacttgttcgacaagagattgtctgaagttgttatcaagatcgttgacgtttcttacggtggtgaaaacggtttctctcaagctatctctttggctgaagacgctttgtctaacgttaagttcgttgaagaaaagaacttgatctctaagtacttcgaagaaatcgctttggactctggtatgatcgttttcggtgttgaagacaccttgcactctttggaagttggtgctttggacttgttgatgtgtttcgaaaacttggaaatcaacagatacgaaatccgcgacccagctaacgacgaaatcaagatctacaacttgaacaaggaacaagaaaaggactctaagtacttcaagaacgaaaagaccggtaccgacttggaaatcgttaagtgtgttgctttgtctgaatggttgtgtgaaaactactctaagtacggtgttaagttggaattcatcaccgacaagtctcaagaaggtttccaattcgttaacggtttcggtggtatcggtggtttcttgagatacaagttggaaatggaaaaccacgactacgacaaggaagacgttggtggtgaagaattcaacccagacgaagacttcatctaatag

SEQ ID NO: 128 - DNA, 1314 bp, synthetic construct
  source 1..1314: mol_type = other DNA
atggaaggtgacgaattgacccaaaacatcgaacaatggaagatcaagagattgatcgacaacttggacaaggctagaggtaacggtacctctttgatctctttgatcatcccaccaagagaacaattgccaatcatcaacaagatgatcaccgaagaatacggtaagtcttctaacatcaagtctagaatcgttagacaagctgttcaatctgctttgacctctaccaaggaaagattgaagttgtacaacaacagattgccagctaacggtttgatcttgtactgtggtgaagttatcaacgaagaaggtgtttgtgaaaagaagtacaccatcgacttccaaccatacagagctatcaacaccaccttgtacatctgtgacaacaagttccacacccaaccattgaaggacttgttggttatggacgacaagttcggtttcatcatcatcgacggtaacggtgctttgttcggtaccttgcaaggtaacaccagagaagttttgcacaagttctctgttgacttgccaaagaagcacagaagaggtggtcaatctgctttgagattcgctagattgagaatggaatctagaaacaactacttgagaaaggttgctgaacaagctgttgttcaattcatctctaacgacaaggttaacgttgctggtttgatcgttgctggttctgctgaattcaagaacgttttggttcaatctgacttgttcgaccaaagattggctgctaaggttttgaagatcgttgacgttgcttacggtggtgaaaacggtttcacccaagctatcgaattgtctgctgacaccttgtctaacatcaagttcatcagagaaaagaaggttatgtctaagttcttcgaagaagttgctcaagacaccaagaagtactgttacggtgttgaagacaccatgaagaccttgatcatgggtgctgttgaagttatcttgttgttcgaaaacttgaacttcaccagatacgttttgaagaacccaaccaccggtgttgaaaagaccttgtacttgaccccagaacaagaagaaaaccacgacaacttcatggaaaacggtgaagaattggaagctttggaaaagggtccattgccagaatggatcgttgacaactacatgaagttcggtgctggtttggaattcatcaccgacagatctcaagaaggtgctcaattcgttagaggtttcggtggtttgggtgctttcttgagataccaagttgacatggctcacttgaacgctggtgaagaagaattggacgaagaatgggacgacgacttcatgtaatag

SEQ ID NO: 129 - DNA, 1311 bp, synthetic construct
  source 1..1311: mol_type = other DNA
atggaagaaaaggaccaaagacaaagaaacatcgaacacttcaagatcaagaagttgatgaccagattgagaaacaccagaggttctggtacctctatggtttctttgatcatcccaccaaagaagcaaatcaacgactctaccaagttgatctctgacgaattctctaaggctaccaacatcaaggacagagttaacagacaatctgttcaagacgctatggtttctgctttgcaaagattgaagttgtaccaaagaaccccaaacaacggtttgatcttgtactgtggtaaggttttgaacgaagaaggtaaggaaatcaagttgttgatcgacttcgaaccatacaagccaatcaacacctctttgtacttctgtgactctaagttccacgttgacgaattgggttctttgttggaaaccgacccaccattcggtttcatcgttatggacggtcaaggtgctttgtacgctaacttgcaaggtaacaccaagaccgttttgaacaagttctctgttgaattgccaaagaagcacggtagaggtggtcaatcttctgttagattcgctagattgagagttgaaaagagacacaactacttgagaaaggtttgtgaagttgctacccaaaccttcatctctcaagacaagatcaacgttcaaggtttggttttggctggttctggtgacttcaagaacgaattgtctaccacccaaatgttcgacccaagattggcttgtaagatcatcaagatcgttgacgtttcttacggtggtgaaaacggtttgaaccaagctatcgaattggctcaagaatctttgaccaacgttaagttcgttcaagaaaagaacgttatctctaagttcttcgactgtatcgctatcgactctggtaccgttgtttacggtgttcaagacaccatgcaattgttgttggacggtgttatcgaaaacatcttgtgtttcgaagaattgaccaccttgagagttaccagaaagaacaaggttaccgaacaaatcacccacatcttcatcccaccaaacgaattgaacaacccaaagcacttcaaggacggtgaacacgaattggaaaagatcgaagttgaaaacttgaccgaatggttggctgaacactactctgaattcggtgctgaattgtacttcatcaccgacaagtctgctgaaggttgtcaattcgttaagggtttctctggtatcggtggtttcttgagatacaaggttgacttggaacacatcgttaacccaaacgacgaatacaactacgaagaagaagaaggtttcatctgatag

SEQ ID NO: 130 - DNA, 1461 bp, synthetic construct
  source 1..1461: mol_type = other DNA
atggaacaaaagccaccattccaaaacccattgcaaaagttgcaagacagaggtaccaagatggaccaatcttctggttcttgtatgtctaagcaagctgaagaacaaaagagattgcaaatctctcaataccaattgagaaagcaattgcaaatgttgagaaacatgagaggtgaacaaacctcttgtgtttctttgtacatcccagaaagaaagaagttgtacgaagttgttaactacttgcaacaagaagaatctggtgctgcttctatcaagaacacccaaaacagaaagtctgttcaatctgctttgtctatgttgagagaaagattgaagaacttcaacttgcacaagaagtacccaaagggtatgatcttcttctgtgctgactctttggactctaagagattgttgatcgaaatcttggacccaccaaaggctgttcaatctttcagatactcttgtaacaccatcttctacttggacgacttggaatacatgttgaaggaccaaccaacctacggtttcgttgttgctgacggtcacggttacttgatcgctaccgtttgtggtttcgacatccaaatcttgcaatctaagcaagaagacttgccaaacaagcacaacaagggtggtcaatcttctttgagattctctagattgtgtgacgctgctagagaaagattggttaagaacatcgctgacgctatgagaagatgttacgctaacgaaaacggtacccaaaccaacttgtctggtatcgttttgtgtggtatgtctgacatcaaggacaaggttcaaaaggaattgcaacaattgtgtccatgtatcgaaaacaagatcgttgcttcttacgacgtttcttactctggtcaagctggtttgaagcaagctttgcaaatgtctaccgaaatgttgaagttggaccaattgttccaagaaatgaacttgttgtctgacttcttcgctaacttctctttggaaacctctaaggttgtttacggtggtgaattgaccgttagagctttggaagaaggtaacgttaagaagttgatcttgtgtcaagactctgaattgcaaagagttaccgtttacaactctaagacccaagaagaaaccatccaatacttgatgccatctcaagttaaggctttgcaagactctatctctaagacctctgaccaagaagctaacaacaagaagaaccaattgcaagtttactctcaacaaaacatcaacgaatggatcgttgaaaacatctcttctttctctcaagacttggaaatcgttttcgtttctgacaagacccaacaaggtgttcaattctctaagtctttccaaggtgttggtgcttacttgaagtactctttggactactcttctttgcacgctcaagaaaaggaaaacgaccaattggaacaagaatactgttacgacgacgaagaaggtttcatctgatag

SEQ ID NO: 131 - DNA, 1362 bp, synthetic construct
  source 1..1362: mol_type = other DNA
atgatcaagaacatcttcaagttgttgccaatctctttgagagctatcccattgaagcaacaacaaaactctttctctcaaatctgttctttgtacaacaccaagttgttcaaggttatcaacttgatccaaaccaacaacaagtgtttcttctctttcagagctaaggaaaccttcaagaagaagacctcttctttggaaatcgaaacccacttccaagtttctgacttgaccagatgtatctacagaagaatgaagcaattccacaacgaatacaccgacatccaaaagatcttgtctcaagaacaacaacaagctgacatcaacttggaacaattgagaaagaagatcaacgttttgcaaccattgaacgacgttttcgaaaagttggaacaaaacatcaagaccttgcaagaattgcaaaagcaaaaggaagaatctgcttctgacccagaaatgttggctttgatcgaagaagaaatggaaaactctaagcaattgatcgacgaattgcaagacgaatgtttggaacaattgttgccaaagggtaagcacgacgactgttctgaaatcaccttggaagttagaggtggtgctggtggttctgaatcttctttgttcgctgaagaagttttcaagatgtaccaagctttcttcgctcaacaaggttaccaattctctatcgactctttccaagttgacatggctatcaacaagggttgtaagttgggtgttttgaaggtttctggtaccaacatctacaagaagatgatgaacgaatctggtgttcacaaggttatcagagttccagaaaccgaatctaagggtagattgcactcttctaccatctctgttgttgttatgccagttgttccaatggacttcaaggttgacgaaaaggacttgaagttcgaattcatgagatctcaaggtgctggtggtcaacacgttaacaaggttgaatctgcttgtagagttacccacttgccaaccggtatctctgttttgtgtcaagacgacagacaacaagaaagaaacaagcaaagagctttgaagttgttgaccgaaaagttgttccaagttgaagttgaaaagtctaaccaacaacaatctgaccaaagaaagtctcaaatcggtggtggtgacagatctgacaagatcagaacctacaacttcccacaaggtagaatcaccgaccacagaaccaacttgaccttgttcggtatcgaaaagatgatgaagggtgaattcttggaagaattcatcgacgaatacgaagaaaaggttaacaacgaattgatcgaatctgttttgaagcaattggaagaagacgaaaaccaatctcaaccaaagaactgatag

SEQ ID NO: 132 - DNA, 1317 bp, synthetic construct
  source 1..1317: mol_type = other DNA
atggaccaaaagttgaacgacgctgaaatcgctttggaacaattcagattgaagaagttgatcaagaccttgtctcaagaaagaaccgctggtacctctgttgtttctgtttacatcccaccaaagagaatcatctctgacatcaccaacagattgaacacccaatacgctgaagctgcttctatcaaggacaagggtaacagaatctctgttcaagaagctatccaagctgctatcttgagactcagaccatacaacaaggctccaaacaacggtttggttgttttctgtggtatcgttcaacaagctgacggtaagggtgaaaagaagatctctgttgttatcgaaccatacagaccattggacttgtctttgtacttctgtgacccacaattccacgttgaagaattgagagctttgttgaacatcgacccaccattcggtttcatcatcatggacggtaacggttctttgttcgctaccatccaaggtaactctaagcaaatcatcaagtctttcgacgttgacttgccaaagaagcacaacaagggtggtcaatcttctgttagattcgctagattgagaatggaaaagagacacaactacttgagaaaggtttgtgaaaccgctaccacctgtttcatcgctgaagacagaccaaacgttaagggtttggttttggctggttctgctgacttcaagaacgacttggctggttctcaattcttcgacaagagattgcaaccattgatcatctctgttgttgacatcaactacggtggtgaacaaggtttgaaccaagctgttcaattgtctcaagaatctttgttggaagttaagtacatcagagaaaagaacttggttggtcaattcttcgaaaacatcgacaaggacaccggtttggttgtttacggtgttcaagacaccatgagagctgttgaatctcaaaccatcaagaccttggtttgtgttgacaccttgcaatacttgagattggaatgtcaatctaagcaaaccgaacaaaaggctatcaagtacatcaagggtaacgaaggttacgaagctggttctttgatcgaagaaaagaacggtgaacaattcgttatcttggttaaggaagacttggttgaacacttgtctgaaaagttcaaggactacggtttggacttccaattgatcaccgaccactctgttgaaggtaaccaattcatgaagggtttctctggtttgggtggtttcttgagattcaagatggacatggactacttggttcaacaagaagactggaaggacgaagacgaagacttcatctgatag

SEQ ID NO: 133 - DNA, 1317 bp, synthetic construct
  source 1..1317: mol_type = other DNA
atgaaccaaaaccaaatccaagaacaagaattggaaatcgaacaattcagattgtctaagatcatcaagaccttgtctaagaccaaggttatcggtacctctgctgtttctttgtacatcccaccaaagaagatcatctctgacatcaccaacagattgaacacccaattctctgaagctgcttctatccaagacaaggttaacagaacctctgttcaagactctatccaaggtgctgttttgaagttgaagaagtacaccaaggctccagcttctggtttggttttgttctctggtttggttgaattcgaaaagggtcaaaagaagatctcttacgttatcgaaccattcagaccattgcaattgtctttgttcttctgtgacaactacttccacatcgaacaattggaaccattgttgaagttggaaccatcttacggtttcatcatcatggacggtaacggtgctttgttcggtaaggttcaaggtatctctaaggaaaccttgaagtctttcaacgttgacttgccaaagaagcacaacaagggtggtcaatcttctttgagattctctagaatcagatactgggctagacacaactacttgatcaaggtttctgaacaagctaagaactgtttcatctctgacgacaagccaaccatcaagggtttggttttggctggtatcgctgacttcaagaacaagttggctgaatctccagctttggacaagagattgcaaccattgatcttgtctatcgttgacgttaactacggtggtgaaaacggtttcaaccaagctatccaatactctcaagaagttttgcaaaaccaaaagttgcaaagagaaaaggacttggttgctaagttcttcttgtctttggacttggacaacggtaagtctgtttacggtgttgttgacaccatgaaggctatcgaacaagaattggttaagcaagttatctgtatccaaaccttggaatactctagagttgaatgtatctctaagcaaaccggtgttaagtctatcaagtacttgaagggtttggacttgtacgaacaaggttctttgttcgaagacaacaagggtgaacaattccaagttacctcttgtcaagacttggttgaatacttggctgaaaactacagagaaaagggtatcgacttccaattgatctctgacaactctgctgaaggtcaccaattctacaagggtttcggtggtatggctggtttcttcagattctctatgaagatgcaatacaacatggactctgaagaagaatggaagtctgaagacgacgaattcatctgatag

SEQ ID NO: 134 - DNA, 1338 bp, synthetic construct
  source 1..1338: mol_type = other DNA
atggttgaatctatcgctgctggtcaagttggtgacaacaagcacatcgaaatgtggaagatcaagagattgatcaacaagttggaaaactgtaagggtaacggtacctctatggtttctttgatcatcccaccaaaggaagacatcaacaagtctggtaagttgttggttggtgaattgtctgctgctcaaaacatcaagtctagaatcaccagacaatctgttatcaccgctatcacctctaccaaggaaaagttgaagttgtacagacaaaccccaaccaacggtttgtgtatctactgtggtgttatcttgatggaagacggtaagaccgaaaagaagatcaacttcgacttcgaaccattcagaccaatcaaccaattcatgtacttctgtggtggtaagttccaaaccgaaccattgaccaccttgttggctgacgacgacaagttcggtttcatcatcgttgacggtaacggtgctttgtacgctaccttgcaaggtaactctagagaaatcttgcaaaagatcaccgttgaattgccaaagaagcacagaaagggtggtcaatcttctgttagattcgctagattgagagaagaaaagagacacaactacttgagaaaggttgctgaattggctggttctaacttcatcaccaacgacaagccaaacgttaccggtttggttttggctggtaacgctggtttcaagaacgaattgtctgaaaccgacatgttggacaagagattgttgccaatcatcgtttctatcgttgacgtttcttacggtggtgaaaacggtttgaacgaagctatcaccttgtctgctgacgctttgaccaacgttaagttcgttgctgaaaagaagttggtttctaagttcttcgaagaaatctctttggacaccggtatgatcgttttcggtgttcaagacaccatgaaggctttggaattgggtgctgttgaaaccatcttgttgttcgaagaattggaaatcaccagatacgttatcaagaacccagttaagggtgacaccagaaccttgttcttgaacccaacccaacaaaaggactctaagtacttcaaggaccaagcttctggtttggacatggacgttatctctgaagaccaattggctgaatggttgtgtcacaactaccaaaactacgctcaagttgaattcatcaccgacaagtctcaagaaggttaccaattcgttaagggtttcggtggtatcggtggtttcttgagatacaaggttgacatggaagaagctttgggtgacgttggtgacggtggtgacgacttcgacccagacaccgacttcatctgatag

SEQ ID NO: 135 - DNA, 1317 bp, synthetic construct
  source 1..1317: mol_type = other DNA
atggacgaagctaagttgttgcaattggaaatgtggagattgagaaagcaattgcaaaagttggacaacaccaacaccaactctacctctgttgtttctttggttatgccaccaggtgaagacatcaacaagatggttcaaatgttgaaccaagaagctacccaagctgactgtatcaagtctagacaaaacagacaagctgttcaaaccgctatcatcttggctgctaacagatgtaagttgtacccaaagatgccaaagaacggtttggctgttttcgctggtgaagtttacgttgacggtaagatcaagaagatcgctgttcacttctctccatgtaagccaatcggtaacttcatgtactcttgtgacggtgttttccacacccaagaagttaaggacttgttgaccgttgaagaagtttacggtttcatcatcatggacggtcacggtaccttgatcgctaccttgtgtggttctcacagagaaatcaagcacagaatgttggttgacttgccaaagaagcacggtagaggtggtcaatcttctgttagattcgctagattgagaatggaagctagaggtaactacgttaagatcatcaccgaattgtgtaccaagtacttcatcaccggtgacagattgaacgtttctggtgttatcttggctggttctgctgacttcaaggacgttttggctggttctgacttcatggacccaagaatcaaggaaggtatcatcaagcaagttgacatcggttacggtatggaacaaggtttgtctcaagctatcgaacaagctggtgacatcttgaaggacgttagattgttcaaggaagttaagatcatcaacgaattcttggaccacatctctagagacaccagaaagtactgtttcggtatcatcgacaccttgagatgtttggaaatgggttctgttgaacacttggttgtttgggaagacttcccatgggacagagttcaatgtagaaactctgaaggtaccgaatactacaagttggttaacaccgttaccggtgctatcaacttggttgacaacgacgaatctatcagacaaggtgaagaagaaatcgaagctgaattgttcgttgaatggttggctcaaaacatcgaaaagttcggtgctgaaatccacttggttaccgaaaactctaacgaaggtacccaattctgttctggtttcggtggtttgggtggtatcttgagatggcaaatggacttgaacgaattgggtaagtacatggacatcggtaaggaaaacaacgacttggacgacttggaagacttcatgtgatag

SEQ ID NO: 136 - DNA, 1317 bp, synthetic construct
  source 1..1317: mol_type = other DNA
atggacaacgaagttgaaaagaacatcgaaatctggaaggttaagaagttggttcaatctttggaaaaggctagaggtaacggtacctctatgatctctttggttatcccaccaaagggtcaaatcccattgtaccaaaagatgttgaccgacgaatacggtaccgcttctaacatcaagtctagagttaacagattgtctgttttgtctgctatcacctctacccaacaaaagttgaagttgtacaacaccttgccaaagaacggtttggttttgtactgtggtgacatcatcaccgaagacggtaaggaaaagaaggttaccttcgacatcgaaccatacaagccaatcaacacctctttgtacttgtgtgacaacaagttccacaccgaagttttgtctgaattgttgcaagctgacgacaagttcggtttcatcgttatggacggtcaaggtaccttgttcggttctgtttctggtaacaccagaaccgttttgcacaagttcaccgttgacttgccaaagaagcacggtagaggtggtcaatctgctttgagattcgctagattgagagaagaaaagagacacaactacgttagaaaggttgctgaagttgctgttcaaaacttcatcaccaacgacaaggttaacgttaagggtttgatcttggctggttctgctgacttcaagaccgacttggctaagtctgaattgttcgacccaagattggcttgtaaggttatctctatcgttgacgtttcttacggtggtgaaaacggtttcaaccaagctatcgaattgtctgctgaagctttggctaacgttaagtacgttcaagaaaagaagttgttggaagcttacttcgacgaaatctctcaagacaccggtaagttctgttacggtatcgacgacaccttgaaggctttggacttgggtgctgttgaaaagttgatcgttttcgaaaacttggaaaccatcagatacaccttcaaggacgctgaagacaacgaagttatcaagttcgctgaaccagaagctaaggacaagtctttcgctatcgacaaggctaccggtcaagaaatggacgttgtttctgaagaaccattgatcgaatggttggctgctaactacaagaacttcggtgctaccttggaattcatcaccgacaagtcttctgaaggtgctcaattcgttaccggtttcggtggtatcggtgctatgttgagatacaaggttaacttcgaacaattggttgacgaatctgaagacgaatactacgacgaagacgaaggttctgactacgacttcatctgatag

SEQ ID NO: 137 - DNA, 2058 bp, synthetic construct
  source 1..2058: mol_type = other DNA
atgtctgactctaaccaaggtaacaaccaacaaaactaccaacaatactctcaaaacggtaaccaacaacaaggtaacaacagataccaaggttaccaagcttacaacgctcaagctcaaccagctggtggttactaccaaaactaccaaggttactctggttaccagcagggggggtatcagcagtataatcctgacgctggttaccaacaacaatacaacccacaaggtggttaccagcagtataatcctcagggcggctatcagcagcaattcaacccacaaggtggtagaggtaactacaagaacttcaactacaacaacaacttgcaaggttaccaagctggtttccaaccacaatctcaaggtatgtctttgaacgacttccaaaagcaacaaaagcaagctgctccaaagccaaagaagaccttgaagttggtttcttcttctggtatcaagttggctaacgctaccaagaaggttgacaccaagccagctgaatctgacaagaaggaagaagaaaagtctgctgaaaccaaggaaccaacgaaagagcctacgaaagttgaagaaccagttaagaaggaagaaaagccagttcaaaccgaagaaaagaccgaagaaaagtctgaattgccaaaggttgaagacttgaagatctctgaatctacccacaacaccaacaacgctaacgttacctctgctgacgctttgatcaaggaacaagaagaagaagttgacgacgaagttgttaacgacatgttcggtggtaaggaccacgtttctttgatcttcatgggtcacgttgacgctggtaagtctaccatgggtggtaacttgttgtacttgaccggttctgttgacaagagaaccatcgaaaagtacgaaagagaagctaaggacgctggtagacaaggttggtacttgtcttgggttatggacaccaacaaggaagaaagaaacgacggtaagaccatcgaagttggtaaggcttacttcgaaaccgaaaagagaagatacaccatcttggacgctccaggtcacaagatgtacgtttctgaaatgatcggtggtgcttctcaagctgacgttggtgttttggttatctctgctagaaagggtgaatacgaaaccggtttcgaaagaggtggtcaaaccagagaacacgctttgttggctaagacccaaggtgttaacaagatggttgttgttgttaacaagatggacgacccaaccgttaactggtctaaggaaagatacgaccaatgtgtttctaacgtttctaacttcttgagagctatcggttacaacatcaagaccgacgttgttttcatgccagtttctggttactctggtgctaacttgaaggaccacgttgacccaaaggaatgtccatggtacaccggtccaaccttgttggaatacttggacaccatgaaccacgttgacagacacatcaacgctccattcatgttgccaatcgctgctaagatgaaggacttgggtaccatcgttgaaggtaagatcgaatctggtcacatcaagaagggtcaatctaccttgttgatgccaaacaagaccgctgttgaaatccaaaacatctacaacgaaaccgaaaacgaagttgacatggctatgtgtggtgaacaagttaagttgagaatcaagggtgttgaagaagaagacatctctccaggtttcgttttgacctctccaaagaacccaatcaagtctgttaccaagttcgttgctcaaatcgctatcgttgaattgaagtctatcatcgctgctggtttctcttgtgttatgcacgttcacaccgctatcgaagaagttcacatcgttaagttgttgcacaagttggaaaagggtaccaacagaaagtctaagaagccaccagctttcgctaagaagggtatgaaggttatcgctgttttggaaaccgaagctccagtttgtgttgaaacctaccaagactacccacaattgggtagattcaccttgcgcgaccaaggtaccaccatcgctatcggtaagatcgttaagatcgctgaatga

SEQ ID NO: 138 - DNA, 1344 bp, synthetic construct; identical to SEQ ID NO: 126
  source 1..1344: mol_type = other DNA

SEQ ID NO: 139 - DNA, 2403 bp, synthetic construct
  source 1..2403: mol_type = other DNA
atggacaagaactctgacttcccagctttgggttctttgaaggttcaaaaccaatcttctaaggacaagaacaagaagaaggaaaaggaagttaagaaggaagacgaaaagaaggacgaagttaaggacgaaccacaaaacaacgaagaagctaagaagaccaagttggaagctaccggtagagttttcgctgctaagtctttcaccccaaaggaaccagttaagatcaactactacatcccacacgaagaaccaaccttgtctgaaatggacttcttcccaaccttgggtggtccagcttctaccgctccagttgtttctgaagctcaaaagagacacgctgaattcttggaaagacacgaattgttcaagccatacatctgtttgatcccaaaggaattgtggtacgttccagacccatacttcgacatgttcggttacccagttatggctttgttgccaggtttgtacgcttacttgtggaacacctacaacatcttcttcaccccagaaggtgcttggaacaccaacaaggaaaccgttatgtctaccttgggtgaattgtggacccacgctgaatggagagacagaatcatcgctaaggaacaaaaggaaaccgaagaatgggaaagacaaatgagagaatgggaagaagaaaacgctgaagacgacgaaggtttgtctatcgacgacatggaaaactacggtaagggtaagaagaagaacaagaacaagaagaagaaggacaaggctaaaagaccaccaccaccaccaccaaagtctttgtcttcttacaagagattcgaaaagaagaaggaagacgttgttgttccaaagaagaacatcggtttcaaggaagtttctgaaatcaccttcgaagaagaagttgttgaagttgacgaaaccagacaaccatcttctttggttttcatcggtccagttgacgctgttaagtctaccatctgtggtaacttgatgttcatgaccggtatggttgacgaaagaaccatcgaaaagttcaagcaagaagctaaggaaaagaacagagactcttggtggttggcttacgttatggacatcaacgacgacgaaaagtctaagggtaagaccgttgaagttggtagagctaccatggaaaccccaaccaagagatacaccatcttcgacgctccaggtcacaagaactacgttccagacatgatcatgggtgctgctatggctgacgttgctgctttggttatctctgctagaaagggtgaattcgaagctggtttcgaacgcgacggtcaaaccagagaacacgctcaattggctagatctttgggtgttaacaagttggttgttgttgttaacgaaatggacgaagaaaccgttcaatggtctgaagaaagatacaacgacatcttgtctggtgttaccccattcttgatcgaccaatgtggttacaagagagaagacttgatcttcgttccaatctctggtttgaacggtcacaacatcgacaagttggcttcttgttgtccatggtacaccggtccaaccttgttggaaatcttggactgtatcgaaccaccaaagagaaacatcgacggtccattgagagttccagttttggacaagatgaaggacagaggtgttgttgctttcggtaaggttgaatctggtgttatcagaatcggtccaaagttggctgttatgccaaacaacaccaagtgtcaagttgttggtatctacaactgtaagttggaattggttagatacgctaacccaggtgaaaacatccaaatcaaggttagaatgatcgaagacgaaaaccaaatcaacaagggtgacgttttgtgtccatacgacaacttggctccaatcaccgacttgttcgaagctgaattgaccatcttggaattgttgccacacagaccaatcatcaccccaggttacaagtctatgatgcacttgcacaccatctctgacgaaatcgttatccaaaccttgaccggtatctacgaattggacggttctggtaaggaatacgttaagaagaacccaaagtactgtaagtctggttctaaggttatcgttaagatctctaccagagttccagtttgtttggaaaagtacgaattcatcgttcacatgggtagattcaccttgcgcgacgaaggtaagaccatcgctttgggtaaggttttgagatacaagccagctgttatcaagaaggttgaagaaatcccaccaggtgttggtgacgaaggtcaagctaagttggaagaatctgaagaattctctggttctagaggtgactctccatctaaggacgacaacaagtacgaagttatcacctacgacccagaagaagacaccatcatcgcttcttctaccggttctgaaaacgctgaataatag

SEQ ID NO: 140 - DNA, 1317 bp, synthetic construct; identical to SEQ ID NO: 127
  source 1..1317: mol_type = other DNA

SEQ ID NO: 141 - DNA, 2403 bp, synthetic construct; identical to SEQ ID NO: 139
  source 1..2403: mol_type = other DNA

SEQ ID NO: 142 - DNA, 1314 bp, synthetic construct; identical to SEQ ID NO: 128
  source 1..1314: mol_type = other DNA

SEQ ID NO: 143 - DNA, 1233 bp, synthetic construct
  source 1..1233: mol_type = other DNA
atggttgactctggtaagtctacctcttgtggtcacttgatctacaagtgtggtggtatcgacaagagaaccatcgaaaagtacgaaaaggaagctaaggaaatgggtaagtcttctttcaagtacgcttgggttttggacaagttgaaggctgaaagagaaagaggtatcaccatcgacatctctttgttcaagttccaaaccgacaagttctacttcaccatcatcgacgctccaggtcacagagacttcatcaagaacatgatcaccggtacctctcaagctgacgttgctatcttgatcatcgctgctggtaagggtgaattcgaagctggttactctaagaacggtcaaaccagagaacacgctttgttggctttcaccttgggtgttaagcaaatggttgttggtgttaacaagatggacgacaagtctgctgaatggaagcaagacagatacttggaaatcaagcaagaagtttctgaatacttgaagaaggttggttacaacccagctaaggttccattcatcccaatctctggttggttgggtgacaacatggttgaaaagtctaccaacatgccatggtacgacggtccaaccttgttgggtgctttggacaacgttcaaccaccaaagagacacgttgacaagccattgagattgccagttcaagacgtttacaagatctctggtatcggtaccgttccagttggtagagttgaaaccggtgttctcagaccaggtaccgttgttgttttcgctccatctggtatctctaccgaagttaagtctgttgaaatgcaccacgaatctttggaagaagctttgccaggtgacaacgttggtttcaacatcaagaacatcgctgttaaccaaatcaagagaggttacgttgcttctgactctagatctgacccagctagagaatctatcgacttcaccgctcaagttatcatcttgcaccacccaggtcaaatctctgttggttacaccccagttttggactgtcacaccgctcacatcgcttgtagattcaacgacttgaagcaaaaggttgacagaagatctggtgctgttttggaacaagaaccaaagttcgttaagtctggtgacgctgctatcgttaccttgatcccaaccaagtctatgtgtgttgaaccattcaccgaatacccaccattgggtagattcgctgttagagacatgagacaaaccgttgctgtttaatag

SEQ ID NO: 144 - DNA, 1311 bp, synthetic construct; identical to SEQ ID NO: 129
  source 1..1311: mol_type = other DNA

SEQ ID NO: 145 - DNA, 1941 bp, synthetic construct
  source 1..1941: mol_type = other DNA
atggactaccaagaagctaagagattggctaaggaagaaaagttgagaaagttgaaggctaagatccaagaaaacaaggttgaagacttccaagttaccgaacaacaaggtccattgccaccatacgacgaccaaggtaaccaacaaggtaagtggttgaccaccaagtacaagttcggtgaatggtactacgacatgaacggtaacccagttttcatctctccagacgacgaatacgaccaaaacggttctttgtacgacgctgaatctaacccatacttggactacgcttacgacaagttcggtaacccatgtccagttatcttgatgacccaagacaacgaatacttcttgttcaccccaatcttcgaaatccaaatcccacaatctatcatcaaggacatcaaccaaaagcaagaaaagccagctacccaaaaggttgaaaccgttgctaaggaaaagaaggctccaatccaaaaggctccagctccaccaaagatcccaaagagattgttgaaggaacaagaagctgctaactcttctgctgttgttgctgactacaagcacgaaaagttggttaccgttcaagaagctatcgacttcgaatctaagaacttcaaggaaggtcaagaaatggttaaggttgacagagaaagagactctgttaacatcgttttcatcggtcacgttgacgctggtaagtctaccttgtctggtagaatcttgaagaactgtggtgaagttgacgaaaccgaaatcagaaagttcgaattggaagctaaggaaaagaacagagaatcttgggttttggcttacatcatggacatcaacgaagaagaaagatctaagggtatcaccgttgaatgtggtaaggctcacttccaattggctaacaagagattcgttttgttggacgctccaggtcacaagaactacgttccaaacatgatcgctggtgcttgtcaagctgacgttgctgctttgatcatctctgctagacaaggtgaattcgaagctggtttcgaaggtggtcaaacccaagaacacgctcacttggctaaggctttgggtgttcaacacatgatctgtgttgtttctaagatggacgaagttaactgggacaagaagagatacgaccacatccacgactctgttgaaccattcttgagaaaccaagttggtatccaatctatcgaatgggttccaatcaacggtttcttgaacgaaaacatcgacaccccaatcccaaccgaaagatgtgaatggtacaagggtgacaccttgttcgacaagttcaacaaggttccagttccattgcgcgacccaaacggtccagttagaatcccagttttggacaagttgaaggaccaaggtcaattcttgttcggtaagatcgaatctggtaccatccgcgacgacttgtgggttaccttgatgccatacagaaagcaattccaaatcttgtctatctacaacaccaaggaccaaagagttttgtacgcttctgctggtgaaaacgttaagatcaagttgaagggtttggaagacaaggacatcgaaagaggttacatggtttgttctaccgaagacttgtgtccaatcacccaattgttcatcgctgaaatcaccatcttgcaattgccagaacacaagccaatcatgtctcaaggttactcttgtgttttgcacatgcacacctctgttgctgaaatcgaaatcgaagaagttgaagctgttcaaaacccagaaaacaagaagttgaccaagaacaccttcttgaagtctaaccaaaccggtgttgttaagatcggtatcaagggtggtttgatgtgtttggaaaagttcgaaaccatctctcaattgggtagattcaccttgcgcgacgaagaaaagaccatcggtttcggtagagttatgaagatcaagccatacaaggtttga

SEQ ID NO: 146 - DNA, 1461 bp, synthetic construct; identical to SEQ ID NO: 130
  source 1..1461: mol_type = other DNA

SEQ ID NO: 147 - DNA, 1941 bp, synthetic construct; identical to SEQ ID NO: 145
  source 1..1941: mol_type = other DNA

SEQ ID NO: 148 - DNA, 1362 bp, synthetic construct; identical to SEQ ID NO: 131
  source 1..1362: mol_type = other DNA

SEQ ID NO: 149 - DNA, 1941 bp, synthetic construct; identical to SEQ ID NO: 145
  source 1..1941: mol_type = other DNA

SEQ ID NO: 150 - DNA, 1317 bp, synthetic construct; identical to SEQ ID NO: 132
  source 1..1317: mol_type = other DNA

SEQ ID NO: 151 - DNA, 2271 bp, synthetic construct
  source 1..2271: mol_type = other DNA
atgtcttaccaatacggtcaacaaatgggtcaatacccatacgacccaaacatgaacatgatgggtttcgacccacaaatgtaccaagaatacgcttactactacttgggtggtccaccaaccccaccaccaccaaagggtccatacccaggtatcacccacgaagactacgaatctttcgacatcaacaagcaaatcttgttccaaagattcttgggtgaaaccgctgcttactacgctaagcacttgccaaagtaccaaaaggaaatggaagaattcttgaacaccaacaccgcttaccaaatgaacgaatctgaaaagcaattgatgcaatcttacttggacttcaagaagaaggaaaaggaatacgaatctttcttgaagcaattggaacaacaagctttgaacccagaattggaacaacaaaagaagttggaacaacaaaagttggaacaacaaaagatcgaacaacaaaagttggaagaacaaaagaagcaacaacaattggaacaacaaaagcaacaacagcaacagcagcaacagcaaccacaacaagaacaaccaaaggaaggtgctaccaccgctgttagaccaaagaagaagttgaacttgaacgctaagccattggaaatcgctttgaacccaccaaagatgccaaacttcccaaagcacccagacttcttggacttcgacaagttctggaacaactactctacctacatctctttgtacaacccatcttgtgaagacgaatacaagaactacccaaagccagaacaattgaagaagaaggaagctgacgaagaagctaagagaaagaagaagcaagaagaaatcgaaagagctatcaagaagagacaagacgctcaagaaagagctaaggacaagccagctcaatctgttaacttggttgaacaagttgttaagttggaaggtgaagttgacttgcaaaagtacgttgacccagacgaaaccagacaaccagttaacttggttttcatcggtcacgttgacgctggtaagtctaccttgtgtggtagattgttgttggaattgggtgaagtttctgaagctgacatcaagaagtacgaacaagaagctgttcaaaacaacagagactcttggtggttggcttacgttatggaccaaaacgaagaagaaaagcaaaagggtaagaccgttgaatgtggtaaggctcaattcgttaccaagcaaaagagattcatcttggctgacgctccaggtcacaagaactacgttccaaacatgatcatgggtgcttgtcaagctgacttggctggtttgatcgtttctgctaagaccggtgaattcgaatctggtttcgaaaagggtggtcaaacccaagaacacgctttgttggctaagtctttgggtgttgaccacatcatcatcatcgttaccaagatggacaccatcgactggaaccaagacagattcaacttgatctctcaaaacatccaagaattcgttttgaagcaatgtaagttcgacaacatctacgttatcccaatcgacgctttgtctggttctaacatcaagtctagagttgacgaatctaagtgtaactggtacaagggtccatctttgatcgacttgatcgacaccgtttctatcccaaagagaaacgaagaaggtccaatcagaatgccaatcttggacaagttcaaggacatgggttctttgtacatctacggtaagttggaatctggtaagatcatcgaaggtttggacgtttctatctacccaaagaagcaaccattccaaatcaccgaattgtacaacatgaaggaccaaaagatgaagtacgctaaggctggtgaaaacatcaagatcaaggttaagaacatcgaagaagaagaaatcaagagaggttacatgatgtgtaacttgacctctaacccatgtttggtttctcaagaattccaagctaagatcagattgttggacttgccagaatctagaagaatcttctctgaaggttaccaatgtatcatgcacttgcactctgctgttgaagaaatcgaaatctcttgtgttgaagctgttatcgacgctgaaaccaagaagtctatcaagcaaaacttcttgaagtctttcaacgaaggtatcgctaagatctctatcaagaacccagtttgtatggaaaagtacgaaaccttggctcaattgggtagattcgctttgcgcgacgacggtaagaccatcggtttcggtgaaatcttgaaggttaagccagttaagcaaggttga

SEQ ID NO: 152 - DNA, 1317 bp, synthetic construct; identical to SEQ ID NO: 133
  source 1..1317: mol_type = other DNA

SEQ ID NO: 153 - DNA, 2271 bp, synthetic construct; identical to SEQ ID NO: 151
  source 1..2271: mol_type = other DNA

SEQ ID NO: 154 - DNA, 1344 bp, synthetic construct; identical to SEQ ID NO: 126
  source 1..1344: mol_type = other DNA

SEQ ID NO: 155 - DNA, 2271 bp, synthetic construct
  source 1..2271: mol_type = other DNA
atggacaagaactctgaccaaggtaacaaccaacaaaactaccaacaatactctcaaaacggtaaccaacaacaaggtaacaacagataccaaggttaccaagcttacaacgctcaagctcaaccagctggtggttactaccaaaactaccaaggttactctggttaccagcagggggggtatcagcagtataatcctgacgctggttaccaacaacaatacaacccacaaggtggttaccagcagtataatcctcagggcggctatcagcagcaattcaacccacaaggtggtagaggtaactacaagaacttcaactacaacaacaacttgcaaggttaccaagctggtttccaaccacaatctcaaggtatgtctttgaacgacttccaaaagcaacaaaagcaagctgctccaaagccaaagaagaccttgaagttggtttcttcttctggtatcaagttggctaacgctaccaagaaggttgacaccaagccagctgaatctgacaagaaggaagaagaaaagtctgctgaaaccaaggaaccaacgaaagagcctacgaaagttgaagaaccagttaagaaggaagaaaagccagttcaaaccgaagaaaagaccgaagaaaagtctgaattgccaaaggttgaagacttgaagatctctgaatctacccacaacaccaacaacgctaacgttacctctgctgacgctttgatcaaggaacaagaagaagaagttgacgacgaagttgttaacgacgttgacgaaaccagacaaccatcttctttggttttcatcggtccagttgacgctgttaagtctaccatctgtggtaacttgatgttcatgaccggtatggttgacgaaagaaccatcgaaaagttcaagcaagaagctaaggaaaagaacagagactcttggtggttggcttacgttatggacatcaacgacgacgaaaagtctaagggtaagaccgttgaagttggtagagctaccatggaaaccccaaccaagagatacaccatcttcgacgctccaggtcacaagaactacgttccagacatgatcatgggtgctgctatggctgacgttgctgctttggttatctctgctagaaagggtgaattcgaagctggtttcgaacgcgacggtcaaaccagagaacacgctcaattggctagatctttgggtgttaacaagttggttgttgttgttaacgaaatggacgaagaaaccgttcaatggtctgaagaaagatacaacgacatcttgtctggtgttaccccattcttgatcgaccaatgtggttacaagagagaagacttgatcttcgttccaatctctggtttgaacggtcacaacatcgacaagttggcttcttgttgtccatggtacaccggtccaaccttgttggaaatcttggactgtatcgaaccaccaaagagaaacatcgacggtccattgagagttccagttttggacaagatgaaggacagaggtgttgttgctttcggtaaggttgaatctggtgttatcagaatcggtccaaagttggctgttatgccaaacaacaccaagtgtcaagttgttggtatctacaactgtaagttggaattggttagatacgctaacccaggtgaaaacatccaaatcaaggttagaatgatcgaagacgaaaaccaaatcaacaagggtgacgttttgtgtccatacgacaacttggctccaatcaccgacttgttcgaagctgaattgaccatcttggaattgttgccacacagaccaatcatcaccccaggttacaagtctatgatgcacttgcacaccatctctgacgaaatcgttatccaaaccttgaccggtatctacgaattggacggttctggtaaggaatacgttaagaagaacccaaagtactgtaagtctggttctaaggttatcgttaagatctctaccagagttccagtttgtttggaaaagtacgaattcatcgttcacatgggtagattcaccttgcgcgacgaaggtaagaccatcgctttgggtaaggttttgagatacaagccagctgttatcaagaaggttgaagaaatcccaccaggtgttggtgacgaaggtcaagctaagttggaagaatctgaagaattctctggttctagaggtgactctccatctaaggacgacaacaagtacgaagttatcacctacgacccagaagaagacaccatcatcgcttcttctaccggttctgaaaacgctgaataatag

SEQ ID NO: 156 - DNA, 1317 bp, synthetic construct; identical to SEQ ID NO: 127
  source 1..1317: mol_type = other DNA

SEQ ID NO: 157 - DNA, 2268 bp, synthetic construct
  source 1..2268: mol_type = other DNA
atgtctgactctaaccaaggtaacaaccaacaaaactaccaacaatactctcaaaacggtaaccaacaacaaggtaacaacagataccaaggttaccaagcttacaacgctcaagctcaaccagctggtggttactaccaaaactaccaaggttactctggttaccagcagggggggtatcagcagtataatcctgacgctggttaccaacaacaatacaacccacaaggtggttaccagcagtataatcctcagggcggctatcagcagcaattcaacccacaaggtggtagaggtaactacaagaacttcaactacaacaacaacttgcaaggttaccaagctggtttccaaccacaatctcaaggtatgtctttgaacgacttccaaaagcaacaaaagcaagctgctccaaagccaaagaagaccttgaagttggtttcttcttctggtatcaagttggctaacgctaccaagaaggttgacaccaagccagctgaatctgacaagaaggaagaagaaaagtctgctgaaaccaaggaaccaacgaaagagcctacgaaagttgaagaaccagttaagaaggaagaaaagccagttcaaaccgaagaaaagaccgaagaaaagtctgaattgccaaaggttgaagacttgaagatctctgaatctacccacaacaccaacaacgctaacgttacctctgctgacgctttgatcaaggaacaagaagaagaagttgacgacgaagttgttaacgacgttgacgaaaccagacaaccatcttctttggttttcatcggtccagttgacgctgttaagtctaccatctgtggtaacttgatgttcatgaccggtatggttgacgaaagaaccatcgaaaagttcaagcaagaagctaaggaaaagaacagagactcttggtggttggcttacgttatggacatcaacgacgacgaaaagtctaagggtaagaccgttgaagttggtagagctaccatggaaaccccaaccaagagatacaccatcttcgacgctccaggtcacaagaactacgttccagacatgatcatgggtgctgctatggctgacgttgctgctttggttatctctgctagaaagggtgaattcgaagctggtttcgaacgcgacggtcaaaccagagaacacgctcaattggctagatctttgggtgttaacaagttggttgttgttgttaacgaaatggacgaagaaaccgttcaatggtctgaagaaagatacaacgacatcttgtctggtgttaccccattcttgatcgaccaatgtggttacaagagagaagacttgatcttcgttccaatctctggtttgaacggtcacaacatcgacaagttggcttcttgttgtccatggtacaccggtccaaccttgttggaaatcttggactgtatcgaaccaccaaagagaaacatcgacggtccattgagagttccagttttggacaagatgaaggacagaggtgttgttgctttcggtaaggttgaatctggtgttatcagaatcggtccaaagttggctgttatgccaaacaacaccaagtgtcaagttgttggtatctacaactgtaagttggaattggttagatacgctaacccaggtgaaaacatccaaatcaaggttagaatgatcgaagacgaaaaccaaatcaacaagggtgacgttttgtgtccatacgacaacttggctccaatcaccgacttgttcgaagctgaattgaccatcttggaattgttgccacacagaccaatcatcaccccaggttacaagtctatgatgcacttgcacaccatctctgacgaaatcgttatccaaaccttgaccggtatctacgaattggacggttctggtaaggaatacgttaagaagaacccaaagtactgtaagtctggttctaaggttatcgttaagatctctaccagagttccagtttgtttggaaaagtacgaattcatcgttcacatgggtagattcaccttgcgcgacgaaggtaagaccatcgctttgggtaaggttttgagatacaagccagctgttatcaagaaggttgaagaaatcccaccaggtgttggtgacgaaggtcaagctaagttggaagaatctgaagaattctctggttctagaggtgactctccatctaaggacgacaacaagtacgaagttatcacctacgacccagaagaagacaccatcatcgcttcttctaccggttctgaaaacgctgaataatag

SEQ ID NO: 158 - DNA, 1317 bp, synthetic construct; identical to SEQ ID NO: 132
  source 1..1317: mol_type = other DNA

SEQ ID NO: 159 - DNA, 2067 bp, synthetic construct
  source 1..2067: mol_type = other DNA
atgtctgactctaaccaaggtaacaaccaacaaaactaccaacaatactctcaaaacggtaaccaacaacaaggtaacaacagataccaaggttaccaagcttacaacgctcaagctcaaccagctggtggttactaccaaaactaccaaggttactctggttaccagcagggggggtatcagcagtataatcctgacgctggttaccaacaacaatacaacccacaaggtggttaccagcagtataatcctcagggcggctatcagcagcaattcaacccacaaggtggtagaggtaactacaagaacttcaactacaacaacaacttgcaaggttaccaagctggtttccaaccacaatctcaaggtatgtctttgaacgacttccaaaagcaacaaaagcaagctgctccaaagccaaagaagaccttgaagttggtttcttcttctggtatcaagttggctaacgctaccaagaaggttgacaccaagccagctgaatctgacaagaaggaagaagaaaagtctgctgaaaccaaggaaccaacgaaagagcctacgaaagttgaagaaccagttaagaaggaagaaaagccagttcaaaccgaagaaaagaccgaagaaaagtctgaattgccaaaggttgaagacttgaagatctctgaatctacccacaacaccaacaacgctaacgttacctctgctgacgctttgatcaaggaacaagaagaagaagttgacgacgaagttgttaacgacccagacgaaaccagacaaccagttaacttggttttcatcggtcacgttgacgctggtaagtctaccttgtgtggtagattgttgttggaattgggtgaagtttctgaagctgacatcaagaagtacgaacaagaagctgttcaaaacaacagagactcttggtggttggcttacgttatggaccaaaacgaagaagaaaagcaaaagggtaagaccgttgaatgtggtaaggctcaattcgttaccaagcaaaagagattcatcttggctgacgctccaggtcacaagaactacgttccaaacatgatcatgggtgcttgtcaagctgacttggctggtttgatcgtttctgctaagaccggtgaattcgaatctggtttcgaaaagggtggtcaaacccaagaacacgctttgttggctaagtctttgggtgttgaccacatcatcatcatcgttaccaagatggacaccatcgactggaaccaagacagattcaacttgatctctcaaaacatccaagaattcgttttgaagcaatgtaagttcgacaacatctacgttatcccaatcgacgctttgtctggttctaacatcaagtctagagttgacgaatctaagtgtaactggtacaagggtccatctttgatcgacttgatcgacaccgtttctatcccaaagagaaacgaagaaggtccaatcagaatgccaatcttggacaagttcaaggacatgggttctttgtacatctacggtaagttggaatctggtaagatcatcgaaggtttggacgtttctatctacccaaagaagcaaccattccaaatcaccgaattgtacaacatgaaggaccaaaagatgaagtacgctaaggctggtgaaaacatcaagatcaaggttaagaacatcgaagaagaagaaatcaagagaggttacatgatgtgtaacttgacctctaacccatgtttggtttctcaagaattccaagctaagatcagattgttggacttgccagaatctagaagaatcttctctgaaggttaccaatgtatcatgcacttgcactctgctgttgaagaaatcgaaatctcttgtgttgaagctgttatcgacgctgaaaccaagaagtctatcaagcaaaacttcttgaagtctttcaacgaaggtatcgctaagatctctatcaagaacccagtttgtatggaaaagtacgaaaccttggctcaattgggtagattcgctttgcgcgacgacggtaagaccatcggtttcggtgaaatcttgaaggttaagccagttaagcaaggttga

SEQ ID NO: 160 - DNA, 1317 bp, synthetic construct; identical to SEQ ID NO: 133
  source 1..1317: mol_type = other DNA

SEQ ID NO: 161 - DNA, 2067 bp, synthetic construct; identical to SEQ ID NO: 159
  source 1..2067: mol_type = other DNA

SEQ ID NO: 162 - protein, 437 aa, Saccharomyces cerevisiae
MDNEVEKNIEIWKVKKLVQSLEKARGNGTSMISLVIPPKGQIPLYQKMLTDEYGTASNIKSRVNRLSVLSAITSTQQKLKLYNTLPKNGLVLYCGDIITEDGKEKKVTFDIEPYKPINTSLYLCDNKFHTEVLSELLQADDKFGFIVMDGQGTLFGSVSGNTRTVLHKFTVDLPKKHGRGGQSALRFARLREEKRHNYVRKVAEVAVQNFITNDKVNVKGLISAGSADFKTDLAKSELFDPRLACKVISIVDVSYGGENGFNQAIELSAEALANVKYVQEKKLLEAYFDEISQDTGKFCYGIDDTLKALDLGAVEKLIVFENLETIRYTFKDAEDNEVIKFAEPEAKDKSFAIDKATGQEMDVVSEEPLIEWLAANYKNFGATLEFITDKSSEGAQFVTGFGGIGAMLRYKVNFEQLVDESEDEYYDEDEGSDYDFI

SEQ ID NO: 163 - protein, 437 aa, Saccharomyces cerevisiae
MDNEVEKNIEIWKVKKLVQSLEKARGNGTSMISLVIPPKGQIPLYQKMLTDEYGTASNIKSRVNRLSVLSAITSTQQKLKLYNTLPKNGLVLYCGDIITEDGKEKKVTFDIEPYKPINTSLYLCDNKFHTEVLSELLQADDKFGFIVMDGQGTLFGSVSGNTRTVLHKFTVDLPKKHGRGGQSALRFARLREEKRHNYVRKVAEVAVQNFITNDKVNVKGLSLAGSADFKTDLAKSELFDPRLACKVISIVDVSYGGENGFNQAIELSAEALANVKYVQEKKLLEAYFDEISQDTGKFCYGIDDTLKALDLGAVEKLIVFENLETIRYTFKDAEDNEVIKFAEPEAKDKSFAIDKATGQEMDVVSEEPLIEWLAANYKNFGATLEFITDKSSEGAQFVTGFGGIGAMLRYKVNFEQLVDESEDEYYDEDEGSDYDFI

SEQ ID NO: 164 - protein, 437 aa, Saccharomyces cerevisiae
MDNEVEKNIEIWKVKKLVQSLEKARGNGTSMISSVIPPKGQIPLYQKMLTDEYGTASNIKSRVNRLSVLSAITSTQQKLKLYNTLPKNGLVLYCGDIITEDGKEKKVTFDIEPYKPINTSLYLCDNKFHTEVLSELLQADDKFGFIVMDGQGTLFGSVSGNTRTVLHKFTVDLPKKHGRGGQSALRFARLREEKRHNYVRKVAEVAVQNFITNDKVNVKGLILAGSADFKTDLAKSELFDPRLACKVISIVDVSYGGENGFNQAIELSAEALANVKYVQEKKLLEAYFDEISQDTGKFCYGIDDTLKALDLGAVEKLIVFENLETIRYTFKDAEDNEVIKFAEPEAKDKSFAIDKATGQEMDVVSEEPLIEWLAANYKNFGATLEFITDKSSEGAQFVTGFGGIGAMLRYKVNFEQLVDESEDEYYDEDEGSDYDFI

SEQ ID NO: 165 - protein, 76 aa, Saccharomyces cerevisiae
MDNEVEKNIEIWKVKKLVQSLEKARGNGTSMISLVIPPKGQIPLYQKMLTDEYGTASNIKSRVNRLSVLSAITSTH

SEQ ID NO: 166 - protein, 437 aa, Saccharomyces cerevisiae
MDNEVEKNIEIWKVKKLVQSLEKARGNGTFMISLVIPPKGQIPLYQKMLTDEYGTASNIKSRVNRLSVLSAITSTQQKLKLYNTLPKNGLVLYCGDIITEDGKEKKVTFDIEPYKPINTSLYLCDNKFHTEVLSELLQADDKFGFIVMDGQGTLFGSVSGNTRTVLHKFTVDLPKKHGRGGQSALRFARLREEKRHNYVRKVAEVAVQNFITNDKVNVKGLILAGSADFKTDLAKSELFDPRLACKVISIVDVSYGGENGFNQAIELSAEALANVKYVQEKKLLEAYFDEISQDTGKFCYGIDDTLKALDLGAVEKLIVFENLETIRYTFKDAEDNEVIKFAEPEAKDKSFAIDKATGQEMDVVSEEPLIEWLAANYKNFGATLEFITDKSSEGAQFVTGFGGIGAMLRYKVNFEQLVDESEDEYYDEDEGSDYDFI

SEQ ID NO: 167 - protein, 437 aa, Saccharomyces cerevisiae
MDNEVEKNIEIWKVKKLVQSLEKARGNGTPMISLVIPPKGQIPLYQKMLTDEYGTASNIKSRVNRLSVLSAITSTQQKLKLYNTLPKNGLVLYCGDIITEDGKEKKVTFDIEPYKPINTSLYLCDNKFHTEVLSELLQADDKFGFIVMDGQGTLFGSVSGNTRTVLHKFTVDLPKKHGRGGQSALRFARLREEKRHNYVRKVAEVAVQNFITNDKVNVKGLILAGSADFKTDLAKSELFDPRLACKVISIVDVSYGGENGFNQAIELSAEALANVKYVQEKKLLEAYFDEISQDTGKFCYGIDDTLKALDLGAVEKLIVFENLETIRYTFKDAEDNEVIKFAEPEAKDKSFAIDKATGQEMDVVSEEPLIEWLAANYKNFGATLEFITDKSSEGAQFVTGFGGIGAMLRYKVNFEQLVDESEDEYYDEDEGSDYDFI

(19) United States
(12) Patent Application Publication
(10) Pub. No.: US 2024/0158735 A1
(43) Pub. Date: May 16, 2024

(54) METHODS AND COMPOSITIONS USING AN ENGINEERED RELEASE FACTOR

(71) Applicant: Opentrons Labworks Inc., Brooklyn, NY (US)

(72) Inventors: Leslie Mitchell, New York, NY (US); Joel S. Bader, Bronx, NY (US)

(21) Appl. No.: 18/501,724

(22) Filed: Nov. 3, 2023

Related U.S. Application Data
(60) Provisional application No. 63/382,156, filed on Nov. 3, 2022.

(51) Int. Cl. C12N 1/18; C12N 9/00; C12N 15/63

(52) U.S. Cl. CPC C12N 1/185; C12N 9/93; C12N 15/635; C12N 2840/002; C12N 2840/107; C12N 2840/704

(57) ABSTRACT

Provided herein are compositions and systems comprising an engineered release factor and methods for producing the same. Also provided herein are compositions, systems, and methods for producing a polypeptide comprising a non-canonical amino acid.

### CROSS REFERENCE

This application claims the benefit of U.S. Provisional Application No. 63/382,156, filed on Nov. 3, 2022, which is incorporated herein by reference in its entirety.

### SEQUENCE LISTING

This instant application contains a Sequence Listing which has been submitted electronically in XML format and is hereby incorporated by reference in its entirety. Said XML copy, created on Oct. 31, 2023, is named 59725-711_201 SL.xml and is 267,875 bytes in size.

### BACKGROUND

Codon rewriting and repurposing translational machinery may be important tools to expand the genetic code artificially. These may also be important tools to enable incorporation of non-canonical amino acids (ncAAs) into proteins. Many methods for ncAA incorporation use a stop codon together with a suppressor tRNA to convert the stop codon into a sense codon. These methods suffer, however, because the suppressor tRNA competes with the native release factor, resulting in early termination and poor readthrough. Methods that control release factor activity to avoid recognizing a defined subset of stop codons, especially in eukaryotic cells, would have great utility in improving the performance of methods for ncAA incorporation into polypeptides without codon rewriting.

### SUMMARY

Provided herein are compositions, systems, and methods for producing a polypeptide comprising a non-canonical amino acid (ncAA) or a population of polypeptides comprising an ncAA. Compositions, systems, and methods described herein can utilize two recombinant release factors, one of which comprises an element that allows selective modulation of function or expression of the recombinant release factor, which can allow higher usage of the other recombinant release factor to introduce an ncAA in the presence of the ncAA and an orthogonal translation system (OTS). Compositions, systems, and methods provided herein can allow producing polypeptides with an ncAA without codon rewriting or replacement.

In some aspects, provided herein is a composition comprising: (a) a first recombinant release factor, wherein the first recombinant release factor is configured to recognize UGA, UAA, UAG, or any combination thereof as a stop codon; and (b) a second recombinant release factor, wherein the second recombinant release factor is configured to recognize UGA, UAA, UAG, or any combination thereof as a stop codon, and wherein the second recombinant release factor comprises an element that allows selective modulation of function of the second recombinant release factor.

In some aspects, provided herein is a composition comprising: (a) a first recombinant nucleic acid sequence comprising a first sequence encoding a first recombinant release factor, wherein the first recombinant release factor is configured to recognize UGA, UAA, UAG, or any combination thereof as a stop codon; and (b) a second recombinant nucleic acid sequence comprising a second sequence encoding a second recombinant release factor, wherein the second recombinant release factor is configured to recognize UGA, UAA, UAG, or any combination thereof as a stop codon, wherein the second nucleic acid sequence comprises an element that allows selective modulation of function or expression of the second recombinant release factor.

In some aspects, provided herein is a method of producing a polypeptide molecule comprising a non-canonical amino acid (ncAA) or a population of polypeptide molecules comprising the ncAA, the method comprising providing: (a) a first nucleic acid sequence comprising a first sequence encoding a first recombinant release factor, wherein the first recombinant release factor is configured to recognize UGA, UAA, UAG, or any combination thereof as a stop codon; (b) a second nucleic acid sequence comprising a second sequence encoding a second recombinant release factor, wherein the second recombinant release factor is configured to recognize UGA, UAA, UAG, or any combination thereof as a stop codon, and wherein the second nucleic acid sequence comprises an element that allows selective modulation of function or expression of the second recombinant release factor; and (c) an aminoacyl-tRNA synthetase (aaRS)/tRNA pair.

In some aspects, provided herein is a method of producing a polypeptide molecule comprising a non-canonical amino acid (ncAA) or a population of polypeptide molecules comprising the ncAA, the method comprising providing: (a) a first recombinant release factor, wherein the first recombinant release factor is configured to recognize UGA, UAA, UAG, or any combination thereof as a stop codon; (b) a second recombinant release factor, wherein the second recombinant release factor is configured to recognize UGA, UAA, UAG, or any combination thereof as a stop codon, and wherein the second recombinant release factor comprises an element that allows selective modulation of function of the second recombinant release factor; and (c) an aminoacyl-tRNA synthetase (aaRS)/tRNA pair.

In some aspects, provided herein is a method of screening a release factor having codon-specific release factor activity, the method comprising: a. providing a cell or a population of cells comprising a first release factor recognizing one or two stop codons; b. introducing the cell or the population of cells a second release factor; c. performing a first assay to detect codon-specific activity of the second release factor; and d. performing a second assay to confirm the second release factor does not recognize the one or two stop codons recognized by the first release factor.

In some aspects, provided herein is a system for screening a release factor for codon-specific release factor activity, the system comprising: a. a cell or a population of cells comprising a first release factor that recognizes one or two stop codons; b. a first assay configured to detect a codon-specific release factor activity of a second release factor via introducing the second release factor to the cell or the population of cells, wherein the second release factor recognizes or is configured to recognize at least one stop codon; and c. a second assay configured to confirm the codon-specific release factor activity of the second release factor is specific for one or two stop codons not recognized by the first release factor; and a computer configured to process a first data set from the first assay and a second data set from the second assay.

### INCORPORATION BY REFERENCE

Each patent, publication, and non-patent literature cited in the application is hereby incorporated by reference in its entirety as if each was incorporated by reference individually. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The features of the present disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings (also “Figure” and “FIG.” herein), of which:

FIG. 1 shows the recognition of the three stop codons UAG, UAA and UGA by prokaryotic (upper line) and eukaryotic (lower line) release factors. Prokaryotes contain two distinct single subunit release factors with the indicated specificities. Eukaryotes contain a single, release factor eRF1 which in conjunction with eRF3 recognizes all three stop codons. In certain species, such as the ciliate Tetrahymena and others, only the UGA stop codon is recognized by eRF1, while in others species such as the ciliate Euplotes only the UAG and UAA stop codons are recognized.

FIG. 2 shows an example embodiment of a shuffle episome system for the yeast S. cerevisiae. In some embodiments, the payload may comprise a SUP45 gene, encoding eRF1. In some embodiments, the payload may comprise a SUP35 gene, encoding eRF3. In some embodiments, the payload may comprise both a SUP45 gene and a SUP35 gene. In other embodiments, additional payload elements may be included such as homologs of the genes MTQ2, TRM112, and genes encoding tRNA T11). The diagram in the center illustrates the generic architecture of a plasmid system used to build yeast strains that can either assess the specificity of a given eRF system or survive solely on one or more ciliate eRF proteins in the absence of the cognate yeast eRF protein or proteins. The diagram indicates the position of the payload (a release factor gene or genes with optionally, additional payload genes) and vector components. The vector components include a selectable marker and may include other sequences such as a centromere and/or an origin of replication. Two types of vector may be used, a payload vector containing a positive selection marker such as LEU2, HIS3, ADE2 intended to host a non-S. cerevisiae payload. A second type of vector is a shuffle vector (shown in the diagram) that includes the S. cerevisiae payload eRF gene or genes and a counter-selectable marker such as one or more copies of URA3. The diagram on the left shows how plasmid shuffling can result in the replacement of the shuffle vector and its S. cerevisiae payload can be replaced by one or more payload plasmids, if and only if those payload plasmids produce one or more eRF1 proteins that are able to substitute for the essential function of the S. cerevisiae eRF1 protein. Further details can be seen in FIGS. 4 and 5.

FIGS. 3A-3B show phylogenetic trees for ciliates. FIG. 3A shows a phylogenetic tree for ciliate organisms. FIG. 3B shows a phylogenetic tree for ciliate organisms with examples of specific ciliates that only recognize the UGA stop codon.

FIG. 4 shows examples of ciliate gene constructs that can be tested for function and stop codon specificity in yeast. A specific example embodiment of how these gene constructs can be deployed is given in FIG. 5.

FIG. 5 shows an example embodiment of a shuffle episome system. This system is specifically designed to evaluate function of ciliate-derived engineered RF sequences in yeast. In this embodiment, a yeast strain is constructed encoding its only copy of the yeast eRF1 gene on a shuffle plasmid, such as a Superloser plasmid, which is marked with a counterselectable marker such as URA3. Into this strain, two separate ciliate-derived engineered eRF constructs (or appropriately marked empty vectors) can be transformed. The first, marked with LEU2, is designed to exclusively recognize the UAA and UAG stop codons, and the second, marked with HIS3, is designed to exclusively recognize UGA. After removal of the shuffle plasmid by selection on 5-FOA, strains carrying vectors either the UGA-specific or the UAG/UAA-specific eRF gene alone will be unable to grow since not all stop codon types can be decoded. A strain carrying vectors expressing both types of ciliate-derived engineered eRF genes will be able to grow because all three stop codons can be decoded.

FIG. 6 shows stop-codon selectivity of ciliate domain/motif-swapped eRF1 proteins in yeast.

FIG. 7 shows stop-codon selectivity of whole-gene ciliate eRF1/eRF3 constructs in yeast.

FIG. 8 shows the assessment of plasmid dependency of erf1Δ strains carrying ciliate release factor constructs.

FIG. 9 shows an example embodiment of a computer system a program configured to implement methods provided herein. In some cases, the program comprises an algorithm. The computer system may be a machine learning-based or statistical learning-based computer system that uses observed patterns of codon usage to select replacement codons. In some cases, the computer system comprises a computer processing unit and a sequence processing unit, wherein the computer processing unit and the sequence processing unit are bilaterally communicatively coupled. In some embodiments, the sequence processing unit and the computer processing unit comprise a storage component. 901: Computer system. 905: Central processing unit (CPU). 910: Memory. 915: Electronic storage unit. 920: Central processing unit of computer system. 925: Peripheral devices. 930: Data storage with files containing the translation tables representing the genetic code of the organism whose genome is being rewritten. 935: electronic display. 940: Instructions describing which translation table to use, the codons to be eliminated, and the locations of input and output files. 950: Computer program implementing the methods to perform the codon rewriting.

FIG. 10 shows temperature sensitivity of genomically integrated mutant alleles of sup45. The indicated sup45 temperature sensitive alleles were introduced into the genome of S. cerevisiae, replacing the wild-type (WT) SUP45 allele. Ten-fold serial dilutions of each strain were spotted on yeast peptone dextrose (YPD) medium at the indicated temperatures and grown for 2-3 days. Only the WT strain was able to grow robustly at all temperatures tested consistent with loss of the essential function of SUP45 in the temperature sensitive alleles.

FIG. 11 shows relative readthrough in strains encoding sup45 temperature sensitive alleles. Relative readthrough efficiency (RRE) was measured in cells expressing a wild-type (WT) SUP45 allele or a temperature sensitive allele, sup45-sl23ts, in the presence (+ncAA) or absence (−ncAA) of a non-canonical amino acid, and presence (+OTS) or absence (−OTS) of an orthogonal translation system. The ncAA used was LysN3 and the OTS comprised a heterologous synthetase and tRNA engineered to function exclusively with the non-canonical amino acid (ncAA) LysN3.

FIG. 12 shows incorporation percentage of LysN3 or other canonical amino acids at a TAG readthrough codon in a sup45 temperature sensitive strain in the absence (−OTS) or presence (+OTS) of an orthogonal translation system engineered for LysN3 specificity.

FIG. 13 shows stop codon specificity of the Bam-SUP45 release factor. Relative readthrough (RRE) was measured in a parental strain (encoding the wild type S. cerevisiae SUP45 gene plus the Blepharisma americanum (Bam) amino acid swapped SUP45 allele, Bam-SUP45) or six individually derived revertant strains (encoding only the Bam-SUP45, with expected stop codon specificity for TAA and TAG, but not TGA). Engineered alanine tRNAs (tRNA-Ala) were expressed (left, with TAA recognition programmed into the anticodon; or right, with TGA recognition programmed into the anticodon). RRE was measured using one of two dual reporter systems, encoding a 5′ BFP coding sequence and a 3′ GFP coding sequence with one of an intervening stop codon (left, BFP-TAA-GFP; right, BFP-TGA-GFP).

FIG. 14 shows read through in revertant strains expressing only a TAG/TAA-recognizing release factor. Relative readthrough (RRE) was measured in a parental strain (encoding the wild type S. cerevisiae SUP45 gene plus the Blepharisma americanum (Bam) amino acid swapped SUP45 allele, Bam-SUP45) or six individually derived revertant strains (encoding only Bam-SUP45, with expected read stop codon specificity for TAA and TAG, but not TGA). Strains either expressed (+OTS) or did not express (−OTS) an orthogonal translation system comprising a heterologous tRNA and synthetase engineered to function exclusively with LysN3, which was included in the growth media for all conditions. The heterologous tRNA was programmed with an anticodon to recognize TGA, which was encoded on the dual reporter system (BFP-TGA-GFP).

### INCORPORATION BY REFERENCE

Each patent, publication, and non-patent literature cited in the application is hereby incorporated by reference in its entirety as if each was incorporated by reference individually.

### DETAILED DESCRIPTION

Provided herein are compositions, systems, and methods for producing a polypeptide comprising a non-canonical amino acid (ncAA) or a population of polypeptides comprising an ncAA. Compositions, systems, and methods described herein can utilize a recombinant release factor comprising an element that allows selective modulation of function or expression of the recombinant release factor. Compositions, systems, and methods provided herein can allow producing polypeptides with an ncAA without codon rewriting or replacement.

Also provided herein are orthogonal translation systems for introducing ncAAs in polypeptides or proteins. In some embodiments, orthogonal systems may utilize tRNAs that recognize a stop codon (e.g., UAG, UGA, or UAA). In some embodiments, orthogonal systems may utilize tRNAs that recognize UAA, UAG, or UGA stop codon. In some embodiments, orthogonal systems may utilize tRNAs that recognize UAA stop codon. In some embodiments, orthogonal systems may utilize tRNAs that recognize UAG stop codon. In some embodiments, orthogonal systems may utilize tRNAs that recognize UGA stop codon. In some embodiments, tRNAs recognizing UAA, UAG, or UGA stop codon may be referred to as suppressor tRNAs as suppressor tRNAs may decode a stop codon as a sense codon, suppressing the termination of protein translation. For example, suppressor tRNAs decode a stop codon in a mRNA transcript and may insert an ncAA in a polypeptide. In this example, a native or natural release factor may compete with suppressor tRNAs. As such, the efficiency of ncAA incorporation may be low. The present disclosure provides systems, compositions and methods that may solve a low efficiency of producing polypeptides with ncAAs based on stop codon suppression. The systems, compositions, and methods described herein may not require genome rewriting e.g., stop codon replacement and/or rewriting.

Compositions, systems, and methods described herein utilizes one or more recombinant release factors, each with release activity for a subset of stop codons. For example, a composition or a system comprising two recombinant release factors may be introduced, wherein each of the two recombinant release factor is engineered or configured to recognize only one or two stop codons. For example, the composition or the system may comprise one recombinant release factor may be configured to recognize UGA and another recombinant release factor may be configured to recognize UAA and/or UAG. For example, the composition or the system may further comprise one or more elements that can selectively modulate function or expression of recombinant release factors. In this example, at least one of the recombinant release factors (e.g., its expression or activity/function) can be modulated, e.g., turned on or off. In this example, at least one of the recombinant release factors or its activity can be turned off to allow suppressor tRNAs (or any other tRNAs described herein) to decode the stop codon (i.e., the stop codon that is normally recognized by the recombinant release factor being turned off) as a sense codon and incorporate an ncAA in a polypeptide chain (and suppress the termination of protein translation). In this example,

In some embodiments, an element that allows selective modulation of function or expression of a recombinant release factor may include, but is not limited to, a temperature sensitive allele, a degron cassette, a conditional or an inducible promoter, or a combination thereof. In one example, the function or the activity of a recombinant release factor configured to recognize UAA and/or UAG may be modulated by using a degron system. In this example, the function or the activity of the recombinant release factor may be turned off by degrading the recombinant release factor by turning on the degron system. In this example, the degradation of the recombinant release factor can allow suppressor tRNAs (or any other tRNAs described herein) to decode UAA and/or UAG stop codon as a sense codon and incorporate an ncAA in a polypeptide chain. In this example, a recombinant release factor configured to recognize UGA as a stop codon may still be present and functional.

In another example, the function or the activity of a recombinant release factor may be modulated by using a temperature sensitive allele. In this example, the function or the activity of the recombinant release factor may be compromised (e.g., reduced activity) by changing the temperature from a permissive to a non-permissive temperature, allowing suppressor tRNAs (or any other tRNAs described herein) to decode one or more stop codons (e.g., UAG, UGA, and/or UAA) as a sense codon and incorporate one or more ncAAs in a polypeptide chain.

In some aspects, compositions, systems, and methods comprising one or more recombinant release factors described herein may be used in a cell for producing a polypeptide comprising one or more ncAAs. Compositions, systems, and methods described herein may be useful in production of therapeutics, for example, any therapeutics comprising one or more ncAAs. For example, compositions, systems, and methods described herein may be used to produce an antibody with ncAAs, which can provide improved control of conjugation sites for conjugates such as antibody-drug conjugates.

Details of release factor engineering and elements that allow selective modulation of function or expression of a recombinant release factor (e.g., a temperature sensitive allele, a degron cassette, or a conditional/inducible promoter) are described herein.

### Definitions

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively, unless the context specifically refers to a disjunctive use.

The term “about” or “approximately” can mean within an acceptable error range for the particular value, which may depend in part on how the value is measured or determined, e.g., the limitations of the measurement system. For example, “about” can mean within 1 or more than 1 standard deviation. Alternatively, “about” can mean a range of up to 20%, up to 10%, up to 5%, or up to 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value should be assumed.

Throughout this disclosure, numerical features are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the present disclosure, subject to any specifically excluded limit in the stated range. Where the stated range includes one or both of the limits, ranges excluding either or both of those included limits are also included in the present disclosure, unless the context clearly dictates otherwise.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures. To facilitate an understanding of the present disclosure, a number of terms and phrases are defined below.

Certain specific details of this description are set forth in order to provide a thorough understanding of various embodiments. However, one skilled in the art will understand that the present disclosure may be practiced without these details. In other instances, well-known techniques or methods have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed disclosure.

The terms “nucleic acid sequence,” “polynucleic acid sequence,” and/or “nucleotide sequence” are used herein interchangeably and have the identical meaning herein and refer to DNA or RNA. In some embodiments, a nucleic acid sequence is a polymer comprising or consisting of nucleotide monomers, which are covalently linked to each other by phosphodiester-bonds of a sugar/phosphate-backbone. The terms “nucleic acid sequence,” “polynucleic acid sequence,” and “nucleotide sequence” may encompass unmodified nucleic acid sequences, i.e., comprise unmodified nucleotides, or natural nucleotides. In some embodiments, “natural nucleotide,” “unmodified nucleotide,” and/or “canonical nucleotide” are used herein interchangeably and have the identical meaning herein and refer to the naturally occurring nucleotide bases adenine (A), guanine (G), cytosine (C), uracil (U), and/or thymine (T). The terms “nucleic acid sequence,” “polynucleic acid sequence,” and “nucleotide sequence” may also encompass modified nucleic acid sequences, such as base-modified, sugar-modified or backbone-modified etc., DNA or RNA.

The nomenclature used to describe polypeptides or proteins follows the conventional practice wherein the amino group is presented to the left (the amino- or N-terminus) and the carboxyl group to the right (the carboxy- or C-terminus) of each amino acid residue. When amino acid residue positions are referred to in a polypeptide or a protein, they are numbered in an amino to carboxyl direction with position one being the residue located at the amino terminal end of the polypeptide or the protein of which it can be a part. The amino acid sequences of peptides set forth herein are generally designated using the standard single letter or three letter symbol. (A or Ala for Alanine; C or Cys for Cysteine; D or Asp for Aspartic Acid; E or Glu for Glutamic Acid; F or Phe for Phenylalanine; G or Gly for Glycine; H or His for Histidine; I or Ile for Isoleucine; K or Lys for Lysine; L or Leu for Leucine; M or Met for Methionine; N or Asn for Asparagine; P or Pro for Proline; Q or Gln for Glutamine; R or Arg for Arginine; S or Ser for Serine; T or Thr for Threonine; V or Val for Valine; W or Trp for Tryptophan; and Y or Tyr for Tyrosine). In some embodiments, the amino acid may be a natural amino acid. In some embodiments, the natural amino acid may include alanine, cysteine, aspartic acid, glutamic acid, phenylalanine, glycine, histidine, isoleucine, lysine, leucine, methionine, asparagine, proline, glutamine, arginine, serine, threonine, valine, tryptophan, or tyrosine.

The term “non-canonical amino acid” or “ncAA” refers to any amino acid other than the 20 standard amino acids (alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine). There are over 700 known ncAA any of which may be used in the methods described herein. In some embodiments, examples of ncAA include, but are not limited to, L-Tryptazan, 5-Fluoro-L-tryptophan, L-Ethionine, L-Selenomethionine, Trifluoro-L-methionine, L-Norleucine, L-Homopropargylglycine, (2S)-2-amino-5-(methylsulfanyl) pentanoic acid, (2S)-2-amino-6-(methylsulfanyl) hexanoic acid, Para-fluoro-L-phenylalanine, Para-iodo-L-phenylalanine, Para-azido-L-phenylalanine, Para-acetyl-L-phenylalanine, Para-benzoyl-L-phenylalanine, Meta-fluoro-L-tyrosine, O-methyl-L-tyrosine, Para-propargyloxy-L-phenylalanine, (2S)-2-aminooctanoic acid, (2S)-2-aminononanoic acid, (2S)-2-aminodecanoic acid, (2S)-2-aminohept-6-enoic acid, (2S)-2-aminooct-7-enoic acid, L-Homocysteine, (2S)-2-amino-5-sulfanylpentanoic acid, (2S)-2-amino-6-sulfanylhexanoic acid, L-S-(2-nitrobenzyl) cysteine, L-S-ferrocenyl-cysteine, L-O-crotylserine, L-O-(pent-4-en-1-yl)serine, L-O-(4,5-dimethoxy-2-nitrobenzyl)serine, (2S)-2-amino-3-({[5-(dimethylamino)naphthalen-1-yl]sulfonyl}amino)propanoic acid, (2S)-3-[(6-acetyl-naphthalen-1-yl)amino]-2-aminopropanoic acid, L-Pyrrolysine, N⁶-[(propargyloxy)carbonyl]-L-lysine, L-N⁶-acetyllysine, N⁶-trifluoroacetyl-L-lysine, N⁶-{[1-(6-nitro-1,3-benzodioxol-5-yl)ethoxy]carbonyl}-L-lysine, N⁶-{[2-(3-methyl-3H-diaziren-3-yl)ethoxy]carbonyl}-L-lysine, p-azidophenylalanine, N⁶-[(2-Azidoethoxy)carbonyl]-L-lysine, p-acetyl-L-phenylalanine (AcF), p-propargyloxy-L-phenylalanine (OPG), 4-azidomethyl-L-phenylalanine (AzMF), 4-borono-L-phenylalanine (BPhe), 3,4-dihydroxy-L-phenylalanine (DOPA), 4-iodo-L-phenylalanine (IPhe), L-α-aminocaprylic acid (AC), N^{ε}-azido-L-lysine (AzK), 3-amino-L-tyrosine (ATyr), 4-amino-L-phenylalanine (APhe), N^{ε}, N^{ε}-dimethyl-L-lysine (DMK), Boc-L-lysine (BocK), (S)-2-amino-6-((2-azidoethoxy)carbonylamino)hexanoic acid (LysN3), (2S)-2-amino-6-(((prop-2-yn-1-yloxy)carbonyl)amino)hexanoic acid (LysAlk), and 2-aminoisobutyric acid. In some embodiments, examples of ncAA include, but are not limited to, AbK (unnatural amino acid for Photo-crosslinking probe), 3-Aminotyrosine (unnatural amino acid for inducing red shift in fluorescent proteins and fluorescent protein-based biosensors), L-Azidohomoalanine hydrochloride (unnatural amino acid for bio-orthogonal labeling of newly synthesized proteins), L-Azidonorleucine hydrochloride (unnatural amino acid for bio-orthogonal or fluorescent labeling of newly synthesized proteins), BzF (photoreactive unnatural amino acid; photo-crosslinker), DMNB-caged-Serine (caged serine; excited by visible blue light), HADA (blue fluorescent D-amino acid for labeling peptidoglycans in live bacteria), NADA-green (fluorescent D-amino acid for labeling peptidoglycans in live bacteria), NB-caged Tyrosine hydrochloride (ortho-nitrobenzyl caged L-tyrosine), RADA (orange-red TAMRA-based fluorescent D-amino acid for labeling peptidoglycans in live bacteria), Rf470DL (blue rotor-fluorogenic fluorescent D-amino acid for labeling peptidoglycans in live bacteria), sBADA (green fluorescent D-amino acid for labeling peptidoglycans in bacteria), and YADA (green-yellow lucifer yellow-based fluorescent D-amino acid for labeling peptidoglycans in live bacteria). In some embodiments, examples of ncAA include, but are not limited to, β-alanine, D-alanine, 4-hydroxyproline, desmosine, D-glutamic acid, γ-aminobutyric acid, β-cyanoalanine, norvaline, 4-(E)-butenyl-4(R)-methyl-N-methyl-L-threonine, N-methyl-L-leucine, selenocysteine, and statine. In some embodiments, an ncAA comprises p-azidophenylalanine or 2-aminoisobutyric acid (also known as α-aminoisobutyric acid, AIB, α-methylalanine, or 2-methylalanine).

The terms “codon” and “anticodon” as used herein may refer to DNA or RNA. In some embodiments, DNA comprises nucleotide bases adenine (A), guanine (G), cytosine (C), or thymine (T). In some embodiments, RNA comprises nucleotide bases adenine (A), guanine (G), cytosine (C), or uracil (U). In some embodiments, DNA or RNA may comprise inosine (I). in some embodiments, inosine (I) may pair with adenine (A), cytosine (C), or uracil (U). In some embodiments, DNA or RNA may comprise queuosine (Q). In some embodiments, queuosine (Q) may pair with cytosine (C) or uracil (U).

Although methods and materials similar or equivalent to those described herein can be used in the practice or testing of the present disclosure, suitable methods, and materials are described below.

### Release Factors (RFs)

In some aspects, release factors (RFs) described herein may modulate polypeptide or protein translation upon recognizing a stop codon. In some embodiments, the stop codon may comprise UGA, UAA, UAG, or a combination thereof. In some embodiments, release factors may modulate terminating translation of a polypeptide or a protein. In some embodiments, release factors may comprise protein adaptors with two major activities. In some embodiments, the first major activity can comprise a Class 1 activity. In some embodiments, the Class 1 activity can comprise mRNA-binding and recognizing the stop codon. In some embodiments, the Class 1 activity may be provided by a Class 1 release factor. In some embodiments, the Class 1 activity may be provided by a release factor 1 (RF1) or an RF2. In some embodiments, the Class 1 activity may be provided by a eukaryotic release factor 1 (eRF1). In some embodiments, the second major activity can comprise a Class 2 activity. In some embodiments, the Class 2 activity may be provided by Class 2 release factor. In some embodiments, the Class 2 activity may be provided by an RF3. In some embodiments, the Class 2 activity may be provided by an eRF3. In some embodiments, the Class 2 activity may comprise protein-binding and recognizing the ribosome to release the translated protein.

Wobble rules can be different for RFs than for tRNAs. Release factors can recognize NNA separately from NNG (anti-codon starts with U) and from NNA/C/U (anti-codon starts with A modified to I). For sense codons, NNA can be either recognized with NNU/A as a two-codon block or with NNT/C/A as a three-codon block, or as part of NNT/C/G/A as a four-codon block.

### Release Factors (RFs) in Prokaryotes and Eukaryotes

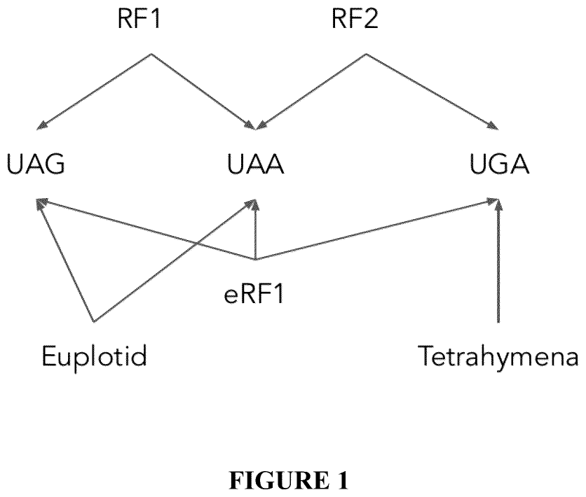

In some embodiments, the release factors can comprise release factors (RFs) from prokaryotes. In some embodiments, the prokaryotic release factors can comprise release factors from Eubacteria and/or mitochondria. In some embodiments, the prokaryotic release factors can comprise two classes (FIG. 1). In some embodiments, the prokaryotic Class 1 release factors can comprise RF1 and RF2. In some embodiments, RF1 can recognize the stop codons UAA and UAG. In some embodiments, RF2 can recognize the stop codons UAA and UGA. In some embodiments, the prokaryotic Class 2 release factors can comprise RF3. In some embodiments, release factors can comprise a recognition domain. In some embodiments, the recognition domain can recognize a stop codon.

In some embodiments, the release factors can comprise release factors from eukaryotes. In some embodiments, the eukaryotic release factors can comprise release factors from Eukaryotes and/or Archaebacteria. In some embodiments, the eukaryotic release factors can comprise two classes (FIG. 1). In some embodiments, the eukaryotic Class 1 release factors can comprise eRF1. In some embodiments, eRF1 can recognize the stop codons UAA, UAG, and UGA. Table 1 shows the activity of eRF1 in different eukaryotic organisms. In some embodiments, the eukaryotic Class 2 release factors can comprise eRF3.

### Evolution

RF1/2 and eRF1 may not be homologous. This lack of homology may suggest that RF activity was provided by RNA adapters prior to the Eubacteria-Archaebacteria split.

Most wild type (WT) eukaryotic RFs (eRFs), including but not limited to yeasts, may recognize all three stop codons, UAG, UAA and UGA. eRFs may form a heterodimer comprising eRF1 and eRF3. In yeast, and more specifically Saccharomyces cerevisiae, eRF1 and eRF3 can be referred to as SUP45 and SUP35, respectively. Some ciliates may have RFs that recognize a subset of the stop codons. For example, a ciliate may have RFs recognizing UAA and UAG. In another example, a ciliate may have RFs recognizing UGA. A yeast system can be engineered with all of the advantages of yeast, for example better suitability for producing certain proteins or other biologics that can be more difficult to produce in bacterial systems. For example, one or more specific domains in yeast eRF1 may be engineered to enable stop codon selectivity conferred in RF of ciliates by replacing one or more yeast amino acids with the corresponding ciliate amino acids. In some embodiments, the yeast eRF1 can be replaced with ciliate eRF1. In some embodiments, the eRF1/eRF3 heterodimer can be replaced with ciliate eRF1/eRF3.

TABLE 1
eRF1 activity in different organisms.

| | | Table 6: | | | | | | |
|---|---|---|---|---|---|---|---|---|
| | | Ciliate | | | | | | |
| | | (Spirotrichea/ | | | | | | |
| | | Oxytricha/ | | | | | | |
| | | Stylonychia, | | | | | | |
| | | Paramecium, | | | | | | |
| | | Tetrahymena, | | | | | | |
| | | Heterotrichea/ | | | | | | |
| | | Blepharisma), | | | | | | |
| | | Green | Table | | Table | | | Table |
| | | algae | 10: | Table | 28: | Table | Table: | 31: |
| | | (Dasycladacean), | Ciliate | 27: | Ciliate | 29: | 30 | Euglenozoa |
| | Table 1: | Flagellate | (Spirotrichea/ | Ciliate | (Heterotrichea/ | Ciliate | Ciliate | (Trypanosomatida/ |
| Codon | Standard | (Hexamita) | Euplotid) | (Karyorelict) | Condylostoma) | (Mesodinium) | (Peritrich) | Blastocrithidia) |
| UAU/C | Tyr | | | | | Tyr | | |
| UAA | Stop | Gln | | Gln | Gln/Stop | Tyr | Glu | Glu/Stop |
| | (Ochre) | | | | | | | |
| UAG | Stop | Gln | | Gln | Gln/Stop | Tyr | Glu | Glu/Stop |
| | (Amber) | | | | | | | |
| UGU/C | Cys | | Cys | | | | | |
| UGA | Stop | | Cys | Trp/Stop | Trp/Stop | | | Trp |
| | (Opal) | | | | | | | |
| UGG | Trp | | | Trip | Trip | | | |
| CAA/G | Gln | | | | | | | |
| GAA/G | Glu | | | | | | | |
| Release | | UGA | UAA/G | UGA | Standard | UGA | UGA | UAA/G |
| factor | | only | only | with 3′ | with 3′ | only | only | with 3′ |
| recognition | | | | UTR | UTR | | | UTR |

Tables here refer to NCBI Genetic Code Tables, which can be found here: https://www.ncbi.nlm.nih.gov/Taxonomy/Utils/wprintgc.cgi.
The Standard scheme shown in Table 1 is used by most organisms.

Stop-codon assignment to sense codon may have happened as multiple independent events (ciliate, flagellate, green algae lineages). For example, ciliates can comprise a unicellular eukaryote that includes several lineages where stop codons in the standard genetic code have been reassigned to amino acids.

In some embodiments, eRF1 can comprise two main patterns of eRF1 activity. In some embodiments, the first pattern of eRF1 activity can comprise the recognition of the stop codon UGA only. In some embodiments, the stop codons UAA and UAG can be captured by wobble (e.g., UAC/U Tyr). In some embodiments, the stop codons UAA and UGA can be captured by a 1^{st} position neighbor (e.g., CAA/G Gln or GAA/G Glu).

In some embodiments, the second pattern of eRF1 activity can comprise the recognition of UAA/UAG only. In some embodiments, the stop codon UGA can be captured by wobble (e.g., UGU/C Cys, UGG Trp).

In some ciliates, the eRF1 recognition can be “clean” and can depend only on the codon. In other ciliates, stop-codon recognition can depend on 3′ UTR structure.

In some embodiments, UAG can be useful for recoding. In some embodiments, the anticodons for UAA and UGA may have too much wobble for recoding.

Unlike prokaryotes where recognition patterns are UAA/UAG and UAA/UGA, in eukaryotic species where stop codons have been captured as sense codons, evolution seems to favor UAA/UAG and UGA alone.

In some embodiments, the release factor may comprise a class 1 release factor. In some embodiments, the class 1 release factor may comprise a prokaryotic release factor 1 (RF1). In some cases, the RF1 may be a eukaryotic RF1 (eRF1). In some embodiments, the eRF1 may be from a ciliate. In some embodiments, the class 1 release factor may comprise a prokaryotic release factor 2 (RF2). In some embodiments, the class 1 release factor may comprise RF1 and RF2. In some embodiments, the release factor may comprise a class 2 release factor. In some embodiments, the class 2 release factor may comprise a release factor 3 (RF3). In some embodiments, the RF3 may be a eukaryotic RF3 (eRF3). In some embodiments, the release factor may be a class 1 release factor or a class 2 release factor. In some embodiments, the release factor may be a class 1 release factor and a class 2 release factor. In some embodiments, the release factor may be a chimeric release factor. In some embodiments, the release factor may be a release factor complex. In some cases, the release factor complex may comprise a release factor 1/release factor 3 (RF1/RF3) complex. In some cases, the release factor complex may comprise a eukaryotic release factor 1/eukaryotic release factor 3 (eRF1/eRF3) complex. In some cases, the release factor complex may comprise a eRF1/chimeric yeast-ciliate eRF3.

Most wild-type eukaryotic release factors can recognize all three stop codons (e.g., UAG/UAA/UGA). In some cases, a ciliate or other eukaryote, may have release factors that may not recognize all the stop codons. In some cases, a ciliate or a eukaryote may have release factors that may require additional sequence at the 3′ of a stop codon for recognition as a stop codon. For example, some release factors may recognize only UGA as a stop codon and UAA/UAG as sense codons. For example, other release factors may recognize UAA/UAG as stop codons and UGA as a sense codon.

In some embodiments, some release factors can recognize UGA as a stop codon. In some embodiments, some release factors can recognize UGA as a stop codon and UAG/UAA as sense codons. In some embodiments, some release factors can recognize UGA/UAG as stop codons. In some embodiments, some release factors can recognize UGA/UAG as stop codons and recognize UAA as a sense codon. In some embodiments, some release factors can recognize UGA/UAA as stop codons. In some embodiments, some release factors can recognize UGA/UAA as stop codons and recognize UAG as a sense codon. In some embodiments, some release factors can recognize UAG as a stop codon. In some embodiments, some release factors can recognize UAG as a stop codon and recognize UGA/UAA as sense codons. In some embodiments, some release factors can recognize UAG/UAA as stop codons. In some embodiments, some release factors can recognize UAG/UAA as stop codons and recognize UGA as a sense codon. In some embodiments, some release factors can recognize UAA as a stop codon. In some embodiments, some release factors can recognize UAA as a stop codon and recognize UGA/UAG as sense codons. In some embodiments, some release factors may recognize UGA/UAG/UAA as stop codons. In some embodiments, some release factors may recognize UGA/UAG/UAA as sense codons.

### Release Factor Engineering

In some aspects, provided herein are compositions, systems, and methods for producing polypeptides comprising one or more non-canonical amino acids (ncAAs). In some embodiments, the compositions, systems, and methods described herein does not comprise replacing or rewriting a codon. In some embodiments, compositions, systems, and methods described herein may utilize tRNAs that recognize a stop codon. In some embodiments, the stop codon may be UAG, UGA, or UAA. In some embodiments, the stop codon may be UAG. In some embodiments, the tRNAs that recognize a stop codon may be called suppressor tRNAs. In some embodiments, the tRNAs that recognize a stop codon may decode the stop codon as a sense codon. For example, when a stop codon (e.g., UAG, UGA, or UAA) appears in a messenger RNA (mRNA) transcript, the stop codon may be decoded by suppressor tRNAs. In this example, suppressor tRNAs may insert an amino acid into a polypeptide chain. The amino acid, in this example, may comprise an ncAA and the polypeptide chain may comprise an ncAA. In this example, a native release factor may be present in the same cell and may compete with suppressor tRNAs. As such, in this example, a stop codon may be read as a sense codon by suppressor tRNAs or may be read as a stop codon by the native release factor and translation may be terminated. As such, in this example, the yield of the polypeptide chain comprising an ncAA may be low. Without wishing to be bound by any theory, each time a stop codon is readthrough as a sense codon by suppressor tRNAs, the probability of the next successful readthrough of a stop codon may be multiplied by an efficiency that is less than 1. As such, a protein designed to have 3 or 4 ncAAs may have overall success rate of 1-in-100 to 1-in-1000 using this system comprising suppressor tRNAs.

In some aspects, provided herein are compositions, systems, and methods for producing polypeptides comprising one or more non-canonical amino acids (ncAAs). In some embodiments, the compositions, systems, and methods described herein does not comprise replacing or rewriting a codon. In some embodiments, the compositions, systems, and methods may comprise altering release activity of a release factor so that different release factor proteins recognize different subsets of stop codons. In some embodiments, one or more subset of release factors may be removed to leave a stop codon without a cognate release factor.

As described above, in eukaryotic cells, a single eukaryotic release factor (eRF1) recognizes all stop codons. In the standard genetic code, eRF1 may recognize UAG, UAA, and UGA. Simple deletion of eRF1 would not lead to a functional translation system for a polypeptide comprising an ncAA because the ncAA could be incorporated but translational termination would be defective. As such, compositions, systems, and methods described herein utilizes release factors with distinct activity from organisms with nonstandard genetic code as the source. In some embodiments, an eRF1 from organisms with NCBI translation table 6 (Translation table 6: The ciliate, dasycladacean and hexamita nuclear code) may be used. In this embodiment, the eRF1 from organisms with NCBI translation table 6 may recognize only UGA as a stop codon. In organisms with NCBI translation table 6, UAA and UAG stop codons may be used as sense codons for glutamine (Glu or Q). In some embodiments, organisms with NCBI translation table 6 may comprise Oxytricha, Paramecium, Stylonychia, or Tetrahymena. In some embodiments, an eRF1 from organisms with NCBI translation table 10 (Translation table 10: The euplotid nuclear code) may be used. In this embodiment, the eRF1 from have an eRF1 may recognizes UAA and UAG as stop codons. In organisms with NCBI translation table 10, UGA stop codon may be used as a sense codon for cysteine (Cys or C). In some embodiments, organisms with NCBI translation table 10 may comprise Euplotes, Euplotoides, or Stentor.

In some embodiments, release factors described herein may be engineered or configured to recognize a stop codon. In some embodiments, release factors described herein may be engineered or configured to recognize UGA, UAA, UAG, or any combination thereof as a stop codon. In one embodiment, release factors described herein may be engineered or configured to recognize UGA as a stop codon. In this embodiment, release factors may not recognize UAA and/or UAG as a stop codon. In another embodiment, release factors described herein may be engineered or configured to recognize UAA, UAG, or any combination thereof as a stop codon. In this embodiment, release factors may not recognize UGA as a stop codon. In some embodiments, release factors described herein may be engineered or configured to recognize at most two or at most one stop codon. In some embodiments, engineered release factors described herein may recognize UAG. In some embodiments, engineered release factors described herein may recognize UAA. In some embodiments, engineered release factors described herein may recognize UAG and UAA. In some embodiments, engineered release factors described herein may recognize UGA and UAA. In some embodiments, engineered release factors described herein may recognize UGA and UAG. In some embodiments, engineered release factors described herein may recognize only UGA. In some embodiments, some release factors may have evolved naturally to recognize at most one stop codon. In some embodiments, a recognition domain of release factors may be swapped. For example, a recognition domain of yeast release factors may be swapped with a recognition domain of a ciliate release factor (e.g., domain/motif-swapped release factor). In some embodiments, a recognition domain of release factors may be swapped as a contiguous segment or as one or more non-contiguous amino acid changes.

In some aspects, compositions, systems, and methods described herein may comprise at least two release factors comprising a first release factor and a second release factor. In some embodiments, release factors described herein may be engineered or recombinant release factors. In one aspect, compositions, systems, and methods described herein may comprise two release factors comprising a first release factor engineered or configured to recognize UGA as a stop codon and a second release factor engineered or configured to recognize UAA, UAG, or any combination thereof as a as stop codon. In some embodiments, compositions, systems, and methods described herein may comprise two release factors comprising a first release factor engineered or configured to recognize UGA as a stop codon and a second release factor engineered or configured to recognize UAA and UAG as as stop codons. In some embodiments, the first recombinant release factor may not recognize UAA or UAG as a stop codon. In some embodiments, the first recombinant release factor may not recognize UAA and UAG as stop codons. In some embodiments, the second release factor may not recognize UGA.

In another aspect, compositions, systems, and methods described herein may comprise two release factors comprising a first release factor engineered or configured to recognize UAA, UAG, or any combination thereof as a stop codon and a second release factor engineered or configured to recognize UGA as a stop codon. In some embodiments, compositions, systems, and methods described herein may comprise two release factors comprising a first release factor engineered or configured to recognize UAA and UAG as stop codons and a second release factor engineered or configured to recognize UGA as a stop codon. In some embodiments, the first recombinant release factor may not recognize UGA as a stop codon. In some embodiments, the second recombinant release factor may not recognize UAA or UAG as a stop codon. In some embodiments, the second release factor may not recognize UAA and UAG as stop codons.

In yet another aspect, compositions, systems, and methods described herein may comprise two release factors comprising a first release factor engineered or configured to recognize UGA, UAA, UAG, or any combination thereof as a stop codon and a second release factor recognizing UGA, UAA, and UAG as stop codons. In some embodiments, the first release factor may recognize UGA as a stop codon. In some embodiments, the first release factor may recognize UAA or UAG as a stop codon. In some embodiments, the first release factor may recognize UAA and UAG as stop codons.

In some embodiments, recombinant release factors described herein may be engineered using one or more embodiments described below.

### Embodiment 1. Amino Acid Swap

In some embodiments, a release factor may comprise one or more mutations. In some cases, the one or more mutations may allow the release factor to recognize only a subset of stop codons (e.g., recognize only one or two stop codons, but not all three stop codons).

In some embodiments, the release factor may comprise at least one, at least two, at least three, at least four, at least five, at least about six, at least about seven, at least about eight, at least about nine, at least about ten, at least about 20, at least about 30, at least about 40, at least about 50, at least about 60, at least about 70, at least about 80, at least about 90, at least about 100, or more mutations. In some embodiments, the mutations may result in the release factor not recognizing a stop codon. In some embodiments, the mutated release factor may not recognize UGA. In some embodiments, the mutated release factor may not recognize UAG. In some embodiments, the mutated release factor may not recognize UAA. In some embodiments, the mutated release factor may not recognize UGA and UAG. In some embodiments, the mutated release factor may not recognize UGA and UAA. In some embodiments, the mutated release factor may not recognize UAG and UAA.

In some embodiments, the mutations may modify a domain or a motif in the endogenous release factor to resemble a domain or motif of a release factor from another organism comprising, but not limited to a ciliate. In some embodiments, a ciliate can comprise any ciliate that uses UGA codons as a termination or stop codon. In some embodiments, a ciliate can comprise, but is not limited to, Blepharisma americanum, Paramecium tetraurelia, Tetrahymena thermophila, Blepharisma japonicum, Euplotes aediculatus, Euplotes octocarinatus, Stentor coeruleus, Nyctotherus ovalis, Stylonychia lemnae, Oxytricha trifallax, Stylonychia pustulata, Stylonychia mytilus, Eschaneustyla sp. HL-2004, Gonostomum so. HL-2004, Holosticha sp. HL-2004, Urostyla sp. HL-2004, Uroleptus sp. WIC-2003, Paraurostyla weissei, Stichotrichida sp Misty, Stichotrichida sp Alaska, Spiromucleus salmonicida, or Loxodes striatus.

### Embodiment 2. Domain/Motif Swap

In some cases, a recognition domain of a release factor can be swapped or replaced with a recognition domain of another release factor (e.g., a recognition domain of a ciliate, green algae, or flagellates). In some cases, one or more recognition domains can be replaced with one or more recognition domains of another release factor (e.g., a ciliate, green algae, or flagellate), for example, via introducing a point mutation or replacing one or more continuous segments of the recognition domain. In some embodiments, the domain/motif swapping of a release factor can result in not recognizing a stop codon. In some embodiments, the domain/motif-swapped release factor may not recognize UGA. In some embodiments, the domain/motif-swapped release factor may not recognize UAG. In some embodiments, the domain/motif-swapped release factor may not recognize UAA. In some embodiments, the domain/motif-swapped release factor may not recognize UGA and UAG. In some embodiments, the domain/motif-swapped release factor may not recognize UGA and UAA.

In some embodiments, the domain/motif-swapped release factor may not recognize UAG and UAA.

In some embodiments, a domain or motif in the release factor may be swapped with a domain or motif of a release factor from another organism comprising, but not limited to, a ciliate. In some embodiments, a ciliate can comprise any ciliate that uses UGA codons as a termination or stop codon. In some embodiments, a release factor may comprise a first recognition domain. In some embodiments, a release factor may comprise a first recognition domain swapped with a second recognition domain. In some embodiments, the second recognition domain may be from a second organism. In some embodiments, the second organism may be from a different species of yeast. In some embodiments, the second organism may comprise a ciliate. In some embodiments, a ciliate may comprise any ciliate that uses UGA codons as a termination or stop codon. In some embodiments, a ciliate may comprise any ciliate that uses UAA and/or UAG codons as a termination or stop codon.

In some embodiments, a ciliate may comprise, but is not limited to, Blepharisma americanum, Blepharisma japonicum, Euplotes aediculatus, Euplotes octocarinatus, Stentor coeruleus, Nyctotherus ovalis, Stylonychia lemnae, Pseudocohnilembus persalinus, Ichthyophthirius multifiliis, Stylonychia lemnae, Oxytricha trifallax, Stylonychia pustulata, Stylonychia mytilus, Eschaneustyla sp. HL-2004, Gonostomum sp. HL-2004, Holosticha sp. HL-2004, Urostyla sp. HL-2004, Uroleptus sp. WIC-2003, Paraurostyla weissei, Stichotrichida sp. Misty, Stichotrichida sp. Alaska, Spironucleus salmonicida, Loxodes striatus, Paramecium tetraurelia, or Tetrahymena thermophila. In some embodiments, the second recognition domain may be identified using phylogenetic screening, directed evolution, library screening, or machine learning.

Domain or motif swapping and mutagenesis experiments in vivo can be allowed in part by using temperature-sensitive mutants of the release factor, eRF1-ts (e.g., sup45-ts). Known mutants can be functional at permissive temperature and have limited or reduced functionality at non-permissive temperature. Engineered RFs can be introduced into a host cell. For example, an engineered eRF1 can be introduced into a yeast cell expressing an eRF1-ts rather than a wild-type, eRF1-wt. After an engineered eRF1 is introduced into the cell expressing eRF1-ts and lacking eRF1-wt at permissive temperature, cell viability can be checked at a non-permissive temperature to test whether the engineered eRF1 can complement the limited or reduced functionality of the eRF1-ts. Permissive and non-permissive temperatures are described in other sections of the present disclosure.

In some embodiments, domain/motif-swapped eRF1 may not recognize stop codon UAA/G in vitro at a non-permissive temperature, but may recognize UAA/G in vivo at a permissive temperature. In some embodiments, recognition of UAA/G stop codon by an release factor may be reduced in the presence of an orthogonal tRNA that can recognize the same stop codon as a sense codon.

### Embodiment 3. Native Ciliate Release Factor Machinery

In some embodiments, compositions, systems, and methods for producing polypeptides comprising an ncAA described herein may utilize release factors from native ciliate machinery. In some embodiments, native ciliate machinery can comprise non-mutated release factors from a ciliate. In some embodiments, the non-mutated ciliate release factors may recognize one or more stop codons. In some embodiments, the non-mutated ciliate release factors may recognize UGA. In some embodiments, the non-mutated ciliate release factors may recognize UAG. In some embodiments, the non-mutated ciliate release factors may recognize UAA. In some embodiments, the non-mutated ciliate release factors may recognize UGA and UAG. In some embodiments, the non-mutated ciliate release factors may recognize UGA and UAA. In some embodiments, the non-mutated ciliate release factors may recognize UAG and UAA. In some embodiments, the non-mutated ciliate release factors may recognize UGA. In some embodiments, a ciliate may comprise any ciliate that uses UAA and UAG as a termination or stop codon. In some embodiments, a ciliate may comprise any ciliate that uses UGA codons as a termination or stop codon. In some embodiments, a ciliate may comprise, but is not limited to, Blepharisma americanum, Blepharisma japonicum, Euplotes aediculatus, Euplotes octocarinatus, Stentor coeruleus, Nyctotherus ovalis, Stylonychia lemnae, Pseudocohnilembus persalinus, Ichthyophthirius multifiliis, Stylonychia lemnae, Oxytricha trifallax, Stylonychia pustulata, Stylonychia mytilus, Eschaneustyla sp. HL-2004, Gonostomum sp. HL-2004, Holosticha sp. HL-2004, Urostyla sp. HL-2004, Uroleptus sp. WIC-2003, Paraurostyla weissei, Stichotrichida sp. Misty, Stichotrichida sp. Alaska, Spironucleus salmonicida, Loxodes striatus, Paramecium tetraurelia, or Tetrahymena thermophila.

In some embodiments, the second recognition domain can comprise an amino acid sequence comprising KSSNIKS (SEQ ID NO: 3). In some embodiments, the second recognition domain can comprise an amino acid sequence comprising YICDNKF (SEQ ID NO: 4). In some embodiments, the second recognition domain can comprise an amino acid sequence comprising KSSNIKS (SEQ ID NO: 3) and YICDNKF (SEQ ID NO: 4). In some embodiments, the second recognition domain can comprise an amino acid sequence comprising YFCDPQF (SEQ ID NO: 10). In some embodiments, the second recognition domain can comprise an amino acid sequence comprising EAASIKD (SEQ ID NO: 11). In some embodiments, the second recognition domain can comprise an amino acid sequence comprising KATNIKD (SEQ ID NO: 12). In some embodiments, the second recognition domain can comprise an amino acid sequence comprising YFCDSKF (SEQ ID NO: 13). In some embodiments, the second recognition domain can comprise an amino acid sequence comprising TAVNIKS (SEQ ID NO: 5). In some embodiments, the second recognition domain can comprise an amino acid sequence comprising KAANIKS (SEQ ID NO: 6). In some embodiments, the second recognition domain can comprise an amino acid sequence comprising KASNIKS (SEQ ID NO: 7). In some embodiments, the second recognition domain can comprise an amino acid sequence comprising YYCGERF (SEQ ID NO: 8). In some embodiments, the second recognition domain can comprise an amino acid sequence comprising TAESIKS (SEQ ID NO: 9). In some embodiments, the second recognition domain can comprise an amino acid sequence comprising FDFDAES (SEQ ID NO: 14). In some embodiments, the second recognition domain can comprise an amino acid sequence comprising TLIKPQF (SEQ ID NO: 15). In some embodiments, the second recognition domain can comprise an amino acid sequence comprising TGDKIKS (SEQ ID NO: 16). In some embodiments, the second recognition domain can comprise an amino acid sequence comprising TIIKNDF (SEQ ID NO: 17). In some embodiments, the second recognition domain can comprise an amino acid sequence comprising EAASIQD (SEQ ID NO: 18). In some embodiments, the second recognition domain can comprise an amino acid sequence comprising FFCDNYF (SEQ ID NO: 19). In some embodiments, the second recognition domain can comprise an amino acid sequence comprising FVIVNKF (SEQ ID NO: 20). In some embodiments, the second recognition domain can comprise an amino acid sequence comprising AAQNIKS (SEQ ID NO: 21). In some embodiments, the second recognition domain can comprise an amino acid sequence comprising YFCGGKF (SEQ ID NO: 22). In some embodiments, the second recognition domain can comprise an amino acid sequence comprising QANSIKD (SEQ ID NO: 23). In some embodiments, the second recognition domain can comprise an amino acid sequence comprising YRCDSKF (SEQ ID NO: 24). In some embodiments, the second recognition domain can comprise an amino acid sequence comprising GAASIKN (SEQ ID NO: 25). In some embodiments, the second recognition domain can comprise an amino acid sequence comprising YSCNTIF (SEQ ID NO: 26). In some embodiments, the second recognition domain can comprise an amino acid sequence comprising SAQNIKS (SEQ ID NO: 27). In some embodiments, the second recognition domain can comprise an amino acid sequence comprising YYCDNRF (SEQ ID NO: 28). In some embodiments, the second recognition domain can comprise an amino acid sequence comprising SAGNIKS (SEQ ID NO: 29). In some embodiments, the second recognition domain can comprise an amino acid sequence comprising YFCDNSF (SEQ ID NO: 30). In some embodiments, the second recognition domain can comprise an amino acid sequence comprising TAQNIKS (SEQ ID NO: 31). In some embodiments, the second recognition domain can comprise an amino acid sequence comprising SAQSIKS (SEQ ID NO: 32). In some embodiments, the second recognition domain can comprise an amino acid sequence comprising AANNIKS (SEQ ID NO: 33). In some embodiments, the second recognition domain can comprise an amino acid sequence comprising YNCSGKF (SEQ ID NO: 34). In some embodiments, the second recognition domain can comprise an amino acid sequence comprising QAQNIKS (SEQ ID NO: 35). In some embodiments, the second recognition domain can comprise an amino acid sequence comprising QADCIKS (SEQ ID NO: 36). In some embodiments, the second recognition domain can comprise an amino acid sequence comprising YSCDGVF (SEQ ID NO: 37). In some embodiments, the second recognition domain can comprise an amino acid sequence comprising RAQNIKS (SEQ ID NO: 38). In some embodiments, the second recognition domain can comprise an amino acid sequence comprising FLCENTF (SEQ ID NO: 39).

In some embodiments, the release factor may comprise a second recognition domain comprising an amino acid sequence listed in Table 3. In some embodiments, the release factor may comprise a second recognition domain comprising an amino acid sequence selected from the group consisting of SEQ ID NOs: 3-39. In some embodiments, the release factor comprising an amino acid sequence listed in Table 3 can be expressed from a nucleic acid sequence selected from the group consisting of SEQ ID NOs: 101-125. In some embodiments, the release factor comprising a second recognition domain comprising an amino acid sequence selected from the group consisting of SEQ ID NOs: 3-39 can be expressed from a nucleic acid sequence selected from the group consisting of SEQ ID NOs: 101-125.

In some embodiments, the release factor described herein may comprise an amino acid sequence with at least 50%, at least 55%, at least 60%, at least 65%, at least 70%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to an amino acid sequence selected from the group consisting of SEQ ID NOs: 40-64. In some embodiments, the release factor described herein may comprise an amino acid sequence selected from the group consisting of SEQ ID NOs: 40-64. In some embodiments, the release factor described herein may be expressed from a nucleic acid sequence selected from the group consisting of SEQ ID NOs: 101-125.

In some embodiments, the release factor described herein may comprise an amino acid sequence with at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to an amino acid sequence selected from the group consisting of SEQ ID NOs: 65-74. In some embodiments, the release factor described herein may comprise an amino acid sequence selected from the group consisting of SEQ ID NOs: 65-74. In some embodiments, the release factor described herein may be expressed from a nucleic acid sequence selected from the group consisting of SEQ ID NOs: 126-135.

In some embodiments, the release factor described herein may comprise an amino acid sequence with at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to an amino acid sequence selected from the group consisting of SEQ ID NOs: 75-92. In some embodiments, the release factor described herein may comprise an amino acid sequence selected from the group consisting of SEQ ID NOs: 75-92. In some embodiments, the release factor described herein may be expressed from a nucleic acid sequence selected from the group consisting of SEQ ID NOs: 136-153.

In some embodiments, the release factor described herein may comprise an amino acid sequence with at least 50%, at least 55%, at least 60%, at least 65%, at least 70%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to an amino acid sequence selected from the group consisting of SEQ ID NOs: 93-100. In some embodiments, the release factor described herein may comprise an amino acid sequence selected from the group consisting of SEQ ID NOs: 93-100. In some embodiments, the release factor described herein may be expressed from a nucleic acid sequence selected from the group consisting of SEQ ID NOs: 154-161.

In some embodiments, the release factor from the second organism can comprise an eRF1. In some embodiments, the eRF1 from the second organism can comprise an amino acid sequence that has between about at least 10% to about at least 50% sequence identity to an amino acid sequence of an eRF1 of the first organism. In some embodiments, the eRF1 from the second organism can comprise an amino acid sequence that has at least 10% sequence identity to an amino acid sequence of an eRF1 of the first organism. In some embodiments, the eRF1 from the second organism can comprise an amino acid sequence that has at least 15% sequence identity to an amino acid sequence of an eRF1 of the first organism. In some embodiments, the eRF1 from the second organism can comprise an amino acid sequence that has at least 20% sequence identity to an amino acid sequence of an eRF1 of the first organism. In some embodiments, the eRF1 from the second organism can comprise an amino acid sequence that has at least 25% sequence identity to an amino acid sequence of an eRF1 of the first organism. In some embodiments, the eRF1 from the second organism can comprise an amino acid sequence that has at least 30% sequence identity to an amino acid sequence of an eRF1 of the first organism. In some embodiments, the eRF1 from the second organism can comprise an amino acid sequence that has at least 35% sequence identity to an amino acid sequence of an eRF1 of the first organism. In some embodiments, the eRF1 from the second organism can comprise an amino acid sequence that has at least 40% sequence identity to an amino acid sequence of an eRF1 of the first organism. In some embodiments, the eRF1 from the second organism can comprise an amino acid sequence that has at least 45% sequence identity to an amino acid sequence of an eRF1 of the first organism. In some embodiments, the eRF1 from the second organism can comprise an amino acid sequence that has at least 50% sequence identity to an amino acid sequence of an eRF1 of the first organism.

In some embodiments, the release factor from the second organism can comprise an eRF1/eRF3 complex. In some embodiments, the eRF1 of the eRF1/eRF3 complex from the second organism can comprise an amino acid sequence that has between about at least 10% to about at least 50% sequence identity to an amino acid sequence of an eRF1 of the first organism. In some embodiments, the eRF1 of the eRF1/eRF3 complex from the second organism can comprise an amino acid sequence that has at least 10% sequence identity to an amino acid sequence of an eRF1 of the first organism. In some embodiments, the eRF1 of the eRF1/eRF3 complex from the second organism can comprise an amino acid sequence that has at least 15% sequence identity to an amino acid sequence of an eRF1 of the first organism. In some embodiments, the eRF1 of the eRF1/eRF3 complex from the second organism can comprise an amino acid sequence that has at least 20% sequence identity to an amino acid sequence of an eRF1 of the first organism. In some embodiments, the eRF1 of the eRF1/eRF3 complex from the second organism can comprise an amino acid sequence that has at least 25% sequence identity to an amino acid sequence of an eRF1 of the first organism. In some embodiments, the eRF1 of the eRF1/eRF3 complex from the second organism can comprise an amino acid sequence that has at least 30% sequence identity to an amino acid sequence of an eRF1 of the first organism. In some embodiments, the eRF1 of the eRF1/eRF3 complex from the second organism can comprise an amino acid sequence that has at least 35% sequence identity to an amino acid sequence of an eRF1 of the first organism. In some embodiments, the eRF1 of the eRF1/eRF3 complex from the second organism can comprise an amino acid sequence that has at least 40% sequence identity to an amino acid sequence of an eRF1 of the first organism. In some embodiments, the eRF1 of the eRF1/eRF3 complex from the second organism can comprise an amino acid sequence that has at least 45% sequence identity to an amino acid sequence of an eRF1 of the first organism. In some embodiments, the eRF1 of the eRF1/eRF3 complex from the second organism can comprise an amino acid sequence that has at least 50% sequence identity to an amino acid sequence of an eRF1 of the first organism.

In some embodiments, the eRF3 of the eRF1/eRF3 complex from the second organism can comprise an amino acid sequence that has between about at least 10% to about at least 50% sequence identity to an amino acid sequence of an eRF3 of the first organism. In some embodiments, the eRF3 of the eRF1/eRF3 complex from the second organism can comprise an amino acid sequence that has at least 10% sequence identity to an amino acid sequence of an eRF3 of the first organism. In some embodiments, the eRF3 of the eRF1/eRF3 complex from the second organism can comprise an amino acid sequence that has at least 15% sequence identity to an amino acid sequence of an eRF3 of the first organism. In some embodiments, the eRF3 of the eRF1/eRF3 complex from the second organism can comprise an amino acid sequence that has at least 20% sequence identity to an amino acid sequence of an eRF3 of the first organism. In some embodiments, the eRF3 of the eRF1/eRF3 complex from the second organism can comprise an amino acid sequence that has at least 25% sequence identity to an amino acid sequence of an eRF3 of the first organism. In some embodiments, the eRF3 of the eRF1/eRF3 complex from the second organism can comprise an amino acid sequence that has at least 30% sequence identity to an amino acid sequence of an eRF3 of the first organism. In some embodiments, the eRF3 of the eRF1/eRF3 complex from the second organism can comprise an amino acid sequence that has at least 35% sequence identity to an amino acid sequence of an eRF3 of the first organism. In some embodiments, the eRF3 of the eRF1/eRF3 complex from the second organism can comprise an amino acid sequence that has at least 40% sequence identity to an amino acid sequence of an eRF3 of the first organism. In some embodiments, the eRF3 of the eRF1/eRF3 complex from the second organism can comprise an amino acid sequence that has at least 45% sequence identity to an amino acid sequence of an eRF3 of the first organism. In some embodiments, the eRF3 of the eRF1/eRF3 complex from the second organism can comprise an amino acid sequence that has at least 50% sequence identity to an amino acid sequence of an eRF3 of the first organism.

In some embodiments, the release factor from the second organism can comprise an eRF1. In some embodiments, the eRF1 from the second organism can form a complex with an eRF3 from the first organism. In some embodiments, the eRF1 from the second organism can form a complex with an eRF3 from the second organism. In some embodiments, the eRF1 from the second organism can form a complex with a chimeric eRF3. In some embodiments, the chimeric eRF3 can comprise an eRF3 from the first organism or a fragment thereof and an eRF3 from a second organism or a fragment thereof. In some embodiments, the second organism can comprise, but is not limited to, Euplotes octocarinatus or Paramecium tetraurelia. In some embodiments, the chimeric eRF3 can comprise an eRF3 from Euplotes octocarinatus. In some embodiments, the chimeric Euplotes octocarinatus eRF3 can comprise amino acids 7-298 of the eRF3 from Euplotes octocarinatus can be replaced with amino acids 6-253 of the eRF3 from the first organism. In some embodiments, the chimeric Euplotes octocarinatus eRF3 can comprise an amino acid sequence with at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to an amino acid sequence comprising SEQ ID NO: 93 or SEQ ID NO: 94. In some embodiments, the chimeric Euplotes octocarinatus eRF3 can comprise an amino acid sequence comprising SEQ ID NO: 93 or SEQ ID NO: 94. In some embodiments, the chimeric Euplotes octocarinatus eRF3 can comprise a nucleic acid sequence comprising SEQ ID NO: 154 or SEQ ID NO: 155. In some embodiments, the chimeric Euplotes octocarinatus eRF3 can comprise amino acids 1-298 of the eRF3 from Euplotes octocarinatus can be replaced with amino acids 1-253 of the eRF3 from the first organism. In some embodiments, the chimeric Euplotes octocarinatus eRF3 can comprise an amino acid sequence with at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to an amino acid sequence comprising SEQ ID NO: 95 or SEQ ID NO: 96. In some embodiments, the chimeric Euplotes octocarinatus eRF3 can comprise an amino acid sequence comprising SEQ ID NO: 95 or SEQ ID NO: 96. In some embodiments, the chimeric Euplotes octocarinatus eRF3 can comprise a nucleic acid sequence comprising SEQ ID NO: 156 or SEQ ID NO: 157. In some embodiments, the chimeric eRF3 can comprise an eRF3 from Paramecium tetraurelia. In some embodiments, the chimeric Paramecium tetraurelia eRF3 can comprise amino acid 1-321 of the eRF3 from Paramecium tetraurelia can be replaced with amino acids 1-253 of the eRF3 from the first organism. In some embodiments, the chimeric Paramecium tetraurelia eRF3 can comprise an amino acid sequence with at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to an amino acid sequence comprising SEQ ID NO: 97, SEQ ID NO: 98, SEQ ID NO: 99, or SEQ ID NO: 100. In some embodiments, the chimeric Paramecium tetraurelia eRF3 can comprise an amino acid sequence comprising SEQ ID NO: 97, SEQ ID NO: 98, SEQ ID NO: 99, or SEQ ID NO: 100. In some embodiments, the chimeric Paramecium tetraurelia eRF3 can comprise a nucleic acid sequence comprising SEQ ID NO: 158, SEQ ID NO: 159, SEQ ID NO: 160, or SEQ ID NO: 161.

In some embodiments, the first organism can comprise a eukaryotic cell. In some embodiments, the first organism can comprise a prokaryotic cell. In some embodiments, the prokaryotic cells can comprise an archaebacteria cell. In some embodiments, the prokaryotic cell can comprise a bacterial cell. In some embodiments, the prokaryotic cell can comprise a bacterial cell and an archaebacteria cell. In some embodiments, the eukaryotic cell can comprise a yeast cell, a fungal cell, a plant cell, an animal cell, an insect cell, a mammalian cell, or any combination thereof. In some embodiments, the yeast cell can comprise Saccharomyces cerevisiae.

### Methods for Screening and Testing Function of Release Factors

In one aspect, provided herein are compositions, systems, and methods for screening a release factor having codon-specific release factor activity. In this aspect, a cell or a population of cells (e.g., cell line) expressing a release factor with release factor activity to recognize one or more, but not all, stop codons may be used. In one example, a cell or a population of cells expressing a release factor with release factor activity to recognize UGA as a stop codon but not UAA and/or UAG as a stop codon may be used to identify another release factor that has release factor activity to recognize UAA and/or UAG as a stop codon. In another example, a cell or a population of cells expressing a release factor with release factor activity to recognize UAA and/or UAG as a stop codon but not UGA as a stop codon may be used to identify another release factor that has release factor activity to recognize UGA a stop codon.

In another aspect, provided herein are compositions, systems, and methods for testing a release factor having codon-specific release factor activity identified from any screening described herein. In this aspect, a cell or a population of cells (e.g., cell line) expressing a release factor with release factor activity to recognize one or more, but not all, stop codons may be used. In one example, a cell or a population of cells expressing a release factor with release factor activity to recognize UGA as a stop codon but not UAA and/or UAG as a stop codon may be used to confirm that one or more other release factors identified from any screen described herein to have release factor activity to recognize UGA as a stop codon does not recognize UAA and/or UAG as a stop codon. In another example, a cell or a population of cells expressing a release factor with release factor activity to recognize UAA and/or UAG as a stop codon but not UGA as a stop codon may be used to confirm one or more release factors identified from any screen described herein to have release factor activity to recognize UAA and/or UAG as a stop codon does not recognize UGA as a stop codon.

In some embodiments, the cell or the population of cells used for screening or testing assays may express a release factor that is functional at permissive temperature but not functional at non-permissive temperature (e.g., a temperature sensitive allele, such as yeast sup45-ts alleles). In some embodiments, the cell or the population of cells used for screening or testing assays may express a release factor with an inducible degron system. Details on temperature sensitive alleles, permissive/non-permissive temperatures, and degron systems are described in other sections of the present disclosure.

Described herein are methods for screening a release factor having codon-specific release factor activity comprising: a. providing a cell or a population of cells comprising a first release factor recognizing one or two stop codons; b. introducing the cell or the population of cells a second release factor; c. performing a first assay to detect codon-specific activity of the second release factor; and d. performing a second assay to confirm the second release factor does not recognize the one or two strop codons recognized by the first release factor. In one embodiment, the first release factor may recognize UGA as a stop codon. In this embodiment, the first release factor may not recognize UAA and/or UAG as a stop codon. In another embodiment, the first release factor may recognize UAA, UAG, or a combination thereof, as a stop codon. In this embodiment, the first release factor may not recognize UGA as a stop codon.

In some embodiments, the first assay or the second assay may be performed at a permissive temperature. In some embodiments, the permissive temperature may comprise from about 20° C. to about 33° C. In some embodiments, the permissive temperature may be about 20° C., about 20.5° C., about 21° C., about 21.5° C., about 22° C., about 22.5° C., about 23° C., about 23.5° C., about 24° C., about 24.5° C., about 25° C., about 25.5° C., about 26° C., about 26.5° C., about 27° C., about 27.5° C., about 28° C., about 28.5° C., about 29° C., about 29.5° C., about 30° C., about 30.5° C., about 31° C., about 31.5° C., about 32° C., about 32.5° C., or about 33° C. In some embodiments, the permissive temperature may be 25° C.

In some embodiments, the first assay or the second assay may be performed at a non-permissive temperature. In some embodiments, the non-permissive temperature may comprise any temperature above about 33.5° C. In some embodiments, the non-permissive temperature may be about 33.5° C., about 34° C., about 34.5° C., about 35° C., about 35.5° C., about 36° C., about 36.5° C., about 37° C., about 37.5° C., about 38° C., about 38.5° C., about 39° C., about 39.5° C., about 40° C., about 40.5° C., about 41° C., about 41.5° C., about 42° C., about 42.5° C., about 43° C., 43.5° C., about 44° C., about 44.5° C., or about 45° C. In some embodiments, the non-permissive temperature may be 37° C.

In some embodiments, the first assay or the second assay may be performed at a temperature from about 30° C. to about 37° C. For example, the first assay or the second assay may be performed at 30° C., about 30.5° C., about 31° C., about 31.5° C., about 32° C., about 32.5° C., about 33° C., about 33.5° C., about 34° C., about 34.5° C., about 35° C., about 35.5° C., about 36° C., about 36.5° C., or about 37° C.

In some aspects, a shuffle episome system may be used for screening or testing methods described herein. In some aspects, a “shuffle episome” or a “shuffle episome system,” refers to one or more plasmids encoding release factors that are subsequently introduced (e.g., via transformation, transfection, and the like) into a cell (e.g., a yeast cell). In some embodiments, the shuffle episome or the shuffle episome system can be used in any methods, systems, or embodiments described herein. Ciliate release factors that exclusively recognize UAA and/or UAG may fail to replace omnipotent release factors because such a strain cannot decode UGA stop codons. Ciliate release factors that exclusively recognize UGA may fail to replace omnipotent yeast release factors because such a strain cannot decode UAA and/or UAG stop codons. In some embodiments, combining two distinct ciliate release factors, one release factor which can recognize UAA and/or UAG and another release factor can recognize UGA in the same stain, can allow “replaceability.” In some embodiments, this “replaceability” can prove the stop codon specificity of the two release factors and simultaneously show that both release factors can function in a cell or an organism. In some embodiments, the experimental readout for testing replaceability of the release factors described herein can be cell viability.

In some embodiments, the release factors tested can be an eukaryotic release factor 1/eukaryotic release factor 3 (eRF1/eRF3) complex. In some embodiments, the release factors tested can be a yeast eRF1/eRF3 complex. In some embodiments, the plasmids can encode a native release factor. In some embodiments, the plasmids can encode a native yeast release factor. In some embodiments, the plasmids can encode a mutated release factor. In some embodiments, the plasmids can encode a mutated yeast release factor. In some embodiments, the plasmids can encode a ciliate release factor. In some embodiments, the plasmids can encode a native ciliate release factor. In some embodiments, the plasmids can encode a mutated ciliate release factor. In some embodiments, the plasmids can encode a mutated endogenous recognition domain for a release factor. In some embodiments, the plasmids can encode a recognition domain from a second organism. In some embodiments, the plasmids can encode a mutated recognition domain from a second organism.

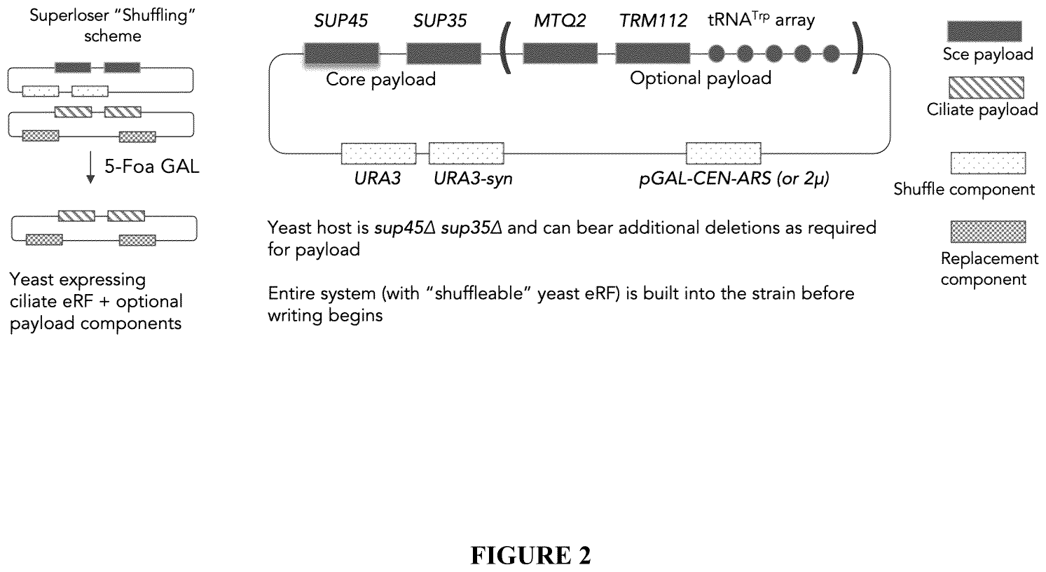

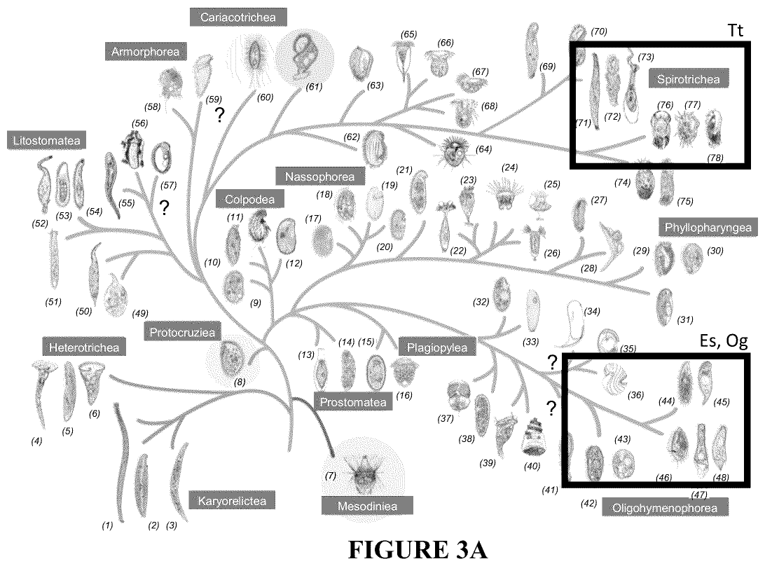

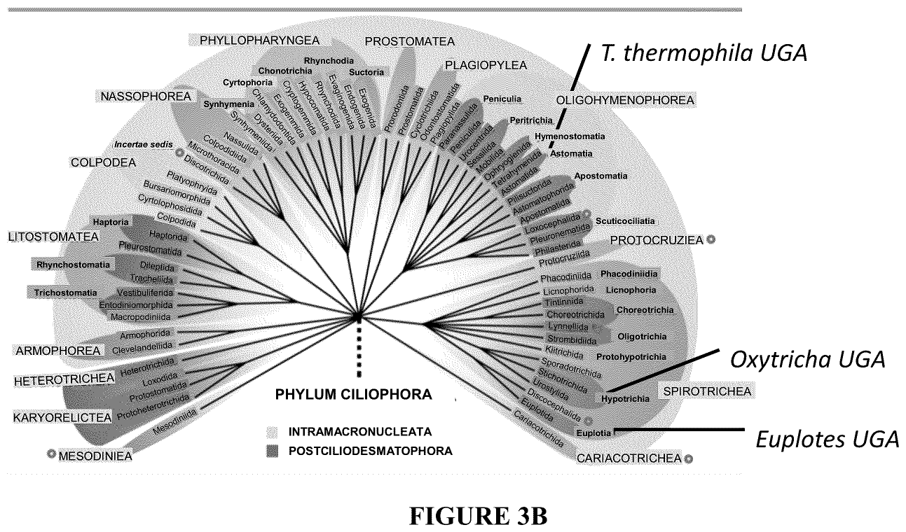

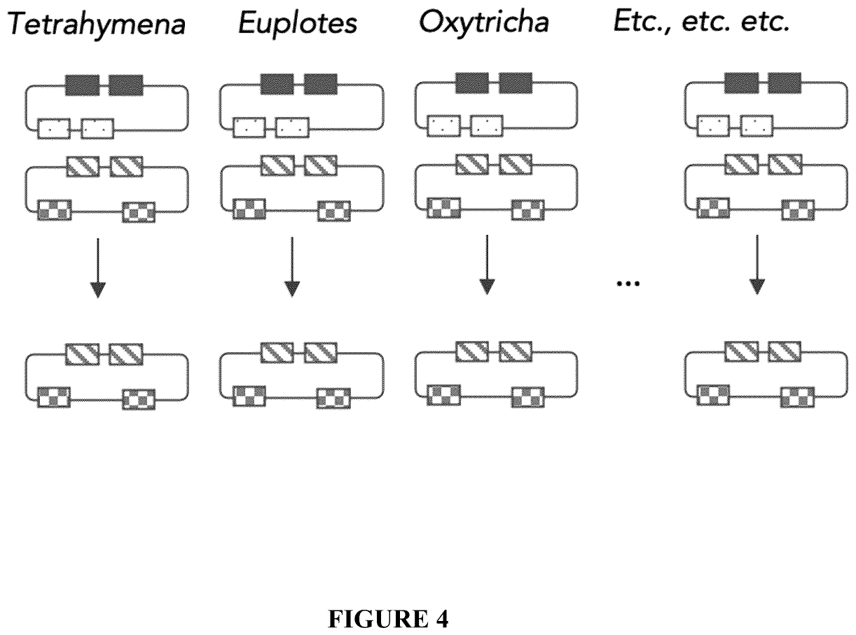

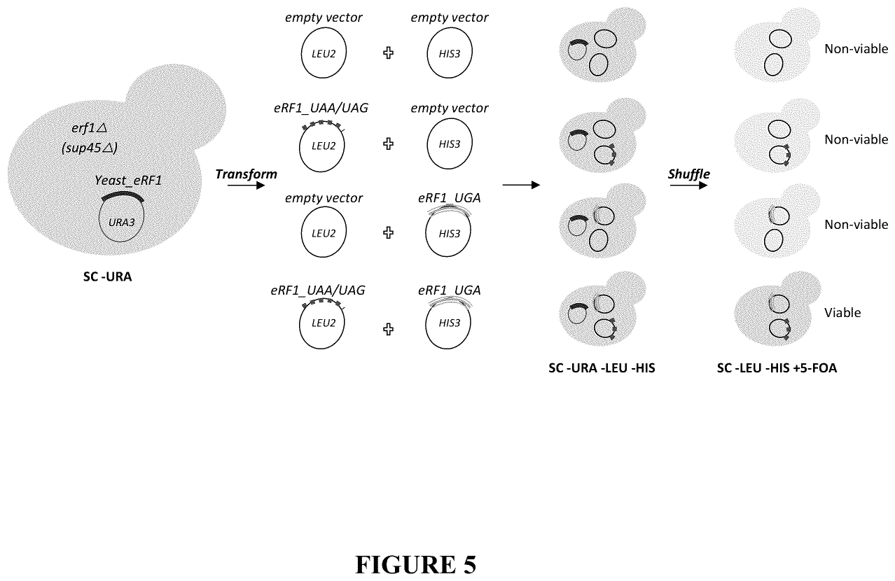

In some embodiments, the expression of the plasmids can be driven by a promoter. In some embodiments, the promoter can comprise an endogenous promoter (e.g., an endogenous eRF1/eRF3 promoter). In some embodiments, the promoter can comprise an inducible promoter system (e.g., GAL1/10 system). In some embodiments, the plasmid can encode a selectable marker (e.g., URA3, LEU2, or HIS3). In some embodiments, the plasmid can encode a counter-selectable marker (e.g., URA3). In some example embodiments, the shuffle episome system can be built with all native proteins and/or tRNAs on a supernumerary designer chromosome. Example embodiments of a shuffle episome system are shown in FIG. 2, FIG. 4, and FIG. 5.

In some aspects, release factors identified from any screens described herein or engineered ciliate-derived eukaryotic release factor (eRF) systems can be tested (FIG. 5). In some embodiments, yeast that only have the UAA/UAG-specific eRF1 constructs may be non-viable, post-shuffling. In some embodiments, the UAA/UGA-specific eRF1 yeast strain may be non-viable because the strain cannot decode UGA stop codons. In some embodiments, yeast that only have the UGA-specific eRF1 constructs will be non-viable, post-shuffling. In some embodiments, the UGA-specific eRF1 yeast strain may be non-viable because the strain cannot decode UAA/UAG stop codons. In some embodiments, yeast strains that have both the UAA/UAG-specific eRF1 and the UGA-specific eRF1 constructs can be viable, post-shuffling. In some embodiments, yeast that have both the UAA/UAG-specific eRF1 and the UGA-specific eRF1 constructs can be viable, which is consistent with stop codon specificity of the two eRF1 constructs and demonstrates that both eRF1 constructs are functional in yeast.

In some embodiments, recombinant nucleic acid sequence comprising a sequence encoding one or more release factors identified from any screens described herein or engineered eRF machineries described herein can be integrated into the host genome using any methods known to skill in the art.

In some embodiments, a release factor recognition domain of a release factor, e.g., eRF1, can be changed by replacing its native recognition domain with a non-native recognition domain, e.g., a recognition domain of a release factor from another organism. In one embodiment, amino acid residues of a release factor can be mutated. For example, mutations that can configure a release factor not to recognize UGA or both UAG and UAA can be introduced to a recognition domain of the release factor. In another embodiment, a recognition domain of a release factor can be swapped with a recognition domain of a release factor of another organism, e.g., ciliate eRF1, that recognizes only UGA as a stop codon. In some embodiments, a recognition domain of a native release factor of a host organism may be swapped with a recognition domain of a release factor from a different organism that is known to work in the host organism. In some embodiments, the entire native release factor (e.g., eRF1) of the host organism can be replaced with a foreign release factor (e.g., eRF1 from another organism) that recognizes only UGA as a stop codon.

These embodiments may include a foreign eRF3 (e.g., eRF3 from another organism), which works with eRF1 to provide release activity, and foreign enzymes that provide post-translational modifications for release factor proteins. For example, a post-translational modification can include, but is not limited to, a methyl-transferase activity. Embodiments described herein may include a tRNA from another organism that recognizes a specific codon and post-transcriptional and/or post-translational modification machinery. Embodiments disclosed herein may further comprise methods for protein engineering. In some embodiments, methods for protein engineering comprise directed evolution, library screens, machine learning, or a combination thereof. In some embodiments, library screens may be enhanced by phylogenetic data mining to identify organisms whose release factor machinery recognizes only one or two stop codons (e.g., recognizes only UGA as a stop codon). Release factor machinery from the identified organisms can then be tested systematically to identify the organism comprising release factors with a high level of fitness in the host organism. Testing the release factor machinery can be accomplished by providing nucleic acid sequences encoding foreign release factor proteins, release factor modifying proteins, and tRNAs either integrated into the host genome or supplied on an episomal element, e.g., a Superloser plasmid. Haase, M., et al. “Superloser: A Plamid Shuffling Vector for Saccharomyces cerevisiae with Exceedingly Low Background.” G3 (Bethesda). 2019 August; 9(8): 2699-2707. In some embodiments, an episomal element comprising a native gene or a gene of the host organism may further comprise a counter-selectable gene (e.g., URA3). In some embodiments, one or more episomal elements comprising a foreign gene(s) may further comprise a selectable gene (e.g., HIS3, LEU2). The loss of the episomal element comprising the native gene or the gene of the host organism may be selected on 5-FOA. In some embodiments, an episomal element or a superloser plasmid may allow highly efficient counterselection.

Methods provided herein describes experimental procedures for testing the ability of release factors from one organism that exclusively recognize either UAA/UAG or UGA to function in another organism. For example, methods described herein may be used test the ability of ciliate release factors (RFs) that exclusively recognize either UAA/UAG or VGA to function in Saccharomyces cerevisiae (hereafter referred to as “yeast”). The methods described herein can test the ability of ciliate release factors, either individually or in combination, to replace the yeast eRF1, which recognizes all three stop codons. In some embodiments, replacement of a native RF of an organism may comprise targeted engineering of specific motifs in the native RF to resemble motifs that can confer stop codon selectivity in another organism (e.g., Amino Acid swap, Domain/Motif swap). For example, replacement of a yeast RF may comprise targeted engineering of specific motifs in the yeast RF to resemble motifs that can confer stop codon selectivity in ciliates. In other embodiments, targeted engineering can involve complete RF gene replacement with an RF gene of another organism. For example, a yeast RF gene may be replaced with a gene encoding ciliate RF (e.g. Native Ciliate Machinery). In the case of gene replacements, the ciliate RFs may be introduced as whole gene ciliate constructs or as chimeric yeast-ciliate constructs. In less preferred embodiments, addition of other ciliate genes that have regulatory functions that act on ciliate RFs may be required. Ciliate RFs that exclusively recognize UAA/UAG may fail to replace omnipotent yeast RFs because such a ciliate strain cannot decode UGA stop codons. Ciliate RFs that exclusively recognize UGA may fail to replace yeast RFs because such a strain cannot decode UAA/UAG stop codons. Combining two distinct ciliate RFs, one of which recognizes UAA/UAG, and the second that recognizes UGA, in the same strain, can allow “replaceability” of the native yeast RF that recognizes all three standard stop codons, demonstrating the stop codon specificity of the two RFs and simultaneously showing that both can function in yeast. In some embodiments, the experimental readout for testing replaceability of the yeast native RFs can be cell viability.

Class 1 and 2 S. cerevisiae RFs can be encoded by the essential genes SUP45 (eRF1) and SUP35 (eRF3), respectively. Replaceability of the yeast RFs by ciliate RFs can be tested tri sup45Δ or sup45Δ sup35Δ mutants.

In some embodiments, the episomal-based shuffle system can be employed to test replaceability of wild-type yeast eRF1 by a motif-swapped yeast eRF1. In some cases, amino acid mutations are introduced into the yeast eRF1 protein's TASNIKS (SEQ ID NO: 1)/YLCDNKF (SEQ ID NO: 2) motifs, such that these motifs can resemble the TASNIKS (SEQ ID NO: 1)/YLCDNKF (SEQ ID NO: 2) motifs of the ciliate eRF1 proteins. In these cases, replaceability is tested in a sup45Δ mutant which lacks yeast eRF1.

In some embodiments, the episomal-based shuffle system can be employed to test replaceability of wild-type yeast eRF1 by the entire ciliate eRF1 protein. In these cases, the ciliate eRF1 protein can be expressed from the yeast endogenous eRF1 promoter. In this embodiment, replaceability can be tested in a sup45Δ mutant. In other embodiments, the corresponding ciliate eRF3 may be required for ciliate eRF1 function in yeast. In these cases, the ciliate eRF1/eRF3 proteins can be expressed from the same vector using the GAL1/10 bi-directional promoter. In other embodiments, the ciliate eRF3 can be modified to create a chimeric yeast-ciliate eRF3 protein. In some cases, the yeast N-terminal domain (residues 1-253), which contains the poly(A)-binding site, can replace the more divergent ciliate N-terminal domain. When testing eRF1 in conjunction with eRF3, replaceability can be tested in a sup45Δ or sup45Δ sup35Δ mutant.

The sup45Δ or sup45Δ sup35Δ deletion mutants can be constructed by replacing the genomic copies of each gene in a diploid strain with selectable markers that confer drug resistance (such as kanMX, natMX or hpMX). Viability of the strain can be maintained by pre-transformation of the counter-selectable vector containing the corresponding yeast gene(s). In the case where expression of the vector-based yeast gene(s) is being driven by their endogenous promoter(s), the strains can be grown in medium with any sugar source (e.g., dextrose, galactose). In the case where expression of the vector-based yeast gene(s) is being driven by the inducible GAL1-10 promoter, the strains can be grown in a medium containing galactose as the sugar source. Following sporulation of the heterozygous diploid. sup45Δ/SUP45 or homozygous diploid sup45Δ/sup45Δ strains, haploids containing the appropriate drug cassettes, as well as the counter-selectable vector, can be isolated by tetrad analysis. Yeast haploid strains bearing genomic deletions of sup45Δ or sup45Δ sup35Δ can be tested for plasmid-dependence by growing on a medium that counter-selects against the vector containing the wild-type yeast genes. In the case that this vector is marked by URA3, this medium can contain 5-FOA. In some embodiments, this vector can comprise a supernumerary designer chromosome. In some embodiments, this vector can comprise a supernumerary designed scaffold or a supernumerary designer chromosome.

### Selective Modulation of Release Factors

In some aspects, release factors described herein may be engineered to comprise an element that allows selective modulation of function or expression of release factors. In some embodiments, the element that allows selective modulation of function or expression of release factors may comprise a temperature sensitive allele. In some embodiments, the temperature sensitive allele may allow a release factor to function only at a permissive temperature. In some embodiments, the temperature sensitive allele may comprise a sup45 allele. In some embodiments, the sup45 allele may comprise sup45-ts, sup45-2, sup45-36ts, sup45-1023ts, or sup45-sl23ts.

In some embodiments, the sup45 allele may comprise one or more amino acid substitutions. In some embodiments, the sup45 allele may comprise one or more amino acid substitutions at I222, L223, Q76, Q77, L34, S30, or a combination thereof. In some embodiments, the one or more amino acid substitutions may comprise I222S, L223S, Q76H, Q77*, L34S, S30F, S30P, or a combination thereof, wherein * denotes a nonsense mutation that leads to a premature termination of translation. For example, a genetic mutation in nucleic acid sequence may change a codon that normally encodes an amino acid into a stop codon or a termination codon such as UAA, UAG, or UGA.

In some embodiments, sup45-ts allele may comprise an amino acid substitution at L223. In some embodiments, sup45-ts allele may comprise an amino acid substitution L223S. In some embodiments, sup45-2 allele may comprise an amino acid substitution at I222. In some embodiments, sup45-2 allele may comprise an amino acid substitution I222S. In some embodiments, sup45-1023ts allele may comprise one or more amino acid substitutions at Q76, Q77, or a combination thereof. In some embodiments, sup45-1023ts allele may comprise one or more amino acid substitution comprising Q76H, Q77*, or a combination thereof, wherein * denotes a nonsense mutation that leads to a premature termination of translation. In some embodiments, sup45-36ts may comprise an amino acid substitution at L34. In some embodiments, sup45-36ts may comprise an amino acid substitution L34S. In some embodiments, sup45-sl23ts may comprise an amino acid substitution at S30. In some embodiments, sup45-sl23ts may comprise an amino acid substitution S30F. In some embodiments, sup45-sl23ts may comprise an amino acid substitution S30P.

In some embodiments, the sup45-ts may comprise a sequence with at least 30%, at least 40%, at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to SEQ ID NO: 162. In some embodiments, the sup45-2 may comprise a sequence with at least 30%, at least 40%, at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to SEQ ID NO: 163. In some embodiments, the sup45-36ts may comprise a sequence with at least 30%, at least 40%, at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to SEQ ID NO: 164. In some embodiments, the sup45-1023ts may comprise a sequence with at least 30%, at least 40%, at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to SEQ ID NO:165. In some embodiments, the sup45-sl23ts may comprise a sequence with at least 30%, at least 40%, at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to SEQ ID NO: 166 or 167.

In some embodiments, the function or activity of a recombinant release factor may be modulated using a temperature sensitive allele. In some embodiments, the function or activity of a recombinant release factor may be turned on at a permissive temperature. In some embodiments, the function of activity of a recombinant release factor may be turned off at a non-permissive temperature.

In some embodiments, the permissive temperature may comprise from about 20° C. to about 33° C. In some embodiments, the permissive temperature may be about 20° C., about 20.5° C., about 21° C., about 21.5° C., about 22° C., about 22.5° C., about 23° C., about 23.5° C., about 24° C., about 24.5° C., about 25° C., about 25.5° C., about 26° C., about 26.5° C., about 27° C., about 27.5° C., about 28° C., about 28.5° C., about 29° C., about 29.5° C., about 30° C., about 30.5° C., about 31° C., about 31.5° C., about 32° C., about 32.5° C., or about 33° C. In some embodiments, the permissive temperature may be 25° C.

In some embodiments, the non-permissive temperature may comprise any temperature above about 33.5° C. In some embodiments, the non-permissive temperature may be about 33.5° C., about 34° C., about 34.5° C., about 35° C., about 35.5° C., about 36° C., about 36.5° C., about 37° C., about 37.5° C., about 38° C., about 38.5° C., about 39° C., about 39.5° C., about 40° C., about 40.5° C., about 41° C., about 41.5° C., about 42° C., about 42.5° C., about 43° C., 43.5° C., about 44° C., about 44.5° C., or about 45° C. In some embodiments, the non-permissive temperature may be 37° C.

In some embodiments, the element that allows selective modulation of function or expression of release factors may comprise a degron cassette. Without wishing to be bound by any theory, a degron is a portion of a protein that is important in regulation of protein degradation rates. Examples include, but are not limited to, short amino acid sequences, structural motifs, and exposed amino acids (e.g., Lys or Arg) that may be located anywhere in the protein. Degrons are present in a variety of organisms including both prokaryotes and eukaryotes. In one example, N-degrons (e.g., N-end Rule) was first characterized in yeast. In another example, the PEST sequence in ornithine decarboxylase was described in mouse. An inducible degron cassette may be generated and inserted into a gene to regulate degradation of the gene product, e.g., a protein. Similar to natural protein degradation, degron-mediated degradation mechanisms can be categorized by their dependence or lack thereof on Ubiquitin, a small protein involved in proteasomal protein degradation. In some embodiments, a degron may be referred to as Ubiquitin-dependent degron. In some embodiments, a degron may be referred to as Ubiquitin-independent degron. In some embodiments, a degron cassette may be inserted at the 5′ end of a gene sequence. In some embodiments, a degron cassette may be inserted at the 3′ end of a gene sequence. In some embodiments, a degron cassette may be located at the N-terminus of the protein encoded by the gene, when translated. In some embodiments, a degron cassette may be located at the C-terminus of the protein encoded by the gene, when translated. In some embodiments, a degron cassette may further comprise a marker, e.g., a selection marker. In some embodiments, the marker gene may be located at the 5′ end of the degron sequence. In some embodiments, the marker gene may be located at the 3′ end of the degron sequence. In some embodiments, any maker known in the art may be used. For example, a marker can include, but is not limited to, Kanamycin (Kan), Hygromycin (Hph), Nourseothricin (Nat), Neomycin (Neo), URA3, LEU2, TRP1, LYS2, and HIS3. In some embodiments, a degron may be codon-optimized. In some embodiments, the degron cassette may comprise a heat-inducible degron cassette or a small molecule-inducible degron cassette. In some embodiments, the small molecule-inducible degron cassette may comprise an auxin or asunaprevir. In some embodiments, a degron cassette may allow degradation of a release factor.

In some embodiments, the degron cassette may comprise a heat-inducible degron cassette. Details of heat-inducible degron cassette are described in Dohmen, et al., 1994, Science, 263(5151): 1273-1276. In some embodiments, the heat-inducible degron cassette may comprise an Arg-DHFR^{ts} N-degron. In some embodiments, the Arg-DHFR^{ts} N-degron may be activated at about 31° C., about 31.5° C., about 32° C., about 32.5° C., about 33° C., about 33.5° C., about 34° C., about 34.5° C., about 35° C., about 35.5° C., about 36° C., about 36.5° C., about 37° C., about 37.5° C., about 38° C., about 37.5° C., or about 39° C. In some embodiments, the Arg-DHFR t s N-degron may be activated at about 37° C. In some embodiments, the Arg-DHFR t s N-degron may not be activated at about 21° C., about 21.5° C., about 22° C., about 22.5° C., about 23° C., about 23.5° C., about 24° C., about 24.5° C., about 25° C., about 25.5° C., about 26.5° C., about 27° C., about 27.5° C., about 28° C., about 28.5° C., about 29° C., about 29.5° C., or about 30° C. In some embodiments, the Arg-DHFR t s N-degron may not be activated at about 23° C.

In some embodiments, the degron cassette may comprise a small molecule-inducible degron cassette. Details of small molecule-inducible degron cassette are described in Morawska & Ulrich, 2013, Yeast, 30: 341-351; Nishimura & Kanemaki, 2014, Current Protocols in Cell Biology, 64: 20.9.1-20.9.16; and Yesbolatova, et al., 2020, Nature Communications, 11: 5701. In some embodiments, a degron cassette may utilize a plant hormone. In some embodiments, the plant hormone may comprise an auxin family hormone. In some embodiments, a small molecule-inducible degron cassette may comprise an auxin or an auxin-inducible degron (AID) system. In some embodiments, a natural auxin may be used. In some embodiments, the natural auxin may comprise, but is not limited to, indole-3-acetic acid (IAA). In some embodiments, a synthetic auxin may be used. In some embodiments, the synthetic auxin may comprise, but is not limited to, 1-naphthaleneacetic acid (NAA). Details of the mechanism of the AID degron system is detailed in Nishimura & Kanemaki, 2014, Current Protocols in Cell Biology, 64: 20.9.1-20.9.16; and Morawska & Ulrich, 2013, Yeast, 30: 341-351. In some embodiments, a shorter AID degron system (mini-AID or mAID) or 3X mAID (3mAID) may be used. In some embodiments, a degron cassette for AID degron system can be inserted at the 5′ end of a gene sequence or at the 3′ end of a gene sequence. In some embodiments, the AID degron system may be located at the N-terminus or at the C-terminus of the protein encoded by the gene, when translated. In some embodiments, the protein encoded by the gene with the AID degron system at the N-terminus or at the C-terminus can be reversibly degraded by the addition of auxin, IAA, NAA, or a combination thereof. In some embodiments, the auxin can be added to the culture medium, e.g., a cell culture medium. In some embodiments, the temperature of the cell growth or the culture medium may be constant.

In some embodiments, a degron cassette may also comprise an epitope tag sequence. In some embodiments, the AID degron system may further comprise an epitope tag. In some embodiments, the epitope tag may be detected by an antibody. Examples of the antibody may include, but is not limited to, 8myc, 9myc, 6FLAG, 6HA, and GFP. In some embodiments, the epitope tag may be detected using fluorescence microscopy.

In some embodiments, a degron cassette may comprise AID version 2 (AID2) degron system. Details of the AID2 degron system is described in Yesbolatova, et al., 2020, Nature Communications, 11: 5701. In some embodiments, an auxin analogue may be used. Examples of the auxin analogue may include, but are not limited to, 5-phenyl-indole-3-acetic acid (5-Ph-IAA) and 5-adamantyl-IAA.

In some embodiments, the function or activity of a recombinant release factor may be modulated using a degron cassette. In some embodiments, the function or activity of a recombinant release factor may be turned on when the degron system is turned off or inactive. In some embodiments, the function of activity of a recombinant release factor may be turned off when a degron system is turned on or active. In some embodiments, recombinant release factors may be degraded when a degron system is turned on or active.

In some embodiments, the element that allows selective modulation of function or expression of release factors may comprise a conditional or an inducible promoter. An inducible promoter can be turned on and off when desired, by adding or removing an inducing agent. In some embodiments, a nucleic acid construct comprising a sequence encoding a release factor described herein may comprise a conditional or an inducible promoter for expressing the release factor. Examples of an inducible promoter can include, but are not limited to, a Lac, tac, trc, trp, araBAD, phoA, recA, proU, cst-1, tetA, cadA, nar, PL, cspA, T7, VHB, Mx, and/or Trex. In some embodiments, the conditional promoter may comprise a galactose inducible promoter, a tetracycline inducible promoter, a methionine inducible promoter, or an estradiol inducible promoter. In some embodiments, the galactose inducible promoter may comprise GAL1. In some embodiments, the tetracycline inducible promoter may comprise tetracycline inducible promoter or doxycycline inducible promoter. In some embodiments, the methionine inducible promoter may comprise MET15. In some embodiments, the estradiol inducible promoter may comprise GEV. In some embodiments the nucleic acid construct described herein may be a recombinant nucleic acid construct.

In some embodiments, the expression of a recombinant release factor may be modulated using a conditional or an inducible promoter. In some embodiments, the expression of a recombinant release factor may be turned on when the conditional or the inducible promoter is turned on by adding an inducing agent. In some embodiments, the expression of a recombinant release factor may be turned off when the conditional or the inducible promoter is turned off by removing an inducing agent.

In some embodiments, an endogenous release factor locus may be altered. In some embodiments, an endogenous release factor locus may be knocked out. In some embodiments, an endogenous release factor locus may be knocked down. Any known methods to skilled in the art may be used, for example, a CRISPR/Cas system, an shRNA system, an siRNA system, RNA interference, a homologous recombination system, or any combination thereof. In some embodiments, an endogenous release factor locus may comprise SUP45 or SUP35 in yeast.

In any of embodiments described herein, a recombinant release factor may comprise more than one element that allows selective modulation of function or expression of release factors. In some embodiments, a recombinant release factor may comprise one element, two elements, or three elements described herein. In one example, a recombinant release factor may comprise one element that allows selective modulation of function of release factors and another element that allows selective modulation of expression of release factors. In another example, a recombinant release factor may comprise two elements that allow selective modulation of function of release factors.

### Recombinant Nucleic Acid Encoding Release Factors

In one aspect, provided herein is a recombinant nucleic acid construct comprising a sequence encoding a recombinant release factor described herein. In some instances, recombinant nucleic acid constructs described herein may further comprise a leader sequence. In some instances, recombinant nucleic acid constructs may further comprise a promoter sequence. In some instances, recombinant nucleic acid constructs may further comprise a sequence encoding a poly(A) tail. In some instances, recombinant nucleic acid constructs may further comprise a 3′UTR sequence. In some instances, nucleic acid constructs described herein may be isolated nucleic acid or non-naturally occurring nucleic acid. Non-naturally occurring nucleic acids are well known to those of skill in the art. In some instances, nucleic acid constructs described herein are in vitro transcribed nucleic acid constructs.

In some embodiments, recombinant nucleic acid constructs described herein may comprise a conditional promoter for expressing a recombinant release factor described herein. A “promoter” or a regulatory sequence may refer to a nucleic acid sequence which can be used for expression of a gene product operably linked to the promoter/regulatory sequence. In some instances, this sequence may be the core promoter sequence. In other instances, this sequence may also include an enhancer sequence and other regulatory elements which are required for expression of the gene product. The promoter or the regulatory sequence may, for example, be one which expresses the gene product in a cell or tissue specific manner. In one aspect, a “constitutive” promoter may refer to a nucleic acid sequence which, when operably linked with a polynucleic acid which encodes or specifies a gene product, causes the gene product to be produced in a cell under most or all physiological conditions of the cell. In another aspect, an “inducible” or a “conditional” promoter may refer to a nucleic acid sequence which, when operably linked with a polynucleic acid sequence which encodes or specifies a gene product, causes the gene product to be produced in a cell substantially only when an inducer which corresponds to the promoter is present in the cell or when a condition for gene expression is met in the cell. For example, an inducible promoter can be turned on and off when desired, by adding or removing an inducing agent.

In some embodiments, the conditional or inducible promoter may comprise any conditional or inducible promoter known in the art. Examples of a conditional or an inducible promoter can include, but is not limited to, a Lac, tac, trc, trp, araBAD, phoA, recA, proU, cst-1, tetA, cadA, nar, PL, cspA, T7, VHB, Mx, and/or Trex. For example, the conditional or inducible promoter may comprise a galactose inducible promoter, a tetracycline inducible promoter, a methionine inducible promoter, or an estradiol inducible promoter. Examples of a galactose inducible promoter may include, but are not limited to GAL1. Examples of a tetracycline inducible promoter may include, but are not limited to tetracycline inducible promoter and doxycycline inducible promoter. Examples of a methionine inducible promoter may include, but are not limited to, MET15. Examples of an estradiol inducible promoter may include, but are not limited to, GEV.

The term “operably linked” may refer to functional linkage between a regulatory sequence and a heterologous nucleic acid sequence resulting in expression of the latter. For example, a nucleic acid sequence A is operably linked with a nucleic acid sequence B when the nucleic acid sequence A is placed in a functional relationship with the nucleic acid sequence B. For instance, a promoter is operably linked to a coding sequence if the promoter affects the transcription or expression of the coding sequence. Operably linked DNA sequences can be contiguous with each other and, e.g., where necessary to join two protein coding regions, are in the same reading frame.

In some aspects, provided herein is a vector or an expression vector comprising a recombinant nucleic acid sequence encoding a release factor described herein. In some embodiments, vectors described herein may comprise expression control sequences operatively linked to a nucleic acid sequence to be expressed. An expression vector may comprise sufficient cis-acting elements for expression; other elements for expression can be supplied by the host cell or in an in vitro expression system. Expression vectors can include all those known in the art, including, but not limited to, a DNA vector, an RNA vector, cosmids, plasmids (e.g., naked or contained in liposomes) and viruses (e.g., lentiviruses, retroviruses, adenoviruses, Rous sarcoma viral (RSV) vectors, herpes simplex viruses, adeno-associated viruses, chimeric viral vectors, viral-like particles, pox viruses, and pseudotyped viruses) that incorporate the recombinant polynucleic acid sequences.

In some embodiments, a recombinant nucleic acid construct may comprise a first recombinant nucleic acid sequence comprising a first sequence encoding a first recombinant release factor, wherein the first recombinant release factor is configured to recognize UGA, UAA, UAG, or any combination thereof as a stop codon. In some embodiments, a nucleic acid construct may comprise a second recombinant nucleic acid sequence comprising a second sequence encoding a second recombinant release factor, wherein the second recombinant release factor is configured to recognize UGA, UAA, UAG, or any combination thereof as a stop codon. In some embodiments, a recombinant nucleic acid construct may further comprise sequence encoding an element that allows selective modulation of function or expression of the second recombinant release factor. In some embodiments, the element that allows selective modulation of expression of a release factor may comprise an inducible promoter. In some embodiments, the element that allows selective modulation of function of a release factor may comprise a temperature sensitive allele. In some embodiments, the temperature sensitive allele may allow a release factor to function only at a permissive temperature. In some embodiments, the element that allows selective modulation of function of a release factor may comprise a degron cassette. In some embodiments, the degron cassette may allow degradation of a release factor. In some embodiments, the recombinant nucleic acid construct may further comprise one or more regulatory element for expressing an element that allows selective modulation of function or expression. Details of temperature sensitive alleles and degron cassettes are described in earlier section of the present disclosure. In some embodiments, the first sequence encoding the first recombinant release factor or the second sequence encoding the second recombinant release factor may comprise a sequence encoding an element that allows selective modulation of expression of a release factor and a sequence encoding an element that allows selective modulation of function of a release factor.

In some embodiments, a recombinant release factor can be expressed from a recombinant nucleic acid sequence comprising a gene encoding the recombinant release factor. In some embodiments, the recombinant nucleic acid sequence may be integrated into a genome. In some cases, the recombinant nucleic acid sequence can be integrated into the genome of a prokaryotic cell. In some cases, the recombinant nucleic acid sequence can be integrated into the genome of an eukaryotic cell. In some cases, the recombinant nucleic acid sequence can be integrated into the genome of a yeast. In some embodiments, the recombinant nucleic acid sequence can be introduced to a cell for genome integration via transformation. In some cases, the transformation can comprise heat-shock transformation. In some cases, the transformation can comprise electroporation. In some cases, the transformation can comprise cell-cell fusion. In some embodiments, the recombinant nucleic acid sequence can be introduced to a cell for genome integration via transfection. In some cases, the transfection can comprise a physical transfection. In some non-limiting example embodiments, physical transfection can include: electroporation, sonoporation, optical transfection, or hydrodynamic delivery. In some cases, the transfection can include a chemical transfection method. In some non-limiting example embodiments, a chemical transfection method can include: calcium phosphate, cationic polymers, lipofection, fugene, or dendrimers. In some embodiments, the gene can be integrated into the genome via transduction (e.g., foreign nucleic DNA introduced into a cell by a virus or viral vector). In some non-limiting example embodiments, viral vectors or viruses that can be used for transduction include: adenoviruses, adeno-associated viral vectors, lentiviruses, retroviruses, herpes simplex viruses, chimeric viral vectors, viral-like particles, pox viruses, or pseudotyped viruses. In some embodiments, the gene can be integrated into the genome via gene editing methods. In some non-limiting example embodiments, gene editing methods include: homologous recombination, site specific recombinases, meganucleases, zinc finger nucleases (ZFNs), transcription activator-like effector nucleases (TALEN), and clustered regularly interspaced short palindromic repeat/CRISPR-associated protein (e.g., CRISPR/Cas). In some non-limiting example embodiments, Cas proteins include: Cas9, Cas12, or Cas13.

In some embodiments, the recombinant release factor can be expressed from an episomal element comprising a recombinant nucleic acid sequence comprising a gene encoding the recombinant release factor. In some cases, the episomal element comprises a plasmid. In some cases, the plasmid can be a Superloser plasmid, a YIp plasmid, a YRp plasmid, a YCp plasmid, YEp plasmid, or a YLp plasmid. In some cases, the episomal element can exist autonomously in the cell (e.g., in the cytoplasm). In some cases, the episomal element can integrate into the genome. In some embodiments, the episomal element comprises regulatory sequences. In some embodiments, the regulatory sequences include: promoters, enhancers, silencers, or operators. In some embodiments, the promoter includes: endogenous RF1 promoter, endogenous RF3 promoter, endogenous eRF1 promoter, endogenous eRF3 promoter, Gal1/10 inducible promoter, In some embodiments, the episomal element further comprise one or more genes encoding a counter-selectable marker. In some embodiments, the counter-selectable gene can be a URA3 gene. In some embodiments, the counter-selectable gene can be a TRP1 gene. In some embodiments, the episomal element may further comprise one or more genes encoding a selectable marker. In some embodiments, the selectable marker gene can be a LEU2 gene. In some embodiments, the selectable gene can be a HIS3 gene.

### Nucleic Acid Construction and Replacing Genome

In some aspects, methods described herein may comprise synthesizing recombinant nucleic acid constructs described herein. Any known method in the art can be used to synthesize the recombinant nucleic acid construct comprising a sequence encoding a recombinant release factor. For example, different segments of a recombinant nucleic acid construct can be synthesized using e.g., a polymerase chain reaction (PCR), and/or restriction enzyme digestion/ligation. In some embodiments, these segments can be assembled into a construct by restriction enzyme cutting and ligation in vitro, or any other methods known in the art. In some embodiments, the recombinant nucleic acid construct can be sequenced to confirm the sequence of the nucleic acid construct and subsequently integrated into the host genome, e.g., a yeast genome, using any known methods in the art to replace the corresponding portion, region, or segment of the wile-type.

In some aspects, methods described herein may further comprise replacing a portion of a genome with a recombinant nucleic acid construct comprising a sequence encoding a recombinant release factor described herein. In some embodiments, site-specific nucleases (SSNs) or homology-directed recombination (HR) can be used to insert the recombinant nucleic acid construct a genome. In some embodiments, HR can be used utilizing an endogenous homologous recombination machinery.

In some embodiments, SSN may comprise meganucleases, zinc-finger nucleases (ZFN), TAL effector nucleases (TALEN), and clustered regularly interspaced short palindromic repeats (CRISPR)/CRISPR-associated (Cas) system. These four major classes of gene-editing techniques, namely, meganucleases, ZFNs, TALENs, CRISPR/Cas systems share a common mode of action in binding a user-defined sequence of DNA and mediating a double-stranded DNA break (DSB). DSB may then be repaired by HR, an event that introduces the homologous sequence from a donor DNA fragment, or by non-homologous end joining (NHEJ), when there is no donor DNA present.

In some embodiments, a CRISPR-Cas system may be used with a guide target sequence for genetic screening, targeted transcriptional regulation, targeted knock-in, and targeted genome editing, including base editing, epigenetic editing, and introducing double strand breaks (DSBs) for homologous recombination-mediated insertion of a nucleotide sequence. CRISPR-Cas system comprises an endonuclease protein whose DNA-targeting specificity and cutting activity can be programmed by a short guide RNA or a duplex crRNA/TracrRNA. A CRISPR endonuclease comprises a caspase effector nuclease, typically microbial Cas9 and a short guide RNA (gRNA) or a RNA duplex comprising a 18 to 20 nucleotide targeting sequence that directs the nuclease to a location of interest in the genome. Genome editing can refer to the targeted modification of a DNA sequence, including but not limited to, adding, removing, replacing, or modifying existing DNA sequences, and inducing chromosomal rearrangements or modifying transcription regulation elements (e.g., methylation/demethylation of a promoter sequence of a gene) to alter gene expression. As described above CRISPR-Cas system requires a guide system that can locate Cas protein to the target DNA site in the genome. In some instances, the guide system comprises a crispr RNA (crRNA) with a 17-20 nucleotide sequence that is complementary to a target DNA site and a trans-activating crRNA (tracrRNA) scaffold recognized by the Cas protein (e.g., Cas9). The 17-20 nucleotide sequence complementary to a target DNA site is referred to as a spacer while the 17-20 nucleotide target DNA sequence is referred to a protospacer. While crRNAs and tracrRNAs exist as two separate RNA molecules in nature, single guide RNA (sgRNA or gRNA) can be engineered to combine and fuse crRNA and tracrRNA elements into one single RNA molecule. Thus, in one embodiment, the gRNA comprises two or more RNAs, e.g., crRNA and tracrRNA. In another embodiment, the gRNA comprises a sgRNA comprising a spacer sequence for genomic targeting and a scaffold sequence for Cas protein binding. In some instances, the guide system naturally comprises a sgRNA. For example, Cas12a/Cpf1 utilizes a guide system lacking tracrRNA and comprising only a crRNA containing a spacer sequence and a scaffold for Cas12a/Cpf1 binding. While the spacer sequence can be varied depending on a target site in the genome, the scaffold sequence for Cas protein binding can be identical for all gRNAs.

CRISPR-Cas systems described herein can comprise different CRISPR enzymes. For example, the CRISPR-Cas system can comprise Cas9, Cas12a/Cpf1, Cas12b/C2cl, Cas12c/C2c3, Cas12d/CasY, Cas12e/CasX, Cas12g, Cas12h, or Cas12i. In some non-limiting example embodiments, Cas enzymes include, but are not limited to, Cas1, Cas1B, Cas2, Cas3, Cas4, Cas5, Cas5d, Cas5t, Cas5h, Cas5a, Cas6, Cas7, Cas8, Cas8a, Cas8b, Cas8c, Cas9 (also known as Csn1 or Csx12), Cas10, Cas10d, Cas12a/Cpf1, Cas12b/C2cl, Cas12c/C2c3, Cas12d/CasY, Cas12e/CasX, Cas12f/Cas14/C2c10, Cas12g, Cas12h, Cas12i, Cas12k/C2c5, Cas13a/C2c2, Cas13b, Cas13c, Cas13d, C2c4, C2c8, C2c9, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csx11, Csf1, Csf2, CsO, Csf4, Csd1, Csd2, Cst1, Cst2, Csh1, Csh2, Csa1, Csa2, Csa3, Csa4, Csa5, GSU0054, Type II Cas effector proteins, Type V Cas effector proteins, Type VI Cas effector proteins, CARF, DinG, homologues thereof, or modified or engineered versions thereof such as dCas9 (endonuclease-dead Cas9) and nCas9 (Cas9 nickase that has inactive DNA cleavage domain). In some cases, the compositions, methods, devices, and systems, described herein, may use the Cas9 nuclease from Streptococcus pyogenes, of which amino acid sequences and structures are well known to those skilled in the art.

In some aspects, described herein, are methods for contacting a genome in a sample with one or more agents configured to cleave the genome at a locus. In some embodiments, the contacting may occur in vitro. In some embodiments, the contacting may occur in vivo, e.g., in a cell. In some embodiments, the one or more agents comprise a polypeptide, a polynucleotide, or a combination thereof. In some embodiments, the polypeptide comprises an enzyme, e.g., a site-specific nuclease. Examples of a site-specific nuclease are shown above. In some embodiments, a site-specific nuclease comprises an engineered homing endonuclease or meganuclease, a zinc-finger nuclease (ZFN), a transcription activator-like effector nuclease (TALEN), a clustered regularly interspaced short palindromic repeat (CRISPR/Cas), or a combination thereof. In some embodiments, the polynucleotide comprises a guide RNA (gRNA). In some embodiments, the one or more agents comprise a site-specific nuclease and a gRNA (e.g., CRISPR/Cas system).

Agents described herein can be delivered into cells in vitro or in vivo by art-known methods or as described herein. Delivery methods such as physical, chemical, and viral methods are also known in the art. In some instances, physical delivery methods can be selected from the methods but not limited to electroporation, microinjection, or use of ballistic particles. On the other hand, chemical delivery methods require use of complex molecules such calcium phosphate, lipid, or protein. In some embodiments, viral delivery methods are applied for gene editing techniques using viruses such as but not limited to adenovirus, lentivirus, and retrovirus. In some embodiments, agents described herein can be delivered via a carrier. In some embodiments, agents described herein can be delivered by, e.g., vectors (e.g., viral or non-viral vectors), non-vector-based methods (e.g., using naked DNA, DNA complexes, lipid nanoparticles, RNA such as mRNA), or a combination thereof. In some embodiments, a carrier can comprise comprises a vector, a messenger RNA (mRNA), double stranded DNA (dsDNA), single stranded DNA (ssDNA), or a plasmid. In some embodiments, agents can be delivered directly to cells as naked DNA or RNA. Direct delivery, in some cases, is facilitated by, for instance by means of transfection or electroporation. In some cases, the agents are, or can be conjugated to molecules (e.g., N-acetylgalactosamine) promoting uptake by cells.

In some embodiments, the recombinant nucleic acid construct can be introduced into a cell in an episomal element. In some embodiments, the episomal element may comprise a vector. In some embodiments, vectors may also deliver one or more sequences encoding one or more agents described herein. Vectors can also comprise a sequence encoding a signal peptide (e.g., for nuclear localization, nucleolar localization, or mitochondrial localization), associated with (e.g., inserted into or fused to) a sequence coding for a protein. As one example, vectors can include a Cas9 coding sequence that includes one or more nuclear localization sequences (e.g., a nuclear localization sequence from SV40). Vectors described herein can also include any suitable number of regulatory/control elements, e.g., promoters, enhancers, introns, polyadenylation signals, Kozak consensus sequences, or internal ribosome entry sites (IRES). These elements are well known in the art. Vectors described herein may include recombinant viral vectors. Any viral vectors known in the art can be used. Examples of viral vectors include, but are not limited to lentivirus (e.g., HIV and FIV-based vectors), Adenovirus (e.g., AD100), Retrovirus (e.g., Maloney murine leukemia virus, MML-V), herpesvirus vectors (e.g., HSV-2), and Adeno-associated viruses (AAVs), or other plasmid or viral vector types. In some embodiments, agents described herein may be delivered in one carrier (e.g., one vector). In some embodiments, agents described herein may be delivered in in multiple carriers (e.g., multiple vectors).

In addition, viral particles can be used to deliver agents in nucleic acid and/or peptide form. For example, “empty” viral particles can be assembled to contain any suitable cargo. Viral vectors and viral particles can also be engineered to incorporate targeting ligands to alter target tissue specificity. Non-viral vectors can be also used to deliver agents according to the present disclosure. One example of non-viral nucleic acid vectors is an nanoparticle, which can be organic or inorganic. Nanoparticles are well known in the art. Any suitable nanoparticle design can be used to deliver agents described herein (e.g., nucleic acids encoding such agents).

In some embodiments, agents described herein can be delivered as a ribonucleoprotein (RNP) to cells. An RNP may comprise a nucleic acid binding protein, e.g., Cas9, in a complex with a gRNA targeting a genome/locus/sequence of interest. RNPs can be delivered to cells using known methods in the art, including, but not limited to electroporation, nucleofection, or cationic lipid-mediated methods, for example, as reported by Zuris, J. A. et al., 2015, Nat. Biotechnology, 33(1):73-80.

In some embodiments, an endogenous release factor gene may not be altered. In some embodiments, an endogenous release factor gene may be altered (e.g., replaced or removed).

### Systems, Cells, Cell Lysates, and Organisms

One aspect of the present disclosure provides a system comprising any of the recombinant release factors described herein, any of the recombinant nucleic acid constructs encoding any of the recombinant release factors described herein, or any of the vectors comprising recombinant nucleic acid constructs described herein. In some embodiments, the system may be an in vitro system. In some embodiments, the system may be an in vivo system. Another aspect of the present disclosure provides a cell or a population of cells comprising any of the recombinant release factors described herein, any of the recombinant nucleic acid constructs encoding any of the recombinant release factors described herein, or any of the vectors comprising recombinant nucleic acid constructs described herein. Another aspect of the present disclosure provides an organism comprising a cell or a population of cells comprising any of the recombinant release factors described herein, any of the recombinant nucleic acid constructs encoding any of the recombinant release factors described herein, or any of the vectors comprising recombinant nucleic acid constructs described herein.

In some embodiments, a cell may comprise a prokaryotic cell or a eukaryotic cell. In some embodiments, a prokaryotic cell may comprise an archaebacteria cell or a bacterial cell. In some embodiments, a eukaryotic cell may comprises a yeast cell, a fungal cell, a plant cell, an animal cell, an insect cell, or a mammalian cell. In some embodiments, a eukaryotic cell may be a yeast cell, e.g., a Saccharomyces cerevisiae cell. In some embodiments, an organism may comprise Saccharomyces cerevisiae. In some embodiments, the mammalian cell system may comprise Chinese Hamster Ovary (CHO) cells or murine myeloma (NS0) cells. In some embodiments, the cell or the population of cells described herein may not comprise a release factor that is expressed from a natural promoter and/or recognizes all of UAG, UAA and UGA as stop codons. For example, the cell or the population of cells may not comprise a release factor expressed from its natural or original genetic locus.

In some embodiments, an organism may be engineered with compositions, systems, and methods described herein to produce one or more cells that can be used for producing one or more polypeptides with ncAAs. In some embodiments, compositions, systems, and methods described herein may be used to develop therapies, for example, using expansion of cell populations that can be used for producing one or more polypeptides with ncAAs. For example, cancer cell therapies comprising cells such as CAR-T cells may be developed. In this example, a recombinant nucleic acid sequence encoding recombinant release factors with element that allows selective modulation of function or expression of recombinant release factors could be introduced into CAR-T cells. In this example, recombinant release factors can be selectively modulated using any methods described herein and one or more ncAAs can be incorporated into proteins that could be used for improved therapies, e.g., cell-drug conjugates or cell-antibody conjugates.

In some embodiments, recombinant nucleic acid construct described herein may be inserted in a genomic safe harbor site. Without wishing to be bound by any theory, genomic safe harbors (GSH) are sites in the genome that are able to accommodate the integration of new genetic material in a manner that ensures that the newly inserted genetic elements: (i) function predictably, and/or (ii) do not cause alternations of the host genome that may pose a risk to the host cell organism. As such, genomic safe harbors may be used as sites for nucleic acid construct insertion. Examples of genomic safe harbors in human include, but are not limited to, an adeno-associated virus site 1 (AAVS1), a chemokine (C—C motif) receptor 5 (CCRS) gene, a hypoxanthine phosphoribosyltransferase 1 (HPRT) locus, and a human ortholog of the mouse Rosa26 locus (or Rosa26 homolog locus). Details of genomic harbor sites are described in Papapetrou and A. Schambach, Molecular Therapy 24 (4): 678-684 (2016).

In some aspects, provided herein is a lysate or a culture of a cell or a population of cells comprising any of the recombinant release factors described herein, any of the recombinant nucleic acid constructs encoding any of the recombinant release factors described herein, or any of the vectors comprising recombinant nucleic acid constructs described herein. In some embodiments, a cell lysate may be obtained from a cell culture of a cell of a population of cells comprising any of the recombinant release factors described herein, any of the recombinant nucleic acid constructs encoding any of the recombinant release factors described herein, or any of the vectors comprising recombinant nucleic acid constructs described herein. In some embodiments, the cell lysate or the cell culture may comprise any of recombinant release factors described herein and other agents introduced to the cell or the population of cells, e.g., agents necessary for ncAA incorporation at one or more stop codons, as described later in the present disclosure. In some embodiments, the cell lysate or the cell culture may comprise any of recombinant release factors expressed from any of recombinant nucleic acid constructs described herein other agents introduced to or expressed from recombinant nucleic acid constructs in the cell or the population of cells, e.g., agents necessary for ncAA incorporation at one or more stop codons, as described later in the present disclosure. Methods for culturing a cell or a population of cells are well known in the art. Any procedure for obtaining cell lysates from a cell culture known in the art may be used. In some embodiments, cell lysates described herein may be used for an in vitro transcription and/or translation system.

Standard procedures of the present disclosure are described, e.g., in Maniatis et al., Molecular Cloning: A Laboratory Manual, Cold Spring Harbor Laboratory Press, Cold Spring Harbor, N.Y., USA (1982); Sambrook et al., Molecular Cloning: A Laboratory Manual (2 ed.), Cold Spring Harbor Laboratory Press, Cold Spring Harbor, N.Y., USA (1989); Davis et al., Basic Methods in Molecular Biology, Elsevier Science Publishing, Inc., New York, USA (1986); or Methods in Enzymology: Guide to Molecular Cloning Techniques Vol. 152, S. L. Berger and A. R. Kimmerl (eds.), Academic Press Inc., San Diego, USA (1987)). Current Protocols in Molecular Biology (CPMB) (Fred M. Ausubel, et al. ed., John Wiley and Sons, Inc.), Current Protocols in Protein Science (CPPS) (John E. Coligan, et. al., ed., John Wiley and Sons, Inc.), Current Protocols in Immunology (CPI) (John E. Coligan, et. al., ed. John Wiley and Sons, Inc.), Current Protocols in Cell Biology (CPCB) (Juan S. Bonifacino et. al. ed., John Wiley and Sons, Inc.), Culture of Animal Cells: A Manual of Basic Technique by R. Ian Freshney, Publisher: Wiley-Liss; 5th edition (2005), and Animal Cell Culture Methods (Methods in Cell Biology, Vol. 57, Jennie P. Mather and David Barnes editors, Academic Press, 1st edition, 1998), which are all incorporated by reference herein in their entireties.

Also described herein are methods for producing a polypeptide molecule comprising an ncAA or a population of polypeptide molecules comprising an ncAA, comprising growing a culture of host cells in a suitable culture medium, and purifying the polypeptide(s) from the cells or the culture in which the cells are grown. For example, the methods can include a process for producing a polypeptide in which a host cell containing a recombinant nucleic acid construct that includes a polynucleotide described herein can be cultured under conditions that allow expression of the encoded polypeptide. The polypeptide can be recovered from the culture, conveniently from the culture medium, or from a lysate prepared from the host cells and further purified. Preferred embodiments include those in which the polypeptide produced by such process can be a full length or mature form of the polypeptide.

One skilled in the art can readily follow known methods for isolating polypeptides and proteins in order to obtain one of the isolated polypeptides or proteins described herein, if isolating the polypeptide is desired. These include, but are not limited to, immunochromatography, HPLC, size-exclusion chromatography, ion-exchange chromatography, and immuno-affinity chromatography. See, e.g., Scopes, Protein Purification: Principles and Practice, Springer-Verlag (1994); Sambrook, et al., in Molecular Cloning: A Laboratory Manual; Ausubel et al., Current Protocols in Molecular Biology. The recombinant release factors, if desired, can be purified from a culture, for example, from culture medium or cell extracts, using known purification processes, such as affinity chromatography, gel filtration, and ion exchange chromatography. The purification can also include an affinity column containing agents which will bind to the protein; one or more column steps over such affinity resins as concanavalin A-agarose, heparin-Toyopearl™ or Cibacron blue 3GA Sepharose™; one or more steps involving hydrophobic interaction chromatography using such resins as phenyl ether, butyl ether, or propyl ether; or immunoaffinity chromatography. Alternatively, the recombinant release factor described herein can also be expressed in a form which will facilitate purification. For example, a protein can be expressed as a fusion protein, such as those of maltose binding protein (MBP), glutathione-S-transferase (GST) or thioredoxin (TRX), or as a His tag. Kits for expression and purification of such fusion proteins are commercially available from New England BioLab (Beverly, Mass.), Pharmacia (Piscataway, N.J.) and Invitrogen, respectively. The protein can also be tagged with an epitope and subsequently purified by using a specific antibody directed to such epitope. One such epitope (“FLAG®”) is commercially available from Kodak (New Haven, Conn.). Finally, one or more reverse-phase high performance liquid chromatography (RP-HPLC) steps employing hydrophobic RP-HPLC media, for example, silica gel having pendant methyl or other aliphatic groups, can be employed to further purify the recombinant release factor. Any combination of the foregoing purification procedures can also be employed to provide a substantially homogeneous isolated or purified recombinant release factor described herein. The recombinant release factors purified can be substantially free of other host cell proteins and can be defined in accordance with the present disclosure as an “isolated protein or polypeptide.”

Methods for Producing Polypeptides Comprising a Non-Canonical Amino Acid (ncAA)

One aspect of the present disclosure provides a method of producing a polypeptide molecule comprising a non-canonical amino acid (ncAA) or a population of polypeptide molecules comprising the ncAA using one or more recombinant release factors described herein. In some embodiments, methods described herein may provide transformational approaches to understand and control one or more biological functions. For example, methods described herein may allow producing polypeptides with amino acids corresponding to post-translationally modified versions of natural amino acids. For example, methods described herein may allow producing photocaged amino acids that may enable the rapid activation of protein function with light to dissect dynamic processes in cells. For example, methods described herein may allow usage of crosslinkers to provide a way to map protein interactions. For example, ncAAs containing fluorophores or other biophysical probes can be used to follow changes in protein structure and/or activity. In some embodiments, ncAAs may be used to alter enzyme function. In some embodiments, ncAAs may be used to trap labile enzyme—substrate intermediates for structural studies and substrate identification. In some embodiments, ncAAs bearing bio-orthogonal and chemically reactive groups may provide strategies for rapidly attaching a wide range of functionalities to proteins to precisely control and image protein function in cells and to create protein conjugates, including defined therapeutic conjugates. In some embodiments, methods described herein to produce polypeptides comprising an ncAA may form the basis of strategies for the reversible control of gene expression in animals and strategies for determining cell type-specific proteomes in animals. In some embodiments, methods described herein may allow incorporating multiple distinct ncAAs into polypeptides or proteins.

In some embodiments, methods described herein may comprise site-specific incorporation of one or more ncAAs into a polypeptide or a protein at a stop codon that is not recognized by one or more recombinant release factors described herein. In some embodiments, methods described herein may not comprise codon replacement and/or rewriting.

In some aspects, the method may comprise providing a first recombinant release factor, wherein the first recombinant release factor is configured to recognize UGA, UAA, UAG, or any combination thereof as a stop codon; a second recombinant release factor, wherein the second recombinant release factor is configured to recognize UGA, UAA, UAG, or any combination thereof as a stop codon, and wherein the second recombinant release factor comprises an element that allows selective modulation of function or expression of the second recombinant release factor; and an aminoacyl-tRNA synthetase (aaRS)/tRNA pair. In some embodiments, engineered aaRS/tRNA pairs may be used to incorporate one or more ncAAs.

In one embodiment, the method may comprise providing a first recombinant release factor configured to recognize UGA as a stop codon, a second recombinant release factor configured to recognize UAA and/or UAG as a stop codon, and an aminoacyl-tRNA synthetase (aaRS)/tRNA pair. In this embodiment, the second release factor may further comprise an element that allows selective modulation of function or the expression of the second recombinant release factor. In this embodiment, an aminoacyl-tRNA synthetase (aaRS)/tRNA pair for an ncAA-of-interest may be provided for ncAA incorporation at UAA and/UAG. In some embodiments, engineered aaRS/tRNA pairs may be used to incorporate one or more ncAAs.

In another embodiment, the method may comprise providing a first recombinant release factor configured to recognize UAA and/or UAG as a stop codon, a second recombinant release factor configured to recognize UGA as a stop codon, and an aminoacyl-tRNA synthetase (aaRS)/tRNA pair. In this embodiment, the second release factor may further comprise an element that allows selective modulation of function or the expression of the second recombinant release factor. In this embodiment, an aminoacyl-tRNA synthetase (aaRS)/tRNA pair for an ncAA-of-interest may be provided for ncAA incorporation at UGA. In some embodiments, engineered aaRS/tRNA pairs may be used to incorporate one or more ncAAs.

In yet another embodiment, the method may comprise providing a first recombinant release factor configured to recognize UAA and/or UGA as a stop codon, a second recombinant release factor configured to recognize UAG as a stop codon, and an aminoacyl-tRNA synthetase (aaRS)/tRNA pair. In this embodiment, the second release factor may further comprise an element that allows selective modulation of function or the expression of the second recombinant release factor. In this embodiment, an aminoacyl-tRNA synthetase (aaRS)/tRNA pair for an ncAA-of-interest may be provided for ncAA incorporation at UAG. In some embodiments, engineered aaRS/tRNA pairs may be used to incorporate one or more ncAAs.

In yet another embodiment, the method may comprise providing a first recombinant release factor configured to recognize UGA and/or UAG as a stop codon, a second recombinant release factor configured to recognize UAA as a stop codon, and an aminoacyl-tRNA synthetase (aaRS)/tRNA pair. In this embodiment, the second release factor may further comprise an element that allows selective modulation of function or the expression of the second recombinant release factor. In this embodiment, an aminoacyl-tRNA synthetase (aaRS)/tRNA pair for an ncAA-of-interest may be provided for ncAA incorporation at UAA. In some embodiments, engineered aaRS/tRNA pairs may be used to incorporate one or more ncAAs.

In yet another embodiment, the method may comprise providing a first recombinant release factor configured to recognize UAA as a stop codon, a second recombinant release factor configured to recognize UGA and/or UAG as a stop codon, and an aminoacyl-tRNA synthetase (aaRS)/tRNA pair. In this embodiment, the second release factor may further comprise an element that allows selective modulation of function or the expression of the second recombinant release factor. In this embodiment, an aminoacyl-tRNA synthetase (aaRS)/tRNA pair for an ncAA-of-interest may be provided for ncAA incorporation at UGA and/UAG. In some embodiments, engineered aaRS/tRNA pairs may be used to incorporate one or more ncAAs.

In yet another embodiment, the method may comprise providing a first recombinant release factor configured to recognize UAG as a stop codon, a second recombinant release factor configured to recognize UAA and/or UGA as a stop codon, and an aminoacyl-tRNA synthetase (aaRS)/tRNA pair. In this embodiment, the second release factor may further comprise an element that allows selective modulation of function or the expression of the second recombinant release factor. In this embodiment, an aminoacyl-tRNA synthetase (aaRS)/tRNA pair for an ncAA-of-interest may be provided for ncAA incorporation at UAA and/UGA. In some embodiments, engineered aaRS/tRNA pairs may be used to incorporate one or more ncAAs.

In some embodiments, one or more tRNA molecules configured to recognize a stop codon are provided. In some embodiments, one or more aminoacyl-tRNA synthetases (aaRSs) for charging the one or more tRNA molecules are provided. In some cases, the aminoacyl-tRNA can charge the one or more tRNA molecules that recognize a stop codon with a natural amino acid. In some cases, the aminoacyl-tRNA can charge the one or more tRNA molecules that recognize a stop codon with an ncAA. Alternatively, the one or more tRNA molecules configured to recognize the stop codon can be pre-charged. In some cases, the pre-charged tRNA can be charged with a natural amino acid. In some cases, the pre-charged tRNA can be charged with an ncAA. In some cases, the natural amino acid can comprise alanine, cysteine, aspartic acid, glutamic acid, phenylalanine, glycine, histidine, isoleucine, lysine, leucine, methionine, asparagine, proline, glutamine, arginine, serine, threonine, valine, tryptophan, or tyrosine. In some embodiments, a stop codon can encode a non-canonical amino acid (ncAA).

Non-Canonical Amino Acid (ncAA)

As used herein, a non-canonical amino acid (ncAA) can refer to any amino acid other than the 20 genetically encoded alpha-amino acids comprising alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine. In some aspects, described herein are non-canonical amino acids (ncAAs) that may comprise side chain chemistries and/or structures that are not available from canonical amino acids (cAAs). In some embodiments, ncAAs may comprise fluorinated amino acids or amino acids comprising a reactive group (e.g., carbonyl, alkene, or alkyne moieties), or photoactivatable group (e.g., azide, benzophenone, or fluorophores). Translation of ncAAs into proteins may allow chemical modification and accordingly, ncAAs may be useful for in vivo structure-function studies, protein-protein interaction studies, protein localization studies, protein activity regulation studies or studies to generate new protein function. ncAA can be incorporated in different cells, including, but not limited to bacterial cells (e.g., Escherichia coli), yeast cells (e.g., Saccharomyces cerevisiae, Pichia pastoris, or Candida albicans), mammalian cells and plant cells or in organisms, including, but not limited to Drosophila melanogaster, Caenorhabditis elegans, Bombyx mori, rabbit and cow.

In some embodiments, an ncAA may comprise Para-fluoro-L-phenylalanine, Para-iodo-L-phenylalanine, Para-azido-L-phenylalanine, Para-acetyl-L-phenylalanine, Para-benzoyl-L-phenylalanine, Meta-fluoro-L-tyrosine, O-methyl-L-tyrosine, Para-propargyloxy-L-phenylalanine, (2S)-2-aminooctanoic acid, (2S)-2-aminononanoic acid, (2S)-2-aminodecanoic acid, (2S)-2-aminohept-6-enoic acid, (2S)-2-aminooct-7-enoic acid, L-Homocysteine, (2S)-2-amino-5-sulfanylpentanoic acid, (2S)-2-amino-6-sulfanylhexanoic acid, L-S-(2-nitrobenzyl) cysteine, L-S-ferrocenyl-cysteine, L-O-crotylserine, L-O-(pent-4-en-1-yl)serine, L-O-(4,5-dimethoxy-2-nitrobenzyl)serine, (2S)-2-amino-3-({[5-(dimethyl amino)naphthal en-1-yl]sulfonyl}amino)propanoic acid, (2 S)-3-[(6-acetyl-naphthalen-1-yl)amino]-2-aminopropanoic acid, L-Pyrrolysine, N6-[(propargyloxy)carbonyl]-L-lysine, L-N6-acetyllysine, N6-trifluoroacetyl-L-lysine, N6-{[1-(6-nitro-1,3-benzodioxo1-5-yl)ethoxy]carbonyl}-L-lysine, N6-{[2-(3-methyl-3H-diaziren-3-yl)ethoxy]carbonyl}-L-lysine, p-azidophenylalanine, N6-[(2-Azidoethoxy)carbonyl]-L-lysine, p-acetyl-L-phenylalanine (AcF), p-propargyloxy-L-phenylalanine (OPG), 4-azidomethyl-L-phenylalanine (AzMF), 4-borono-L-phenylalanine (BPhe), 3,4-dihydroxy-L-phenylalanine (DOPA), 4-iodo-L-phenylalanine (IPhe), L-α-aminocaprylic acid (AC), N^{ε}-azido-L-lysine (AzK), 3-amino-L-tyrosine (ATyr), 4-amino-L-phenylalanine (APhe), N^{ε}, N^{ε}-dimethyl-L-lysine (DMK), Boc-L-lysine (BocK), (S)-2-amino-6-((2-azidoethoxy)carbonylamino)hexanoic acid (LysN3), (2S)-2-amino-6-(((prop-2-yn-1-yloxy)carbonyl)amino)hexanoic acid (LysAlk), N^{ε}-Allyloxycarbonyl-L-lysine, N-ε-propargyloxycarbonyl-L-lysine, L-2-Allylglycine, or O-Allyl-L-Tyrosine, or 2-aminoisobutyric acid (also known as α-aminoisobutyric acid, AIB, α-methylalanine, or 2-methylalanine).

In some embodiments, an ncAA may comprise AbK (unnatural amino acid for Photo-crosslinking probe), 3-Aminotyrosine (unnatural amino acid for inducing red shift in fluorescent proteins and fluorescent protein-based biosensors), L-Azidohomoalanine hydrochloride (unnatural amino acid for bio-orthogonal labeling of newly synthesized proteins), L-Azidonorleucine hydrochloride (unnatural amino acid for bio-orthogonal or fluorescent labeling of newly synthesized proteins), BzF (photoreactive unnatural amino acid; photo-crosslinker), DMNB-caged-Serine (caged serine; excited by visible blue light), HADA (blue fluorescent D-amino acid for labeling peptidoglycans in live bacteria), NADA-green (fluorescent D-amino acid for labeling peptidoglycans in live bacteria), NB-caged Tyrosine hydrochloride (ortho-nitrobenzyl caged L-tyrosine), RADA (orange-red TAMRA-based fluorescent D-amino acid for labeling peptidoglycans in live bacteria), Rf470DL (blue rotor-fluorogenic fluorescent D-amino acid for labeling peptidoglycans in live bacteria), sBADA (green fluorescent D-amino acid for labeling peptidoglycans in bacteria), or YADA (green-yellow lucifer yellow-based fluorescent D-amino acid for labeling peptidoglycans in live bacteria).

In some embodiments, an ncAA may comprise an O-methyl-L-tyrosine, an L-3-(2-naphthyl)alanine, a 3-methyl-phenylalanine, an O-4-allyl-L-tyrosine, a 4-propyl-L-tyrosine, a tri-O-acetyl-GlcNAcβ-serine, an L-Dopa, a fluorinated phenylalanine, an isopropyl-L-phenylalanine, a p-azido-L-phenylalanine, a p-acyl-L-phenylalanine, a p-benzoyl-L-phenylalanine, an L-phosphoserine, a phosphonoserine, a phosphonotyrosine, a p-iodo-phenylalanine, a p-bromophenylalanine, a p-amino-L-phenylalanine, or an isopropyl-L-phenylalanine.

In some embodiments, an ncAA may comprise an unnatural analogue of a canonical amino acid. For example, an ncAA may comprise an unnatural analogue of a tyrosine amino acid, an unnatural analogue of a glutamine amino acid, an unnatural analogue of a phenylalanine amino acid, an unnatural analogue of a serine amino acid, an unnatural analogue of a threonine amino acid. In some embodiments, an ncAA may comprise an alkyl, aryl, acyl, azido, cyano, halo, hydrazine, hydrazide, hydroxyl, alkenyl, alkynl, ether, thiol, sulfonyl, seleno, ester, thioacid, borate, boronate, phospho, phosphono, phosphine, heterocyclic, enone, imine, aldehyde, hydroxylamine, keto, or amino substituted amino acid, or any combination thereof.

In some embodiments, an ncAA may comprise an amino acid with a photoactivatable cross-linker, a spin-labeled amino acid, a fluorescent amino acid, an amino acid with a novel functional group, an amino acid that covalently or noncovalently interacts with another molecule, a metal binding amino acid, a metal-containing amino acid, a radioactive amino acid, a photocaged amino acid, a photoisomerizable amino acid, a biotin or biotin-analogue containing amino acid, a glycosylated or carbohydrate modified amino acid, a keto containing amino acid, an amino acid comprising polyethylene glycol, an amino acid comprising polyether, a heavy atom substituted amino acid, a chemically cleavable or photocleavable amino acid, an amino acid with an elongated side chain, an amino acid containing a toxic group, or a sugar substituted amino acid. In some embodiments, a sugar substituted amino acid may comprise a sugar substituted serine. In some embodiments, an ncAA may comprise a carbon-linked sugar-containing amino acid, a redox-active amino acid, an α-hydroxy containing amino acid, an amino thio acid containing amino acid, an α,α-disubstituted amino acid, a β-amino acid, or a cyclic amino acid other than proline.

In some embodiments, an ncAA may comprise p-azidophenylalanine or 2-aminoisobutyric acid (also known as α-aminoisobutyric acid, AIB, α-methylalanine, or 2-methylalanine). In some embodiments, an ncAA may comprise an azide-containing ncAA. Nonlimiting examples of an azide-containing ncAA include (S)-2-amino-6-((2-azidoethoxy)carbonylamino)hexanoic acid (LysN3), L-azidohomoalanine (L-AHA), 4-(6-(3-azidopropyl)-s-tetrazin-3-yl) phenylalanine (pTAF), or 3-(6-(3-azidopropyl)-s-tetrazin-3-yl) phenylalanine (mTAF). In some embodiments, an ncAA may comprise an alkyne-containing ncAA. In some embodiments, an ncAA may comprise an alkene-containing ncAA. In some embodiments, an alkene-containing ncAA or an alkyne-containing ncAA may comprise (2S)-2-amino-6-(((prop-2-yn-1-yloxy)carbonyl)amino)hexanoic acid (LysAlk), N^{ε}-Allyloxycarbonyl-L-lysine, N-ε-propargyloxycarbonyl-L-lysine, L-2-Allylglycine, or O-Allyl-L-Tyrosine. In some embodiments, an alkyne group or an alkene group can react with an azide group in a copper catalyzed click chemistry reaction.

In some embodiments, introducing recombinant release factor can modulate protein translation. In some embodiments, protein translation can be modulated by incorporating an ncAA at a stop codon. In some embodiments, protein translation can be modulated by incorporating one or more ncAAs at one or more stop codons. For example, one type of ncAA can be incorporated at one stop codon and another type of ncAA can be incorporated at another stop codon. In some embodiments, incorporating one or more ncAA may utilize an orthogonal translation system. In some embodiments, the orthogonal translation system may decode a stop codon (e.g., UAG, UAA, and/or UGA) as a sense codon.

### Orthogonal Translation System

In some aspects, provided herein are compositions, systems, and methods for producing a polypeptide molecule comprising a non-canonical amino acid (ncAA) or a population of polypeptide molecules comprising the ncAA using one or more recombinant release factors described herein. In some embodiments, the method may comprise providing a first recombinant release factor, wherein the first recombinant release factor is configured to recognize UGA, UAA, UAG, or any combination thereof as a stop codon; a second recombinant release factor, wherein the second recombinant release factor is configured to recognize UGA, UAA, UAG, or any combination thereof as a stop codon, and wherein the second recombinant release factor comprises an element that allows selective modulation of function or expression of the second recombinant release factor; and an aminoacyl-tRNA synthetase (aaRS)/tRNA pair.

In some embodiments, a ribosome may use tRNA adaptors, aminoacylated with their cognate amino acids by specific aminoacyl-tRNA synthetases (aaRSs), to progressively decode the triplet codons in a coding sequence and polymerize the corresponding sequence of amino acids into a protein. 64 triplet codons are used to encode the 20 canonical or natural amino acids, and the initiation and termination of protein synthesis. In some aspects, methods described herein may allow using one or two stop codons to encode one or more ncAAs. In some embodiments, one or two stop codons may be decoded by an additional aminoacyl-tRNA synthetase (aaRS)/tRNA pair. In some embodiments, these aaRS/tRNA pairs may be engineered. In some embodiments, these aaRS/tRNA pairs may uniquely decode distinct codons and recognize distinct ncAAs. In some embodiments, these aaRS/tRNA pairs may uniquely decode one or more distinct stop codons and recognize distinct ncAAs.

In some aspects, compositions, systems, and methods described herein may comprise orthogonal aaRS/tRNA pairs. In some embodiments, each orthogonal aaRS may aminoacylate its cognate orthogonal tRNA, and/or minimally aminoacylate the other tRNAs in an organism. In some embodiments, the orthogonal tRNA may be aminoacylated by its cognate synthetase and/or minimally be aminoacylated by the aaRSs of the organism. In some embodiments, the orthogonal tRNA may be engineered to recognize a stop codon, while maintaining selective aminoacylation by the orthogonal synthetase. In some embodiments, an active site of the orthogonal synthetase may be engineered.

In some aspects, provided herein are methods for incorporating an ncAA at a stop codon to encode an amino acid comprising an ncAA. For example, a stop codon may encode an ncAA instead of terminating protein translation. Over 100 ncAAs with diverse chemistries may be synthesized and co-translationally incorporated into polypeptides and proteins using evolved orthogonal aminoacyl-tRNA synthetase (aaRSs)/tRNA pairs. Non-limiting examples of ncAAs are described in the previous section. In some embodiments, an ncAA may comprise p-azidophenylalanine or 2-aminoisobutyric acid (also known as α-aminoisobutyric acid, AIB, α-methylalanine, or 2-methylalanine). In some embodiments, an ncAA may comprise an azide-containing ncAA, an alkene-containing ncAA, or an alkyne-containing ncAA. In some embodiments, an azide-containing ncAA may comprise (S)-2-amino-6-((2-azidoethoxy)carbonylamino)hexanoic acid (LysN3), L-azidohomoalanine (L-AHA), 446-(3-azidopropyl)-s-tetrazin-3-yl) phenylalanine (pTAF), or 3-(6-(3-azidopropyl)-s-tetrazin-3-yl) phenylalanine (mTAF). In some embodiments, an alkene-containing ncAA or an alkyne-containing ncAA may comprise (2S)-2-amino-6-(((prop-2-yn-1-yloxy)carbonyl)amino)hexanoic acid (LysAlk), N^{ε}-Allyloxycarbonyl-L-lysine, N-ε-propargyloxycarbonyl-L-lysine, L-2-Allylglycine, or O-Allyl-L-Tyrosine. Various aaRS/tRNA pairs can be used for methods described herein. In some embodiments, an ncAA may be designed based on tyrosine or pyrrolysine. In some embodiments, an aaRS/tRNA pair may be provided on a plasmid or into the genome of a cell or an organism comprising recombinant release factor. In some embodiments, an orthogonal aaRS/tRNA pair can be used to bioorthogonally incorporate ncAAs into polypeptides or proteins.

In some embodiments, vector-based over-expression systems may be used to introduce aaRS/tRNA pairs. In some embodiments, genome-based aaRS/tRNA pairs (i.e., aaRS/tRNA pairs incorporated into the genome of the cell or organism) may be used to reduce the chance of an early protein termination at the stop codon intended for ncAA incorporation in the absence of available ncAAs. In some embodiments, ncAA incorporation into polypeptides or proteins may involve supplementing the growth media with the ncAA described herein and an inducer for the aaRS expression. Alternatively, the aaRS may be expressed constitutively.

In some embodiments, aaRS/tRNA pairs may be imported from evolutionarily divergent organisms, wherein the sequence has diverged from that of the aaRS/tRNA pairs in the host organism or cell of interest (e.g., archaeal and eukaryotic pairs in an E. coli host). In some embodiments, derivatives of the Methanocaldococcus janaschii tyrosyl-tRNA synthetase (MjTyrRS)/MjtRNA^{Tyr} pair may be used to incorporate a wide variety of ncAAs into polypeptides or proteins. In some embodiments, derivatives of the E. coli leucyl-tRNA synthetase (EcLeuRS)/EctRNA^{Leu}, E. coli tryptophanyl-tRNA synthetase (EcTrpRS)/EctRNA^{Trp}), or EcTyrRS/EctRNA^{Tyr} pairs may be used to incorporate one or more ncAAs into polypeptides or proteins. In some embodiments, EcTyrRS/EctRNA^{Tyr} pair or EcTrpRS/EctRNA^{Trp} pair may be directly evolved for a new ncAA specificity. In some embodiments, endogenous copies of aaRS/tRNA pairs may be replaced with pairs that are orthogonal in another host organism.

In some embodiments, evolved derivatives of a Methanococcus maripaludis phosphoseryl-tRNA synthetase (MmpSepRS)/MARNA^{Sep} pair may be used to incorporate phosphoserine, its non-hydrolysable analogue, or phosphothreonine. In some embodiments, Methanosarcina mazei pyrrolysyl-tRNA synthetase (MmPylRS)/MmtRNA^{Pyl}_{CUA} pair, Methanosarcina barkeri PylRS (MbPylRS)/MbtRNA^{Pyl}_{CUA} pair, or derivatives thereof, may be used to incorporate one or more ncAAs. In some embodiments, Archaeoglobus fulgidus (Af)TyrRS/AftRNA^{Tyr}_{CUA} may be used to incorporate one or more ncAAs. In some embodiments, engineered aaRS/tRNA pairs may be used to incorporate one or more ncAAs.

In some embodiments, an organism or a host organism described herein can comprise an animal. In some embodiments, the animal may comprise a mammal. In some embodiments, the mammal comprises a human, non-human primate, rodent, caprine, bovine, ovine, equine, canine, feline, mouse, rat, rabbit, horse or goat. In some embodiments, an organism or a host organism may comprise E. coli, Salmonella enterica subsp. enterica serovar Typhimurium, Saccharomyces cerevisiae, cultured mammalian cells, Arabidopsis thaliana, Caenorhabditis elegans, Drosophila melanogaster or Mus musculus.

A cell or a host cell described herein can be a bacterial cell, a yeast cell, a fungal cell, an insect cell, or a mammalian cell. In some embodiments, a cell may comprise a mammalian cell. Mammalian cells can be derived or isolated from a tissue of a mammal. In some embodiments, mammalian cells may comprise COS cells, BHK cells, 293 cells, 3T3 cells, NS0 hybridoma cells, baby hamster kidney (BHK) cells, PER.C6™ human cells, HEK293 cells or Cricetulus griseus (CHO) cells. In some embodiments, a mammalian cell may comprise a human cell, a rodent cell, or a mouse cell. Examples of mammalian cells can also include but are not limited to cells from humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In some embodiments, a mammalian cell is a human cell. In some embodiments, a mammalian cell is a mouse cell. In some embodiments, a mammalian cell comprises an embryonic stem cell (ESC), a pluripotent stem cell (PSC), or an induced pluripotent stem cell (iPSC). In some embodiments, a cell or a host cell may comprise a eukaryotic cell or a prokaryotic cell. In some embodiments, the prokaryotic cell comprises an archaebacteria cell, a bacterial cell, or a combination thereof. In some embodiments, the eukaryotic cell comprises a yeast cell, a fungal cell, a plant cell, an animal cell, an insect cell, a mammalian cell, or a combination thereof. In some embodiments, the mammalian cell comprises a rodent cell, a mouse cell, or a human cell, or a combination thereof.

Methods for incorporating non-canonical amino acids in yeast are described in, for example, Stieglitz J. T., Van Deventer J. A. (2022) Incorporating, Quantifying, and Leveraging Noncanonical Amino Acids in Yeast. In: Rasooly A., Baker H., Ossandon M. R. (eds) Biomedical Engineering Technologies. Methods in Molecular Biology, vol 2394. Humana, New York, NY (doi.org/10.1007/978-1-0716-1811-0_21), which is incorporated by reference herein in its entirety.

Applications of proteins with non-canonical amino acids are described in, for example, Jeremiah A Johnson, Ying Y Lu, James A Van Deventer, David A Tirrell, Residue-specific incorporation of non-canonical amino acids into proteins: recent developments and applications, Current Opinion in Chemical Biology, Volume 14, Issue 6, 2010, Pages 774-780, ISSN 1367-5931, doi.org/10.1016/j.cbpa.2010.09.013 (www.sciencedirect.com/science/article/pii/S1367593110001390), which is incorporated by reference herein in its entirety.

Examples of orthogonal translation in E. coli are described in, for example, Robertson W E, Funke L F H, de la Torre D, Fredens J, Elliott T S, Spinck M, Christova Y, Cervettini D, Boge F L, Liu K C, Buse S, Maslen S, Salmond G P C, Chin J W. Sense codon reassignment enables viral resistance and encoded polymer synthesis. Science. 2021 Jun. 4; 372(6546):1057-1062. doi: 10.1126/science.abg3029. PMID: 34083482; PMCID: PMC7611380, which is incorporated by reference herein in its entirety.

Additional examples of orthogonal translation are described in, for example, de la Torre, D., Chin, J. W. Reprogramming the genetic code. Nat Rev Genet 22, 169-184 (2021) (doi.org/10.1038/s41576-020-00307-7), which is incorporated by reference herein in its entirety.

### Machine Learning-Based Computer Systems

In some aspects, methods described herein may comprise utilizing a machine learning-based computer system. In some embodiments, machine learning-based computer systems described herein may comprise one or more storage units comprising, respectively, one or more storage devices included within respective storage arrays controlled by a respective one or more storage controllers; and one or more computer processing units, wherein the one or more computer processing units are configured to communicate with the one or more storage units over a communication interface.

In some non-limiting example embodiments, machine learning can include: supervised machine learning, Random Forest, support vector machine, neural network, regression tree, or unsupervised machine learning.

In some embodiments, the machine learning-based computer system provides the plurality of intermediate scores to a machine learning algorithm that processes the plurality of intermediate scores to generate the rewritten stop codons (e.g., the first plurality of stop codons that are selected to be rewritten into a second stop codon). The machine learning algorithm may comprise a function that determines how intermediate scores are combined and weighted. The machine learning algorithm may comprise a supervised machine learning algorithm. The supervised machine learning algorithm may be trained on prior data from a reference genome, or on prior data from multiple genomes. The prior data may include observed fitness values for genomes, including growth rates on different media. The machine learning-based computer system can train the supervised machine learning algorithm by providing examples of fitness values to an untrained or partially trained version of the algorithm to generate replacement codons for one or more of the input genomes or of a different genome. The system can compare the predicted fitness to the measured fitness (i.e., whether the cell growth rate was maintained), and if there is a difference, the system can perform training at least in part by updating the parameters of the supervised machine learning algorithm. The supervised machine learning algorithm may comprise a regression algorithm, a support vector machine, a decision tree, a neural network, or the like. In cases in which the machine learning algorithm comprises a regression algorithm, the weights may be regression parameters. The supervised machine learning algorithm may comprise a classifier or a predictor that determines a prediction of which replacement codons (e.g., selected from among a plurality of possible replacement codons) are least likely to result in a fitness deficit. The predictor may generate a fitness risk score that is indicative of a likelihood of being indicative of a fitness risk (e.g., probabilistic fitness risk score between 0 and 1). In some cases, the machine learning-based computer system may map the probabilistic risk score to a qualitative risk category (e.g., selected from among a plurality of risk categories). For example, a fitness risk score that is at least 0.5 may be considered a high risk, while a fitness risk score that is less than 0.5 may be considered a low risk. Alternatively, the supervised machine learning algorithm may be a multi-class classifier (e.g., binary classifier) that predicts a qualitative risk category directly.

The machine learning algorithm may comprise unsupervised machine learning algorithm. The unsupervised machine learning algorithm may identify patterns in a genome or multiple genomes of interest. For example, it may identify a set of codon usage contexts that are an outlier as compared to other sets of codon usage for the same amino acid. If the unsupervised machine learning algorithm determines that a particular context-dependent codon usage is an outlier, the machine learning-based computer system may determine that relying on genome-wide codon usage for codon selection may lead to a fitness deficit. On the other hand, a set of codon usage scores that is consistent with overall codon usage for the genome may indicate that codon replacement has lower risk of generating a fitness defect. The unsupervised machine learning algorithm may comprise a clustering algorithm, an isolation forest, an autoencoder, or the like.

### Computer Systems

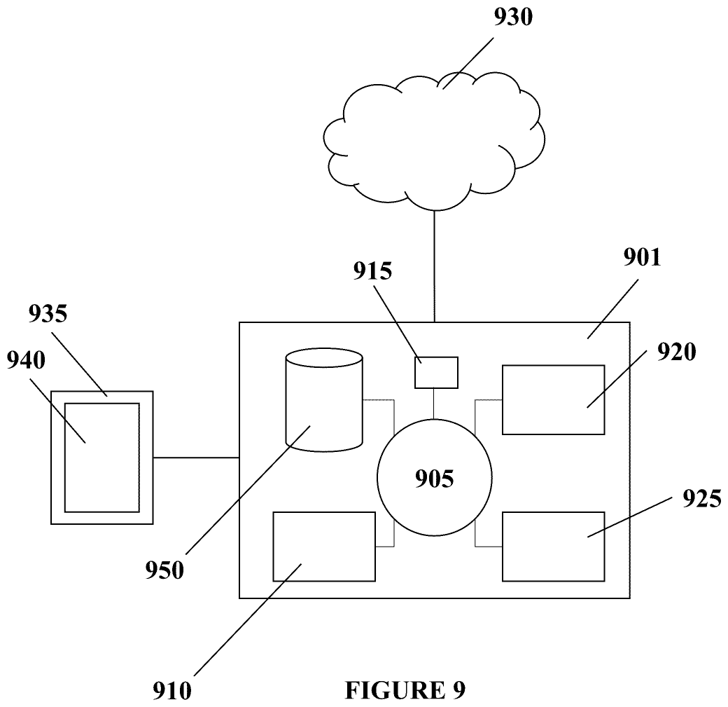

The present disclosure provides computer systems that are programmed to implement methods of the disclosure. FIG. 9 shows a computer system 901.

The computer system 901 includes a central processing unit (CPU, also “processor” and “computer processor” herein) 905, which can be a single core or multi core processor, or a plurality of processors for parallel processing. The computer system 901 also includes memory or memory location 910 (e.g., random-access memory, read-only memory, flash memory), electronic storage unit 915 (e.g., hard disk), communication interface 920 (e.g., network adapter) for communicating with one or more other systems, and peripheral devices 925, such as cache, other memory, data storage and/or electronic display adapters. The memory 910, storage unit 915, interface 920 and peripheral devices 925 are in communication with the CPU 905 through a communication bus (solid lines), such as a motherboard. The storage unit 915 can be a data storage unit (or data repository) for storing data. The computer system 901 can be operatively coupled to a computer network (“network”) 930 with the aid of the communication interface 920. The network 930 can be the Internet, an internet and/or extranet, or an intranet and/or extranet that is in communication with the Internet.

The network 930 in some cases is a telecommunication and/or data network. The network 930 can include one or more computer servers, which can enable distributed computing, such as cloud computing. For example, one or more computer servers may enable cloud computing over the network 930 (“the cloud”) to perform various aspects of analysis, calculation, and generation of the present disclosure. Such cloud computing may be provided by cloud computing platforms such as, for example, Amazon Web Services (AWS), Microsoft Azure, Google Cloud Platform, and IBM cloud. The network 930, in some cases with the aid of the computer system 901, can implement a peer-to-peer network, which may enable devices coupled to the computer system 901 to behave as a client or a server.

The CPU 905 may comprise one or more computer processors and/or one or more graphics processing units (GPUs). The CPU 905 can execute a sequence of machine-readable instructions, which can be embodied in a program or software. The instructions may be stored in a memory location, such as the memory 910. The instructions can be directed to the CPU 905, which can subsequently program or otherwise configure the CPU 905 to implement methods of the present disclosure. Examples of operations performed by the CPU 905 can include fetch, decode, execute, and writeback.

The CPU 905 can be part of a circuit, such as an integrated circuit. One or more other components of the system 901 can be included in the circuit. In some cases, the circuit is an application specific integrated circuit (ASIC).

The storage unit 915 can store files, such as drivers, libraries and saved programs. The storage unit 915 can store user data, e.g., user preferences and user programs. The computer system 901 in some cases can include one or more additional data storage units that are external to the computer system 901, such as located on a remote server that is in communication with the computer system 901 through an intranet or the Internet.

The computer system 901 can communicate with one or more remote computer systems through the network 930. For instance, the computer system 901 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 901 via the network 930.

Methods as described herein can be implemented by way of machine (e.g., computer processor) executable code stored on an electronic storage location of the computer system 901, such as, for example, on the memory 910 or electronic storage unit 915. The machine-executable or machine-readable code can be provided in the form of software. During use, the code can be executed by the processor 905. In some cases, the code can be retrieved from the storage unit 915 and stored on the memory 910 for ready access by the processor 905. In some situations, the electronic storage unit 915 can be precluded, and machine-executable instructions are stored on memory 910.

The code can be pre-compiled and configured for use with a machine having a processer adapted to execute the code or can be compiled during runtime. The code can be supplied in a programming language that can be selected to enable the code to execute in a pre-compiled or as-compiled fashion.

Aspects of the systems and methods provided herein, such as the computer system 901, can be embodied in programming. Various aspects of the technology may be thought of as “products” or “articles of manufacture” typically in the form of machine (or processor) executable code and/or associated data that is carried on or embodied in a type of machine-readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Hence, a machine-readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer-readable media may be involved in carrying one or more sequences of one or more instructions to a processor for execution.

The computer system 901 can include or be in communication with an electronic display 935 that comprises a user interface (UI) 940.

### Trained Algorithms

In some aspects, methods and systems described herein may employ one or more trained algorithms. The trained algorithm(s) may process or operate on one or more datasets comprising information about a codon-of-interest, a codon upstream of (or 5′ to) the stop codon-of-interest, a codon downstream of (or 3′ to) the stop codon-of-interest, or any combination thereof. The trained algorithm(s) may process or operate on one or more datasets comprising information about a stop codon-of-interest. In some embodiments, the datasets comprise structural or sequence information about codons. In some embodiments, the datasets comprise one or more datasets of codons. The one or more datasets may be observed empirically, derived from computational studies, be derived or retrieved from one or more databases, be artificially generated (e.g., as in silico variants of empirically observed datasets), or any combination thereof.

The trained algorithm may comprise an unsupervised machine learning algorithm. The trained algorithm may comprise a supervised machine learning algorithm. The trained algorithm may comprise a classification and regression tree (CART) algorithm. The supervised machine learning algorithm may comprise, for example, a Random Forest, a support vector machine (SVM), a neural network, or a deep learning algorithm. The trained algorithm may comprise a self-supervised machine learning algorithm. The trained algorithm may comprise a statistical model, statistical analysis, or statistical learning.

In some embodiments, a machine learning algorithm (or software module) of a platform as described herein utilizes one or more neural networks. In some embodiments, a neural network is a type of computational system that can learn the relationships between an input dataset and a target dataset. A neural network may be a software representation of a human neural system (e.g., cognitive system), intended to capture “learning” and “generalization” abilities as used by a human. In some embodiments, the machine learning algorithm (or software module) comprises a neural network comprising a convolutional neural network (CNN). In some non-limiting example embodiments, structural components of embodiments of the machine learning software described herein include: CNNs, recurrent neural networks, dilated CNNs, fully-connected neural networks, deep generative models, and Boltzmann machines.

In some embodiments, a neural network comprises a series of layers termed “neurons.” In some embodiments, a neural network comprises an input layer, to which data is presented; one or more internal, and/or “hidden”, layers; and an output layer. A neuron may be connected to neurons in other layers via connections that have weights, which are parameters that control the strength of the connection. The number of neurons in each layer may be related to the complexity of the problem to be solved. The minimum number of neurons required in a layer may be determined by the problem complexity, and the maximum number may be limited by the ability of the neural network to generalize. The input neurons may receive data being presented and then transmit that data to the first hidden layer through connections' weights, which are modified during training. The first hidden layer may process the data and transmit its result to the next layer through a second set of weighted connections. Each subsequent layer may “pool” the results from a set of the previous layers into more complex relationships. In addition, whereas some software programs require writing specific instructions to perform a task, neural networks are programmed by training them with a known sample set and allowing them to modify themselves during (and after) training so as to provide a desired output such as an output value (e.g., predicted value). After training, when a neural network is presented with new input data, it generalizes what was “learned” during training and applies what was learned from training to the new, previously unseen, input data in order to generate an output associated with that input (e.g., a predicted value). The output may be generated in order to minimize an expected error or loss function between the output value and an expected value.

In some embodiments, the neural network comprises artificial neural networks (ANNs). ANNs may be machine learning algorithms that may be trained to map an input dataset to an output dataset, where the ANN comprises an interconnected group of nodes organized into multiple layers of nodes. For example, the ANN architecture may comprise at least an input layer, one or more hidden layers, and an output layer. The ANN may comprise any total number of layers, and any number of hidden layers, where the hidden layers function as trainable feature extractors that allow mapping of a set of input data to an output value or set of output values. As used herein, a deep learning algorithm (such as a deep neural network, or DNN) is an ANN comprising a plurality of hidden layers, e.g., two or more hidden layers. Each layer of the neural network may comprise a number of nodes (or “neurons”). A node receives a set of inputs that are retrieved from either directly from the input data or the output of nodes in previous layers, and performs a specific operation, e.g., a summation operation, on the set of inputs. A connection from an input to a node is associated with a weight (or weighting factor). The node may determine a sum of the products of all pairs of inputs and their associated weights. The weighted sum may be offset with a bias. The output of a node or neuron may be gated using a threshold or activation function. The activation function may be a linear or non-linear function. The activation function may be, for example, a rectified linear unit (ReLU) activation function, a Leaky ReLU activation function, or other function such as a saturating hyperbolic tangent, identity, binary step, logistic, arctan, softsign, parametric rectified linear unit, exponential linear unit, softplus, bent identity, soft exponential, sinusoid, sinc, Gaussian, or sigmoid function, or any combination thereof.

The weighting factors, bias values, and threshold values, or other computational parameters of the neural network, may be “taught” or “learned” in a training phase using one or more sets of training data. For example, the parameters may be trained using the input data from a training dataset and a gradient descent or backward propagation method so that the output value(s) that the ANN determines are consistent with the examples included in the training dataset.

The number of nodes used in the input layer of the ANN or DNN may be at least about 10, 50, 100, 200, 300, 400, 500, 600, 700, 800, 900, 1,000, 2,000, 3,000, 4,000, 5,000, 6,000, 7,000, 8,000, 9,000, 10,000, 20,000, 30,000, 40,000, 50,000, 60,000, 70,000, 80,000, 90,000, 100,000, or greater. In other instances, the number of node used in the input layer may be at most about 100,000, 90,000, 80,000, 70,000, 60,000, 50,000, 40,000, 30,000, 20,000, 10,000, 9,000, 8,000, 7,000, 6,000, 5,000, 4,000, 3,000, 2,000, 1,000, 900, 800, 700, 600, 500, 400, 300, 200, 100, 50, 10, or fewer. In some instances, the total number of layers used in the ANN or DNN (including input and output layers) may be at least about 3, 4, 5, 10, 15, 20, or greater. In other instances, the total number of layers may be at most about 20, 15, 10, 5, 4, 3, or fewer.

In some instances, the total number of learnable or trainable parameters, e.g., weighting factors, biases, or threshold values, used in the ANN or DNN may be at least about 10, 50, 100, 200, 300, 400, 500, 600, 700, 800, 900, 1,000, 2,000, 3,000, 4,000, 5,000, 6,000, 7,000, 8,000, 9,000, 10,000, 20,000, 30,000, 40,000, 50,000, 60,000, 70,000, 80,000, 90,000, 100,000, or greater. In other instances, the number of learnable parameters may be at most about 100,000, 90,000, 80,000, 70,000, 60,000, 50,000, 40,000, 30,000, 20,000, 10,000, 9,000, 8,000, 7,000, 6,000, 5,000, 4,000, 3,000, 2,000, 1,000, 900, 800, 700, 600, 500, 400, 300, 200, 100, 50, 10, or fewer.

In some embodiments described herein, a machine learning software module comprises a neural network such as a deep CNN. In some embodiments in which a CNN is used, the network is constructed with any number of convolutional layers, dilated layers, or fully connected layers. In some embodiments, the number of convolutional layers is between 1-10, and the number of dilated layers is between 0-10. The total number of convolutional layers (including input and output layers) may be at least about 1, 2, 3, 4, 5, 10, 15, 20, or greater, and the total number of dilated layers may be at least about 1, 2, 3, 4, 5, 10, 15, 20, or greater. The total number of convolutional layers may be at most about 20, 15, 10, 5, 4, 3, or fewer, and the total number of dilated layers may be at most about 20, 15, 10, 5, 4, 3, or fewer. In some embodiments, the number of convolutional layers is between 1-10 and the fully connected layers between 0-10. The total number of convolutional layers (including input and output layers) may be at least about 1, 2, 3, 4, 5, 10, 15, 20, or greater, and the total number of fully connected layers may be at least about 1, 2, 3, 4, 5, 10, 15, 20, or greater. The total number of convolutional layers may be at most about 20, 15, 10, 5, 4, 3, 2, 1, or less, and the total number of fully connected layers may be at most about 20, 15, 10, 5, 4, 3, 2, 1, or fewer.

In some embodiments, the input data for training of the ANN may comprise a variety of input values depending on whether the machine learning algorithm is used for processing sequence or structural data. In some embodiments, the ANN or deep learning algorithm may be trained using one or more training datasets comprising the same or different sets of input and paired output data.

In some embodiments, a machine learning software module comprises a neural network comprising a CNN, recurrent neural network (RNN), dilated CNN, fully connected neural networks, deep generative models, and deep restricted Boltzmann machines.

In some embodiments, a machine learning algorithm comprises CNNs. The CNN may be deep and feedforward ANNs. The CNN may be applicable to analyzing visual imagery. The CNN may comprise an input, an output layer, and multiple hidden layers. The hidden layers of a CNN may comprise convolutional layers, pooling layers, fully connected layers, and normalization layers. The layers may be organized in 3 dimensions: width, height, and depth.

The convolutional layers may apply a convolution operation to the input and pass results of the convolution operation to the next layer. For processing sequence data, the convolution operation may reduce the number of free parameters, allowing the network to be deeper with fewer parameters. In neural networks, each neuron may receive input from some number of locations in the previous layer. In a convolutional layer, neurons may receive input from only a restricted subarea of the previous layer. The convolutional layer's parameters may comprise a set of learnable filters (or kernels). The learnable filters may have a small receptive field and extend through the full depth of the input volume. During the forward pass, each filter may be convolved across the length of the input sequence, determine the dot product between the entries of the filter and the input, and produce a two-dimensional activation map of that filter. As a result, the network may learn filters that activate when it detects some specific type of feature at some spatial position in the input.

In some embodiments, the pooling layers comprise global pooling layers. The global pooling layers may combine the outputs of neuron clusters at one layer into a single neuron in the next layer. For example, max pooling layers may use the maximum value from each of a cluster of neurons in the prior layer; and average pooling layers may use the average value from each of a cluster of neurons at the prior layer.

In some embodiments, the fully connected layers connect every neuron in one layer to every neuron in another layer. In neural networks, each neuron may receive input from some number locations in the previous layer. In a fully connected layer, each neuron may receive input from every element of the previous layer.

In some embodiments, the normalization layer is a batch normalization layer. The batch normalization layer may improve the performance and stability of neural networks. The batch normalization layer may provide any layer in a neural network with inputs that are zero mean/unit variance. The advantages of using batch normalization layer may include faster trained networks, higher learning rates, easier to initialize weights, more activation functions viable, and simpler process of creating deep networks.

In some embodiments, a machine learning software module comprises a RNN software module. A RNN software module may receive sequential data as an input, such as consecutive data inputs, and the RNN software module updates an internal state at every time step. A RNN can use internal state (memory) to process sequences of inputs. The RNN may be applicable to tasks such as codon selection. The RNN may also be applicable to next codon prediction, and codon usage anomaly detection. In some embodiments, a RNN may comprise a fully recurrent neural network, an independently recurrent neural network, Elman networks, Jordan networks, an Echo state, a neural history compressor, a long short-term memory, a gated a recurrent unit, a multiple timescales model, neural Turing machines, a differentiable neural computer, and a neural network pushdown automata.

In some embodiments, a machine learning software module comprises a supervised or unsupervised learning method such as, for example, support vector machines (“SVMs”), random forests, clustering algorithm (or software module), gradient boosting, linear regression, logistic regression, and/or decision trees. The supervised learning algorithms may be algorithms that rely on the use of a set of labeled, paired training data examples to infer the relationship between an input data and output data. The unsupervised learning algorithms may be algorithms used to draw inferences from training datasets to the output data. The unsupervised learning algorithm may comprise cluster analysis, which may be used for exploratory data analysis to find hidden patterns or groupings in process data. One example of unsupervised learning method may comprise principal component analysis. The principal component analysis may comprise reducing the dimensionality of one or more variables. The dimensionality of a given variable may be at least 1, 5, 10, 50, 100, 200, 300, 400, 500, 600, 700, 800, 900, 1,000, 1,100, 1,200 1,300, 1,400, 1,500, 1,600, 1,700, 1,800, or greater. The dimensionality of a given variables may be at most 1,800, 1,700, 1,600, 1,500, 1,400, 1,300, 1,200, 1,100, 1,000, 900, 800, 700, 600, 500, 400, 300, 200, 100, 50, 10, or fewer.

In some embodiments, the machine learning algorithm may comprise reinforcement learning algorithms. The reinforcement learning algorithm may be used for optimizing Markov decision processes (i.e., mathematical models used for studying a wide range of optimization problems where future behavior cannot be accurately predicted from past behavior alone, but rather also depends on random chance or probability). One example of reinforcement learning may be Q-learning. Reinforcement learning algorithms may differ from supervised learning algorithms in that correct training data input/output pairs are not presented, nor are sub-optimal actions explicitly corrected. The reinforcement learning algorithms may be implemented with a focus on real-time performance through finding a balance between exploration of possible outcomes (e.g., correct compound identification) based on updated input data and exploitation of past training.

In some embodiments, training data resides in a cloud-based database that is accessible from local and/or remote computer systems on which the machine learning-based sensor signal processing algorithms are running. The cloud-based database and associated software may be used for archiving electronic data, sharing electronic data, and analyzing electronic data. In some embodiments, training data generated locally may be uploaded to a cloud-based database, from which it may be accessed and used to train other machine learning-based detection systems at the same site or a different site.

In some embodiments, the trained algorithm may accept a plurality of input variables and produce one or more output variables based on the plurality of input variables. The input variables may comprise one or more datasets of codons. For example, the input variables may comprise information about a codon-of-interest, a codon upstream of (or 5′ to) the codon-of-interest, a codon downstream of (or 3′ to) the codon-of-interest, or any combination thereof. For example, the input variables may comprise a stop codon.

In some embodiments, the trained algorithm may be trained with a plurality of independent training samples. Each of the independent training samples may comprise information about a codon-of-interest, a codon upstream of (or 5′ to) the codon-of-interest, a codon downstream of (or 3′ to) the codon-of-interest, or a combination thereof. Each of the independent training samples may comprise information about a stop codon. The trained algorithm may be trained with at least about 5, at least about 10, at least about 15, at least about 20, at least about 25, at least about 30, at least about 35, at least about 40, at least about 45, at least about 50, at least about 100, at least about 150, at least about 200, at least about 250, at least about 300, at least about 350, at least about 400, at least about 450, at least about 500, at least about 1,000, at least about 1,500, at least about 2,000, at least about 2,500, at least about 3,000, at least about 3,500, at least about 4,000, at least about 4,500, at least about 5,000, at least about, 5,500, at least about 6,000, at least about 6,500, at least about 7,000, at least about 7,500, at least about 8,000, at least about 8,500, at least about 9,000, at least about 9,500, at least about 10,000, or more independent training samples.

In some embodiments, the trained algorithm may associate information about a codon-of-interest, a codon upstream of (or 5′ to) the codon-of-interest, a codon downstream of (or 3′ to) the codon-of-interest, or a combination thereof for the best selection of codons for rewriting/replacement at an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The trained algorithm may associate information about a stop codon for the best selection of codons for rewriting/replacement at an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The trained algorithm may be adjusted or tuned to improve a performance or accuracy of determining the prediction or classification. The trained algorithm may be adjusted or tuned by adjusting parameters of the trained algorithm. The trained algorithm may be adjusted or tuned continuously during the training process or after the training process has completed.

In some embodiments, after the trained algorithm is initially trained, a subset of the inputs may be identified as most influential or most important to be included for making high-quality predictions. For example, a subset of the data may be identified as most influential or most important to be included for making high-quality choice for selecting codons for rewriting and/or replacement. The data or a subset thereof may be ranked based on classification metrics indicative of each parameter's influence or importance toward making high-quality selection of codons for rewriting and/or replacement. Such metrics may be used to reduce, in some embodiments significantly, the number of input variables (e.g., predictor variables) that may be used to train the trained algorithm to a desired performance level (e.g., based on a desired minimum accuracy). For example, if training the trained algorithm with a plurality comprising several dozen or hundreds of input variables in the trained algorithm results in an accuracy of classification of more than 99%, then training the trained algorithm instead with only a selected subset of no more than about 5, no more than about 10, no more than about 15, no more than about 20, no more than about 25, no more than about 30, no more than about 35, no more than about 40, no more than about 45, no more than about 50, or no more than about 100 such most influential or most important input variables among the plurality can yield decreased but still acceptable accuracy of classification (e.g., at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%). The subset may be selected by rank-ordering the entire plurality of input variables and selecting a predetermined number (e.g., no more than about 5, no more than about 10, no more than about 15, no more than about 20, no more than about 25, no more than about 30, no more than about 35, no more than about 40, no more than about 45, no more than about 50, or no more than about 100) of input variables with the best association metrics.

Systems and methods as described herein may use more than one trained algorithm to determine an output. Systems and methods may comprise 1, 2, 3, 4, 5, 6, 7, 8, 9, 10, 11, 12, 13, 14, 15, 16, 17, 18, 19, 20, or more trained algorithms. A trained algorithm of the plurality of trained algorithms may be trained on a particular type of data (e.g., sequence data, structural data). Alternatively, a trained algorithm may be trained on more than one type of data. The inputs of one trained algorithm may comprise the outputs of one or more other trained algorithms. Additionally, a trained algorithm may receive as its input the output of one or more trained algorithms. A set of outputs generated using one or more trained algorithms may be combined into a single output (e.g., by determining a sum, an average, a minimum, a maximum, or any other function applied to the set of outputs).

### OTHER EMBODIMENTS

In some aspects, provided herein is a composition comprising: (a) a first recombinant release factor, wherein the first recombinant release factor is configured to recognize UGA, UAA, UAG, or any combination thereof as a stop codon; and (b) a second recombinant release factor, wherein the second recombinant release factor is configured to recognize UGA, UAA, UAG, or any combination thereof as a stop codon, and wherein the second recombinant release factor comprises an element that allows selective modulation of function of the second recombinant release factor.

In some embodiments, the first recombinant release factor recognizes UGA as the stop codon. In some embodiments, the first recombinant release factor does not recognize UAA and/or UAG as the stop codon. In some embodiments, the first recombinant release factor recognizes UAA, UAG, or any combination thereof as the stop codon. In some embodiments, the first recombinant release factor does not recognize UGA as the stop codon. In some embodiments, the second recombinant release factor recognizes UAA and UAG as stop codons. In some embodiments, the second recombinant release factor recognizes UGA as the stop codon. In some embodiments, the second recombinant release factor recognizes UGA, UAA, and UAG as stop codons.

In some embodiments, the element that allows selective modulation of the function of the second recombinant release factor comprises a temperature sensitive allele that allows the second recombinant release factor to function only at a permissive temperature or a degron cassette that allows degradation of the second recombinant release factor.

In some embodiments, the temperature sensitive allele comprises sup45-ts, sup45-2, sup45-36ts, sup45-1023ts, or sup45-sl23ts. In some embodiments, the sup45-ts comprises a sequence with at least 70% sequence identity to SEQ ID NO: 162. In some embodiments, the sup45-2 comprises a sequence with at least 70% sequence identity to SEQ ID NO: 163. In some embodiments, the sup45-36ts comprises a sequence with at least 70% sequence identity to SEQ ID NO: 164. In some embodiments, the sup45-1023ts comprises a sequence with at least 70% sequence identity to SEQ ID NO: 165. In some embodiments, the sup45-sl23ts comprises a sequence with at least 70% sequence identity to SEQ ID NO: 166 or 167. In some embodiments, the permissive temperature comprises from about 20° C. to about 33° C. In some embodiments, the permissive temperature is 25° C.

In some embodiments, the degron cassette comprises a heat-inducible degron cassette or a small molecule-inducible degron cassette. In some embodiments, the degron cassette comprises the small molecule-inducible degron cassette. In some embodiments, the small molecule comprises an auxin or asunaprevir.

In some embodiments, the first or the second recombinant release factor modulates protein translation upon recognizing UGA, UAA, or UAG as the stop codon. In some embodiments, the modulation comprises terminating protein translation.

In some embodiments, the first or the second recombinant release factor comprises a class 1 release factor, a class 2 release factor, or a combination thereof. In some embodiments, the class 1 release factor is a eukaryotic release factor 1 (eRF1). In some embodiments, the class 2 release factor comprises a release factor 3. In some embodiments, the class 2 release factor is a eukaryotic release factor 3 (eRF3). In some embodiments, the first or the second recombinant release factor comprises a release factor 1/release factor 3 complex. In some embodiments, the first or the second recombinant release factor is a eukaryotic release factor 1/release factor 3 (eRF1/eRF3) complex.

In some embodiments, the first or the second recombinant release factor comprises a recognition domain comprising one or more mutations that allow the first or the second recombinant release factor to recognize only (i) UGA, (ii) UAA, (iii) UAG, or (iv) any combination thereof.

In some embodiments, the first or the second recombinant release factor comprises a first recognition domain swapped with a second recognition domain. In some embodiments, the second recognition domain is from a release factor of a second organism. In some embodiments, the second recognition domain is identified using a phylogenetic screening, directed evolution, library screening, machine learning, or a combination thereof.

In some embodiments, the first or the second recombinant release factor is from a first organism. In some embodiments, the first organism comprises a eukaryotic cell or a prokaryotic cell. In some embodiments, the prokaryotic cell comprises an archaebacteria cell, a bacterial cell, or a combination thereof. In some embodiments, the eukaryotic cell comprises a yeast cell, a fungal cell, a plant cell, an animal cell, an insect cell, a mammalian cell, or a combination thereof. In some embodiments, the yeast cell comprises Saccharomyces cerevisiae.

In some embodiments, the first or the second recombinant release factor is from a second organism. In some embodiments, the second organism comprises a ciliate. In some embodiments, the ciliate comprises Blepharisma americanum, Blepharisma japonicum, Euplotes aediculatus, Euplotes octocarinatus, Stentor coeruleus, Nyctotherus ovalis, Stylonychia lemnae, Pseudocohnilembus persalinus, Ichthyophthirius multifiliis, Stylonychia lemnae, Oxytricha trifallax, Stylonychia pustulata, Stylonychia mytilus, Eschaneustyla sp. HL-2004, Gonostomum sp. HL-2004, Holosticha sp. HL-2004, Urostyla sp. HL-2004, Uroleptus sp. WIC-2003, Paraurostyla weissei, Stichotrichida sp. Misty, Stichotrichida sp. Alaska, Spironucleus salmonicida, Loxodes striatus, Paramecium tetraurelia, or Tetrahymena thermophila.

In some embodiments, the second recognition domain comprises an amino acid sequence with at least 50% sequence identity to KSSNIKS (SEQ ID NO: 3), YICDNKF (SEQ ID NO: 4), TAVNIKS (SEQ ID NO: 5), KAANIKS (SEQ ID NO: 6), KASNIKS (SEQ ID NO: 7), YYCGERF (SEQ ID NO: 8), TAESIKS (SEQ ID NO: 9), YFCDPQF (SEQ ID NO: 10), EAASIKD (SEQ ID NO: 11), KATNIKD (SEQ ID NO: 12), YFCDSKF (SEQ ID NO: 13), FDFDAES (SEQ ID NO: 14), TLIKPQF (SEQ ID NO: 15), TGDKIKS (SEQ ID NO: 16), TIIKNDF (SEQ ID NO: 17), EAASIQD (SEQ ID NO: 18), FFCDNYF (SEQ ID NO: 19), FVIVNKF (SEQ ID NO: 20), AAQNIKS (SEQ ID NO: 21), YFCGGKF (SEQ ID NO: 22), QANSIKD (SEQ ID NO: 23), YRCDSKF (SEQ ID NO: 24), GAASIKN (SEQ ID NO: 25), YSCNTIF (SEQ ID NO: 26), SAQNIKS (SEQ ID NO: 27), YYCDNRF (SEQ ID NO: 28), SAGNIKS (SEQ ID NO: 29), YFCDNSF (SEQ ID NO: 30), TAQNIKS (SEQ ID NO: 31), SAQSIKS (SEQ ID NO: 32), AANNIKS (SEQ ID NO: 33), YNCSGKF (SEQ ID NO: 34), QAQNIKS (SEQ ID NO: 35), QADCIKS (SEQ ID NO: 36), YSCDGVF (SEQ ID NO: 37), RAQNIKS (SEQ ID NO: 38), FLCENTF (SEQ ID NO: 39), or a combination thereof.

In some embodiments, the second recognition domain comprises an amino acid sequence comprising KSSNIKS (SEQ ID NO: 3), YICDNKF (SEQ ID NO: 4), TAVNIKS (SEQ ID NO: 5), KAANIKS (SEQ ID NO: 6), KASNIKS (SEQ ID NO: 7), YYCGERF (SEQ ID NO: 8), TAESIKS (SEQ ID NO: 9), YFCDPQF (SEQ ID NO: 10), EAASIKD (SEQ ID NO: 11), KATNIKD (SEQ ID NO: 12), YFCDSKF (SEQ ID NO: 13), FDFDAES (SEQ ID NO: 14), TLIKPQF (SEQ ID NO: 15), TGDKIKS (SEQ ID NO: 16), TIIKNDF (SEQ ID NO: 17), EAASIQD (SEQ ID NO: 18), FFCDNYF (SEQ ID NO: 19), FVIVNKF (SEQ ID NO: 20), AAQNIKS (SEQ ID NO: 21), YFCGGKF (SEQ ID NO: 22), QANSIKD (SEQ ID NO: 23), YRCDSKF (SEQ ID NO: 24), GAASIKN (SEQ ID NO: 25), YSCNTIF (SEQ ID NO: 26), SAQNIKS (SEQ ID NO: 27), YYCDNRF (SEQ ID NO: 28), SAGNIKS (SEQ ID NO: 29), YFCDNSF (SEQ ID NO: 30), TAQNIKS (SEQ ID NO: 31), SAQSIKS (SEQ ID NO: 32), AANNIKS (SEQ ID NO: 33), YNCSGKF (SEQ ID NO: 34), QAQNIKS (SEQ ID NO: 35), QADCIKS (SEQ ID NO: 36), YSCDGVF (SEQ ID NO: 37), RAQNIKS (SEQ ID NO: 38), FLCENTF (SEQ ID NO: 39), or a combination thereof.

In some embodiments, the first or the second recombinant release factor comprises an amino acid sequence selected from the group consisting of SEQ ID NOs: 40-64. In some embodiments, the first or the second recombinant release factor from the second organism comprises an eRF1. In some embodiments, the eRF1 from the second organism comprises an amino acid sequence that has at least 20% sequence identity to an eRF1 of the first organism. In some embodiments, the first or the second recombinant release factor comprises an amino acid sequence selected from the group consisting of SEQ ID NOs: 65-74.

In some embodiments, the first or the second recombinant release factor from the second organism comprises an eRF1/eRF3 complex. In some embodiments, the eRF1 of the eRF1/eRF3 complex from the second organism comprises an amino acid sequence that has at least 20% sequence identity to an eRF1 of the first organism. In some embodiments, the eRF3 of the eRF1/eRF3 complex comprises an amino acid sequence selected from the group consisting of SEQ ID NOs: 75, 77, 79, 81, 83, 85, 87, 89, and 91. In some embodiments, the eRF3 of the eRF1/eRF3 complex from the second organism comprises an amino acid sequence that has at least 25% sequence identity to an eRF3 of the first organism. In some embodiments, the eRF3 of the eRF1/eRF3 complex comprises an amino acid sequence selected from the group consisting of SEQ ID NOs: 76, 78, 80, 82, 84, 86, 88, 90, and 92.

In some embodiments, the first or the second recombinant release factor from the second organism comprises an eRF1 and forms a complex with a chimeric eRF3. In some embodiments, the eRF1 of the second organism comprises an amino acid sequence that has at least 40% sequence identity to an eRF1 of the first organism. In some embodiments, the chimeric eRF3 comprises (i) an eRF3 from the first organism or a fragment thereof and (ii) an eRF3 from a second organism or a fragment thereof. In some embodiments, the second organism comprises Euplotes octocarinatus or Paramecium tetraurelia. In some embodiments, the chimeric eRF3 comprises an eRF3 of Euplotes octocarinatus, wherein amino acids 7-298 of the eRF3 of Euplotes octocarinatus is replaced with amino acids 6-253 of the eRF3 from the first organism. In some embodiments, the chimeric eRF3 comprises an amino acid sequence comprising SEQ ID NO: 93 or SEQ ID NO: 94. In some embodiments, the chimeric eRF3 comprises an eRF3 of Euplotes octocarinatus, wherein amino acids 1-298 of the eRF3 of Euplotes octocarinatus is replaced with amino acids 1-253 of the eRF3 from the first organism. In some embodiments, the chimeric eRF3 comprises an amino acid sequence comprising SEQ ID NO: 95 or SEQ ID NO: 96. In some embodiments, the chimeric eRF3 comprises an eRF3 of Paramecium tetraurelia, wherein amino acids 1-321 of the eRF3 of Paramecium tetraurelia is replaced with amino acids 1-253 of the eRF3 from the first organism. In some embodiments, the chimeric eRF3 comprises an amino acid sequence comprising SEQ ID NO: 97, SEQ ID NO: 98, SEQ ID NO: 99, or SEQ ID NO: 100.

In some embodiments, the composition further comprises one or more tRNA molecules that recognize UAG, UAA, or UGA and one or more aminoacyl-tRNA synthetases (aaRSs) for charging the one or more tRNA molecules with a non-canonical amino acid (ncAA). In some embodiments, the ncAA comprises p-azidophenylalanine, 2-aminoisobutyric acid (Aib), an azide-containing ncAA, an alkene-containing ncAA, an alkyne-containing ncAA, or a combination thereof. In some embodiments, the azide-containing ncAA comprises (S)-2-amino-6-((2-azidoethoxy)carbonylamino)hexanoic acid (LysN3). In some embodiments, the alkene-containing ncAA or the alkyne-containing ncAA comprises (2S)-2-amino-6-(((prop-2-yn-1-yloxy)carbonyl)amino)hexanoic acid (LysAlk), N^{ε}-Allyloxycarbonyl-L-lysine, N-ε-propargyloxycarbonyl-L-lysine, L-2-Allylglycine, or O-Allyl-L-Tyrosine. In some embodiments, the one or more tRNA molecules recognize UAG and the one or more aaRSs charges the one or more tRNA molecules with a first ncAA. In some embodiments, the one or more tRNA molecules recognize UAA and the one or more aaRSs charges the one or more tRNA molecules with a second ncAA. In some embodiments, the one or more tRNA molecules recognize UGA and the one or more aaRSs charges the one or more tRNA molecules with a third ncAA. In some embodiments, the first ncAA, the second ncAA, and/or the third ncAA are different from each other.

In some aspects, provided herein is a recombinant nucleic acid construct comprising a sequence encoding any of the first recombinant release factor described herein. In some aspects, provided herein is a recombinant nucleic acid construct comprising a sequence encoding any of the second recombinant release factor described herein. In some embodiments, the sequence comprises a conditional promoter for expressing the first recombinant release factor or the second recombinant release factor. In some embodiments, the conditional promoter comprises a galactose inducible promoter, a tetracycline inducible promoter, a methionine inducible promoter, or an estradiol inducible promoter. In some embodiments, the galactose inducible promoter comprises GAL1. In some embodiments, the tetracycline inducible promoter comprises tetracycline inducible promoter or doxycycline inducible promoter. In some embodiments, the methionine inducible promoter comprises MET15. In some embodiments, the estradiol inducible promoter comprises GEV.

In some aspects, provided herein is a vector comprising any of the recombinant nucleic acid construct described herein.

In some aspects, provided herein is a composition comprising: (a) a first recombinant nucleic acid sequence comprising a first sequence encoding a first recombinant release factor, wherein the first recombinant release factor is configured to recognize UGA, UAA, UAG, or any combination thereof as a stop codon; and (b) a second recombinant nucleic acid sequence comprising a second sequence encoding a second recombinant release factor, wherein the second recombinant release factor is configured to recognize UGA, UAA, UAG, or any combination thereof as a stop codon, wherein the second nucleic acid sequence comprises an element that allows selective modulation of function or expression of the second recombinant release factor.

In some embodiments, the first recombinant release factor recognizes UGA as the stop codon. In some embodiments, the first recombinant release factor does not recognize UAA and/or UAG as the stop codon. In some embodiments, the first recombinant release factor recognizes UAA, UAG, or any combination thereof as the stop codon. In some embodiments, the first recombinant release factor does not recognize UGA as the stop codon. In some embodiments, the second recombinant release factor recognizes UAA and UAG as stop codons. In some embodiments, the second recombinant release factor recognizes UGA as the stop codon. In some embodiments, the second recombinant release factor recognizes UGA, UAA, and UAG as stop codons.

In some embodiments, the second recombinant nucleic acid sequence comprises the element that allows selective modulation of the function of the second recombinant release factor. In some embodiments, the element that allows selective modulation of the function comprises a temperature sensitive allele that allows the second recombinant release factor to function only at a permissive temperature or a degron cassette that allows degradation of the second recombinant release factor.

In some embodiments, the temperature sensitive allele comprises sup45-ts, sup45-2, sup45-36ts, sup45-1023ts, or sup45-sl23ts. In some embodiments, the sup45-ts comprises a sequence with at least 70% sequence identity to SEQ ID NO: 162. In some embodiments, the sup45-2 comprises a sequence with at least 70% sequence identity to SEQ ID NO: 163. In some embodiments, the sup45-36ts comprises a sequence with at least 70% sequence identity to SEQ ID NO: 164. In some embodiments, the sup45-1023ts comprises a sequence with at least 70% sequence identity to SEQ ID NO: 165. In some embodiments, the sup45-sl23ts comprises a sequence with at least 70% sequence identity to SEQ ID NO: 166 or 167. In some embodiments, the permissive temperature comprises from about 20° C. to about 33° C. In some embodiments, the permissive temperature is 25° C.

In some embodiments, the degron cassette comprises a heat-inducible degron cassette or a small molecule-inducible degron cassette. In some embodiments, the degron cassette comprises the small molecule-inducible degron cassette. In some embodiments, the small molecule comprises an auxin or asunaprevir.

In some embodiments, the second recombinant nucleic acid sequence comprises the element that allows selective modulation of the expression of the second recombinant release factor. In some embodiments, the element that allows selective modulation of the expression of the second recombinant release factor comprises a conditional promoter. In some embodiments, the conditional promoter comprises a galactose inducible promoter, a tetracycline inducible promoter, a methionine inducible promoter, or an estradiol inducible promoter. In some embodiments, the galactose inducible promoter comprises GAL1. In some embodiments, the tetracycline inducible promoter comprises tetracycline inducible promoter or doxycycline inducible promoter. In some embodiments, the methionine inducible promoter comprises MET15. In some embodiments, the estradiol inducible promoter comprises GEV.

In some embodiments, the first or the second recombinant release factor modulates protein translation upon recognizing UGA, UAA, or UAG as the stop codon. In some embodiments, the modulation comprises terminating protein translation. In some embodiments, the first or the second recombinant release factor comprises a class 1 release factor, a class 2 release factor, or a combination thereof. In some embodiments, the class 1 release factor is a eukaryotic release factor 1 (eRF1). In some embodiments, the class 2 release factor comprises a release factor 3. In some embodiments, the class 2 release factor is a eukaryotic release factor 3 (eRF3). In some embodiments, the first or the second recombinant release factor comprises a release factor 1/release factor 3 complex. In some embodiments, the first or the second recombinant release factor is a eukaryotic release factor 1/release factor 3 (eRF1/eRF3) complex.

In some embodiments, the first or the second recombinant release factor comprises a recognition domain comprising one or more mutations that allow the first or the second recombinant release factor to recognize only (i) UGA, (ii) UAA, (iii) UAG, or (iv) any combination thereof.

In some embodiments, the first or the second recombinant release factor comprises a first recognition domain swapped with a second recognition domain. In some embodiments, the second recognition domain is from a release factor of a second organism. In some embodiments, the second recognition domain is identified using a phylogenetic screening, directed evolution, library screening, machine learning, or a combination thereof.

In some embodiments, the first or the second recombinant release factor is from a first organism. In some embodiments, the first organism comprises a eukaryotic cell or a prokaryotic cell. In some embodiments, the prokaryotic cell comprises an archaebacteria cell, a bacterial cell, or a combination thereof. In some embodiments, the eukaryotic cell comprises a yeast cell, a fungal cell, a plant cell, an animal cell, an insect cell, a mammalian cell, or a combination thereof. In some embodiments, the yeast cell comprises Saccharomyces cerevisiae.

In some embodiments, the first or the second recombinant release factor is from a second organism. In some embodiments, the second organism comprises a ciliate. In some embodiments, the ciliate comprises Blepharisma americanum, Blepharisma japonicum, Euplotes aediculatus, Euplotes octocarinatus, Stentor coeruleus, Nyctotherus ovalis, Stylonychia lemnae, Pseudocohnilembus persalinus, Ichthyophthirius multifiliis, Stylonychia lemnae, Oxytricha trifallax, Stylonychia pustulata, Stylonychia mytilus, Eschaneustyla sp. HL-2004, Gonostomum sp. HL-2004, Holosticha sp. HL-2004, Urostyla sp. HL-2004, Uroleptus sp. WIC-2003, Paraurostyla weissei, Stichotrichida sp. Misty, Stichotrichida sp. Alaska, Spironucleus salmonicida, Loxodes striatus, Paramecium tetraurelia, or Tetrahymena thermophila.

In some embodiments, the second recognition domain comprises an amino acid sequence with at least 50% sequence identity to KSSNIKS (SEQ ID NO: 3), YICDNKF (SEQ ID NO: 4), TAVNIKS (SEQ ID NO: 5), KAANIKS (SEQ ID NO: 6), KASNIKS (SEQ ID NO: 7), YYCGERF (SEQ ID NO: 8), TAESIKS (SEQ ID NO: 9), YFCDPQF (SEQ ID NO: 10), EAASIKD (SEQ ID NO: 11), KATNIKD (SEQ ID NO: 12), YFCDSKF (SEQ ID NO: 13), FDFDAES (SEQ ID NO: 14), TLIKPQF (SEQ ID NO: 15), TGDKIKS (SEQ ID NO: 16), TIIKNDF (SEQ ID NO: 17), EAASIQD (SEQ ID NO: 18), FFCDNYF (SEQ ID NO: 19), FVIVNKF (SEQ ID NO: 20), AAQNIKS (SEQ ID NO: 21), YFCGGKF (SEQ ID NO: 22), QANSIKD (SEQ ID NO: 23), YRCDSKF (SEQ ID NO: 24), GAASIKN (SEQ ID NO: 25), YSCNTIF (SEQ ID NO: 26), SAQNIKS (SEQ ID NO: 27), YYCDNRF (SEQ ID NO: 28), SAGNIKS (SEQ ID NO: 29), YFCDNSF (SEQ ID NO: 30), TAQNIKS (SEQ ID NO: 31), SAQSIKS (SEQ ID NO: 32), AANNIKS (SEQ ID NO: 33), YNCSGKF (SEQ ID NO: 34), QAQNIKS (SEQ ID NO: 35), QADCIKS (SEQ ID NO: 36), YSCDGVF (SEQ ID NO: 37), RAQNIKS (SEQ ID NO: 38), FLCENTF (SEQ ID NO: 39), or a combination thereof.

In some embodiments, the second recognition domain comprises an amino acid sequence comprising KSSNIKS (SEQ ID NO: 3), YICDNKF (SEQ ID NO: 4), TAVNIKS (SEQ ID NO: 5), KAANIKS (SEQ ID NO: 6), KASNIKS (SEQ ID NO: 7), YYCGERF (SEQ ID NO: 8), TAESIKS (SEQ ID NO: 9), YFCDPQF (SEQ ID NO: 10), EAASIKD (SEQ ID NO: 11), KATNIKD (SEQ ID NO: 12), YFCDSKF (SEQ ID NO: 13), FDFDAES (SEQ ID NO: 14), TLIKPQF (SEQ ID NO: 15), TGDKIKS (SEQ ID NO: 16), TIIKNDF (SEQ ID NO: 17), EAASIQD (SEQ ID NO: 18), FFCDNYF (SEQ ID NO: 19), FVIVNKF (SEQ ID NO: 20), AAQNIKS (SEQ ID NO: 21), YFCGGKF (SEQ ID NO: 22), QANSIKD (SEQ ID NO: 23), YRCDSKF (SEQ ID NO: 24), GAASIKN (SEQ ID NO: 25), YSCNTIF (SEQ ID NO: 26), SAQNIKS (SEQ ID NO: 27), YYCDNRF (SEQ ID NO: 28), SAGNIKS (SEQ ID NO: 29), YFCDNSF (SEQ ID NO: 30), TAQNIKS (SEQ ID NO: 31), SAQSIKS (SEQ ID NO: 32), AANNIKS (SEQ ID NO: 33), YNCSGKF (SEQ ID NO: 34), QAQNIKS (SEQ ID NO: 35), QADCIKS (SEQ ID NO: 36), YSCDGVF (SEQ ID NO: 37), RAQNIKS (SEQ ID NO: 38), FLCENTF (SEQ ID NO: 39), or a combination thereof.

In some embodiments, the first or the second recombinant release factor comprises an amino acid sequence selected from the group consisting of SEQ ID NOs: 40-64. In some embodiments, the first or the second recombinant release factor from the second organism comprises an eRF1. In some embodiments, the eRF1 from the second organism comprises an amino acid sequence that has at least 20% sequence identity to an eRF1 of the first organism. In some embodiments, the first or the second recombinant release factor comprises an amino acid sequence selected from the group consisting of SEQ ID NOs: 65-74.

In some embodiments, the first or the second recombinant release factor from the second organism comprises an eRF1/eRF3 complex. In some embodiments, the eRF1 of the eRF1/eRF3 complex from the second organism comprises an amino acid sequence that has at least 20% sequence identity to an eRF1 of the first organism. In some embodiments, the eRF3 of the eRF1/eRF3 complex comprises an amino acid sequence selected from the group consisting of SEQ ID NOs: 75, 77, 79, 81, 83, 85, 87, 89, and 91. In some embodiments, the eRF3 of the eRF1/eRF3 complex from the second organism comprises an amino acid sequence that has at least 25% sequence identity to an eRF3 of the first organism. In some embodiments, the eRF3 of the eRF1/eRF3 complex comprises an amino acid sequence selected from the group consisting of SEQ ID NOs: 76, 78, 80, 82, 84, 86, 88, 90, and 92.

In some embodiments, the first or the second recombinant release factor from the second organism comprises an eRF1 and forms a complex with a chimeric eRF3. In some embodiments, the eRF1 of the second organism comprises an amino acid sequence that has at least 40% sequence identity to an eRF1 of the first organism. In some embodiments, the chimeric eRF3 comprises (i) an eRF3 from the first organism or a fragment thereof and (ii) an eRF3 from a second organism or a fragment thereof. In some embodiments, the second organism comprises Euplotes octocarinatus or Paramecium tetraurelia. In some embodiments, the chimeric eRF3 comprises an eRF3 of Euplotes octocarinatus, wherein amino acids 7-298 of the eRF3 of Euplotes octocarinatus is replaced with amino acids 6-253 of the eRF3 from the first organism. In some embodiments, the chimeric eRF3 comprises an amino acid sequence comprising SEQ ID NO: 93 or SEQ ID NO: 94. In some embodiments, the chimeric eRF3 comprises an eRF3 of Euplotes octocarinatus, wherein amino acids 1-298 of the eRF3 of Euplotes octocarinatus is replaced with amino acids 1-253 of the eRF3 from the first organism. In some embodiments, the chimeric eRF3 comprises an amino acid sequence comprising SEQ ID NO: 95 or SEQ ID NO: 96. In some embodiments, the chimeric eRF3 comprises an eRF3 of Paramecium tetraurelia, wherein amino acids 1-321 of the eRF3 of Paramecium tetraurelia is replaced with amino acids 1-253 of the eRF3 from the first organism. In some embodiments, the chimeric eRF3 comprises an amino acid sequence comprising SEQ ID NO: 97, SEQ ID NO: 98, SEQ ID NO: 99, or SEQ ID NO: 100.

In some embodiments, the composition further comprises one or more tRNA molecules that recognize UAG, UAA, or UGA and one or more aminoacyl-tRNA synthetases (aaRSs) for charging the one or more tRNA molecules with a non-canonical amino acid (ncAA). In some embodiments, the ncAA comprises p-azidophenylalanine, 2-aminoisobutyric acid (Aib), an azide-containing ncAA, an alkene-containing ncAA, an alkyne-containing ncAA, or a combination thereof. In some embodiments, the azide-containing ncAA comprises (S)-2-amino-6-((2-azidoethoxy)carbonylamino)hexanoic acid (LysN3). In some embodiments, the alkene-containing ncAA or the alkyne-containing ncAA comprises (2S)-2-amino-6-(((prop-2-yn-1-yloxy)carbonyl)amino)hexanoic acid (LysAlk), N^{ε}-Allyloxycarbonyl-L-lysine, N-ε-propargyloxycarbonyl-L-lysine, L-2-Allylglycine, or O-Allyl-L-Tyrosine. In some embodiments, the one or more tRNA molecules recognize UAG and the one or more aaRSs charges the one or more tRNA molecules with a first ncAA. In some embodiments, the one or more tRNA molecules recognize UAA and the one or more aaRSs charges the one or more tRNA molecules with a second ncAA. In some embodiments, the one or more tRNA molecules recognize UGA and the one or more aaRSs charges the one or more tRNA molecules with a third ncAA. In some embodiments, the first ncAA, the second ncAA, and/or the third ncAA are different from each other.

In some aspects, provided herein is a cell or a population of cells comprising any of the composition described herein, any of the recombinant nucleic acid construct described herein, or any of the vector described herein. In some embodiments, the recombinant nucleic acid construct is inserted in a genomic safe harbor site. In some embodiments, the cell or the population of cells does not comprise a release factor that is expressed from a natural promoter and/or recognizes all of UAG, UAA and UGA as stop codons.

In some aspects, provided herein is an organism comprising any of the cell or the population of cells described herein. In some aspects, provided herein is a cell culture comprising any of the cell or the population of cells described herein. In some aspects, provided herein is a cell lysate comprising any of the composition described herein. In some aspects, provided herein is a cell lysate obtained from any of the cell culture described herein.

In some aspects, provided herein is a system for producing a polypeptide molecule comprising a non-canonical amino acid (ncAA) or a population of polypeptide molecules comprising the ncAA, the system comprising any of the composition described herein, any of the cell or the population of cells described herein, any of the cell culture described herein, or any of the cell lysate described herein. In some embodiments, the system is an in vitro system. In some embodiments, the system is an in vivo system. In some embodiments, the in vivo system comprises a yeast cell, an insect cell, or a mammalian cell system. In some embodiments, the mammalian cell system comprises Chinese Hamster Ovary (CHO) cells or murine myeloma (NS0) cells.

In some aspects, provided herein is a method of producing a polypeptide molecule comprising a non-canonical amino acid (ncAA) or a population of polypeptide molecules comprising the ncAA, the method comprising providing: (a) a first nucleic acid sequence comprising a first sequence encoding a first recombinant release factor, wherein the first recombinant release factor is configured to recognize UGA, UAA, UAG, or any combination thereof as a stop codon; (b) a second nucleic acid sequence comprising a second sequence encoding a second recombinant release factor, wherein the second recombinant release factor is configured to recognize UGA, UAA, UAG, or any combination thereof as a stop codon, and wherein the second nucleic acid sequence comprises an element that allows selective modulation of function or expression of the second recombinant release factor; and (c) an aminoacyl-tRNA synthetase (aaRS)/tRNA pair.

In some embodiments, the second nucleic acid sequence comprises the element that allows selective modulation of the expression of the second recombinant release factor. In some embodiments, the element that allows selective modulation of the expression of the second recombinant release factor comprises a conditional promoter. In some embodiments, the conditional promoter comprises a galactose inducible promoter, a tetracycline inducible promoter, a methionine inducible promoter, or an estradiol inducible promoter. In some embodiments, the galactose inducible promoter comprises GAL1. In some embodiments, the tetracycline inducible promoter comprises tetracycline inducible promoter or doxycycline inducible promoter. In some embodiments, the methionine inducible promoter comprises MET15. In some embodiments, the estradiol inducible promoter comprises GEV.

In some aspects, provided herein is a method of producing a polypeptide molecule comprising a non-canonical amino acid (ncAA) or a population of polypeptide molecules comprising the ncAA, the method comprising providing: (a) a first recombinant release factor, wherein the first recombinant release factor is configured to recognize UGA, UAA, UAG, or any combination thereof as a stop codon; (b) a second recombinant release factor, wherein the second recombinant release factor is configured to recognize UGA, UAA, UAG, or any combination thereof as a stop codon, and wherein the second recombinant release factor comprises an element that allows selective modulation of function of the second recombinant release factor; and (c) an aminoacyl-tRNA synthetase (aaRS)/tRNA pair.

In some embodiments, the first recombinant release factor recognizes UGA as a stop codon. In some embodiments, the first recombinant release factor does not recognize UAA and/or UAG as a stop codon. In some embodiments, the first recombinant release factor recognizes UAA, UAG, or any combination thereof as the stop codon. In some embodiments, the first recombinant release factor does not recognize UGA as the stop codon. In some embodiments, the second recombinant release factor recognizes UAA and UAG as stop codons. In some embodiments, the second recombinant release factor recognizes UGA as the stop codon. In some embodiments, the second recombinant release factor recognizes UGA, UAA, and UAG as stop codons. In some embodiments, the aaRS/tRNA pair is configured to recognize the UAG, UAA, or UGA and incorporate the ncAA into an amino acid sequence of the polypeptide or the population of the polypeptide molecules.

In some embodiments, the ncAA comprises p-azidophenylalanine, 2-aminoisobutyric acid (Aib), an azide-containing ncAA, an alkene-containing ncAA, an alkyne-containing ncAA, or a combination thereof. In some embodiments, the azide-containing ncAA comprises (S)-2-amino-6-((2-azidoethoxy)carbonylamino)hexanoic acid (LysN3). In some embodiments, the alkene-containing ncAA or the alkyne-containing ncAA comprises (2S)-2-amino-6-(((prop-2-yn-1-yloxy)carbonyl)amino)hexanoic acid (LysAlk), N^{ε}-Allyloxycarbonyl-L-lysine, N-ε-propargyloxycarbonyl-L-lysine, L-2-Allylglycine, or O-Allyl-L-Tyrosine. In some embodiments, the aaRS/tRNA pair is configured to recognize the UAG and incorporate a first ncAA into an amino acid sequence of the polypeptide or the population of the polypeptide molecules. In some embodiments, the aaRS/tRNA pair is configured to recognize the UAA and incorporate a second ncAA into an amino acid sequence of the polypeptide or the population of the polypeptide molecules. In some embodiments, the aaRS/tRNA pair is configured to recognize the UGA and incorporate a third ncAA into an amino acid sequence of the polypeptide or the population of the polypeptide molecules. In some embodiments, the first ncAA, the second ncAA, and/or the third ncAA are different from each other. In some embodiments, the producing occurs in vivo. In some embodiments, the producing occurs in vitro.

In some embodiments, the second recombinant release factor is conditionally expressed from a nucleic acid sequence comprising a conditional promoter. In some embodiments, the conditional promoter comprises a galactose inducible promoter, a tetracycline inducible promoter, a methionine inducible promoter, or an estradiol inducible promoter. In some embodiments, the galactose inducible promoter comprises GAL1. In some embodiments, the tetracycline inducible promoter comprises tetracycline inducible promoter or doxycycline inducible promoter. In some embodiments, the methionine inducible promoter comprises MET15. In some embodiments, the estradiol inducible promoter comprises GEV.

In some embodiments, the element that allows selective modulation of the function comprises a temperature sensitive allele that allows the second recombinant release factor to function only at a permissive temperature or a degron cassette that allows degradation of the second recombinant release factor. In some embodiments, the temperature sensitive allele comprises sup45-ts, sup45-2, sup45-36ts, sup45-1023ts, or sup45-sl23ts. In some embodiments, the sup45-ts comprises a sequence with at least 70% sequence identity to SEQ ID NO: 162. In some embodiments, the sup45-2 comprises a sequence with at least 70% sequence identity to SEQ ID NO: 163. In some embodiments, the sup45-36ts comprises a sequence with at least 70% sequence identity to SEQ ID NO: 164. In some embodiments, the sup45-1023ts comprises a sequence with at least 70% sequence identity to SEQ ID NO: 165. In some embodiments, the sup45-sl23ts comprises a sequence with at least 70% sequence identity to SEQ ID NO: 166 or 167. In some embodiments, the permissive temperature comprises from about 25° C. to about 33° C. In some embodiments, the permissive temperature is 25° C.

In some embodiments, the degron cassette comprises a heat-inducible degron cassette or a small molecule-inducible degron cassette. In some embodiments, the degron cassette comprises the small molecule-inducible degron cassette. In some embodiments, the small molecule comprises an auxin.

In some aspects, provided herein is a method of screening a release factor having codon-specific release factor activity, the method comprising: a. providing a cell or a population of cells comprising a first release factor recognizing one or two stop codons; b. introducing the cell or the population of cells a second release factor; c. performing a first assay to detect codon-specific activity of the second release factor; and d. performing a second assay to confirm the second release factor does not recognize the one or two stop codons recognized by the first release factor.

In some embodiments, the first release factor recognizes UGA as a stop codon. In some embodiments, the first release factor does not recognize UAA and/or UAG as the stop codon. In some embodiments, the first release factor recognizes UAA and/or UAG as a stop codon. In some embodiments, the first release factor does not recognize UGA as the stop codon. In some embodiments, the first assay or the second assay is performed at a temperature from about 30° C. to about 37° C.

In some aspects, provided herein is a system for screening a release factor for codon-specific release factor activity, the system comprising: a. a cell or a population of cells comprising a first release factor that recognizes one or two stop codons; b. a first assay configured to detect a codon-specific release factor activity of a second release factor via introducing the second release factor to the cell or the population of cells, wherein the second release factor recognizes or is configured to recognize at least one stop codon; and c. a second assay configured to confirm the codon-specific release factor activity of the second release factor is specific for one or two stop codons not recognized by the first release factor; and a computer configured to process a first data set from the first assay and a second data set from the second assay.

In some embodiments, the system is an in vitro system. In some embodiments, the system is an in vivo system. In some embodiments, the in vivo system comprises a yeast cell, an insect cell, or a mammalian cell system. In some embodiments, the mammalian cell system comprises Chinese Hamster Ovary (CHO) cells or murine myeloma (NS0) cells.

In some embodiments, the first release factor recognizes UGA as a stop codon. In some embodiments, the first release factor does not recognize UAA and/or UAG as the stop codon. In some embodiments, the first release factor recognizes UAA and/or UAG as a stop codon. In some embodiments, the first release factor does not recognize UGA as the stop codon. In some embodiments, the first assay or the second assay is performed at a temperature from about 30° C. to about 37° C.

In some aspects, provided herein is a use of any of the composition described herein, any of the recombinant nucleic acid construct described herein, any of the vector described herein, any of the cell or a population of cells described herein, any of the organism described herein, any of the cell culture described herein, any of the cell lysate described herein, or any of the system described herein for producing a polypeptide molecule comprising a non-canonical amino acid (ncAA) or a population of polypeptide molecules comprising the ncAA.

In some embodiments, the use further comprises providing an aminoacyl-tRNA synthetase (aaRS)/tRNA pair. In some embodiments, the aaRS/tRNA pair is configured to recognize the UAG, UAA, or UGA and incorporate the ncAA into an amino acid sequence of the polypeptide or the population of the polypeptide molecules. In some embodiments, the ncAA comprises p-azidophenylalanine, 2-aminoisobutyric acid (Aib), an azide-containing ncAA, an alkene-containing ncAA, an alkyne-containing ncAA, or a combination thereof. In some embodiments, the azide-containing ncAA comprises (S)-2-amino-6-((2-azidoethoxy)carbonylamino)hexanoic acid (LysN3). In some embodiments, the alkene-containing ncAA or the alkyne-containing ncAA comprises (2S)-2-amino-6-(((prop-2-yn-1-yloxy)carbonyl)amino)hexanoic acid (LysAlk), Nε-Allyloxycarbonyl-L-lysine, N-ε-propargyloxycarbonyl-L-lysine, L-2-Allylglycine, or O-Allyl-L-Tyrosine.

In some embodiments, the aaRS/tRNA pair is configured to recognize the UAG and incorporate a first ncAA into an amino acid sequence of the polypeptide or the population of the polypeptide molecules. In some embodiments, the aaRS/tRNA pair is configured to recognize the UAA and incorporate a second ncAA into an amino acid sequence of the polypeptide or the population of the polypeptide molecules. In some embodiments, the aaRS/tRNA pair is configured to recognize the UGA and incorporate a third ncAA into an amino acid sequence of the polypeptide or the population of the polypeptide molecules. In some embodiments, the first ncAA, the second ncAA, and/or the third ncAA are different from each other. In some embodiments, the producing occurs in vivo. In some embodiments, the producing occurs in vitro.

### EXAMPLES

These examples are provided for illustrative purposes only and not to limit the scope of the claims provided herein.

### Example 1: Release Factor (RF) Engineering—Mutagenesis

A release factor (RF) that recognizes all three stop codons (e.g., UAA, UAG, and UGA) can be mutated to recognize only one or two stop codons. Such mutation(s) can be made in a recognition domain of an RF.

First, a three-dimensional structure of one or more RFs of interest or a domain of one or more RFs of interest can be obtained. A domain with semi-conserved and invariant amino acid residues located near known amino acid residues important for functional role (e.g., NIKS or YCF mini domain) can be identified. One or more semi-conserved and invariant amino acids in the aforementioned domain can be selected for mutagenesis.

The mutagenesis of selected amino acids can be performed according to any known methods in the art, including PCR-based megaprimer methods or site-directed mutagenesis. The PCR primers can be designed to contain relevant amino acid substitutions and restriction enzyme digestion sites for cloning. DNA amplifications can be carried out according to any methods in the art. The amplified DNA fragments can be digested by restriction enzymes selected for cloning and ligated into the same restriction sites of the host system (e.g., a plasmid containing a host RF gene). The ligated mixture can be transformed into Escherichia coli. The cloned DNAs can be sequenced to confirm that the cloned DNAs have the desired mutations.

The RF can be expressed and purified in vitro and the RF activity can be measured in vitro.

### Example 2: Release Factor (RF) Engineering—Domain/Motif Swapping I

A recognition domain of a release factor (RF) from an organism (e.g., a ciliate) can be swapped into an RF of a host (e.g., a eukaryotic platform, such as a yeast).

First, a three-dimensional structure of one or more RFs of interest can be obtained. Hinge regions (e.g., hinge 1 and hinge 2) and recognition domains (e.g., domain 1, domain 2, and domain 3) can be identified. Conserved amino acid sequences at the junctions of domain 1 and domain 2 (e.g., hinge 1), and at the junctions of domain 2 and domain 3 (e.g., hinge 2) of the RFs can be identified. Each domain can be swapped at the hinge.

Restriction enzyme sites at the conserved amino acid sequences at the junctions can be analyzed to identify a restriction enzyme site for domain swapping. PCR primers for amplifying one or more recognition domains can be designed to include the restriction enzyme site of choice. DNA amplifications can be carried out according to any methods in the art. The amplified recognition domain fragments can be digested with restriction enzymes and ligated into the same restriction sites of the host system (e.g., a plasmid comprising a host RF gene) to give rise to a hybrid RF gene.

The RF can be expressed and purified in vitro and the RF activity can be measured in vitro.

### Example 3: Release Factor (RF) Engineering—Domain Swapping II

Recognition domains in yeast eRF1 (encoded by SUP45 gene) were engineered to introduce the corresponding recognition domains of ciliate eRF1s. The resulting domain-swapped yeast eRF1 was tested in yeast for the ability to confer the stop codon selectivity of ciliate eRF1s. An episomal-based shuffle system was employed (FIG. 2). A yeast strain which lacks SUP45 gene (sup4.54) was generated. As the SUP45 gene is essential, the strain was introduced with the wild-type (WT) SUP45 gene on a counter-selectable plasmid. In this case, this counter-selectable marker is URA3, which can be selected against in media containing 5-FOA. Next, a set of “domain-swapped” sup45 constructs (see Table 3), which were under the control of the SUP45 promoter (SUP45pr), were generated with LEU2 or HIS3 markers. In an example of such a system, the candidate UAA/UAG-specific domain-swapped yeast eRF1 was cloned on a vector marked with LEU2, while the candidate UGA-specific eRF1 was cloned on a vector marked with HIS3. Once vectors were transformed into the yeast sup4561 mutant, strains were maintained on media that selected for all three vectors (e.g., Synthetic complete medium which lacked uracil, leucine, and histidine, aka SC-URA-LEU Viability of the sup4561 strain without the WT URA3-marked SUP45 was assessed post-shuffle on media containing 5-FOA.

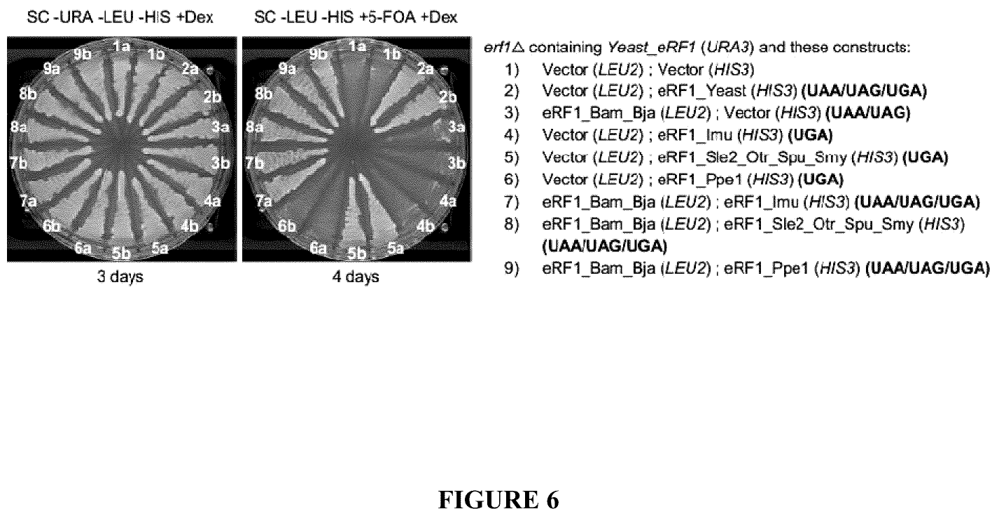

FIG. 6 illustrates an example of testing for stop-codon selectivity and functionality of a domain/motif-swapped yeast eRF1. A yeast erf1Δ strain pre-transformed with the endogenously regulated yeast eRF1 (URA3-marked plasmid), was subsequently transformed with the endogenously regulated, domain-swapped UAA/UAG-specific construct “eRF1_Bam_Bja” (LEU2-marked plasmid) or an empty LEU2 vector, and the endogenously regulated candidate UGA-specific motif-swapped ciliate eRF1 constructs (HIS3-marked plasmid) or an empty HIS3 vector. Yeast strains, post transformation, were maintained on dextrose media that selects for all three plasmid constructs (SC-URA-LEU-HIS+Dex). The same strains were also streaked on dextrose medium supplemented with 5-FOA, selecting for only the motif-swapped ciliate constructs (SC-LEU-HIS+5-FOA+Dex). Three different candidate UGA-specific constructs (different only in their yeast TASNIKS (SEQ ID NO: 1)/YLCDNKF (SEQ ID NO: 2) motifs) were tested for their ability to complement erf1 deletion on 5-FOA media. The eRF1_Sle2_Otr_Spu_Smy construct (isolates #5a, #5b) supported viability of an erf1Δ strain in the absence of eRF1_Bam_Bja, suggesting that this construct was not specific to UGA in vivo. The other two UGA-specific constructs, eRF1_Imu and eRF1_Ppe1, suppressed the lethality of an erf1. A mutant on 5-FOA media, only when combined with eRF1_Bam_Bja. Two independent transformants were tested for each strain (labelled a and b). Isolate #2 provided a positive control sample where the native yeast eRF1 gene was expressed from the HIS3-marked plasmid alongside a LEU2 empty vector (FIG. 6).

### Example 4: Release Factor (RF) Engineering—Whole-Gene Swap

The native whole-gene release factor (RF) from an organism (e.g., a ciliate) can replace the RF of a host (e.g., a eukaryotic platform, such as a yeast).

The wild-type yeast eRF1 can be replaced by the entire ciliate eRF1 protein. In this case, replaceability is tested in a sup454 mutant. In some cases, the corresponding ciliate eRF3 may be required for ciliate eRF1 function in yeast. In this case, replaceability can be tested in a sup454 or sup454 sup354 mutant.

An episomal-based shuffle system was employed (FIG. 2). The yeast genes, SUP45 and SUP35, (separate or together) were cloned on a vector that carries a counter-selectable marker (such as URA3), and their expression was driven using either the native endogenous promoters or an inducible promoter system (such as the bi-directional GAL1/10 system). Codon-optimized ciliate UAA/UAG- and UGA-specific RFs (eRF1 or eRF1/eRF3) were cloned on two separate vectors that carry different auxotrophic markers (such as LEU2 and HIS3), and their expression was driven using either the corresponding yeast endogenous promoters or an inducible promoter system (such as the bi-directional GAL1/10 system). In an example of such a system, the UAA/UAG-specific ciliate RFs were cloned on a vector marked with LEU2, while the UGA-specific ciliate RFs were cloned on a vector marked with HIS3. In the cases where ciliate eRF3 was not included, endogenous yeast eRF3 (SUP35) must be included in the host strain, and the yeast eRF3 protein may function with the ciliate eRF1. In cases where ciliate eRF3 was included, the experiments could be done with or without yeast eRF3. The episomal shuffle strains were derived by transformation of vectors (such as those marked by LEU2 or HIS3) containing ciliate RFs into the yeast haploid deletion mutants that already contain the counter-selectable vector. Examples of these episomal shuffle strains included, but were not limited to, the sup45Δ or sup45Δ sup35Δ haploids containing 3 vectors: the counter-selectable URA3-marked vector that contained the corresponding wildtype yeast RFs, the LEU2-marked vector contained the UAA/UAG-specific ciliate RFs, and the HIS3-marked vector contained the UGA-specific ciliate RFs. Once vectors were transformed, strains were maintained on media that selected for all three vectors (e.g., Synthetic complete medium which lacked uracil, leucine, and histidine, or also known as SC-URA-LEU-HIS).

The episomal shuffle strategy tested viability of strains on media supplemented with 5-FOA. In the case where expression of the vector-based ciliate gene(s) was driven by the corresponding yeast endogenous promoter(s), the 5-FOA medium contained any sugar source (preferably dextrose). In the case where expression of the vector-based ciliate gene(s) was driven by the inducible GAL/10 promoter, the 5-FOA medium contained galactose as the sugar source and constructs were induced on galactose media before plating on 5-FOA.

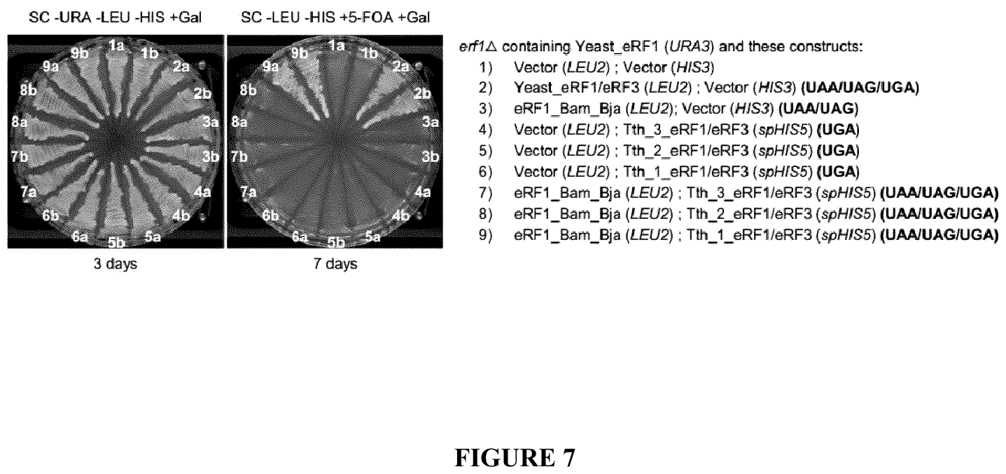

FIG. 7 illustrates an example of testing for stop-codon selectivity and functionality of whole-gene ciliate eRF1/eRF3 in yeast. A yeast erf1Δ strain pre-transformed with the endogenously regulated yeast eRF1 (URA3-marked plasmid), was subsequently transformed with the endogenously regulated, motif-swapped UAA/UAG-specific construct “eRF1_Bam_Bja” (LEU2-marked plasmid, or an empty vector) and/or the galactose-inducible candidate UGA-specific whole-gene ciliate eRF1/eRF3 constructs (spHIS5- or HIS3-marked plasmid, or an empty vector). Yeast strains, post transformation, are maintained on dextrose media that selects for all three plasmid constructs (SC-URA-LEU-HIS+Dex; not pictured). Galactose-regulated ciliate ORFs were induced on the same selective media containing galactose for 3 days (SC-URA-LEU-HIS+Gal), before re-streaking on galactose media containing 5-FOA, while selecting for only the whole-gene ciliate constructs (SC-LEU-HIS+5-FOA+Gal). Three different galactose-inducible Tth_eRF1/eRF3 constructs (different only in their eRF1 ORFs) were tested for their ability to complement deletion of erf1Δ deletion on 5-FOA media. Only Tth_1_eRF1/eRF3 (Tth_eRF1_XP_001018735.1/Tth_eRF3_XP_001011280.3), in combination with the UAA/UAG-specific construct, suppressed the lethality of an erf1Δ mutant on 5-FOA media. The results suggested that the whole-gene ciliate Tth_1_eRF1 construct was functional and UGA-specific, while the other two Tth_eRF1 constructs were non-functional in yeast. Two independent transformants were tested for each strain (labeled a and b). Isolate #2 provided a positive control sample where the native yeast eRF1/eRF3 gene was expressed from the LEU2-marked plasmid (FIG. 7).

The 5-FOA media selects for two of the vector constructs (ex. LEU2-marked UAA/UAG-specific construct and HIS3-marked UGA-specific constructs) (FIGS. 6 and 7). Given that both eRF1 and eRF3 of yeast are essential genes, upon counter-selection on 5-FOA in the episomal shuffle system, if an expression of a single ciliate-derived engineered RF results in viability, this indicates that this RF recognizes all three stop codons in vivo in yeast (FIGS. 6, 5a and 5b). In this case, stop codon selectivity is not achieved (Table 3, “wild-type” result).

### Example 5: Plasmid-Dependency of erf1Δ Strains

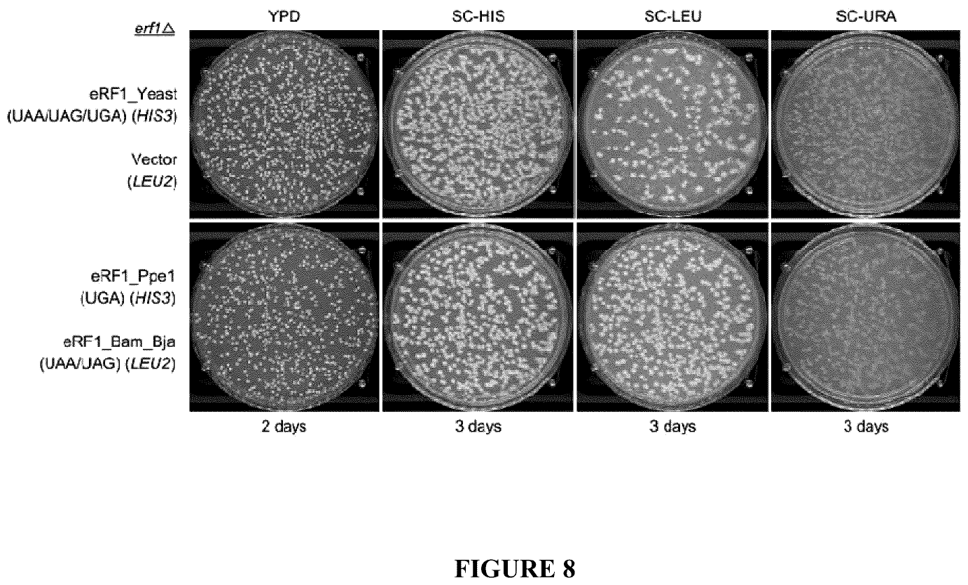

To test whether strains that are viable on 5-FOA are dependent on both the UAA/UAG- and UGA-specific constructs, colonies were isolated from the selective media (SC-LEU-HIS+5-FOA) and grown in non-selective YPD media. Only strains that required both plasmid constructs to decode all three stop codons formed viable LEU⁺ and HIS⁺ colonies after growth in YPD. As a control, these strains should not grow on −URA plates, given that they were isolated from media containing 5-FOA (FIG. 8).

FIG. 8 illustrates an example for assessing the plasmid-dependency of erf1Δ strains carrying ciliate release factor constructs. Yeast erf1Δ strains containing different combinations of plasmid constructs were isolated from SC-LEU-HIS+5-FOA plates. Strains were grown to saturation in non-selective liquid YPD medium at 30° C. for 1 day, and then re-inoculated in the same medium and grown to saturation for a second day. Cells were plated for single colonies on YPD and incubated for 2 days at 30° C., and then replica-plated to SC-HIS, SC-LEU, or SC-URA agar plates (all dextrose). Viability was assessed after 3 days. In the first example, the HIS3-marked plasmid encoding the endogenously regulated (SUP45pro) yeast eRF1 gene construct (UAA/UAG/UGA) was required for viability of an erf1Δ mutant. The LEU2-marked empty vector control was not required for viability and thus could be lost, resulting in colonies unable to grow on medium lacking leucine (SC-LEU). No growth was observed on SC-URA plates given that the strains were isolated from media supplemented with 5-FOA. In the second example, both the HIS3- and LEU2-marked plasmids encoding the endogenously regulated (SUP45pro) eRF1_Ppe1 (UGA) and the eRF1_Bam_Bja (UAA/UAG) gene constructs, respectively, were required for viability of an erf1Δ mutant. No growth was observed on SC-URA plates given that the strains were isolated from media supplemented with 5-FOA (FIG. 8).

### Example 6: Phylogenetic Screening for eRF1 Domain/Motif Swapping

This example described below was performed for eRF1 domain/motif swapping experiments, specifically the TASNIKS (SEQ ID NO: I) and YCF domains.

To identify additional ciliate eRF1s for domain/motif swapping and functional testing in yeast, we extracted all proteins annotated in Gene Ontology as codon-specific release factors plus all proteins annotated as eRF1 by Uniprot's annotation system. We then narrowed down the list to organisms that use a subset of the 3 stop codons. And then we looked for the overlap with NCBI translation tables 4, 6, and 10. NCBI translation tables 4, 6, and 10 can be found: https://www.ncbi.nlm.nih.gov/Taxonomy/Utils/wprintgc.cgi#SG4.

NCBI Translation Table 4. The Mold, Protozoan, and Coelenterate Mitochondrial Code and the Mycoplasma/Spiroplasma Code (transl_table=4)

NCBI Translation Table 6. The ciliate, Dasycladacean and hexamita Nuclear Code (transl_table=6)

NCBI Translation Table 10. The Euplotid Nuclear Code (trans' table=10) This analysis uncovered:
1 example of NCBI translation table 4: Blepharisma; Mold Mitochondrial; Protozoan Mitochondrial; Coelenterate Mitochondrial; Mycoplasma; Spiroplasma
24 examples of NCBI translation table 6: ciliate Nuclear; Dasycladacean Nuclear; hexamita Nuclear
9 examples of NCBI translation table 10: Euplotid Nuclear

Within the 34 uncovered examples, there were 24 unique TASNIKS (SEQ ID NO: 1)/YCF motifs, which were tested using the episome-shuffle system (Table 3).

### Example 7: Stop Codon Capture

A Saccharomyces cerevisiae strain with the following genotype is built:
1. Inducibly expressed dual fluorescent reporter construct
2. p-azidophenylalanine (pAzF) orthogonal translation system (tRNA and synthetase)
3. deleted for yeast eRF1
4. a downregulatable yeast eRF1 UAA/UAG specific-construct
5. a constitutively expressed yeast eRF1 UGA specific-construct

Readthrough signals of the dual fluorescent reporter under all combination of the following conditions are evaluated:
1. Presence of the ncAA pAzF
2. Absence of the ncAA pAzF
3. Presence of the downregulatable yeast eRF1 UAA/UAG specific-construct
4. Absence of the downregulatable yeast eRF1 UAA/UAG specific-construct

Expected result: Increased readthrough signal in the presence of pAzF and in the absence of downregulatable yeast eRF1 UAA/UAG specific-construct as a function of eliminating competition between the pAzF orthogonal translation system and the release factor.

### Example 8: UAA/UAG-Specific Constructs

### Domain/Motif-Swap

Table 3 highlights all the UAA/UAG-specific domain-swapped yeast eRF1 constructs tested in yeast. A yeast erf1Δ strain pre-transformed with the endogenously regulated yeast eRF1 (UR43-marked plasmid), was subsequently transformed with the endogenously-regulated (SUP45pro) motif-swap UAA/UAG-specific construct (eRF1_Bam__Bja)(LEU2) and the indicated HIS3-marked candidate UGA-specific constructs, or with the endogenously-regulated (SUP45 pro) motif-swap UGA-specific construct (eRF1_Pte1_(m1)) (HIS3) and the indicated LEU2-marked candidate UAA/UAG-specific constructs. Yeast strains were maintained on SC-URA-LEU-HIS+Dex media, before testing for replaceability on SC-LEU-HIS4-5-FOA+Dex media (Table 3).

The eRF1 protein has two “motifs” or highly conserved amino acid sequences important for specifying what stop codons are recognized. In yeast, the omnipotent eRF1 recognizes all three stop codons, and the motifs in question are TASNIKS (SEQ ID NO: 1) and YLCDNKF (SEQ ID NO: 2). Prior work has suggested that specific changes to these motifs underlie the exclusive recognition of either UGA or UAA/UAG found in ciliates. In these examples, the impact of introducing these motifs into the yeast protein is tested in the yeast cell. Two parameters are measured: the stop codon specificity of the construct in the context of the yeast cell, and the ability of the construct to function in yeast.

The eRF1_Bam_Bja construct was UAA/UAG-specific and could function in yeast. The eRF1_Bam_Bja construct was derived by swapping the TASNIKS (SEQ ID NO: 1)/YLCDNKF (SEQ ID NO: 2) motifs in yeast eRF1 to KSSNIKS (SEQ ID NO: 3)/YICDNKF (SEQ ID NO: 4) (as found in the eRF1 protein sequences of both organisms Blepharisma americanum and Blepharisma japonicum). The episomal-based shuffle system, which utilized 5-FOA to counter-select against the URA3-marked omnipotent (e.g., recognizing UGA. UAA and UAG) wild-type yeast eRF1, was employed to test the stop codon specificity and functionality of the construct when expressed in yeast. To provide UGA-specificity, another construct (eRF1_Pte1_(m1)) was derived by swapping the YLCDNKF (SEQ ID NO: 2) motif in yeast eRF1 to YFCDPQF (SEQ ID NO: 10) (as found in the eRF1 protein sequence of the organism Paramecium tetraurelia). When individually expressed, the eRF1_Bam__Bja and eRF1_Pte1_(m1) eRF1 constructs did not support viability of a mutant on 5-FOA media, suggesting that these strains post-shuffle cannot decode UGA or UAA/UAG, respectively. When expressed in combination, the eRF1_Bam_Bja and eRF1_Pte1_(m1) constructs together supported viability of a sup45Δ mutant on 5-FOA media, consistent with the predicted exclusive stop codon specificity of the two eRF1 constructs and simultaneously demonstrating that each was functional in yeast (Table 3).

The eRF1_Eae1_Eoc1 construct was UAA/UAG-specific and could function in yeast. The eRF1_Eae1_Eoc1 construct was derived by swapping the TASNIKS (SEQ ID NO: 1)/YLCDNKF (SEQ ID NO: 2) motifs in yeast eRF1 to TAVNIKS (SEQ ID NO: 5)/YICDNKF (SEQ NO: 4) (as found in the eRF1 protein sequences of the organisms Euplotes aediculatus and Euplotes octocarinatus). The episomal-based shuffle system, which utilized 5-FOA to counter-select against the URA43-marked omnipotent wild-type yeast eRF1, was employed to test the stop codon specificity and functionality of the construct when expressed in yeast. To provide UGA-specificity, another construct (eRF1_Pte1_(m1)) was derived by swapping the YLCDNKF (SEQ ID NO: 2) motif in yeast eRF1 to YFCDPQF (SEQ ID NO: 10) (as found in the eRF1 protein sequence of the organism Paramecium tetraurelia). When expressed individually, the eRF1_Eae1_Eoc1 and eRF1_Pte1_(m1) eRF1 constructs did not support viability of a sup45Δ mutant on 5-FOA media, suggesting that these strains post-shuffle cannot decode UGA or UAA/UAG respectively. When expressed in combination, the eRF1_Eae1_Eoc1 and eRF1_Pte1_(m1) constructs together supported viability of a sup45Δ mutant on 5-FOA media, consistent with the predicted stop codon specificity of the two eRF1 constructs and simultaneously demonstrating that each was functional in yeast (Table 3).

TABLE 3
Summary of motif-swapped construct replacements.

| | | Motif (underlined amino | | |
|---|---|---|---|---|
| | | acids: mutations introduced | | |
| Stop Codon | Construct Name | for each construct) | | notes |
| UAA/UAG/UGA | eRF1_Yeast | TASNIKS | YLCDNKF | Status *** |
| | | (SEQ ID NO: 1) | (SEQ ID NO: 2) | |
| UAA/UAG | eRF1_Bam_Bja * | KSSNIKS | YICDNKF | Replaceable |
| | | (SEQ ID NO: 3) | (SEQ ID NO: 4) | |
| UAA/UAG | eRF1_Eae1_Eoc1 * | TAVNIKS | YICDNKF | Replaceable |
| | | (SEQ ID NO: 5) | (SEQ ID NO: 4) | |
| UAA/UAG | eRF1_Sco * | KAANIKS | YLCDNKF | WT |
| | | (SEQ ID NO: 6) | (SEQ ID NO: 2) | |
| UAA/UAG | eRF1_Nov | KASNIKS | YYCGERF | WT |
| | | (SEQ ID NO: 7) | (SEQ ID NO: 8) | |
| UAA/UAG | eRF1-Eae2_Eoc2 | TAESIKS | YICDNKF | Non- |
| | | (SEQ ID NO: 9) | (SEQ ID NO: 4) | replaceable |
| UGA | eRF1_Pte1_(m1) * | TASNIKS | YFCDPQF | Replaceable |
| | | (SEQ ID NO: 1) | (SEQ ID NO: | |
| | | | 10) | |
| UGA | eRF1_Pte1_(m2) ** | EAASIKD | YFCDPQF | Replaceable |
| | | (SEQ ID NO: | (SEQ ID NO: | |
| | | 11) | 10) | |
| UGA | eRF1_Tth1 | KATNIKD | YFCDSKF | WT |
| | | (SEQ ID NO: | (SEQ ID NO: | |
| | | 12) | 13) | |
| UGA | eRF1_Sle1 | FDFDAES | TLIKPQF | Non- |
| | | (SEQ ID NO: | (SEQ ID NO: | replaceable |
| | | 14) | 15) | |
| UAA/UAG/UGA | eRF1_Yeast | TASNIKS | YLCDNKF | Status *** |
| | | (SEQ ID NO: 1) | (SEQ ID NO: 2) | |
| UGA | eRF1_Ppe2 ** | TGDKIKS | TIIKNDF | Non- |
| | | (SEQ ID NO: | (SEQ ID NO: | replaceable |
| | | 16) | 17) | |
| UGA | eRF1_Pte2 | EAASIQD | FFCDNYF | Non- |
| | | (SEQ ID NO: | (SEQ ID NO: | replaceable |
| | | 18) | 19) | |
| UGA | eRF1_Imu | KATNIKD | FVIVNKF | Replaceable |
| | | (SEQ ID NO: | (SEQ ID NO: | |
| | | 12) | 20) | |
| UGA | eRF1_Sle2_Otr_ | AAQNIKS | YFCGGKF | WT |
| | Spu_Smy ** | (SEQ ID NO: | (SEQ ID NO: | |
| | | 21) | 22) | |
| UGA | eRF1_Ppe1 * | QANSIKD | YRCDSKF | Replaceable |
| | | (SEQ ID NO: | (SEQ ID NO: | |
| | | 23) | 24) | |
| UGA | eRF1_Tth2 ** | GAASIKN | YSCNTIF | Replaceable |
| | | (SEQ ID NO: | (SEQ ID NO: | |
| | | 25) | 26) | |
| UGA | eRF1_Ehl ** | SAQNIKS | YYCDNRF | WT |
| | | (SEQ ID NO: | (SEQ ID NO: | |
| | | 27) | 28) | |
| UGA | eRF1_Ghl ** | SAGNIKS (SEQ | YECDNSF | WT |
| | | ID NO: 29) | (SEQ ID NO: | |
| | | | 30) | |
| UGA | eRF1_Hhl | TAQNIKS | YFCGGKF | WT |
| | | (SEQ ID NO: | (SEQ ID NO: | |
| | | 31) | 22) | |
| UGA | eRF1_Uhl ** | SAQSIKS | YFCDNSF | Replaceable |
| | | (SEQ ID NO: | (SEQ ID NO: | |
| | | 32) | 30) | |
| UGA | eRF1_Uwj_Pwe ** | AANNIKS | YFCGGKF | WT |
| | | (SEQ ID NO: | (SEQ ID NO: | |
| | | 33 | 22) | |
| UGA | eRF1_Smi ** | TASNIKS | YNCSGKF | WT |
| | | (SEQ ID NO: 1) | (SEQ ID NO: | |
| | | | 34) | |
| UGA | eRF1_Sal ** | QAQNIKS | YFCGGKF | WT |
| | | (SEQ ID NO: | (SEQ ID NO: | |
| | | 35) | 22) | |
| UGA | eRF1_Ssa ** | QADCIKS | YSCDGVF | Replaceable |
| | | (SEQ ID NO: | (SEQ ID NO: | |
| | | 36) | 37) | |
| UGA | eRF1_Lst ** | RAQNIKS | FLCENTF | Replaceable |
| UAA/UAG/UGA | eRF1_Yeast | TASNIKS | YLCDNKF | Status *** |
| | | (SEQ ID NO: 1) | (SEQ ID NO: 2) | |
| | | (SEQ ID NO: | (SEQ ID NO: | |
| | | 38 | 39) | |

* Candidate UAA/UAG-specific constructs tested against the UGA-specific eRF1_Ptel_(ml); all constructs regulated by a SUP 45pro
** Candidate UGA-specific constructs tested against the UAA/UAG-specific eRF1_Bam Bja; all constructs regulated by a SUP 45pro
*** Status of construct when tested in an erf1Δ mutant:
Replaceable: Functional in yeast and confers stop codon selectivity, supports growth on 5-FOA only when expressed with the opposite construct
Non-replaceable: Not functional in yeast, unknown status on stop codon selectivity, does not support growth on 5-FOA when expressed with the opposite construct.
WT: Functional in yeast but does not confer stop codon selectivity, supports growth on 5-FOA when expressed either individually or with the opposite construct.

### Whole Gene Swaps

Table 4 highlights the UAA/UAG whole-gene ciliate eRF1 constructs tested in yeast. Ciliate eRF1 constructs, under the transcriptional control of the yeast eRF1 endogenous promoter (SUP45pro), were tested against the motif-swap constructs. A yeast erf1Δ strain pre-transformed with the endogenously regulated yeast eRF1 (URA3-marked plasmid), was subsequently transformed with the endogenously regulated (SUP45pro) motif-swap UAA/UAG-specific construct (eRF1_Bam_Bja) (LEU2) and the indicated HIS3-marked UGA-specific whole-gene constructs, or with the endogenously regulated (SUP45pro) motif-swap UGA-specific construct (eRF1_Pte1_(m1)) (HIS3) and the indicated LEU2-marked UAA/UAG-specific whole-gene constructs. Yeast strains were maintained on SC-URA-LEU-HIS+Dex media, before testing for replaceability on SC-LEU-HIS+5-FOA+Dex media.

The Eoc_eRF1_CAC14170.1 construct coded for a UAA/UAG-specific eRF1 protein that could function in yeast. The whole gene eRF1 construct was derived from the organism Euplotes octocarinatus. The episomal-based shuffle system, which utilized 5-FOA to counter-select against the URA3-marked omnipotent wild-type yeast eRF1, was employed to test the stop codon specificity and functionality of the ciliate construct when expressed in yeast. To provide UGA-specificity, another construct (eRF1_Pte1_(m1)) was derived by swapping the YLCDNKF (SEQ ID NO: 2) motif in yeast eRF1 to YFCDPQF (SEQ ID NO: 10) (as found in the eRF1 protein sequence of the organism Paramecium tetraurelia). When expressed individually, the Eoc_eRF1_CAC14170.1 and eRF1_Pte1_(m1) eRF1 constructs did not support viability of a sup45Δ mutant on 5-FOA media, suggesting that these strains post-shuffle cannot decode either UGA or UAA/UAG, respectively. When expressed in combination, the Eoc_eRF1_CAC14170.1 and eRF1_Pte1_(m1) constructs supported viability of a sup45Δ mutant on 5-FOA media, consistent with the stop codon specificity of the two eRF1 constructs and simultaneously demonstrating that both could function in yeast (Table 4).

The Eoc_eRF1_AAG25924.1 construct coded for a UAA/UAG-specific eRF1 protein that could function in yeast. The whole gene-RF1 construct was derived from the organism Euplotes octocarinatus, The episomal-based shuffle system, which utilized 5-FOA to counter-select against the URA3-marked omnipotent wild-type yeast eRF1, was employed to test the stop codon specificity and functionality of the construct when expressed in yeast. To provide UGA-specificity, another construct (eRF1_Pte1_(m1)) was derived by swapping the YLCDNKF (SEQ iD NO: 2) motif in yeast eRF1 to YFCDPQF (SEQ ID NO: 10) (as found in the eRF1 protein sequence of the organism Paramecium tetraurelia). When expressed separately, the Eoc_eRF1_AAG25924.1 and eRF1_Pte1_(m1) eRF1 constructs did not support viability of a sup45Δ mutant on 5-FOA media, suggesting that these strains post-shuffle cannot decode either UGA or UAA/UAG, respectively. When expressed together, the Eoc_eRF1_AAG25924.1 and eRF1_Pte1_(m1) constructs supported viability of a sup45Δ mutant on 5-FOA media, consistent with the stop codon specificity of the two eRF1 constructs and simultaneously demonstrating that both could function in yeast (Table 4).

TABLE 4
Summary of ciliate eRF1 whole-gene replacements.

| | | % Sequence | |
|---|---|---|---|
| | | identity to | |
| UAA/UAG/UGA | Yeast_eRF1_NP_009701.3 | Yeast eRF1 | Status *** |
| UAA/UAG | Eoc_eRF1_CAC14170.1 * | 57 | Replaceable |
| UAA/UAG | Eoc_eRF1_AAG25924.1 * | 56 | Replaceable |
| UAA/UAG | Bja_eRF1_CAC16186.2 * | 59 | Non- |
| | | | replaceable |
| UGA | Tth_eRF1_XP_001018735.1 ** | 55 | Non- |
| | | | replaceable |
| UGA | Tth_eRF1_XP_001018211.4 ** | 35 | Non- |
| | | | replaceable |
| UGA | Tth_eRF1_XP_001008252.2 ** | 20 | Non- |
| | | | replaceable |
| UGA | Pte_eRF1_XP_001425245.1 ** | 45 | Non- |
| | | | replaceable |
| UGA | Pte_eRF1_XP_001448143.1 ** | 42 | Non- |
| | | | replaceable |
| UGA | Smy_eRF1_Q9BMM1.1 ** | 56 | Non- |
| | | | replaceable |
| UGA | Ssa_eRF1_EST45466.1 ** | 41 | Non- |
| | | | replaceable |

* UAA/UAG-specific constructs tested against the UGA-specific eRF1_Pte1_(m1); all constructs regulated by a SUP45pro
** UGA-specific constructs tested against the UAA/UAG-specific eRF1_Bam_Bja; all constructs regulated by a SUP45pro
*** Status of construct when tested in an erf1 Δ mutant:
Replaceable: Functional in yeast and confers stop codon selectivity, supports growth on 5-FOA only when expressed with the opposite construct.
Non-replaceable: Not functional in yeast, unknown status on stop codon selectivity, does not support growth on 5-FOA when expressed with the opposite construct.

Table 5 highlights the UAA/UAG whole-gene ciliate eRF1 constructs that were tested in conjunction with ciliate eRF3 in yeast. Ciliate eRF1 and eRF3 constructs, under the transcriptional control of the yeast bi-directional GAL1/10 promoter, were tested against the motif-swap constructs, A yeast erf1Δ strain pre-transformed with the endogenously regulated yeast eRF1 (URA3-marked plasmid), was subsequently transformed with the endogenously-regulated (SUP45pro) motif-swap UAA/UAG-specific construct (eRF1_Bam_Bja)(LEU2) and the indicated spHIS5-marked UGA-specific whole-gene eRF1/eRF3 constructs, or with the endogenously-regulated (SUP45pro) motif-swap UGA-specific construct (eRF1_Pte1_(m1)) (HIS3) and the indicated LEU2-marked UAA/UAG-specific whole-gene eRF1/eRF3 constructs. Yeast strains were maintained on SC-URA-LEU-HIS+Dex media, Ciliate ORFs were induced on the same selective media containing galactose for 3 days, before re-streaking on media supplemented with 5-FOA, while selecting for only two of the plasmid constructs (LEU2- and spHIS5/HIS3-marked).

The Eoc_eRF1_CAC14170 construct coded for a UAA/UAG-specific eRF1 protein that could function in yeast. The Eoc_eRF3_AAL33628.1 construct coded for the corresponding eRF3 protein. The whole gene eRF1/eRF3 constructs were derived from the organism Euplotes octocarinatus. The episomal-based shuffle system, which utilized 5-FOA to counter-select against the URA3-marked omnipotent wild-type yeast eRF1, was employed to test the stop codon specificity and functionality of the construct when expressed in yeast. To provide UGA-specificity, another construct (eRF1_Pte1_(m1)) was derived by swapping the YLCDNKF (SEQ ID NO: 2) motif in yeast eRF1 to YFCDPQF (SEQ ID NO: 10) (as found in the eRF1 protein sequence of the organism Paramecium tetraurelia). When expressed separately, the Eoc_eRF1_CAC14170.1/Eoc_eRF3_AAL33628.1 eRF1/eRF3 and eRF1_Pte1_(m1) eRF1 constructs did not support viability of a sup45Δ mutant on 5-FOA media, suggesting that these strains post-shuffle cannot decode either UGA or UAA/UAG, respectively. When expressed together, the Eoc_eRF1_CAC14170.1/Eoc_eRF3_AAL33628.1 eRF1/eRF3 and eRF1_Pte1_(m1) constructs supported viability of a sup45Δ mutant on 5-FOA media, consistent with the stop codon specificity of the two eRF1 constructs and simultaneously demonstrating that both could function in yeast (Table 5).

The Eoc_eRF1_AAG25924.1 construct coded for a UAA/UAG-specific eRF1 protein that could function in yeast. The Eoc_eRF3_AAL33628.1 construct coded for the corresponding eRF3 protein. The whole gene eRF1/eRF3 constructs were derived from the organism Euplotes octocarinatus. The episomal-based shuffle system, which utilized 5-FOA to counter-select against the URA3-marked omnipotent wild-type yeast eRF1, was employed to test the stop codon specificity and functionality of the construct when expressed in yeast, To provide UGA-specificity, another construct (eRF1_Pte1_(m1)) was derived by swapping the YLCDNKF (SEQ ID NO: 2) motif in yeast eRF1 to YFCDPQF (SEQ ID NO: 10) (as found in the eRF1 protein sequence of the organism Paramecium tetraurelia). When expressed separately, the Eoc_eRF1_AAG25924.1/Eoc_eRF3_AAL33628.1 eRF1/eRF3 and eRF1_Pte1_(m1) eRF1 constructs did not support viability of a sup45Δ mutant on 5-FOA media, suggesting that these strains post-shuffle cannot decode either UGA or UAA/UAG, respectively. When expressed together, the Eoc_eRF1_AAG25924.1/Eoc_eRF3_AAL33628.1 eRF1/eRF3 and eRF1_Pte1_(m1) constructs supported viability of a sup45Δ mutant on 5-FOA media, consistent with the stop codon specificity of the two eRF1 constructs and simultaneously demonstrating that both could function in yeast (Table 5).

TABLE 5
Summary of ciliate eRF1 whole-gene replacements
expressed in conjugation with ciliate eRF3.

| | | % Sequence | |
|---|---|---|---|
| | Yeast_eRF1_NP_009701.3; | identity to | |
| UAA/UAG/UGA | Yeast_eRF3_NP_010457.3 | Yeast_eRF1; eRF3 | Status *** |
| UAA/UAG | Eoc_eRF1_CAC14170.1; | 57; | Replaceable |
| | Eoc_eRF3_AAL33628.1 * | 25 | |
| UAA/UAG | Eoc_eRF1_AAG25924.1; | 56; | Replaceable |
| | Eoc_eRF3_AAL33628.1 * | 25 | |
| UAA/UAG | Bja_eRF1_CAC16186.2; | 59; | Non- |
| | Bja_eRF3_AAD03251.1 * | 25 | replaceable |
| UGA | Tth_eRF1_XP_001018735.1; | 55; | Replaceable |
| | Tth_eRF3_XP_001011280.3 ** | 33 | |
| UGA | Tth_eRF1_XP_001018211.4; | 35; | Non- |
| | Tth_eRF3_XP_001011280.3 ** | 33 | replaceable |
| UGA | Tth_eRF1_XP_001008252.2; | 20; | Non- |
| | Tth_eRF3_XP_001011280.3 ** | 33 | replaceable |
| UGA | Pte_eRF1_XP_001425245.1; | 45; | Non- |
| | Pte_eRF3_XP_001459190.1 ** | 36 | replaceable |
| UGA | Pte_eRF1_XP_001448143.1; | 42; | Non- |
| | Pte_eRF3_XP_001459190.1 ** | 36 | replaceable |

* UAA/UAG-specific constructs tested against the UGA-specific eRF1_Pte1_(m1); UAA/UAG constructs regulated by a GAL1/10pro, UGA-specific construct regulated by a SUP45pro
** UGA-specific constructs tested against the UAA/UAG-specific eRF1_Bam_Bja; UGA constructs regulated by a GAL1/10pro, UAA/UAG-specific construct regulated by a SUP45pro
*** Status of construct when tested in an erf1 Δ mutant:
Replaceable: Functional in yeast and confers stop codon selectivity, supports growth on 5-FOA only when expressed with the opposite construct.
Non-replaceable: Not functional in yeast, unknown status on stop codon selectivity, does not support growth on 5-FOA when expressed with the opposite construct.

Table 6 highlights the UAA/UAG whole-gene ciliate eRF1 constructs that were tested in conjunction with N-terminally-modified ciliate eRF3 in yeast. Ciliate eRF1 and eRF3 constructs, under the transcriptional control of the yeast bi-directional GAL1/10 promoter, were tested against the motif-swap constructs. Ciliate eRF3 ORFs were modified by replacing their N-terminal domain with the N-terminal domain of yeast eRF3, thereby creating a chimeric yeast ciliate eRF3 gene construct. A yeast erf1Δ strain pre-transformed with the endogenously regulated yeast eRF1 (URA3-marked plasmid), was subsequently transformed with the endogenously-regulated (SUP45pro) motif-swap UAA/UAG-specific construct (eRF1_Bam_Bja) (LEU2) and the indicated spHIS5-marked UGA-specific whole-gene eRF1/eRF3 constructs, or with the endogenously-regulated (SUP45pro) motif-swap UGA-specific construct (eRF1_Pte1_(m1)) (HIS3) and the indicated LEU2-marked UAA/UAG-specific whole-gene eRF1/eRF3 constructs. Yeast strains were maintained on SC-URA-LEU-HIS+Dex media. Ciliate ORFs were induced on the same selective media containing galactose for 3 days, before re-streaking on media supplemented with 5-FOA, while selecting for only two of the plasmid constructs (LEU2- and spHIS5/HIS3-marked).

The Eoc_eRF1_CAC14170.1 construct coded for a UAA/UAG-specific eRF1 protein that could function in yeast. The N_Yeast_eRF3_Eoc_eRF3_AAL33628.1 construct coded for the corresponding eRF3 protein that was modified by swapping the divergent N-terminal domain of the ciliate eRF3 with the N-terminal domain of yeast eRF3. This chimeric yeast-ciliate eRF3 protein was a fusion of amino acid residues (6-253) from yeast eRF3 with amino acid residues (1-6 and 299-799) of ciliate eRF3. The whole gene eRF1 and C-terminal domain of the chimeric eRF3 constructs were derived from the organism Euplotes octocarinatus. The episomal-based shuffle system, which utilized 5-FOA to counter-select against the URA3-marked omnipotent wild-type yeast eRF1, was employed to test the stop codon specificity and functionality of the construct when expressed in yeast. To provide UGA-specificity, another construct (eRF1_Pte1_(m1)) was derived by swapping the YLCDNKF (SEQ ID NO: 2) motif in yeast eRF1 to YFCDPQF (SEQ ID NO: 10) (as found in the eRF1 protein sequence of the organism Paramecium tetraurelia). When expressed separately, the Eoc_eRF1_CAC14170.1/N_Yeast_eRF3 Eoc_eRF3_AAL33628.1 eRF1/eRF3 and eRF1_Pte1_(m1) eRF1 constructs did not support viability of a sup45Δ mutant on 5-FOA media, suggesting that these strains post-shuffle cannot decode either UGA or UAA/UAG, respectively. When expressed together, the Eoc_eRF1_CAC14170.1/N_Yeast_eRF3_Eoc_eRF3_AAL33628.1 eRF1/eRF3 and eRF1_Pte1_(m1) constructs supported viability of a sup45Δ mutant on 5-FOA media, consistent with the stop codon specificity of the two eRF1 constructs and simultaneously demonstrating that both could function in yeast (Table 6).

TABLE 6
Summary of ciliate eRF1 whole-gene replacements expressed
in conjunction with N-terminally modified ciliate eRF3.

| | | % Sequence | |
|---|---|---|---|
| | Yeast_eRF1_NP_009701.3; | identity to | |
| UAA/UAG/UGA | Yeast_eRF3_NP_010457.3 | Yeast_eRF1; eRF3 | Status *** |
| UAA/UAG | Eoc_eRF1_CAC14170.1; | 57; | Replaceable |
| | N_Yeast_eRF3_Eoc_eRF3_AAL33628.1 * | 67 | |
| UAA/UAG | Eoc_eRF1_AAG25924.1; | 56; | Non-replaceable |
| | N_Yeast_eRF3_Eoc_eRF3_AAL33628.1 * | 67 | |
| UGA | Pte_eRF1_XP_001425245.1; | 45; | Non-replaceable |
| | N_Yeast_eRF3_Pte_eRF3_XP_001459190.1 ** | 63 | |
| UGA | Pte_eRF1_XP_001448143.1; | 42; | Non-replaceable |
| | N_Yeast_eRF3_Pte_eRF3_XP_001459190.1 ** | 63 | |

* UAA/UAG-specific constructs tested against the UGA-specific eRF1_Pte1_(m1); UAA/UAG constructs regulated by a GAL1/10pro, UGA-specific construct regulated by a SUP45pro
** UGA-specific constructs tested against the UAA/UAG-specific eRF1_Bam_Bja; UGA constructs regulated by a GAL1/10pro, UAA/UAG-specific construct regulated by a SUP45pro
*** Status of construct when tested in an erf1 Δ mutant:
Replaceable: Functional in yeast and confers stop codon selectivity, supports growth on 5-FOA only when expressed with the opposite construct.
Non-replaceable: Not functional in yeast, unknown status on stop codon selectivity, does not support growth on 5-FOA when expressed with the opposite construct.

### Example 9: UGA-Specific Constructs

### Domain/Motif-Swap

Table 3 highlights the UGA-specific domain-swapped yeast eRF1 constructs tested in yeast. A yeast erf1Δ strain pre-transformed with the endogenously regulated yeast eRF1 (URA3-marked plasmid), was subsequently transformed with the endogenously-regulated (SUP45pro) motif-swap UAA/UAG-specific construct (eRF1_Bam_Bja) (LEU2) and the indicated HIS3-marked candidate UGA-specific constructs, or with the endogenously-regulated (SUP45pro) motif-swap UGA-specific construct (eRF1_Pte1_(m1)) (HIS3) and the indicated LEU2-marked candidate UAA/UAG-specific constructs. Yeast strains were maintained on SC-URA-LEU-HIS+Dex media, before testing for replaceability on SC-LEU-HIS+5-FOA+Dex media (Table 3).

The eRF1_Pte1_(m1) construct was UGA-specific and could function in yeast. This construct was derived by swapping the YLCDNKF (SEQ ID NO: 2) motif in yeast eRF1 to YFCDPQF (SEQ ID NO: 10) (as found in the eRF1 protein sequence of the organism Paramecium tetraurelia). The episomal-based shuffle system, which utilized 5-FOA to counter-select against the URA3-marked omnipotent wild-type yeast eRF1, was employed to test the stop codon specificity and functionality of the construct when expressed in yeast. To provide UAA/UAG-specificity, another construct (eRF1_Bam_Bja) was derived by swapping the TASNIKS (SEQ ID NO: 1)/YLCDNKF (SEQ ID NO: 2) motifs in yeast eRF1 to KSSNIKS (SEQ ID NO: 3)/YICDNKF (SEQ ID NO: 4) (as found in the eRF1 protein sequences of the organisms Blepharisma americanum and Blepharisma japonicum). When expressed separately, the eRF1_Pte1_(m1) and eRF1_Bam_Bja eRF1 constructs did not support viability of a sup45Δ mutant on 5-FOA media, suggesting that these strains post-shuffle cannot decode either UAA/UAG or VGA, respectively. When expressed together, the eRF1_Pte1_(m1) and eRF1_Bam_Bja constructs supported viability of a sup45Δ mutant on 5-FOA media, consistent with the predicted stop codon specificity of the two eRF1 constructs and simultaneously demonstrating that both could function in yeast (Table 3).

The eRF1_Pte1_(m2) construct was UGA-specific and could function in yeast. This construct was derived by swapping the TASNIKS (SEQ ID NO: 1)/YLCDNKF (SEQ ID NO: 2) motifs in yeast eRF1 to EAASIKD (SEQ ID NO: 11)/YFCDPQF (SEQ ID NO: 10) (as found in the eRF1 protein sequence of the organism Paramecium tetraurelia). The episomal-based shuffle system, which utilized 5-FOA to counter-select against the URA-marked omnipotent wild-type yeast eRF1, was employed to test the stop codon specificity and functionality of the construct when expressed in yeast. To provide UAA/UAG-specificity, another construct (eRF1_Bam_Bja) was derived by swapping the TASNIKS (SEQ ID NO: 1)/YLCDNKF (SEQ ID NO: 2) motifs in yeast eRF1 to KSSNIKS (SEQ ID NO: 3)/YICDNKF (SEQ ID NO: 4) (as found in the eRF1 protein sequences of the organisms Blepharisma americanum and Blepharisma japonicum). When expressed separately, the eRF1_Pte1_(m2) and eRF1_Bam_Bja eRF1 constructs did not support viability of a sup45Δ mutant on 5-FOA media, suggesting that these strains post-shuffle cannot decode either UAA/UAG or UGA, respectively. When expressed together, the eRF1_Pte1_(m2) and eRF1_Bam_Bja constructs supported viability of a sup45Δ mutant on 5-FOA media, consistent with the predicted stop codon specificity of the two eRF1 constructs and simultaneously demonstrating that both could function in yeast (Table 3).

The eRF1_Imu construct was UGA-specific and could function in yeast. This construct was derived by swapping the TASNIKS (SEQ ID NO: 1)/YLCDNKF (SEQ ID NO: 2) motifs in yeast eRF1 to KATNIKD (SEQ ID NO: 12)/FVIVNKF (SEQ ID NO: 20) (as found in the eRF1 protein sequence of the organism Ichthyophthirius multifiliis). The episomal-based shuffle system, which utilized 5-FOA to counter-select against the URA3-marked omnipotent wild-type yeast eRF1, was employed to test the stop codon specificity and functionality of the construct when expressed in yeast. To provide UAA/UAG-specificity, another construct (eRF1_Bam_Bja) was derived by swapping the TASNIKS (SEQ ID NO: 1)/YLCDNKF (SEQ ID NO: 2) motifs in yeast eRF1 to KSSNIKS (SEQ ID NO: 3)/YICDNKF (SEQ ID NO: 4) (as found in the eRF1 protein sequences of the organisms Blepharisma americanum and Blepharisma japonicum). When expressed separately, the eRF1_Imu and eRF1_Bam_Bja eRF1 constructs did not support viability of a sup45Δ mutant on 5-FOA media, suggesting that these strains post-shuffle cannot decode either UAA/UAG or UGA, respectively. When expressed together, the eRF1_Imu and eRF1_Bam_Bja constructs supported viability of a sup45Δ mutant on 5-FOA media, consistent with the predicted stop codon specificity of the two eRF1 constructs and simultaneously demonstrating that both could function in yeast (Table 3).

The eRF1_Ppe1 construct was UGA-specific and could function in yeast. This construct was derived by swapping the TASNIKS (SEQ ID NO: 1)/YLCDNKF (SEQ ID NO: 2) motifs in yeast eRF1 to QANSIKD (SEQ ID NO: 23)/YRCDSKF (SEQ ID NO: 24) (as found in the eRF1 protein sequence of the organism Pseudocohnilembus persalinus). The episomal-based shuffle system, which utilized 5-FOA to counter-select against the URA3-marked omnipotent wild-type yeast eRF1, was employed to test the stop codon specificity and functionality of the construct when expressed in yeast. To provide UAA/UAG-specificity, another construct (eRF1_Bam_Bja) was derived by swapping the TASNIKS (SEQ ID NO: 1)/YLCDNKF (SEQ ID NO: 2) motifs in yeast eRF1 to KSSNIKS (SEQ ID NO: 3)/YICDNKF (SEQ ID NO: 4) (as found in the eRF1 protein sequences of the organisms Blepharisma americanum and Blepharisma japonicum). When expressed separately, the eRF1_Ppe1 and eRF1_Bam_Bja eRF1 constructs did not support viability of a sup45Δ mutant on 5-FOA media, suggesting that these strains post-shuffle cannot decode either UAA/UAG or UGA, respectively. When expressed together, the eRF1_Ppe1 and eRF1_Bam_Bja constructs supported viability of a sup45Δ mutant on 5-FOA media, consistent with the predicted stop codon specificity of the two dal constructs and simultaneously demonstrating that both could function in yeast (Table 3).

The eRF1_Tth2 construct was UGA-specific and could function in yeast. This construct was derived by swapping the TASNIKS (SEQ ID NO: 1)/YLCDNKF (SEQ ID NO: 2) motifs in yeast eRF1 to GAASIKN (SEQ ID NO: 25)/YSCNTIF (SEQ ID NO: 26) (as found in the eRF1 protein sequence of the organism Tetrahymena thermophila). The episomal-based shuffle system, which utilized 5-FOA to counter-select against the URA marked omnipotent wild-type yeast eRF1, was employed to test the stop codon specificity and functionality of the construct when expressed in yeast. To provide UAA/UAG-specificity, another construct (eRF1_Bam_Bja) was derived by swapping the TASNIKS (SEQ ID NO: 1)/YLCDNKF (SEQ ID NO: 2) motifs in yeast eRF1 to KSSNIKS (SEQ ID NO: 3)/YICDNKF (SEQ ID NO: 4) (as found in the eRF1 protein sequences of the organisms Blepharisma americanum and Blepharisma japonicum). When expressed separately, the eRF1_Tth2 and eRF1_Bam_Bja eRF1 constructs did not support viability of a sup45Δ mutant on 5-FOA media, suggesting that these strains post-shuffle cannot decode either UAA/UAG or UGA, respectively. When expressed together, the eRF1_Tth2 and eRF1_Bam_Bja constructs supported viability of a sup45Δ mutant on 5-FOA media, consistent with the predicted stop codon specificity of the two eRF1 constructs and simultaneously demonstrating that both could function in yeast (Table 3).

The eRF1_Uhl construct was UGA-specific and could function in yeast. This construct was derived by swapping the TASNIKS (SEQ ID NO: 1)/YLCDNKF (SEQ ID NO: 2) motifs in yeast eRF1 to SAQSIKS (SEQ ID NO: 32)/YFCDNSF (SEQ ID NO: 30) (as found in the eRF1 protein sequence of the organism Urostyla sp. HL-2004). The episomal-based shuffle system, which utilized 5-FOA to counter-select against the URA3-marked omnipotent wild-type yeast eRF1 was employed to test the stop codon specificity and functionality of the construct when expressed in yeast. To provide UAA/UAG-specificity, another construct (eRF1_Bam_Bja) was derived by swapping the TASNIKS (SEQ ID NO: 1)/YLCDNKF (SEQ ID NO: 2) motifs in yeast eRF1 to KSSNIKS (SEQ ID NO: 3)/YICDNKF (SEQ ID NO: 4) (as found in the eRF1 protein sequences of the organisms Blepharisma americanum and Blepharisma japonicum). When expressed separately, the eRF1_Uhl1 and eRF1_Bam_Bja eRF1 constructs did not support viability of a sup45Δ mutant on 5-FOA media, suggesting that these strains post-shuffle cannot decode either UAA/UAG or UGA, respectively. When expressed together, the eRF1_Uhl1 and eRF1_Bam_Bja constructs supported viability of a sup45Δ mutant on 5-FOA media, consistent with the predicted stop codon specificity of the two eRF1 constructs and simultaneously demonstrating that both could function in yeast (Table 3).

The eRF1_Ssa construct was UGA-specific and could function in yeast. This construct was derived by swapping the TASNIKS (SEQ ID NO: 1)/YLCDNKF (SEQ ID NO: 2) motifs in yeast eRF1 to QADCIKS (SEQ ID NO: 36)/YSCDGVF (SEQ ID NO: 37) (as found in the eRF1 protein sequence of the organism Spironucleus salmonicida). The episomal-based shuffle system, which utilized 5-FOA to counter-select against the URA3-marked omnipotent wild-type yeast eRF1, was employed to test the stop codon specificity and functionality of the construct when expressed in yeast. To provide UAA/UAG-specificity, another construct (eRF1_Bam_Bja) was derived by swapping the TASNIKS (SEQ ID NO: 1)/YLCDNKF (SEQ ID NO: 2) motifs in yeast eRF1 to KSSNIKS (SEQ ID NO: 3)/YICDNKF (SEQ ID NO: 4) (as found in the eRF1 protein sequences of the organisms Blepharisma americanum and Blepharisma japonicum). When expressed separately, the eRF1 Ssa and eRF1_Bam_BjaeRF1 constructs did not support viability of a sup45Δ mutant on 5-FOA media, suggesting that these strains post-shuffle cannot decode either UAA/UAG or UGA, respectively. When expressed together, the eRF1_Ssa and eRF1_Bam_Bja constructs supported viability of a sup45Δ mutant on 5-FOA media, consistent with the stop codon specificity of the two eRF1 constructs and simultaneously demonstrating that both could function in yeast (Table 3).

The eRF1_Lst construct was UGA-specific and could function in yeast. This construct was derived by swapping the TASNIKS (SEQ ID NO: 1)/YLCDNKF (SEQ ID NO: 2) motifs in yeast eRF1 to RAQNIKS (SEQ ID NO: 38)/FLCENTF (SEQ ID NO: 39) (as found in the eRF1 protein sequence of the organism Loxodes striatus). The episomal-based shuffle system, which utilized 5-FOA to counter-select against the URA3-marked omnipotent wild-type yeast eRF1, was employed to test the stop codon specificity and functionality of the construct when expressed in yeast. To provide UAA/UAG-specificity, another construct (eRF1_Bam_Bja was derived by swapping the TASNIKS (SEQ ID NO: 1)/YLCDNKF (SEQ ID NO: 2) motifs in yeast eRF1 to KSSNIKS (SEQ ID NO: 3)/YICDNKF (SEQ ID NO: 4) (as found in the eRF1 protein sequences of the organisms Blepharisma americanum and Blepharisma japonicum). When expressed separately, the eRF1_Lst and eRF1_Bam_Bja eRF1 constructs did not support viability of a sup45Δ mutant on 5-FOA media, suggesting that these strains cannot decode either UAA/UAG or UGA, respectively. When expressed together, the eRF1_Lst and eRF1_Bam_Bja constructs supported viability of a sup45Δ mutant on 5-FOA media, consistent with the stop codon specificity of the two eRF1 constructs and simultaneously demonstrating that both could function in yeast (Table 3).

### Whole Gene Swaps

Table 5 highlights all the UGA-specific whole-gene ciliate eRF1 constructs that were tested in conjunction with ciliate eRF3 in yeast. Ciliate eRF1 and eRF3 constructs, under the transcriptional control of the yeast bi-directional GAL1/10 promoter, were tested against the motif-swap constructs. A yeast erf1Δ strain pre-transformed with the endogenously regulated yeast eRF1 (URA3-marked plasmid), was subsequently transformed with the endogenously-regulated (SUP45pro) motif-swap UAA/UAG-specific construct (eRF1_Bam_bja) (LEU2) and the indicated spHIS5-marked UGA-specific whole-gene eRF1/eRF3 constructs, or with the endogenously-regulated (SUP45pro) motif-swap UGA-specific construct (eRF1_Pte1_(m1)) (HIS3) and the indicated LEU2-marked UAA/UAG-specific whole-gene eRF1/eRF3 constructs. Yeast strains were maintained on SC-URA-LEU-HIS+Dex media. Ciliate ORFs were induced on the same selective media containing galactose for 3 days, before re-streaking on media supplemented with 5-FOA, while selectins for only two of the plasmid constructs (LEU2- and spHIS5/HIS3-marked).

The Tth_eRF1_XP_001018735.1 construct coded for a UGA-specific eRF1 protein that could function in yeast when combined with the corresponding Tth_eRF3_XP_001011280.3 eRF3 construct. The whole gene eRF1/eRF3 constructs were derived from the organism Tetrahymena thermophila. The episomal-based shuffle system, which utilized 5-FOA to counter-select against the URA3-marked omnipotent wild-type yeast eRF1, was employed to test the stop codon specificity and functionality of the ciliate eRF1 construct upon expression in yeast. To provide UAA/UAG-specificity, another construct (eRF1_Bam_Bja) was derived by swapping the TASNIKS (SEQ ID NO: 1)/YLCDNKF (SEQ ID NO: 2) motifs in yeast eRF1 to KSSNIKS (SEQ ID NO: 3)/YICDNKF (SEQ ID NO: 4) (as found in the eRF1 protein sequences of the organisms Blepharisma americanum and Blepharisma japonicum). When expressed separately, the Tth_eRF1_XP_001018735.1 and eRF1_Bam_Bja eRF1 constructs did not support viability of a sup45Δ mutant on 5-FOA media, suggesting that these strains post-shuffle could not decode either UAA/UAG or UGA, respectively (Table 4). When expressed separately, the UGA-specific Tth_eRF1_XP_001018735.1/Tth_eRF3_XP_001011280.3 eRF1/eRF3 construct did not support viability of a sup45Δ mutant on 5-FOA media, suggesting that this strain could not decode UAA/UAG (Table 5). When expressed together, the Tth_eRF1_XP_001018735.1 and eRF1_Bam_Bja eRF1 constructs did not support viability of a sup45Δ mutant on 5-FOA media (Table 4). However, concurrent expression of the Tth_eRF3_XP_001011280.3 eRF3 construct with the Tth_eRF1_XP_001018735.1 and eRF1 Rain eRF1 constructs supported viability of a sup45Δ mutant on 5-FOA media (Table 5). These results are consistent with the stop codon specificity of the two eRF1 constructs and simultaneously demonstrated that both can function in yeast. In the case of the UGA-specific Tth_eRF1_XP_001018735.1 eRF1 construct, its function required the corresponding Tth_eRF3_XP_001011280.3 eRF3 construct.

### Example 10: Production of a Polypeptide Containing ncAAs

A cell (e.g., CAR-T cell) is transfected with a recombinant nucleic acid comprising a sequence encoding a recombinant release factor configured to recognize UGA with a cassette for an inducible degron system using methods known to those of skill in the art. Integration of the recombinant nucleic acid is confirmed using any known method, e.g., PCR. Once integration is confirmed, the cell is expanded to generate a population of cells or a cell line expressing recombinant release factors with degrons. Inducing agents can be introduced to the population of cells or the cell line to turn on the degron system and degrade the recombinant release factors. Orthogonal aminoacyl-tRNA synthetase (aaRS)/tRNA pairs that specifically and efficiently decode UGA codon are developed and introduced into the population of cells or the cell line for incorporation of ncAA at UGA codons to generate polypeptides with ncAAs (e.g., cell-drug conjugates or cell-antibody conjugates).

### Example 11: Screening a Release Factor for Stop Codon-Specific Release Factor Activity

A sup45-ts cell (i.e., a cell that expresses temperature-sensitive release factor) is transfected with a first recombinant nucleic acid sequence comprising a sequence encoding a first release factor configured to recognize UGA as a stop codon using methods known to those of skill in the art. Integration of the recombinant nucleic acid sequence is confirmed using any known method, e.g., PCR. Once integration is confirmed, the cell is expanded to generate a population of cells or a cell line at permissive temperature so the endogenous release factor expressed from sup45-ts is functional. One or more second release factors (e.g., release factors with different mutations, etc.) are introduced to the population of cells or the cell line. The population of cells or the cell line is then incubated at non-permissive temperature so the endogenous release factor expressed from sup45-ts is non-functional. Only the population of cells or the cell line with a second release factor with a release factor activity that can complement that of the first release factor can be viable at the non-permissive temperature. For example, as the first release factor is configured to UGA as the stop codon, only the population of cells or the cell line with second release factors that can recognize UAA and UAG as stop codons can be viable at non-permissive temperature. The second release factor(s) are then isolated from the population of the cells or the cell line and analyzed and characterized using any method known in the art.

A second assay is then performed to test if the second release factor(s) can recognize UGA as a stop codon. For example, a sup45-ts cell can be transfected with a recombinant nucleic acid comprising a sequence encoding a release factor configured to recognize UAA and UAG as stop codons using methods known to those of skill in the art. Integration of the recombinant nucleic acid sequence is confirmed using any known method, e.g., PCR. Once integration is confirmed, the cell is expanded to generate a population of cells or a cell line at permissive temperature so the endogenous release factor expressed from sup45-ts is functional. The isolated second release factor(s) (e.g., release factors with different mutations, etc.) is introduced to the population of cells or the cell line. The population of cells or the cell line is then incubated at non-permissive temperature so the endogenous release factor expressed from sup45-ts is non-functional. If the isolated second release factor does not recognize UGA as a stop codon, the population of cells or the cell line cannot be viable at non-permissive temperature. The second release factor(s) that can recognize UAA and UAG as stop codons but not UGA can then be selected for other studies or compositions, systems, and methods described herein.

### Example 12: Stop Codon Readthrough and ncAA Incorporation

Relative Readthrough in Strains Encoding Sup45 Temperature Sensitive Alleles

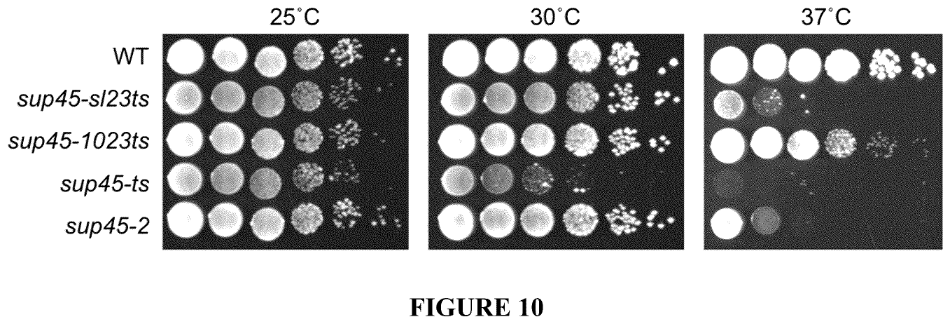

Sup45 temperature sensitive alleles (sup45-ts, sup45-2, sup45-1023ts, or sup45-sl23ts) were introduced into the genome of S. cerevisiae, replacing the wild-type (WT) SUP45 allele. Ten-fold serial dilutions of each of the resulting mutant strains (expressing sup45-ts, sup45-2, sup45-1023ts, or sup45-sl23ts) were spotted on YPD medium and incubated at temperatures of 25° C., 30° C., or 37° C., and grown for 2-3 days. As shown in FIG. 10, only the WT strain was able to grow robustly at all temperatures tested consistent with loss of the essential function of SUP45 in the temperature sensitive alleles. The loss of viability of the mutant strains at higher temperature indicates loss of function of the sup45 mutant proteins in cells. The mutant strains can be used to evaluate readthrough and orthogonal translation.

Three different dual reporter systems were built to evaluate relative readthrough efficiency (RRE) in mutant strains encoding sup45 temperature sensitive alleles. Each system uses a dual blue fluorescent protein (BFP) and green fluorescent protein (GFP) reporter in which the two fluorescence coding sequences were separated by a linker that contains a stop codon. Each system encodes in a 5′ to 3′ direction, a BFP coding sequence, a stop codon (TAA, TAG, or TGA), and a GFP coding sequence (e.g., 5′ BFP-TAA-GFP 3′, 5′ BFP-TAG-GFP 3′, or 5′ BFP-TGA-GFP 3′). The stop codon in the three reporters was the target of readthrough for this experiment.

The three reporter systems were individually transformed into either a WT strain or a mutant strain encoding the temperature sensitive sup45-sl23ts allele that replaced the WT SUP45 allele. RRE evaluation was performed in the presence or absence of an orthogonal translation system (OTS), comprising a heterologous tRNA and synthetase pair engineered for specificity to the non-canonical amino acid (S)-2-amino-6-((2-azidoethoxy)carbonylamino)hexanoic acid (LysN3). The heterologous tRNA was engineered to match the read through stop codon in all cases. RRE is thus defined as the relative BFP:GFP signal, normalized to the relative BFP:GFP signal in a strain carrying the reporter with a sense codon in place of the readthrough stop codon. For comparison, RRE was also measured in strains that did not encode the OTS. All experiments were performed at 30° C. in synthetic complete drop out media selecting for the appropriate OTS and dual reporter system constructs.

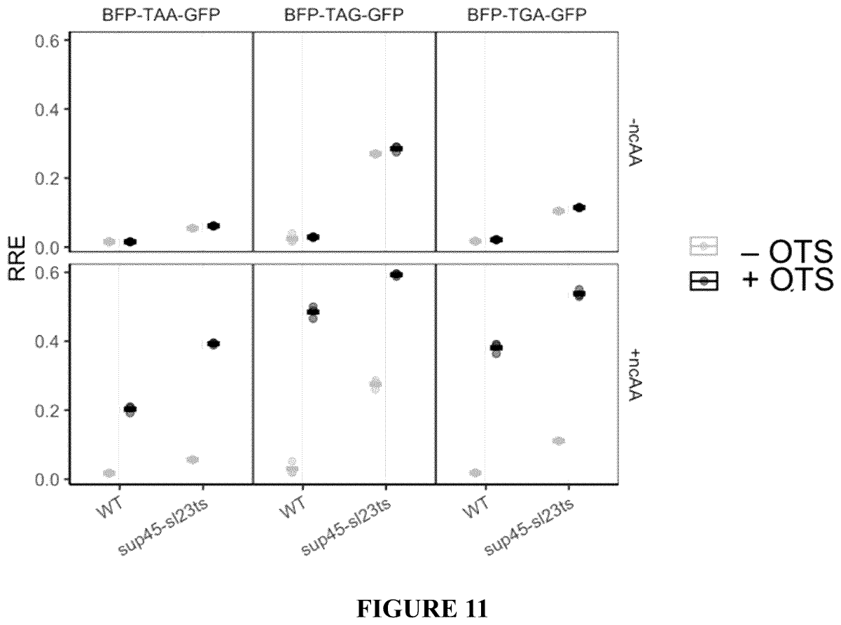

As shown in FIG. 11, there is an increased level of readthrough of any of the three stop codons in the context of a temperature sensitive allele of sup45-sl23ts. The presence of both an OTS and ncAA (LysN3) leads to even higher level of stop codon readthrough, consistent with a further promotion of readthrough due to the presence of OTS and ncAA.

Incorporation Percentage of ncAA LysN3 at a TAG Readthrough Codon in a Sup45 Temperature Sensitive Strain in the Absence or Presence of an OTS Engineered for LysN3 Specificity

Mass spectrometric analysis (LC-MS/MS) was conducted with BFP-TAG-GFP samples purified from two experiments (−OTS and +OTS) performed in a sup45-sl23ts strain background and in the presence of the ncAA LysN3 in the media, using the BFP-TAG-GFP dual reporter system. The identity of the amino acid at the readthrough stop codon was calculated as a percentage.

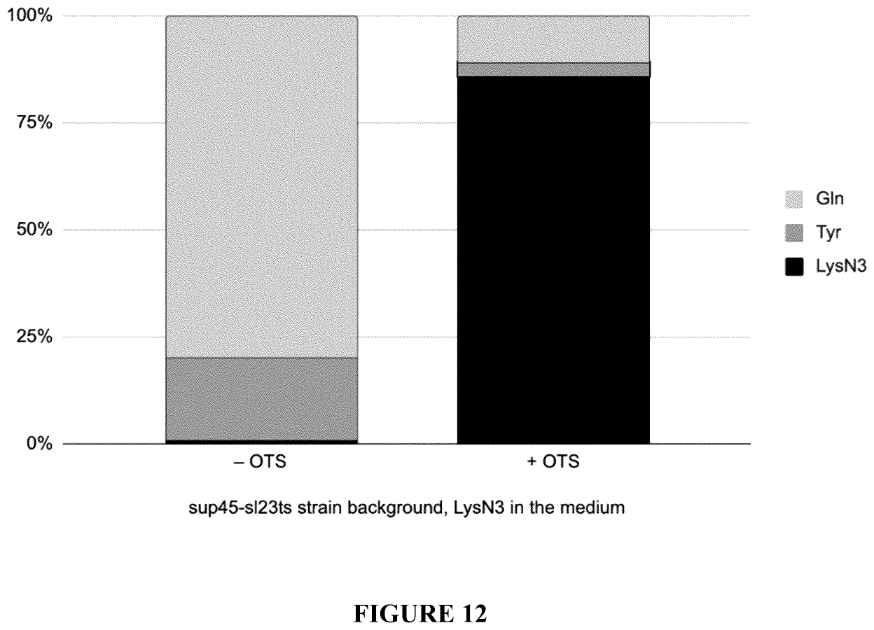

As shown in FIG. 12, about 85% of the BFP-TAG-GFP dual reporter system contained a LysN3 at the readthrough position (TAG) and 15% of the BFP-TAG-GFP dual reporter system contained either a glutamine or tyrosine at the readthrough position (TAG) when an OTS was present. This indicates that when a defective version of the release factor is present in yeast (sup45-sl23ts), the suppressor tRNA recognizing the TAG stop codon of the dual reporter is the dominant species that enters the ribosome to support translation and effectively outcompetes wobble pairing from Gln and Tyr tRNAs.

Stop Codon Specificity of the Bam-SUP45 Release Factor

The anticodon of an alanine tRNA was engineered to encode TAA or TGA stop codon recognition. These tRNAs were expressed in either the parental or revertant strains in the presence of the corresponding dual reporter (BFP-TAA-GFP or BFP-TGA-GFP).

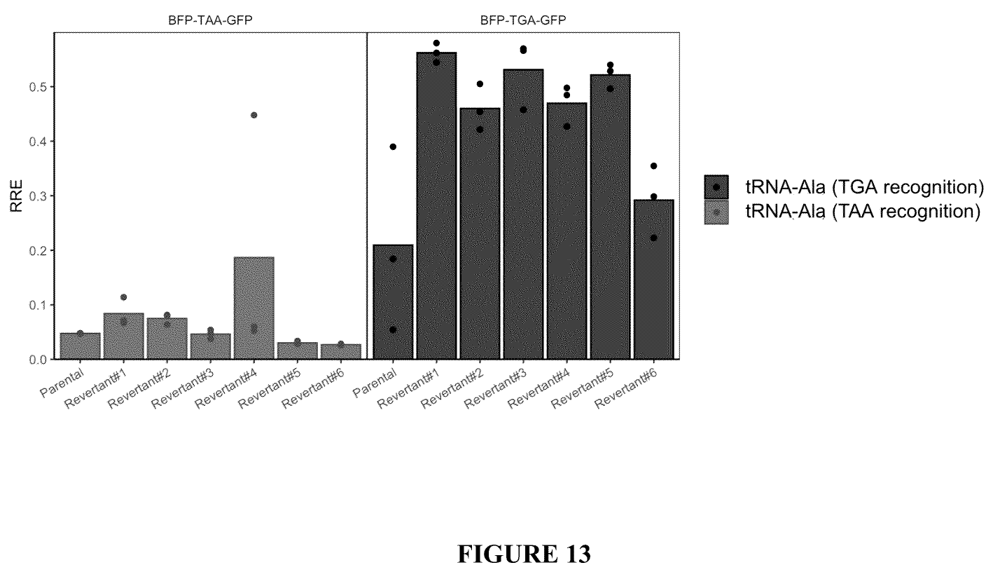

The revertant strains express Bam-SUP45 (protein sequence: SEQ ID NO: 41, nucleic acid sequence: SEQ ID NO: 102), which was shown to demonstrate specificity for TAA/TAG but not TGA in S. cerevisiae (see Example 3 and FIG. 6). The parental strain also expresses the wild-type S. cerevisiae (Sce) SUP45 gene, which provides release factor specificity for all three stop codons. As shown in FIG. 13, when the BFP-TAA-GFP reporter was used, a low level of RRE was observed in both parental and revertant strains, consistent with the Bam-SUP45 providing release factor activity at TAA as expected. When the BFP-TGA-GFP report was used, a high level of RRE was observed, consistent with an absence of release factor activity at TGA and a presence of readthrough activity via incorporation of alanine by the tRNA-Ala engineered for TGA recognition. It was noted that that readthrough level in the parental strain is lower than in the revertant strains, likely due to competition between the Sce-Sup45 release factor and the tRNA-Ala engineered for TGA recognition. The result is consistent with the Bam-SUP45 release factor specificity for TAA but not for TGA.

This experiment shows that the readthrough assay is useful in determining release factor specificity.

Read Through in Revertant Strains Expressing Only a TAG/TAA-Recognizing Release Factor

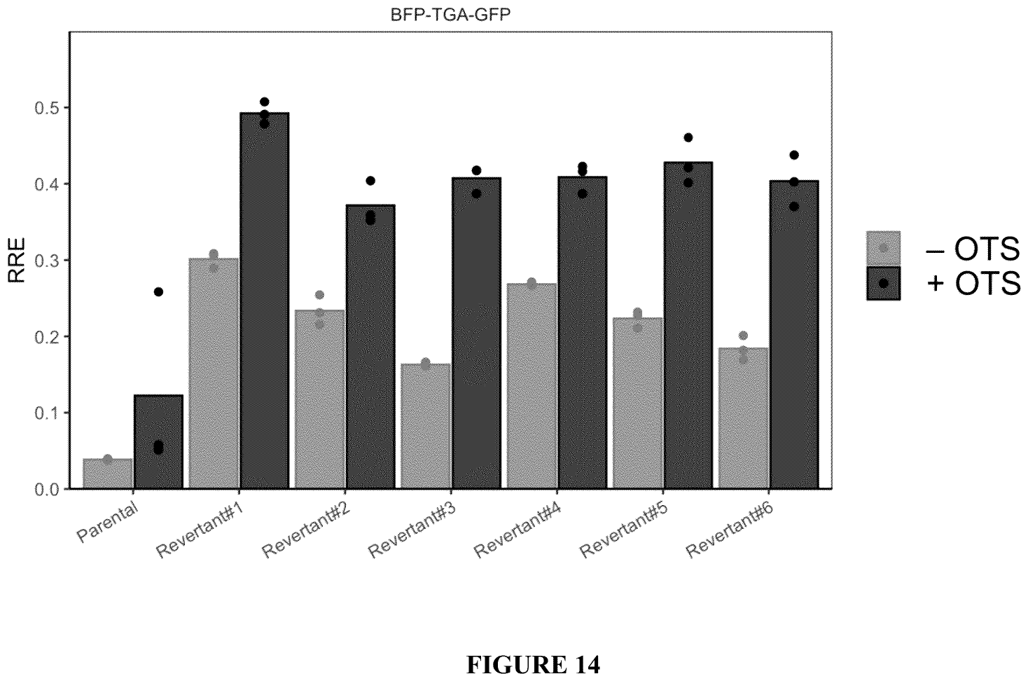

Stop codon readthrough was tested in strains deleted for the genomic SUP45 gene and (i) episomally encoding Sce-SUP45 and Bam-SUP45 (parental strain) or (ii) Bam-SUP45 alone (six individually derived revertant strains). Readthrough was tested using a dual BFP-GFP reporter in which the two fluorescence coding sequences were separated by a linker that contained a TGA stop codon (BFP-TGA-GFP). RRE was evaluated in the absence (−OTS) or presence (+OTS) of an orthogonal translation system (OTS), comprising a heterologous tRNA and synthetase engineered to function specifically with the ncAA LysN3. LysN3 was included in the growth media for all conditions. The tRNA of the OTS was designed with an anticodon complementary to the TGA stop codon. The results, as shown in FIG. 14, demonstrates that readthrough of the dual reporter TGA stop codon increases in the revertant strains, and increases further in the revertant strain expressing the OTS, consistent with this stop codon recognition pattern of Bam-SUP45.

This experiment also shows that it is possible to generate yeast strains that express a single ciliate-derived release factor (Bam-SUP45), which recognizes TAG and TAA and does not recognize TGA.

TABLE 7
Constructs used in examples

| | | | | | Modifications/ | | | | | |
|---|---|---|---|---|---|---|---|---|---|---|
| | | | | | Underlined | | | | | |
| | | | | | Sequences | | | | | Nucleic |
| | | | Ciliate source | Ciliate | modified from | Stop codon | ciliate_eRF1 | eRF3 | Protein | acid |
| No. | Construct ID | category | organism(s) | nickname(s) | original | specificity | accession # | accession # | sequence | sequence |
| 1 | eRF1_Yeast | S. | n/a | n/a | n/a | UAA/UAG/ | NP_009701.3 | | SEQ ID | SEQ ID |
| | | cerevisiae | | | | UGA | | | NO: 40 | NO: 101 |
| | | wild- | | | | | | | | |
| | | type | | | | | | | | |
| 2 | eRF1_Bam_ | Motif- | Blepharisma | Bam | KSSNIKS | UAA/UAG | AAK12089.1 | | SEQ ID | SEQ ID |
| | Bja | swap | americanum | Bja | (SEQ ID NO: | | CAC16186.2 | | NO: 41 | NO: 102 |
| | | | Blepharisma | | 3)/YICDNKF | | | | | |
| | | | japonicum | | (SEQ ID NO: | | | | | |
| | | | | | 4) | | | | | |
| 3 | eRF1_Eae1_ | Motif- | Euplotes | Eae | TAVNIKS | UAA/UAG | AAK07830.1 | | SEQ ID | SEQ ID |
| | Eoc1 | swap | aediculatus | Eoc | (SEQ ID NO: | | AAG25924.1 | | NO: 42 | NO: 103 |
| | | | Euplotes | | 5)/YICDNKF | | | | | |
| | | | octocarinatus | | (SEQ ID NO: | | | | | |
| | | | | | 4) | | | | | |
| 4 | eRF1_Sco | Motif- | Stentor | Sco | KAANIKS | UAA/UAG | OMJ89313.1 | | SEQ ID | SEQ ID |
| | | swap | coeruleus | | (SEQ ID NO: | | OMJ91237.1 | | NO: 43 | NO: 104 |
| | | | | | 6) | | OMJ79310.1 | | | |
| 5 | eRF1_Nov | Motif- | Nyctotherus | Nov | KASNIKS | UAA/UAG | AAX19092.1 | | SEQ ID | SEQ ID |
| | | swap | ovalis | | (SEQ ID NO: | | AAX19093.1 | | NO: 44 | NO: 105 |
| | | | | | 7)/YYCGERF | | | | | |
| | | | | | (SEQ ID NO: | | | | | |
| | | | | | 8) | | | | | |
| 6 | eRF1_Eae2_ | Motif- | Euplotes | Eae | TAESIKS | UAA/UAG | AAK07829.1 | | SEQ ID | SEQ ID |
| | Eoc2 | swap | aediculatus | Eoc | (SEQ ID NO: | | CAC14170.1 | | NO: 45 | NO: 106 |
| | | | Euplotes | | 9)/YICDNKF | | | | | |
| | | | octocarinatus | | (SEQ ID NO: | | | | | |
| | | | | | 4) | | | | | |
| 7 | eRF1_Pte1_ | Motif- | Paramecium | Pte | YFCDPQF | UGA | AAK66860.1 | | SEQ ID | SEQ ID |
| | (m1) | swap | tetraurelia | | (SEQ ID NO: | | AAK66861.1 | | NO: 46 | NO: 107 |
| | | | | | 10) | | | | | |
| 8 | eRF1_Pte1_ | Motif- | Paramecium | Pte | EAASIKD | UGA | AAK66860.1 | | SEQ ID | SEQ ID |
| | (m2) | swap | tetraurelia | | (SEQ ID NO: | | AAK66861.1 | | NO: 47 | NO: 108 |
| | | | | | 11)/ | | | | | |
| | | | | | YFCDPQF | | | | | |
| | | | | | (SEQ ID NO: | | | | | |
| | | | | | 10) | | | | | |
| 9 | eRF1_Tth1 | Motif- | Tetrahymena | Tth | KATNIKD | UGA | XP_001018735.1 | | SEQ ID | SEQ ID |
| | | swap | thermophila | | (SEQ ID NO: | | | | NO: 48 | NO: 109 |
| | | | | | 12)/ | | | | | |
| | | | | | YFCDSKF | | | | | |
| | | | | | (SEQ ID NO: | | | | | |
| | | | | | 13) | | | | | |
| 10 | eRF1_Sle1 | Motif- | Stylonychia | Sle | FDFDAES | UGA | CDW74559.1 | | SEQ ID | SEQ ID |
| | | swap | lemnae | | (SEQ ID NO: | | | | NO: 49 | NO: 110 |
| | | | | | 14)/TLIKPQF | | | | | |
| | | | | | (SEQ ID NO: | | | | | |
| | | | | | 15) | | | | | |
| 11 | eRF1_Ppe2 | Motif- | Pseudocohnil | Ppe | TGDKIKS | UGA | KRW99069.1 | | SEQ ID | SEQ ID |
| | | swap | embus | | (SEQ ID NO: | | | | NO: 50 | NO: 111 |
| | | | persalinus | | 16)/TIIKNDF | | | | | |
| | | | | | (SEQ ID NO: | | | | | |
| | | | | | 17) | | | | | |
| 12 | eRF1_Pte2 | Motif- | Paramecium | Pte | EAASIQD | UGA | CAK80746.1 | | SEQ ID | SEQ ID |
| | | swap | tetraurelia | | (SEQ ID NO: | | | | NO: 51 | NO: 112 |
| | | | | | 18)/ | | | | | |
| | | | | | FFCDNYF | | | | | |
| | | | | | (SEQ ID NO: | | | | | |
| | | | | | 19) | | | | | |
| 13 | eRF1_Imu | Motif- | Ichthyophthirius | Imu | KATNIKD | UGA | XP_004032541.1 | | SEQ ID | SEQ ID |
| | | swap | multifiliis | | (SEQ ID NO: | | | | NO: 52 | NO: 113 |
| | | | | | 12)/FVIVNKF | | | | | |
| | | | | | (SEQ ID NO: | | | | | |
| | | | | | 20) | | | | | |
| 14 | eRF1_Sle2_ | Motif- | Stylonychia | Sle | AAQNIKS | UGA | CDW89307.1 | | SEQ ID | SEQ ID |
| | Otr_Spu_Smy | | lemnae | Otr | (SEQ ID NO: | | AAK07828.1 | | | |
| | | swap | Oxytricha | Spu | 21)/ | | AAN62568.1 | | NO: 53 | NO: 114 |
| | | | trifallax | Smy | YFCGGKF | | AAK12091.1 | | | |
| | | | Stylonychia | | (SEQ ID NO: | | | | | |
| | | | pustulata | | 22) | | | | | |
| | | | Stylonychia | | | | | | | |
| | | | mytilus | | | | | | | |
| 15 | eRF1_Ppe1 | Motif- | Pseudocohnil | Ppe | QANSIKD | UGA | KRX05899.1 | | SEQ ID | SEQ ID |
| | | swap | embus | | (SEQ ID NO: | | | | NO: 54 | NO: 115 |
| | | | persalinus | | 23)/ | | | | | |
| | | | | | YRCDSKF | | | | | |
| | | | | | (SEQ ID NO: | | | | | |
| | | | | | 24) | | | | | |
| 16 | eRF1_Tth2 | Motif- | Tetrahymena | Tth | GAASIKN | UGA | XP_001018211.4 | | SEQ ID | SEQ ID |
| | | swap | thermophila | | (SEQ ID NO: | | | | NO: 55 | NO: 116 |
| | | | | | 25)/YSCNTIF | | | | | |
| | | | | | (SEQ ID NO: | | | | | |
| | | | | | 26) | | | | | |
| 17 | eRF1_Ehl | Motif- | Eschaneustyla | Ehl | SAQNIKS | UGA | AAT39331.1 | | SEQ ID | SEQ ID |
| | | swap | sp. HL-2004 | | (SEQ ID NO: | | | | NO: 56 | NO: 117 |
| | | | | | 27)/ | | | | | |
| | | | | | YYCDNRF | | | | | |
| | | | | | (SEQ ID NO: | | | | | |
| | | | | | 28) | | | | | |
| 18 | eRF1_Ghl | Motif- | Gonostomum | Ghl | SAGNIKS | UGA | AAT39330.1 | | SEQ ID | SEQ ID |
| | | swap | sp. HL-2004 | | (SEQ ID NO: | | | | NO: 57 | NO: 118 |
| | | | | | 29)/ | | | | | |
| | | | | | YFCDNSF | | | | | |
| | | | | | (SEQ ID NO: | | | | | |
| | | | | | 30) | | | | | |
| 19 | eRF1_Hhl | Motif- | Holosticha | Hh1 | TAQNIKS | UGA | AAT39329.1 | | SEQ ID | SEQ ID |
| | | swap | sp. HL-2004 | | (SEQ ID NO: | | | | NO: 58 | NO: 119 |
| | | | | | 31)/ | | | | | |
| | | | | | YFCGGKF | | | | | |
| | | | | | (SEQ ID NO: | | | | | |
| | | | | | 22) | | | | | |
| 20 | eRF1_Uh1 | Motif- | Urostyla sp. | Uhl | SAQSIKS | UGA | AAT39328.1 | | SEQ ID | SEQ ID |
| | | swap | HL-2004 | | (SEQ ID NO: | | | | NO: 59 | NO: 120 |
| | | | | | 32)/ | | | | | |
| | | | | | YFCDNSF | | | | | |
| | | | | | (SEQ ID NO: | | | | | |
| | | | | | 30) | | | | | |
| 21 | eRF1_Uwj_ | Motif- | Uroleptus sp. | Uwj | AANNIKS | UGA | AAT39327.1 | | SEQ ID | SEQ ID |
| | Pwe | swap | WJC-2003 | Pwe | (SEQ ID NO: | | AAT39326.1 | | NO: 60 | NO: 121 |
| | | | Paraurostyla | | 33)/ | | | | | |
| | | | weissei | | YFCGGKF | | | | | |
| | | | | | (SEQ ID NO: | | | | | |
| | | | | | 22) | | | | | |
| 22 | eRF1_Smi | Motif- | Stichotrichida | Smi | YNCSGKF | UGA | AAN62567.1 | | SEQ ID | SEQ ID |
| | | swap | sp. misty | | (SEQ ID NO: | | | | NO: 61 | NO: 122 |
| | | | | | 34) | | | | | |
| 23 | eRF1_Sal | Motif- | Stichotrichida | Sal | QAQNIKS | UGA | AAN62563.1 | | SEQ ID | SEQ ID |
| | | swap | sp. Alaska | | (SEQ ID NO: | | AAN62564.1 | | NO: 62 | NO: 123 |
| | | | | | 35)/ | | | | | |
| | | | | | YFCGGKF | | | | | |
| | | | | | (SEQ ID NO: | | | | | |
| | | | | | 22) | | | | | |
| 24 | eRF1_Ssa | Motif- | Spironucleus | Ssa | QADCIKS | UGA | EST45466.1 | | SEQ ID | SEQ ID |
| | | swap | salmonicida | | (SEQ ID NO: | | | | NO: 63 | NO: 124 |
| | | | | | 36)/ | | | | | |
| | | | | | YSCDGVF | | | | | |
| | | | | | (SEQ ID NO: | | | | | |
| | | | | | 37) | | | | | |
| 25 | eRF1_Lst | Motif- | Loxodes | Lst | RAQNIKS | UGA | BAD90946.1 | | SEQ ID | SEQ ID |
| | | swap | striatus | | (SEQ ID NO: | | | | NO: 64 | NO: 125 |
| | | | | | 38)/FLCENTF | | | | | |
| | | | | | (SEQ ID NO: | | | | | |
| | | | | | 39) | | | | | |
| 26 | Eoc_eRF1_ | Whole- | Euplotes | Eoc | | UAA/UAG | CAC14170.1 | | SEQ ID | SEQ ID |
| | CAC14170.1 | gene | octocarinatus | | | | | | NO: 65 | NO: 126 |
| | | eRF1 | | | | | | | | |
| 27 | Eoc_eRF1_ | Whole- | Euplotes | Eoc | | UAA/UAG | AAG25924.1 | | SEQ ID | SEQ ID |
| | AAG25924.1 | gene | octocarinatus | | | | | | NO: 66 | NO: 127 |
| | | eRF1 | | | | | | | | |
| 28 | Bja_eRF1_ | Whole- | Blepharisma | Bja | | UAA/UAG | CAC16186.2 | | SEQ ID | SEQ ID |
| | CAC16186.2 | gene | japonicum | | | | | | NO: 67 | NO: 128 |
| | | eRF1 | | | | | | | | |
| 29 | Tth_eRF1_ | Whole- | Tetrahymena | Tth | | UGA | XP_001018735.1 | | SEQ ID | SEQ ID |
| | XP_001018735.1 | gene | thermophila | | | | | | NO: 68 | NO: 129 |
| | | eRF1 | | | | | | | | |
| 30 | Tth_eRF1_ | Whole- | Tetrahymena | Tth | | UGA | XP_001018211.4 | | SEQ ID | SEQ ID |
| | XP_001018211.4 | gene | thermophila | | | | | | NO: 69 | NO: 130 |
| | | eRF1 | | | | | | | | |
| 31 | Tth_eRF1_ | Whole- | Tetrahymena | Tth | | UGA | XP_001008252.2 | | SEQ ID | SEQ ID |
| | XP_001008252.2 | gene | thermophila | | | | | | NO: 70 | NO: 131 |
| | | eRF1 | | | | | | | | |
| 32 | Pte_eRF1_ | Whole- | Paramecium | Pte | | UGA | XP_001425245.1 | | SEQ ID | SEQ ID |
| | XP_001425245.1 | gene | tetraurelia | | | | | | NO: 71 | NO: 132 |
| | | eRF1 | | | | | | | | |
| 33 | Pte_eRF1_ | Whole- | Paramecium | Pte | | UGA | XP_001448143.1 | | SEQ ID | SEQ ID |
| | XP_001448143.1 | gene | tetraurelia | | | | | | NO: 72 | NO: 133 |
| | | eRF1 | | | | | | | | |
| 34 | Smy_eRF1_ | Whole- | Stylonychia | Smy | | UGA | Q9BMM1.1 | | SEQ ID | SEQ ID |
| | Q9BMM1.1 | gene | mytilus | | | | | | NO: 73 | NO: 134 |
| | | eRF1 | | | | | | | | |
| 35 | Ssa_eRF1_ | Whole- | Spironucleus | Ssa | | UGA | EST45466.1 | | SEQ ID | SEQ ID |
| | EST45466.1 | gene | salmonicida | | | | | | NO: 74 | NO: 135 |
| | | eRF1 | | | | | | | | |
| 36 | Yeast_eRF1_ | Whole- | Saccharomyces | Sce | | UAA/UAG/ | NP_009701.3 | | SEQ ID | SEQ ID |
| | eRF3 | gene | cerevisiae | | | UGA | | | NO: 75 | NO: 136 |
| | | eRF1/ | | | | | | | | |
| | | eRF3 | | | | | | | | |
| 37 | Yeast_eRF1_ | Whole- | Saccharomyces | Sce | | UAA/UAG/ | | NP_010457.3 | SEQ ID | SEQ ID |
| | eRF3 | gene | cerevisiae | | | UGA | | | NO: 76 | NO: 137 |
| | | eRF1/ | | | | | | | | |
| | | eRF3 | | | | | | | | |
| 38 | Eoc_eRF1_ | Whole- | Euplotes | Eoc | | UAA/UAG | CAC14170.1 | | SEQ ID | SEQ ID |
| | CAC14170.1/ | gene | octocarinatus | | | | | | NO: 77 | NO: 138 |
| | Eoc_eRF3_ | eRF1/ | | | | | | | | |
| | AAL33628.1 | ReF3 | | | | | | | | |
| 39 | Eoc_eRF1_ | Whole- | Euplotes | Eoc | | UAA/UAG | | AAL33628.1 | SEQ ID | SEQ ID |
| | CAC14170.1/ | gene | octocarinatus | | | | | | NO: 78 | NO: 139 |
| | Eoc_eRF3_ | eRF1/ | | | | | | | | |
| | AAL33628.1 | eRF3 | | | | | | | | |
| 40 | Eoc_eRF1_ | Whole- | Euplotes | Eoc | | UAA/UAG | AAG25924.1 | | SEQ ID | SEQ ID |
| | AAG25924.1/ | gene | octocarinatus | | | | | | NO: 79 | NO: 140 |
| | Eoc_eRF3_ | eRF1/ | | | | | | | | |
| | AAL33628.1 | eRF3 | | | | | | | | |
| 41 | Eoc_eRF1_ | Whole- | Euplotes | Eoc | | UAA/UAG | | AAL33628.1 | SEQ ID | SEQ ID |
| | AAG25924.1/ | gene | octocarinatus | | | | | | NO: 80 | NO: 141 |
| | Eoc_eRF3_ | eRF1/ | | | | | | | | |
| | AAL33628.1 | eRF3 | | | | | | | | |
| 42 | Bja_eRF1_ | Whole- | Blepharisma | Bja | | UAA/UAG | CAC16186.2 | | SEQ ID | SEQ ID |
| | CAC16186.2/ | gene | japonicum | | | | | | NO: 81 | NO: 142 |
| | Bja_eRF3_ | eRF1/ | | | | | | | | |
| | AAD03251.1 | eRF3 | | | | | | | | |
| 43 | Bja_eRF1_ | Whole- | Blepharisma | Bja | | UAA/UAG | | AAD03251.1 | SEQ ID | SEQ ID |
| | CAC16186.2/ | gene | japonicum | | | | | | NO: 82 | NO: 143 |
| | Bja_eRF3_ | eRF1/ | | | | | | | | |
| | AAD03251.1 | eRF3 | | | | | | | | |
| 44 | Tth_eRF1_ | Whole- | Tetrahymena | Tth | | UGA | XP_001018735.1 | | SEQ ID | SEQ ID |
| | XP_001018735.1/ | gene | thermophila | | | | | | NO: 83 | NO: 144 |
| | Tth_eRF3_ | eRF1/ | | | | | | | | |
| | XP_001011280.3 | eRF3 | | | | | | | | |
| 45 | Tth_eRF1_ | Whole- | Tetrahymena | Tth | | UGA | | XP_001011280.3 | SEQ ID | SEQ ID |
| | XP_001018735.1/ | gene | thermophila | | | | | | NO: 84 | NO: 145 |
| | Tth_eRF3_ | eRF1/ | | | | | | | | |
| | XP_001011280.3 | eRF3 | | | | | | | | |
| 46 | Tth_eRF1_ | Whole- | Tetrahymena | Tth | | UGA | XP_001018211.4 | | SEQ ID | SEQ ID |
| | XP_001018211.4/ | gene | thermophila | | | | | | NO: 85 | NO: 146 |
| | Tth_eRF3_ | eRF1/ | | | | | | | | |
| | XP_001011280.3 | eRF3 | | | | | | | | |
| 47 | Tth_eRF1_ | Whole- | Tetrahymena | Tth | | UGA | | XP_001011280.3 | SEQ ID | SEQ ID |
| | XP_001018211.4/ | gene | thermophila | | | | | | NO: 86 | NO: 147 |
| | Tth_eRF3_ | eRF1/ | | | | | | | | |
| | XP_001011280.3 | eRF3 | | | | | | | | |
| 48 | Tth_eRF1_ | Whole- | Tetrahymena | Tth | | UGA | XP_001008252.2 | | SEQ ID | SEQ ID |
| | XP_001008252.2/ | gene | thermophila | | | | | | NO: 87 | NO: 148 |
| | Tth_eRF3_ | eRF1/ | | | | | | | | |
| | XP_001011280.3 | eRF3 | | | | | | | | |
| 49 | Tth_eRF1_ | Whole- | Tetrahymena | Tth | | UGA | | XP_001011280.3 | SEQ ID | SEQ ID |
| | XP_001008252.2/ | gene | thermophila | | | | | | NO: 88 | NO: 149 |
| | Tth_eRF3_ | eRF1/ | | | | | | | | |
| | XP_001011280.3 | eRF3 | | | | | | | | |
| 50 | Pte_eRF1_ | Whole- | Paramecium | Pte | | UGA | XP_001425245.1 | | SEQ ID | SEQ ID |
| | XP_001425245.1/ | gene | tetraurelia | | | | | | NO: 89 | NO: 150 |
| | Pte_eRF3_ | eRF1/ | | | | | | | | |
| | XP_001459190.1 | eRF3 | | | | | | | | |
| 51 | Pte_eRF1_ | Whole- | Paramecium | Pte | | UGA | | XP_001459190.1 | SEQ ID | SEQ ID |
| | XP_001425245.1/ | gene | tetraurelia | | | | | | NO: 90 | NO: 151 |
| | Pte_eRF3_ | eRF1/ | | | | | | | | |
| | XP_001459190.1 | eRF3 | | | | | | | | |
| 52 | Pte_eRF1_ | Whole- | Paramecium | Pte | | UGA | XP_001448143.1 | | SEQ ID | SEQ ID |
| | XP_001448143.1/ | gene | tetraurelia | | | | | | NO: 91 | NO: 152 |
| | Pte_eRF3_ | eRF1/ | | | | | | | | |
| | XP_001459190.1 | eRF3 | | | | | | | | |
| 53 | Pte_eRF1_ | Whole- | Paramecium | Pte | | UGA | | XP_001459190.1 | SEQ ID | SEQ ID |
| | XP_001448143.1/ | gene | tetraurelia | | | | | | NO: 92 | NO: 153 |
| | Pte_eRF3_ | eRF1/ | | | | | | | | |
| | XP_001459190.1 | eRF3 | | | | | | | | |
| 54 | Eoc_eRF1_ | Whole- | Euplotes | Eoc | Replace 7-298 | UAA/UAG | CAC14170.1 | | SEQ ID | SEQ ID |
| | CAC14170.1/ | gene | octocarinatus | | a.a. of | | | | NO: 93 | NO: 154 |
| | N_Yeast_eRF3_ | eRF1/ | | | Eoc_eRF3 with | | | | | |
| | Eoc_eRF3_ | eRF3 | | | 6-253 of | | | | | |
| | AAL33628.1 | | | | Sce_eRF3 | | | | | |
| 55 | Eoc_eRF1_ | Whole- | Euplotes | Eoc | Replace 7-298 | UAA/UAG | | AAL33628.1 | SEQ ID | SEQ ID |
| | CAC14170.1/ | gene | octocarinatus | | a.a. of | | | | NO: 94 | NO: 155 |
| | N_Yeast_eRF3_ | eRF1/ | | | Eoc_eRF3 with | | | | | |
| | Eoc_eRF3_ | eRF3 | | | 6-253 of | | | | | |
| | AAL33628.1 | | | | Sce_eRF3 | | | | | |
| 56 | Eoc_eRF1_ | Whole- | Euplotes | Eoc | Replace 1-298 | UAA/UAG | AAG25924.1 | | SEQ ID | SEQ ID |
| | AAG25924.1/ | gene | octocarinatus | | a.a. of | | | | NO: 95 | NO: 156 |
| | N_Yeast_eRF3_ | eRF1/ | | | Eoc_eRF3 with | | | | | |
| | Eoc_eRF3_ | eRF3 | | | 1-253 of | | | | | |
| | AAL33628.1 | | | | Sce_eRF3 | | | | | |
| 57 | Eoc_eRF1_ | Whole- | Euplotes | Eoc | Replace 1-298 | UAA/UAG | | AAL33628.1 | SEQ ID | SEQ ID |
| | AAG25924.1/ | gene | octocarinatus | | a.a. of | | | | NO: 96 | NO: 157 |
| | N_Yeast_eRF3_ | eRF1/ | | | Eoc eRF3 with | | | | | |
| | Eoc_eRF3_ | eRF3 | | | 1-253 of | | | | | |
| | AAL33628.1 | | | | Sce_eRF3 | | | | | |
| 58 | Pte_eRF1_ | Whole- | Paramecium | Pte | Replace 1-321 | UGA | XP_001425245.1 | | SEQ ID | SEQ ID |
| | XP_001425245.1/ | gene | tetraurelia | | a.a. of | | | | NO: 97 | NO: 158 |
| | N_Yeast_eRF3_ | eRF1/ | | | Pte_eRF3 with | | | | | |
| | Pte_eRF3_ | eRF3 | | | 1-253 of | | | | | |
| | XP_001459190.1 | | | | Sce_eRF3 | | | | | |
| 59 | Pte_eRF1_ | Whole- | Paramecium | Pte | Replace 1-321 | UGA | | XP_001459190.1 | SEQ ID | SEQ ID |
| | XP_001425245.1/ | gene | tetraurelia | | a.a. of | | | | NO: 98 | NO: 159 |
| | N_Yeast_eRF3_ | eRF1/ | | | Pte_eRF3 with | | | | | |
| | Pte_eRF3_ | eRF3 | | | 1-253 of | | | | | |
| | XP_001459190.1 | | | | Sce_eRF3 | | | | | |
| 60 | Pte_eRF1_ | Whole- | Paramecium | Pte | Replace 1-321 | UGA | XP_001448143.1 | | SEQ ID | SEQ ID |
| | XP_001448143.1/ | gene | tetraurelia | | a.a. of | | | | NO: 99 | NO: 160 |
| | N_Yeast_eRF3_ | eRF1/ | | | Pte_eRF3 with | | | | | |
| | Pte_eRF3_ | eRF3 | | | 1-253 of | | | | | |
| | XP_001459190.1 | | | | Sce_eRF3 | | | | | |
| 61 | Pte_eRF1_ | Whole- | Paramecium | Pte | Replace 1-321 | UGA | | XP_001459190.1 | SEQ ID | SEQ ID |
| | XP_001448143.1/ | gene | tetraurelia | | a.a. of | | | | NO: 100 | NO: 161 |
| | N_Yeast_eRF3_ | eRF1/ | | | Pte_eRF3 with | | | | | |
| | Pte_eRF3_ | eRF3 | | | 1-253 of | | | | | |
| | XP_001459190.1 | | | | Sce_eRF3 | | | | | |

The examples and embodiments described herein are for illustrative purposes only and various modifications or changes suggested to persons skilled in the art are to be included within the spirit and purview of this application and scope of the appended claims.

1. A composition comprising:
(a) a first recombinant release factor, wherein the first recombinant release factor is configured to recognize UGA, UAA, UAG, or any combination thereof as a stop codon; and
(b) a second recombinant release factor, wherein the second recombinant release factor is configured to recognize UGA, UAA, UAG, or any combination thereof as a stop codon, and
wherein the second recombinant release factor comprises an element that allows selective modulation of function of the second recombinant release factor.

2. (canceled)

3. (canceled)

4. The composition of claim 1, wherein the first recombinant release factor recognizes UAA, UAG, or any combination thereof as the stop codon.

5.-7. (canceled)

8. The composition of claim 1, wherein the second recombinant release factor recognizes UGA, UAA, UAG, or any combinations thereof as stop codons.

9. The composition of claim 1, wherein the element that allows selective modulation of the function of the second recombinant release factor comprises a temperature sensitive allele that allows the second recombinant release factor to function only at a permissive temperature or a degron cassette that allows degradation of the second recombinant release factor.

10. The composition of claim 9, wherein the temperature sensitive allele comprises sup45-ts, sup45-2, sup45-36ts, sup45-1023ts, or sup45-sl23ts.

11.-15. (canceled)

16. The composition of claim 9, wherein the permissive temperature comprises from about 20° C. to about 33° C.

17. The composition of claim 16, wherein the permissive temperature is 25° C.

18. The composition of claim 9, wherein the degron cassette comprises a heat-inducible degron cassette or a small molecule-inducible degron cassette.

19. The composition of claim 18, wherein the degron cassette comprises the small molecule-inducible degron cassette comprising an auxin or asunaprevir.

20. (canceled)

21. The composition of claim 1, wherein the first or the second recombinant release factor modulates protein translation upon recognizing UGA, UAA, or UAG as the stop codon, wherein the modulation comprises terminating protein translation.

22.-32. (canceled)

33. The composition of claim 1, wherein the second recombinant release factor is from a first organism, wherein the first organism comprises a eukaryotic cell or a prokaryotic cell.

34. (canceled)

35. (canceled)

36. The composition of claim 33, wherein the eukaryotic cell comprises a yeast cell, a fungal cell, a plant cell, an animal cell, an insect cell, a mammalian cell, or a combination thereof.

37. The composition of claim 36, wherein the yeast cell comprises Saccharomyces cerevisiae.

38. The composition of claim 1, wherein the first recombinant release factor is from a second organism.

39. The composition of claim 38, wherein the second organism comprises a ciliate comprising Blepharisma americanum, Blepharisma japonicum, Euplotes aediculatus, Euplotes octocarinatus, Stentor coeruleus, Nyctotherus ovalis, Stylonychia lemnae, Pseudocohnilembus per salinus, Ichthyophthirius multifiliis, Stylonychia lemnae, Oxytricha trifallax, Stylonychia pustulata, Stylonychia Mytilus, Eschaneustyla sp. HL-2004, Gonostomum sp. HL-2004, Holosticha sp. HL-2004, Urostyla sp. HL-2004, Uroleptus sp. WIC-2003, Paraurostyla weissei, Stichotrichida sp. Misty, Stichotrichida sp. Alaska, Spironucleus salmonicida, Loxodes striatus, Paramecium tetraurelia, or Tetrahymena thermophila.

40.-42. (canceled)

43. The composition of claim 40, wherein the first or the second recombinant release factor comprises an amino acid sequence selected from the group consisting of SEQ ID NOs: 40-64.

44.-61. (canceled)

62. The composition of claim 1, further comprising one or more tRNA molecules that recognize UAG, UAA, or UGA and one or more aminoacyl-tRNA synthetases (aaRSs) for charging the one or more tRNA molecules with a non-canonical amino acid (ncAA).

63. The composition of claim 62, wherein the ncAA comprises p-azidophenylalanine, 2-aminoisobutyric acid (Aib), an azide-containing ncAA, an alkene-containing ncAA, an alkyne-containing ncAA, or a combination thereof.

64. The composition of claim 63, wherein the azide-containing ncAA comprises (S)-2-amino-6-((2-azidoethoxy)carbonylamino)hexanoic acid (LysN3).

65. The composition of claim 63, wherein the alkene-containing ncAA or the alkyne-containing ncAA comprises (2S)-2-amino-6-(((prop-2-yn-1-yloxy)carbonyl)amino)hexanoic acid (LysAlk), N^{ε}-Allyloxycarbonyl-L-lysine, N-ε-propargyloxycarbonyl-L-lysine, L-2-Allylglycine, or O-Allyl-L-Tyrosine.

66. The composition of claim 62, wherein the one or more tRNA molecules recognize UAG, UAA, UGA, or a combination thereof and the one or more aaRSs charges the one or more tRNA molecules with a first ncAA, a second ncAA, or a third ncAA, wherein the first ncAA, the second ncAA, and/or the third ncAA are different from each other.

67.-239. (canceled)
